(19) United States
(12) Patent Application Publication
(10) Pub. No.: US 2019/0031650 A1
(43) Pub. Date: Jan. 31, 2019

(54) DNA ALKYLATION AND CROSS-LINKING AGENTS AS COMPOUNDS AND PAYLOADS FOR TARGETED THERAPIES

(71) Applicant: YALE UNIVERSITY, NEW HAVEN, CT (US)

(72) Inventors: Seth Herzon, Madison, CT (US); Alan Healy, New Haven, CT (US); Jason Crawford, Shelton, CT (US); Maria Vizcaino, Fort Washington, PA (US); Herman Nikolayevskiy, North Bethesda, MD (US)

(21) Appl. No.: 16/073,076

(22) PCT Filed: Jan. 27, 2017
(86) PCT No.: PCT/US2017/015258

§ 371 Date: Jul. 26, 2018

Related U.S. Application Data
(60) Provisional application No. 62/288,572, filed on Jan. 29, 2016.
(60) Provisional application No. 62/417,650, filed on Nov. 4, 2016.

(51) Int. Cl. C07D 417/14; C07D 417/04; A61K 47/55; A61K 47/64; A61K 47/54; A61K 47/60; A61K 45/06; A61K 31/427

(52) U.S. Cl. CPC C07D 417/14; C07D 417/04; A61K 47/55; A61K 31/427; A61K 47/549; A61K 47/60; A61K 45/06; A61K 47/64

(57) ABSTRACT

The present invention is directed to compounds related to precolibactin pharmaceutical compositions based upon these compounds and methods of synthesis which are employed to provide intermediates and final compounds, which are principally alkylating agents and anticancer compounds. The chemical synthetic approach disclosed facilitates the synthesis of numerous precolibactin analogs which can be used in the treatment of cancer.

### RELATED APPLICATIONS AND GRANT SUPPORT

This application claims the benefit of priority of United States provisional applications nos. U.S. 62/288,572, filed Jan. 29, 2017 and U.S. 62/417,650, filed Nov. 4, 2016, entitled “DNA Alkylation and Cross-linking Agents as Paylods for Targeted Therapies”, each of said applications being incorporated by reference in its entirety herein.

This invention was made with government support under grant nos. IDP2-CA86575, R01GM110506 and 5T32GM06754 awarded by National Institutes of Health. The government has certain rights in the invention

### FIELD OF THE INVENTION

The present invention is directed to novel small molecules that alkylate and cross-link DNA. These molecules are easily-prepared and modified to adjust DNA binding and alkylation properties. The molecules may contain a cleavable protecting group (prodrug) to allow for specific activation in selected settings and often comprise a targeting element attached to the molecules by way of a cleavable linker which may be cleaved to facilitate therapy. Methods of synthesizing these compounds are also disclosed as are important intermediates in the process of synthesis.

### BACKGROUND AND OVERVIEW OF THE INVENTION

Precolibactins and colibactins are natural products produced by select commensal, extraintestinal, and probiotic E. coli. The metabolites are encoded by a hybrid polyketide synthase-nonribosomal peptide synthetase (PKS-NRPS) gene cluster termed clb or pks.¹ clb⁺ E. coli strains induce DNA damage in eukaryotic cells and are thought to promote colorectal cancer formation,^{1c,2} but the gene cluster is also found in the probiotic strain Nissle 1917, which is used in Europe for the treatment of ulcerative colitis, diarrhea, and other gastrointestinal disorders.³ Mature precolibactins are substrates for the 12-transmembrane multidrug and toxic compound extrusion transporter ClbM, which mediates their transfer to the bacterial periplasm.⁴ There, the colibactin peptidase ClbP converts precolibactins to genotoxic colibactins via removal of an N-acyl-D-asparagine side chain.⁵ Mutation of ClbP abolishes cellular DNA damaging-activity,^{2a,5b} and N-myristoyl-D-asparagine and closely related analogs have been identified in wild type clb⁺ E. coli cultures.^{5d,5e} Whether the differential production of biosynthetically-related but distinct metabolites, or other factors (such as the requirement for cell-to-cell contact to observe cytopathic effects^{2a,6}) underlie the seemingly contradictory phenotypes associated with the clb gene cluster, remains unresolved.

Despite extensive efforts, fully mature (pre)colibactins prior to the present invention, had not yet been isolated in homogenous form. This has been attributed to the low levels of natural production of the metabolites, their instability under fermentation conditions, and the inflammation-dependant expression of the clb gene cluster. The metabolites 1,^{5c} 2,⁹ 3,^{9b} and 4^{9b} were obtained in vanishingly small quantities (2.5-55 μg/L for 2-4) from the fermentation broth of wild-type or genetically-engineered clb⁺ E. coli and implicated as intermediates or shunt metabolites in the colibactin biosynthetic pathway. Based on the isolation of 2 (FIG. 1), as well as HRMS analysis, retrospective bioinformatics, and isotopic labelling, the structure of precolibactin A was proposed as 5.^{9A} Key elements within 5 include a hydrophobic N-terminal fragment, a spirocyclic aminocyclopropane, and a thiazoline-thiazole tail. The presence of the thiazoline-thiazole fragment was inferred by MS/MS analysis;^{9a} the alternate isomer, containing a thiazole-thiazoline sequence, could not be excluded. In addition, the absolute stereochemistry of the thiazoline ring was not determined. A compound with an exact mass corresponding to 5 was observed in unpurified extracts, but all efforts to isolate this structure were hampered by decomposition.^{9a} The pyridone structure 6 was also recently proposed based on biosynthetic considerations, isolation of 4, and HRMS analysis, but, like 5, was obtained in quantities too minute¹⁰ to permit full characterization.¹¹ 4 was shown to weakly cross-link DNA in vitro,⁹ suggesting that the colibactins may damage DNA by induction of replication-dependant DSBs.¹² Although detailed structure-function analyses of the colibactins had not been conducted prior to the present invention, the aminocyclopropane fragment of 4 is reminiscent of yatakemycin, CC-1065, and the duocarmycins, which have been shown to alkylate DNA via nucleophilic ring-opening,¹³ and the biheterocyclic fragment may serve as a DNA intercalation motif.¹⁴

Pursuant to the present invention, the inventors have focused on understanding the molecular basis of colibactin-induced DNA damage. Advanced precolibactins arise from linear precursors of the generalized structure shown as 1 (Scheme 6, FIG. 15A). The linear precursors were suggested to transform to unsaturated lactams 2 that are processed by ClbP to generate unsaturated iminium ions 3 (colibactins), which alkylate DNA by cyclopropane ring-opening (grey pathway).^{7,8} However, this mechanistic hypothesis is ostensibly incompatible with subsequent isolation⁹ and synthesis¹⁰ efforts that lead to the identification and unequivocal structural assignment of precolibactins A (7),¹¹ B (8), and C (9), which contain a pyridone residue (FIGS. 1 and 15B). Precolibactins A-C (7-9, FIG. 15B) were obtained from clbP mutant strains; these deletion strains were employed to promote accumulation of the precolibactin metabolites. If 7-9 are the genotoxic precursors, the data outlined above⁵ suggests that amines such as 5 resulting from ClbP-mediated processing in the wild type strains are responsible for the cytopathicity of the clb cluster, as these cannot readily convert to unsaturated iminium ions such as 3. Precolibactin C (9, FIG. 15B) was demonstrated to be a substrate for ClbP.¹⁰

In earlier synthetic work, the inventors showed that the double dehydrative cyclization of the relatively stable N-acylated linear precursors (1) to pyridones such as 7-9 (FIGS. 1 and 15B) was facile under mildly acidic or basic conditions (c.f., 1→2→4, Scheme 6, FIG. 15A).¹⁰ The unsaturated lactam intermediates (2) could be detected by LC/MS analysis, but they were not isolable, arguing against their interception by ClbP in the biosynthesis. The inventors reasoned that the colibactins may instead form by ClbP processing of isolable linear precursors 1 directly. Sequential cyclodehydration reactions proceeding through the vinylogous ureas 6 would then provide 3 (red pathway). It follows from this analysis that precolibactins A-C (7-9) are non-natural cyclization products deriving from the absence of ClbP in the producing organisms, and are unlikely to be genotoxic. To test this hypothesis, the inventors modified their synthetic strategy¹⁰ to allow access to the deacylated pyridone derivatives 5 and the analogous unsaturated iminium ions 3. The inventors show that the iminium ions are potent DNA alkylation agents while the corresponding pyridone structures are not. In addition, the inventors rigorously define the structure-function relationships of 3 that are required for or enhance DNA alkylation activity. Finally, the synthetic studies support the alternative biosynthetic pathway involving the intermediacy of the vinylogous amide 6 en route to 3. Collectively, our data lend further support to the hypothesis that unsaturated iminium ions 3 are responsible for the genotoxic effects of the clb gene cluster and support the conclusion that precolibactins A-C (7-9) (and other pyridone-containing isolates) are off-pathway fermentation products derived from the absence of a functional clbP gene. This work constitutes the first structure-function studies of colibactin metabolites and provides a foundation to begin to connect the disparate phenotypic effects of the clb cluster with metabolite structure.

### BRIEF DESCRIPTION OF THE FIGURES

FIG. 1 shows the structures of isolated, predicted and synthesized colibactin metabolites. Fermentation yields (micrograms of product per liter of fermentation broth) are shown in parentheses.

FIGS. 2-7 shows numerous synthetic chemical steps to afford compounds according to the present invention (which includes intermediates).

FIG. 8, Scheme 1, shows the chemical synthesis of carboxylic acid 10. Reagents and conditions: a. (S)-hex-5-en-2-amine hydrogen chloride (8), 1-ethyl-3-(3-dimethylaminopropyl)carbodiimide (EDC.HCl), hydroxybenzotriazole (HOBT), N,N-diisopropylethylamine (DIPEA), DMF, 23° C., 84%; b. HCl, CH₂Cl₂-1,4-dioxane (7:1), 23° C., >99%; c. Myristoyl chloride, triethylamine (Et₃N), DMF, 23° C., 82%; d. RuCl₃, NaIO₄, H₂O-EtOAc—CH₃CN (3:2:2), 50° C., 95%.

FIG. 9, Scheme 2. A. Shows the Synthesis of the thiazoline-thiazole 17. B. Shows the Synthesis of the thiazole-thiazoline 23. C. Shows the Synthesis of the bithiazole 27. Reagents and conditions: a. L-(+)-cysteine ethyl ester hydrochloride, Et₃N, CH₃OH, 23° C., 85%; b. NH₃, CH₃OH—H₂O (2:1), 23° C., >99%; c. Lawesson's reagent, CH₂Cl₂, 23° C., >99%; d. bromopyruvic acid, Et₃N, CH₃OH, reflux, 71%; e. HCl, CH₂Cl₂-1,4-dioxane (4:1), 23° C., >99%; f. silver trifluoroacetate (AgOTFA), Et₃N, DMF, 0° C., 63%; g. HCl, CH₂Cl₂-1,4-dioxane (4:1), 23° C., >99%; h. ethyl bromopyruvate, CaCO₃, EtOH, 23° C., 74%; i. NH₃, CH₃OH—H₂O (2:1), 23° C., >99%; j. trifluoroacetic anhydride (TFAA), Et₃N, CH₂Cl₂, 0→23° C., 84%; k. L-(+)-cysteine, Et₃N, CH₃OH, reflux, 97%; l. HCl, CH₂Cl₂-1,4-dioxane (8:1), 23° C., >99%; m. AgOTFA, Et₃N, DMF, 0° C., 69%; n. HCl, CH₂Cl₂-1,4-dioxane (4:1), 23° C., >99%; o. NH₃, CH₃OH—H₂O (2:1), 23° C., >99%; p. Lawesson's reagent, CH₂Cl₂, 23° C., >99%; q. bromopyruvic acid, CaCO₃, EtOH, 23° C., 58%; r. HCl, CH₂Cl₂-1,4-dioxane (4:1), 23° C., >99%; s. AgOTFA, Et₃N, DMF, 0° C., 72%; t. HCl, CH₂Cl₂-1,4-dioxane (4:1), 23° C., >99%.

FIG. 10, Scheme 3, Shows the Synthesis of the acyclic advanced precursors 29a-c. Reagents and conditions: a. Carbonyl diimidazole (CDI), 4 Å molecular sieves, DMF, then malonic acid half-thioester, magnesium ethoxide (Mg(OEt)₂), 23° C., 95%; b. 17, 23, or 27, AgOTFA, Et₃N, DMF, 0° C., 90% (29a); 87% (29b); 86% (29c).

FIG. 11, Scheme 4, A. Cyclodehydration of the linear precursors 29a-c. Reagents and conditions: a. K₂CO₃, CH₃OH, 0° C., 79% (30a); 80% (30b); 83% (30c) or b. K₂CO₃, dimethyl sulfoxide, 24° C. (for 29b). B. UV trace (254 nm) of the cyclization of 29b using potassium carbonate in dimethyl sulfoxide at 24° C.

FIG. 12 A. Mass-selective (m/z=816.3788) LC/HRMS-QTOF analysis of the ethyl acetate extracts of clb⁺ E. coli ΔclbP (top), synthetic 5a (middle), and co-injection (bottom). B. Mass-selective (m/z=816.3788) LC/HRMS-QTOF analysis of the ethyl acetate extracts of clb⁺ E. coli ΔclbP (top), synthetic 5b (middle), and co-injection (bottom). Y axis corresponds to relative ion intensity (×10⁴).

FIG. 13, Scheme 5. A. The originally predicted (5a) and revised (7) structures of precolibactin A. B. Synthesis of the revised structure of precolibactin A (7). Reagents and conditions: a. bromopyruvic acid, CaCO₃, EtOH, 23° C., 74%; b. HCl, CH₂Cl₂-dioxane (3:1), 23° C., >99%; c. AgOTFA, Et₃N, DMF, 0° C., 55%; d. HCl, CH₂Cl₂-1,4-dioxane (3:1), 23° C., >99%; e. AgOTFA, Et₃N, DMF, 0° C.; f. K₂CO₃, CH₃OH, 0° C., 67% (two steps); g. L-cysteine, N-hydroxysuccinimide (NHS), EDC.HCl, Et₃N, DMF, 0→23° C., 89%.

FIG. 14 shows the Mass-selective (m/z=816.3788) LC/HRMS-QTOF analysis of the ethyl acetate extracts of clb⁺ E. coli ΔclbP (top), synthetic 7 (middle), and co-injection (bottom).

FIG. 15A, Scheme 6 shows proposed mechanisms of action and divergent reactivity of precolibactin precursors. The gray ball in each of compounds 1-6 in the scheme denotes a variable region of the compound.

FIG. 15B shows the chemical Structures of precolibactins A (7), B (8), and C (9).

FIG. 16, Scheme 7 shows the synthesis of the unsaturated imine 15a and the pyridone 17a. Structures of the N-methylamides 15b and 17b. Reagents and conditions: (a) silver trifluoroacetate (AgOTFA), Et₃N, DMF, 0° C.; (b) concentrate from 0.5% HCO₂H-5% CH₃OH—CH₃CN, 23° C., 87% (two steps); (c) propylphosphonic anhydride solution (T3P), N-methylmorpholine, N,N-dimethylethylenediamine, THF, 23° C., 93%; (d) trifluoroacetic acid (TFA), CH₂Cl₂, 0° C.; aqueous NaHCO₃, 23° C., 62%; (f) K₂CO₃, CH₃OH, 0→23° C., 78% (two steps); (g) T3P, N-methylmorpholine, N,N-dimethylethylenediamine, THF, 23° C., 46%; (h) TFA, CH₂Cl₂, 0° C., 86%.

FIG. 17 A. DNA alkylation assay employing linearized pBR322 DNA and the pyridone derivatives 17a or 17b or the unsaturated imines 15a or 15b. Conditions: Linearized pBR322 DNA (20 μM in base pairs (bps)), 15a (100, 10, 1, 0.5, or 0.1 μM), 15b (500, 100, 10, or 1 μM), 17a (500 or 100 μM) or 17b (500 or 100 μM), 37° C., 15 h. Cisplatin (CP; 100 μM) and methyl methanesulfonate (MMS; 500, 100, or 10 μM) were used as positive controls for DNA cross-linking and alkylation, respectively. DNA was visualized using SybrGold. B. DNA alkylation assay employing linearized pBR322 DNA and the unsaturated imine 15a. Conditions: Linearized pBR322 DNA (20 μM in base pairs), 15a (100, 10, 1, 0.1, 0.05, 0.01, or 0.005 μM), 37° C., 15 h.

FIG. 18 A. Increase in the melting temperature of calf thymus DNA treated with increasing amounts of the pyridone 17a. Conditions: 2.09 mM NaH₂PO₄, 7.13 mM Na₂HPO₄, 928 μM Na₂EDTA, 1.01 mM DMSO, pH 7.18. The pyridone 17a was incubated with ctDNA for 3 h prior to UV thermal denaturation experiments (260 nm, heating rate: 0.5° C./min). [DNA]=32.0 mM bps. B. Time-dependent modulation of the melting temperature of calf thymus DNA treated with 1 or 2 bp equiv of the imine 15a. Conditions: 2.09 mM NaH₂PO₄, 7.13 mM Na₂HPO₄, 928 μM Na₂EDTA, 1.01 mM DMSO, pH 7.18. The imine 15a was incubated with ctDNA for 5 min, 1 h, 3 h, 6 h, or 15 h prior to UV thermal denaturation experiments (260 nm, heating rate: 0.5° C./min). [DNA]=32.0 mM bps. C. Time-dependent DNA alkylation assay employing linearized pBR322 DNA and the unsaturated imine 15a. Conditions: Linearized pBR322 DNA (20 μM in base pairs), 15a (1 μM), 37° C., 0.1-15 h.

FIG. 19 A. Structures of the dimer 15c and the gem-dimethyl derivative 15d. B. DNA alkylation assay employing linearized pBR322 DNA and the dimer 15c or the gem-dimethyl derivative 15d. Conditions: Linearized pBR322 DNA (20 μM in base pairs), 15c (10 μM) or 15d (500 or 100 μM), 37° C., 0.1-15 h.

FIG. 20 shows the Ring-opening of the unsaturated imine 15b by propanethiol. Conditions: p-toluenesulfonic acid monohydrate, CH₃CN-propanethiol-DMF (6:2:1), 23° C., 34%.

FIG. 21 shows A. Structures of the analogs 15e-i, 19, and 15j. B. DNA alkylation assay employing linearized pBR322 DNA and the unsaturated lactam 19 or the unsaturated imine 15j. Conditions: Linearized pBR322 DNA (20 μM in base pairs), 19 (500, 100, 10, or 1 μM) or 15j (500, 100, 10, or 1 μM), 37° C., 15 h.

FIG. 22 shows the various chemical moieties on a representative generic chemical structure of compounds according to the present invention.

FIG. 23, Scheme S1, shows the synthesis of the dimeric unsaturated imine 15c. Reagents and conditions: (a) propylphosphonic anhydride solution (T3P), N-methylmorpholine, N,N-bis(3-aminopropyl)methylamine, THF, 23° C., 58%; (b) trifluoroacetic acid (TFA), CH₂Cl₂, 0° C., then aqueous NaHCO₃, 23° C., 73%.

FIG. 24, Scheme S2 shows the synthesis of the unsaturated imine 15d. Reagents and conditions: (a) silver trifluoroacetate (AgOTFA), Et₃N, DMF, 0° C., 40%; (b) HCl, CH₂Cl₂-1,4-dioxane (1:1), 0→23° C., >99%; (c) 10, AgOTFA, Et₃N, DMF, 0° C., 34%; (d) propylphosphonic anhydride solution (T3P), N-methylmorpholine, N,N-dimethylethylenediamine, THF, 23° C., 83%; (e) trifluoroacetic acid (TFA), CH₂Cl₂, 0° C., then aqueous NaHCO₃, 23° C., 67%.

FIG. 25, Scheme S3 shows the synthesis of the unsaturated imines 15b, 15e-i. Reagents and conditions: (a) methylamine, N,N-dimethyl-1,3-diaminopropane, N-(tert-butoxycarbonyl)-1,2-diaminoethane, N-(tert-butoxycarbonyl)-1,5-diaminopentane, N,N′-bis-(tert-butoxycarbonyl)-N″-(2-aminoethyl)-guanidine (S11), or N,N′-bis-(ter-butoxycarbonyl)-N″-(4-aminobutyl)-guanidine (S12), propylphosphonic anhydride solution (T3P), N-methylmorpholine, THF, 23° C.; 79% (14b), 95% (14e), 81% (14f), 48% (14g), 67% (14h), 72% (14i); (b) trifluoroacetic acid (TFA), CH₂Cl₂, 0° C., then aqueous NaHCO₃, 23° C.; 39% (15b), 76% (15e), 49% (15f), 45% (15g), 34% (15h), 48% (15i).

FIG. 26, Scheme S4 show the synthesis of the unsaturated imine 19. Reagents and conditions: (a) ethyl bromopyruvate, iso-propanol, 83° C., then di-tert-butyl dicarbonate, aqueous KHCO₃, 1,4-dioxane, 0° C., 66%; (b) NaH, iodomethane, DMF, −5→15° C., then LiOH, H₂O, 15° C., 98%; (c) HCl, CH₂Cl₂-1,4-dioxane (3:1), 23° C., >99%; (d) AgOTFA, Et₃N, DMF, 0° C., 95%; (e) HCl, CH₂Cl₂-1,4-dioxane (3:1), 23° C., >99%; (f) AgOTFA, Et₃N, DMF, 0° C., then K₂CO₃, CH₃OH, 0→23° C., 63% (g) propylphosphonic anhydride solution (T3P), N-methylmorpholine, N,N-dimethylethylenediamine, THF, 23° C., 58%; (b) trifluoroacetic acid (TFA), CH₂Cl₂, 0° C., 81%.

FIG. 27 shows the Time-dependent modulation of the melting temperature of calf thymus DNA treated with 1 or 2 bp equiv of the imine 15a. Conditions: 2.09 mM NaH₂PO₄, 7.13 mM Na₂HPO₄, 928 μM Na₂EDTA, 1.01 mM DMSO, pH 7.18. The imine 15a was incubated with ctDNA for 5 min, 1 h, 3 h, 6 h, or 15 h prior to UV thermal denaturation experiments (260 nm, heating rate: 0.5° C./min). [DNA]=32.0 mM bps. The Tₘ was defined as the temperature at which half of the duplex DNA was unwound, and was determined by the maximum of the first derivative of the thermal denaturation profile.

FIG. 28 shows the increase in the melting temperature of calf thymus DNA after treatment with increasing amounts of the pyridone 17a. Conditions: 2.09 mM NaH₂PO₄, 7.13 mM Na₂HPO₄, 928 μM Na₂EDTA, 1.01 mM DMSO, pH 7.18. The pyridone 17a was incubated with ctDNA for 3 h prior to UV thermal denaturation experiments (260 nm, heating rate: 0.5° C./min). [DNA]=32.0 mM bps. The Tₘ was defined as the temperature at which half of the duplex DNA was unwound, and was determined by the maximum of the first derivative of the thermal denaturation profile.

FIG. 29 shows no significant modulation of the melting temperature of calf thymus DNA was observed on treatment with 2 bp equiv of the pyridone 17b or 1 bp equiv of the imine 15b. Conditions: 2.09 mM NaH₂PO₄, 7.13 mM Na₂HPO₄, 928 μM Na₂EDTA, 1.01 mM DMSO, pH 7.18. The pyridone 17b and the imine 15b were incubated with ctDNA for 5 min, 3 h, or 15 h prior to UV thermal denaturation experiments (260 nm, heating rate: 0.5° C./min). [DNA]=32.0 mM bps.

FIG. 30 shows no significant modulation of the melting temperature of calf thymus DNA was observed on treatment with 2 bp equiv of the unsaturated imine 15b. Conditions: 2.09 mM NaH₂PO₄, 7.13 mM Na₂HPO₄, 928 μM Na₂EDTA, 1.01 mM DMSO, pH 7.18. The imine 15b was incubated with ctDNA for 5 min, 1 h, 3 h, 6 h, or 15 h prior to UV thermal denaturation experiments (260 nm, heating rate: 0.5° C./min). [DNA]=32.0 mM bps. The Tₘ was defined as the temperature at which half of the duplex DNA was unwound, and was determined by the maximum of the first derivative of the thermal denaturation profile.

FIG. 31 shows no significant modulation of the melting temperature of calf thymus DNA was observed on treatment with 2 bp equiv of the pyridone 17b. Conditions: 2.09 mM NaH₂PO₄, 7.13 mM Na₂HPO₄, 928 μM Na₂EDTA, 1.01 mM DMSO, pH 7.18. The pyridone 17b was incubated with ctDNA for 5 min, 1 h, 3 h, 6 h, or 15 h prior to UV thermal denaturation experiments (260 nm, heating rate: 0.5° C./min). [DNA]=32.0 mM bps. The Tₘ was defined as the temperature at which half of the duplex DNA was unwound, and was determined by the maximum of the first derivative of the thermal denaturation profile.

FIG. 32 shows a comparison of the ¹H NMR of the unsaturated imine 15b (top) and the propanethiol adduct product 18 (bottom). ¹H spectroscopic data were recorded in DMSO-d₆ (600 MHz (15b), 500 MHz (18), 23° C.).

FIG. 33 shows the results of a DNA alkylation assay DNA alkylation assay employing linearized pBR322 DNA and the derivatives 15a and 15e-i to probe the influence of the cationic residue on DNA alkylation activity. Conditions: Linearized pBR322 DNA (20 μM in base pairs), 15h (1, 0.1, or 0.01 μM), 15i (1, 0.1, or 0.01 μM), 15f (1, 0.1, or 0.01 μM), 15g (1, 0.1, or 0.01 μM), 15a (1, 0.1, or 0.01 μM), 15e (1, 0.1, or 0.01 μM), 37° C., 15 h. Methyl methanesulfonate (MMS; 500 or 100 μM) was used as a positive control for DNA alkylation. DNA was visualized using SybrGold.

FIG. 34, Table S1 shows a comparison of selected ¹H and ¹³C NMR Data of 15b and 18.

### BRIEF DESCRIPTION OF THE INVENTION

In embodiments, the present invention is directed to compounds according to the chemical structure I:

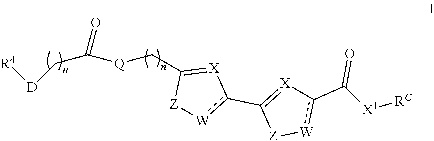

### Where X is N or C—R;

W is N, N—R^{N}, C—R or CR(R) (preferably the variable bond between W and the adjacent carbon atom is a double bond and W is N or C—R);
Each Z is independently S, O, N—R^{N} or C—R(R);
Each R is independently H, a C₁-C₆ (preferably C₁-C₃) alkyl group optionally substituted with one or two hydroxyl groups or up to three halogen (F, Cl, Br, I, preferably F or Cl, most often F) groups, or a O—(C₁-C₃) alkoxy group;
Each R^{N} is independently H or a C₁-C₆ (preferably C₁-C₃) alkyl group optionally substituted with one or two hydroxyl groups or up to three halogen groups, preferably H or methyl;
Q is O, S, N(R^{Q}) or C(R^{Q})R^{Q};
X¹ is O, S, N(R³) or C(R^{X})R^{X};

### D is

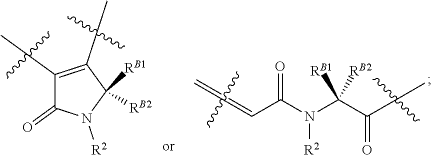

R¹, R² and R³ are each independently H or a C₁-C₆ (preferably C₁-C₃) alkyl group which is optionally substituted with one or two hydroxyl groups;

### R⁴ is

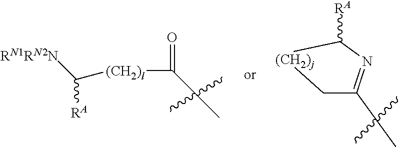

### When D is

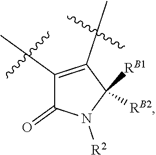

### R⁴ is

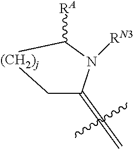

when D is

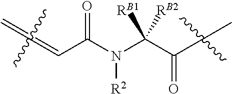

(the double bond is the same in both moieties);
Where R^{A} is H or an optionally substituted C₁-C₈ alkyl or alkene group, preferably H or a C₁-C₃ alkyl, most often methyl;
R^{N1} and R^{N2} are each independently H, a C₁-C₆ (preferably C₁-C₃) alkyl group which is optionally substituted with one or two hydroxyl groups or up to three halogen groups, a protecting group (P_{G}), preferably a BOC group) or a targeting element T_{E} which is linked to the nitrogen by linker group L_{C} which is optionally cleavable;
R^{N3} is absent, H, a C₁-C₆ (preferably C₁-C₃) alkyl group which is optionally substituted with one or two hydroxyl groups, a protecting (P_{G}), preferably a BOC group) or a targeting element T_{E} which is linked to the nitrogen by a linker group L_{C} which is optionally cleavable;
R^{Q} and R^{X} are each independently H or a C₁-C₆ (preferably C₁-C₃) alkyl group which is optionally substituted with one or two hydroxyl groups or up to three halogen groups;
i is 1-4, preferably 2-4;
j is 1-3;
each n is independently 1, 2 or 3 (preferably 1);
R^{B1} and R^{B2} are each independently H, a C₁-C₆ (preferably C₁-C₃) group which is optionally substituted with one or two hydroxyl groups or up to three halogen groups or together R^{B} and R^{B2} form a cyclopropyl or cyclobutyl group (preferably, R^{B1} and R^{B2} are each independently H, methyl or together form a cyclopropyl group);
R^{C} is H, a C₁-C₁₂ optionally substituted alkyl or alkene group (preferably substituted with one or two hydroxyl groups, up to five halo groups) or a —(CH₂)ₙ₁NR₁R₂ group where R₁ and R₂ are each independently H or a C₁-C₆ optionally substituted alkyl group and n1 is 1-8 (preferably 1, 2, 3, 4 or 5), a protecting group (P_{G}) (preferably a BOC group) or a targeting element T_{E} which is linked to X¹ by a linker group L_{C} which is optionally cleavable, or R^{C} forms a dimer compound through a covalent linker group L which is optionally cleavable, said dimer compound having the general chemical structure:

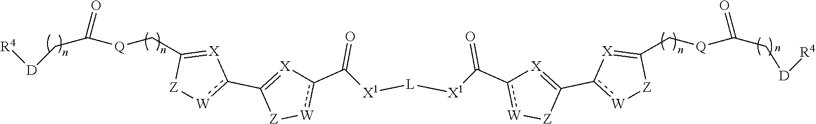

Where X, W, Z, Q, X¹, D, R², R⁴, n, R^{B1} and R^{B2} are the same as above; and L is a linker group which is optionally cleavable and covalently links the dimeric portions of the molecule to each other, or
a pharmaceutically acceptable salt, stereoisomer, solvate or polymorph thereof.
In preferred aspects of compound I, each n is 1, W is C—R, R is H or methyl, X is N, Z is S or N—R^{N}, R^{N} is H or methyl, Q is C(R^{Q})R^{Q}, each R^{Q} is independently H or methyl, preferably both are H, X¹ is NH or N-methyl, R² is H or methyl (preferably H), R^{A} in R⁴ is methyl, R^{B1} and R^{B2} are each independently H or methyl or together form a cyclopropyl group and L is a linker as otherwise described herein, preferably L is a polyethylene glycol linker having between 2 and 12 ethylene glycol units or a —(CH₂)ₘN(R)(CH₂)ₘ— group where R is H or a C₁-C₃ alkyl group (preferably H or methyl) and each m is independently from 1-12 (preferably, 1-10, more preferably 1, 2, 3, 4, 5, or 6).
In another embodiment, the invention is directed to compounds according to the chemical structure II:

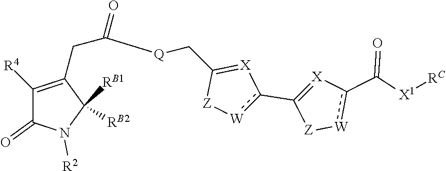

### Where X is N or C—R;

W is N, N—R^{N}, C—R or CR(R) (preferably the variable bond between W and the adjacent carbon atom is a double bond);
Each Z is independently S, O, N—R^{N} or C—R(R);
Each R is independently H, a C₁-C₆ (preferably C₁-C₃) alkyl group optionally substituted with one or two hydroxyl groups or up to three halogen groups, or a O—(C₁-C₃) alkoxy group;
Each R^{N} is independently H or a C₁-C₆ (preferably C₁-C₃) alkyl group optionally substituted with one or two hydroxyl groups or up to three halogen groups, preferably H or methyl;
Q is O, S, N(R¹) or C(R^{Q})R^{Q};
X¹ is O, S, N(R³) or C(R^{X})R^{X};
R¹, R² and R³ are each independently H or a C₁-C₆ (preferably C₁-C₃) alkyl group which is optionally substituted with one or two hydroxyl groups or up to three halogens groups;

### R⁴ is

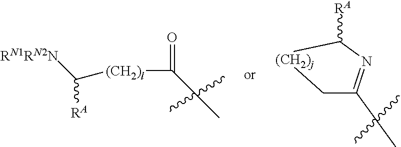

Where R^{A} is H or an optionally substituted C₁-C₈ alkyl or alkene group, preferably H or a C₁-C₃ alkyl, most often methyl;
R^{N1} and R^{N2} are each independently H, a C₁-C₆ (preferably C₁-C₃) alkyl group which is optionally substituted with one or two hydroxyl groups or up to three halogen groups, a protecting (P_{G}) (preferably a BOC group) or a targeting element T_{E} which is linked to the nitrogen by a linker L_{C} which is optionally cleavable;
R^{Q} and R^{X} are each independently H or a C₁-C₆ (preferably C₁-C₃) alkyl group which is optionally substituted with one or two hydroxyl groups or up to three halogen groups;
i is 1-4, preferably 2-4;
j is 1-3;
R^{B1} and R^{B2} are each independently H, a C₁-C₆ (preferably C₁-C₃) group which is optionally substituted with one or two hydroxyl groups or up to three halogen groups, or together R^{B1} and R^{B2} form a cyclopropyl or cyclobutyl group (preferably, R^{B1} and R^{B2} are each independently H, methyl or together form a cyclopropyl group);
R^{C} is H, a C₁-C₁₂ optionally substituted alkyl or alkene group (preferably substituted with one or two hydroxyl groups, up to five halo groups) or a —(CH₂)ₙNR₁R₂ group where R₁ and R₂ are each independently H or a C₁-C₆ optionally substituted alkyl group and n is 1-8 (preferably 1, 2, 3, 4 or 5), a protecting (P_{G}) (preferably a BOC group) or a targeting element T_{E} which is linked to X¹ (preferably through a nitrogen) by a linker L_{C} which is optionally cleavable, or R^{C} forms a dimer compound through a covalent linker group L which is optionally cleavable, said dimer compound having the general chemical structure:

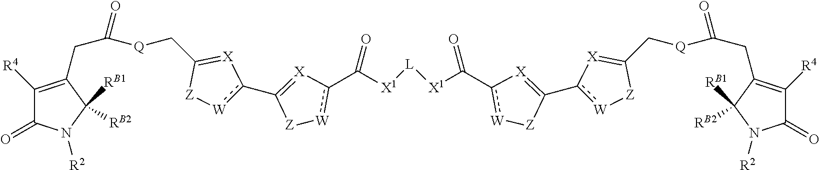

Where X, W, Z, Q, X¹, R², R⁴, R^{B1} and R^{B2} are the same as above; and L is a linker group which is optionally cleavable and which covalently links the dimeric portions of the molecule to each other, or
a pharmaceutically acceptable salt, stereoisomer, solvate or polymorph thereof.
In preferred aspects of compound II, W is C—R, R is H or methyl, X is N, Z is S or N—R^{N}, R^{N} is H or methyl, Q is N—H or C(R^{Q})R^{Q} where each R^{Q} is independently H or methyl, preferably both are H, X¹ is NH or N-methyl, R² is H or methyl (preferably H), R^{A} in R⁴ is methyl, R^{B1} and R^{B2} are each independently H or methyl or together form a cyclopropyl group and L is a linker as otherwise described herein, preferably L is a polyethylene glycol linker having between 2 and 12 ethylene glycol units or a —(CH₂)ₘN(R)(CH₂)ₘ— group where R is H or a C₁-C₃ alkyl group (preferably H or methyl) and each m is independently from 1-12 (preferably, 1-10, more preferably 1, 2, 3, 4, 5, or 6).
In another embodiment, the present invention is directed to certain preferred compounds according to the general chemical structure III:

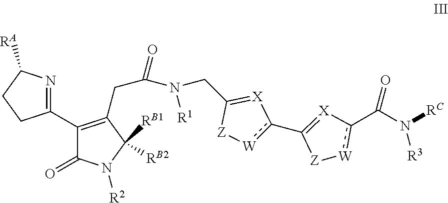

### Where X is N or C—R;

W is N, N—R^{N}, C—R or CR(R) (preferably the bond between W and the adjacent carbon atom is a double bond);
Each Z is independently S, O, N—R^{N} or C—R(R);
Each R is independently H, a C₁-C₃ alkyl group optionally substituted with one or two hydroxyl groups or up to three halogen groups, or a O—(C₁-C₃) alkoxy group;
Each R^{N} is independently H or a C₁-C₃ alkyl group optionally substituted with one or two hydroxyl groups or up to three halogen groups, preferably H or methyl;
R¹, R² and R³ are each independently H or a C₁-C₃ alkyl group which is optionally substituted with one or two hydroxyl groups or up to three halogen groups;
R^{A} is H or an optionally substituted C₁-C₈ alkyl or alkene group, preferably H or a C₁-C₃ alkyl, most often methyl;
R^{B1} and R^{B2} are each independently H, a C₁-C₃ alkyl group which is optionally substituted with one or two hydroxyl groups or up to three halo groups (F, Cl, Br or I, preferably Cl or F, most often F) or together R^{B1} and R^{B2} form a cyclopropyl or cyclobutyl group (preferably, R^{B1} and R^{B2} are each independently H, methyl or together form a cyclopropyl group);
R^{C} is H, a C₁-C₁₂ optionally substituted alkyl or alkene group (preferably substituted with one or two hydroxyl groups, up to five halo groups) or a —(CH₂)ₙNR₁R₂ group where R₁ and R₂ are each independently H or a C₁-C₆ optionally substituted alkyl group and n is 1-8 (preferably 1, 2, 3, 4 or 5), a protecting (P_{G}) (preferably a BOC group) or a targeting element T_{E} which is linked to X¹ (preferably a nitrogen) by a linker L_{C} which is optionally cleavable or R^{C} forms a dimer compound through a covalent linker group L which is optionally cleavable, said dimer compound having the general chemical structure:

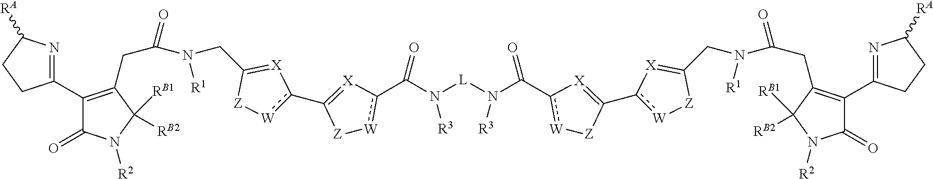

Where X, W, Z, R, R^{N}, R¹, R², R³, R^{A}, R^{B1} and R^{B2} are the same as above; and L is a linker group which covalently links the dimeric portions of the molecule to each other, or a pharmaceutically acceptable salt, stereoisomer, solvate or polymorph thereof. In preferred aspects of compound III, W is C—R, R is H or methyl, X is N, Z is S or N—R^{N}, R^{N} is H or methyl, Q is C(R^{Q})R^{Q}, each R^{Q} is independently H or methyl, preferably both are H, X¹ is NH or N-methyl, R² is H or methyl (preferably H), R^{A} in R⁴ is methyl, R^{B1} and R^{B2} are each independently H or methyl or together form a cyclopropyl group and L is a linker as otherwise described herein, preferably L is a polyethylene glycol group having between 2 and 12 ethylene glycol units or a —(CH₂)ₘN(R)(CH₂)ₘ— group where R is H or a C₁-C₃ alkyl group (preferably H or methyl) and each m is independently from 1-12 (preferably, 1-10, more preferably 1, 2, 3, 4, 5, or 6).
Preferably, the compound is according to the chemical structure IV:

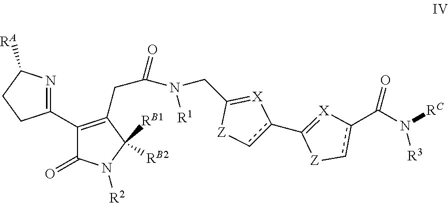

Where X, Z, R¹, R², R³, R^{A}, R^{B1}, R^{B2} and R^{C} are the same as for compound III above (the bond between the two carbons is preferably a double bond),
or a pharmaceutically acceptable salt, stereoisomer thereof. In preferred embodiments, the variable bond between carbons is a double bond. In preferred embodiments, X is preferably N; Z is preferably S, O, N—H or N—CH₃ (more preferably S); R is preferably H, methyl or OMe; R^{N} is preferably H or methyl; R¹, R² and R³ are each independently preferably H or methyl; R^{A} is preferably H or a C₁-C₃ alkyl, preferably methyl; R^{B1} and R^{B2} are each independently H, methyl or together form a cyclopropyl group and R^{C} is methyl, a —(CH₂)ₙ—N(CH₃)₂ group where n is 1, 2, 3 or 4 (preferably 2), forms a guanidine group with the nitrogen to which it is attached or R^{C} forms a dimer compound through linker L where L is preferably a —(CH₂)ₘN(R)(CH₂)ₘ— group where R is H or a C₁-C₃ alkyl group (preferably H or methyl) and each m is independently from 1-12 (preferably, 1-10, more preferably 1, 2, 3, 4, 5, or 6).

In one embodiment, preferred compounds according to the present invention include compounds of the chemical structure:

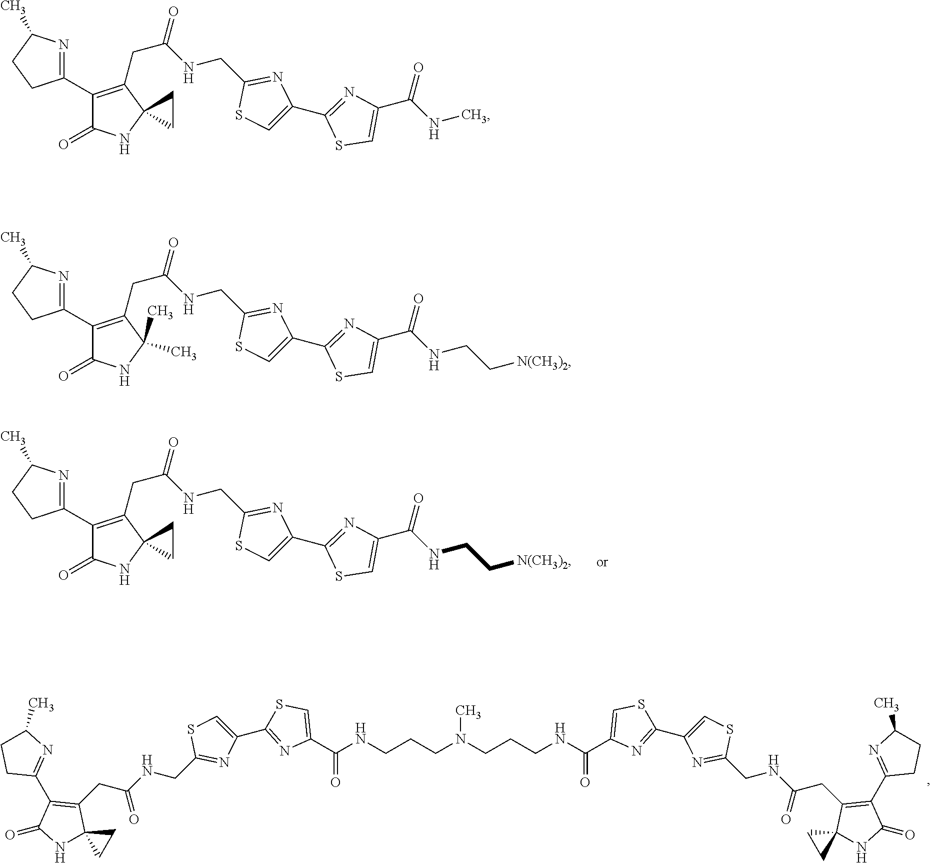

a pharmaceutical salt, stereoisomer, solvate or polymorph thereof.

In alternative embodiments, the compound is according to the chemical structure V:

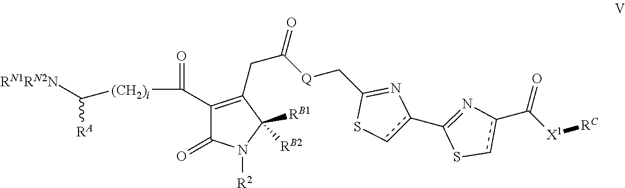

### Where Q is CH₂, N—H or N-Me;

X¹ is O, S, N(R³) or C(R^{X})R^{X};
R² and R³ are each independently H or a C₁-C₆ (preferably C₁-C₃) alkyl group which is optionally substituted with one or two hydroxyl groups or up to three halogen groups;
R^{A} is H or an optionally substituted C₁-C₈ alkyl or alkene group, preferably H or a C₁-C₃ alkyl, most often methyl;
R^{N1} and R^{N2} are each independently H, a C₁-C₆ (preferably C₁-C₃) alkyl group which is optionally substituted with one or two hydroxyl groups or up to three halogen groups, a protecting (P_{G}), preferably a BOC group, or a targeting element T_{E} which is linked to the nitrogen by a linker L_{C}; which is optionally cleavable;
Each R^{X} is independently H or a C₁-C₆ (preferably C₁-C₃) alkyl group which is optionally substituted with one or two hydroxyl groups or up to three halogen groups (preferably F or Cl, more often F);
i is 1-4, preferably 2-4;
R^{B1} and R^{B2} are each independently H, a C₁-C₆ (preferably C₁-C₃) group which is optionally substituted with one or two hydroxyl groups or up to three halogen groups (preferably F, Cl, Br or I, preferably Cl or F, most often F) or together R^{B1} and R^{B2} form a cyclopropyl or cyclobutyl group (preferably, R^{B1} and R^{B2} are each independently H, methyl or together form a cyclopropyl group);
R^{C} is H, a C₁-C₁₂ optionally substituted alkyl or alkene group (preferably substituted with one or two hydroxyl groups or up to five halo groups), a —(CH₂)ₙ₁NR₁R₂ group where R₁ and R₂ are each independently H or a C₁-C₆ optionally substituted alkyl group and n1 is 1-8 (preferably 1, 2, 3, 4 or 5), a protecting P_{G}, preferably a BOC group, or a targeting element T_{E} which is linked to X¹ (preferably a nitrogen) by linker L_{C} which is optionally cleavable, or R^{C} forms a dimer compound through a covalent linker group L which is optionally cleavable, said dimer compound having the general chemical structure:

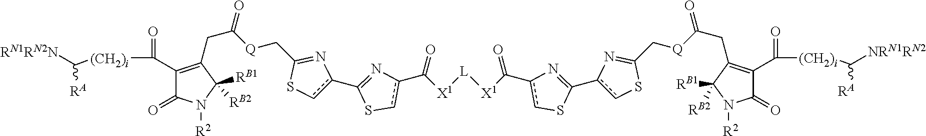

Where Q, R², R^{A}, i, R^{B1}, R^{B2}, R^{N1}, R^{N2}, X¹ and L are the same as for compound V above, and L is a linker group which is optionally cleavable and which covalently links the dimeric portions of the molecule to each other, or
a pharmaceutically acceptable salt, stereoisomer, solvate or polymorph thereof. In preferred embodiments, the variable bond between carbons is a double bond, R² is H or methyl, R^{A} is methyl, i is 1, R^{B1} and R^{B2} are each H, methyl or together form a cyclopropyl group, NR^{N1} and NR^{N2} are each independently H, methyl, a protecting group (preferably a BOC) or a targeting element T_{E} which is linked to the nitrogen by an optionally cleavable linker L_{C}, X¹ is N—H or N-methyl and L is a linker group —(CH₂)ₘN(R)CH₂)ₘ— where R is H or a C₁-C₃ alkyl group (preferably H or methyl) and each m is independently from 1-12 (preferably, 1-10, more preferably 1, 2, 3, 4, 5, or 6).

In another embodiment, the present invention is directed to compounds according to the chemical structure:

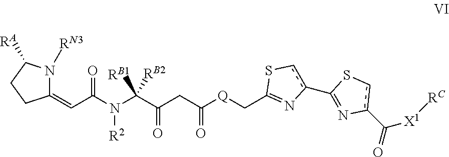

### Where Q is CH₂ or N—H;

X¹ is O, S, N(R³) or C(R^{X})R^{X};
R² and R³ are each independently H or a C₁-C₆ (preferably C₁-C₃) alkyl group which is optionally substituted with one or two hydroxyl groups;
Each R^{X} is independently H or a C₁-C₆ (preferably C₁-C₃) alkyl group which is optionally substituted with one or two hydroxyl groups;
R^{A} is H or an optionally substituted C₁-C₈ alkyl or alkene group, preferably H or a C₁-C₃ alkyl, most often methyl;
R^{N3} is H, a C₁-C₆ (preferably C₁-C₃) alkyl group which is optionally substituted with one or two hydroxyl groups, a protecting (P_{G}), preferably a BOC group, or a targeting element T_{E} which is linked to the nitrogen by an optionally cleavable linker L_{C};
R^{B1} and R^{B2} are each independently H, a C₁-C₆ (preferably C₁-C₃) group which is optionally substituted with one or two hydroxyl groups or up to three halo groups (F, Cl, Br or I, preferably Cl or F, most often F) or together R^{B1} and R^{B2} form a cyclopropyl or cyclobutyl group (preferably, R^{B1} and R^{B2} are each independently H, methyl or together form a cyclopropyl group); and
R^{C} is H, a C₁-C₁₂ optionally substituted alkyl or alkene group (preferably substituted with one or two hydroxyl groups, up to five halo groups) or a —(CH₂)ₙ₁NR₁R₂ group where R₁ and R₂ are each independently H or a C₁-C₆ optionally substituted alkyl group and n1 is 1-8 (preferably 1, 2, 3, 4 or 5), a protecting (P_{G}), preferably a BOC group) or a targeting element T_{E} which is linked to X¹ (preferably a nitrogen) by a linker L_{C} which is optionally cleavable, or R^{C} forms a dimer compound through a covalent linker group L which is optionally cleavable, said dimer compound having the general chemical structure:

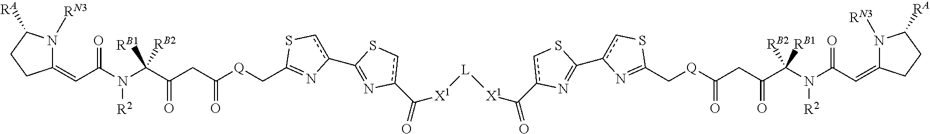

Where Q, R², R^{A}, R^{B1}, R^{B2}, R^{N3} and X¹ are the same as above for compound VI, and L is a linker group which covalently links the dimeric portions of the molecule to each other, or a pharmaceutically acceptable salt, stereoisomer, solvate or polymorph thereof. In preferred embodiments, the variable bond between the two carbons is a double bond. In preferred embodiments, R² is H or methyl, R^{A} is H or methyl, R^{B1} and R^{B2} are each independently H, methyl or together form a cyclopropyl group, R^{N3} is H, methyl, a protecting group (P_{G}), preferably a BOC group, or a targeting element T_{E} which is linked to the nitrogen by an optionally cleavable linker L_{C}, X¹ is N—H or N-methyl and L is a linker group —(CH₂)ₘN(R)(CH₂)ₘ— where R is H or a C₁-C₃ alkyl group (preferably H or methyl) and each m is independently from 1-12 (preferably, 1-10, more preferably 1, 2, 3, 4, 5, or 6).

In other embodiments, the present invention is directed to pharmaceutical compositions which are principally used for treating cancer comprising an effective amount of a compound having anticancer activity as otherwise described herein in combination with a pharmaceutically acceptable carrier, additive or excipient, optionally in combination with at least one additional bioactive agent, in most instances at least one additional anticancer agent.

In further embodiments, the present invention is directed to methods of treating cancer comprising administering to a patient in need an effective amount of compound or pharmaceutical composition as described herein.

In still other embodiments, the present invention is directed to methods of chemical synthesis as set forth in the schemes which are presented herein, including the attached figures and the compounds which are also described herein.

In still other embodiments, compounds according to the present invention include one or more of the following:

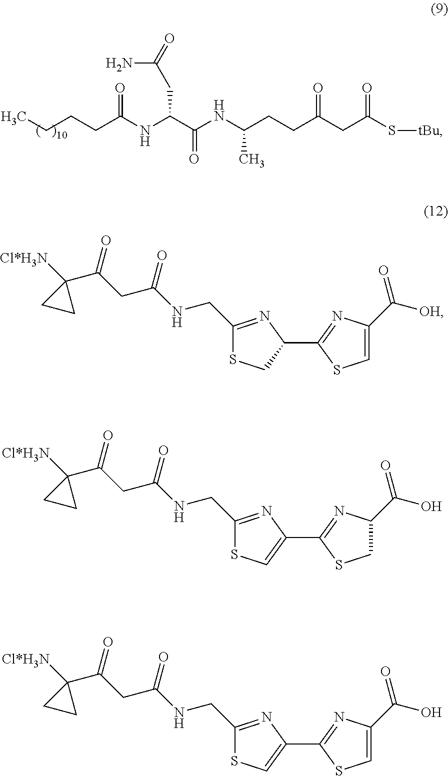

Or a non-salt or alternative salt form, including a pharmaceutically acceptable salt form or stereoisomer thereof,

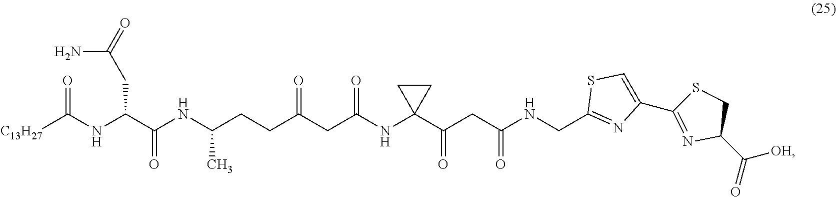

Or a salt form, including a pharmaceutically acceptable salt form or stereoisomer thereof.

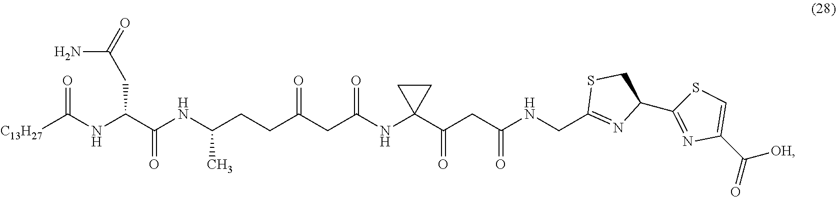

Or a salt form, including a pharmaceutically acceptable salt form or stereoisomer thereof.

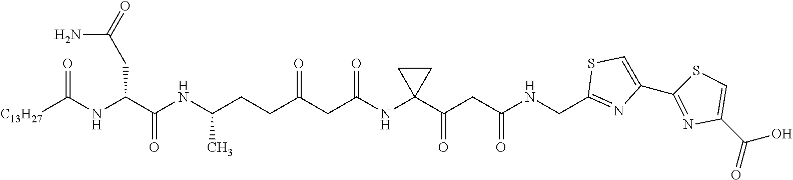

Or a salt form, including a pharmaceutically acceptable salt form or stereoisomer thereof.
The present invention also is direct to anticancer compounds of according to the general chemical structures:

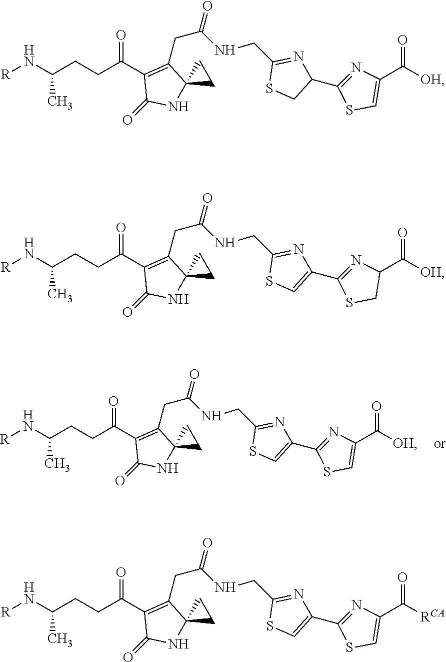

Where R is a targeting element T_{E} such as a non-cleavable or cleavable moiety which optionally has anticancer activity or a moiety such as an acid labile moiety including an acid labile peptide (e.g. a low pH insertion peptide), an antibody or antibody fragment, a group

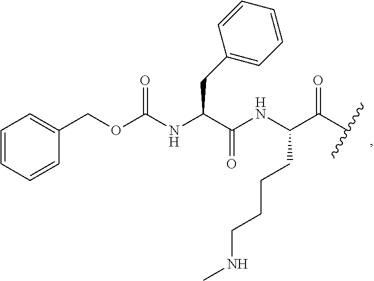 [SMILES: *C(=O)[C@H](CCCCNC)NC(=O)[C@H](Cc1ccccc1)NC(=O)OCc1ccccc1]

or a cysteine-cathepsin labile moiety; and R^{CA} is an amine group, preferably a diamine, triamine, tetramine, even more preferably a —NH(CH₂)ₘNR^{1D}R^{2D} group where m is an integer from 1-6 (1, 2, 3, 4, 5 or 6) and R^{1D} and R^{2D} are each independently H, C₁-C₆ alkyl which is optionally substituted with one or two hydroxyl groups or is a

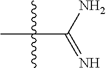 [SMILES: *C(=N)N]

groups which forms a guanidine group with the nitrogen to which it is attached
or a pharmaceutically acceptable salt or stereoisomer thereof.

In embodiments, the present invention is directed to compounds according to the chemical structure:

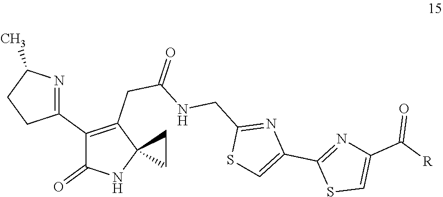

Where R is an amine group, preferably a diamine, triamine, tetramine, even more preferably a s-NH(CH₂)ₘNR^{1D}R^{2D} group where m is an integer from 1-6 (1, 2, 3, 4, 5 or 6) and R^{1D} and R^{2D} are each independently H, C₁-C₆ alkyl which is optionally substituted with one or two hydroxyl groups or is a

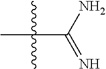 [SMILES: *C(=N)N]

groups which forms a guanidine group with the nitrogen to which it is attached,
or a pharmaceutically acceptable salt or stereoisomer thereof

The present invention is also directed to methods of chemical synthesis as described in the schemes presented herein and in the figures attached hereto.

In certain embodiments the present invention is directed to a chemical synthesis as set forth below:

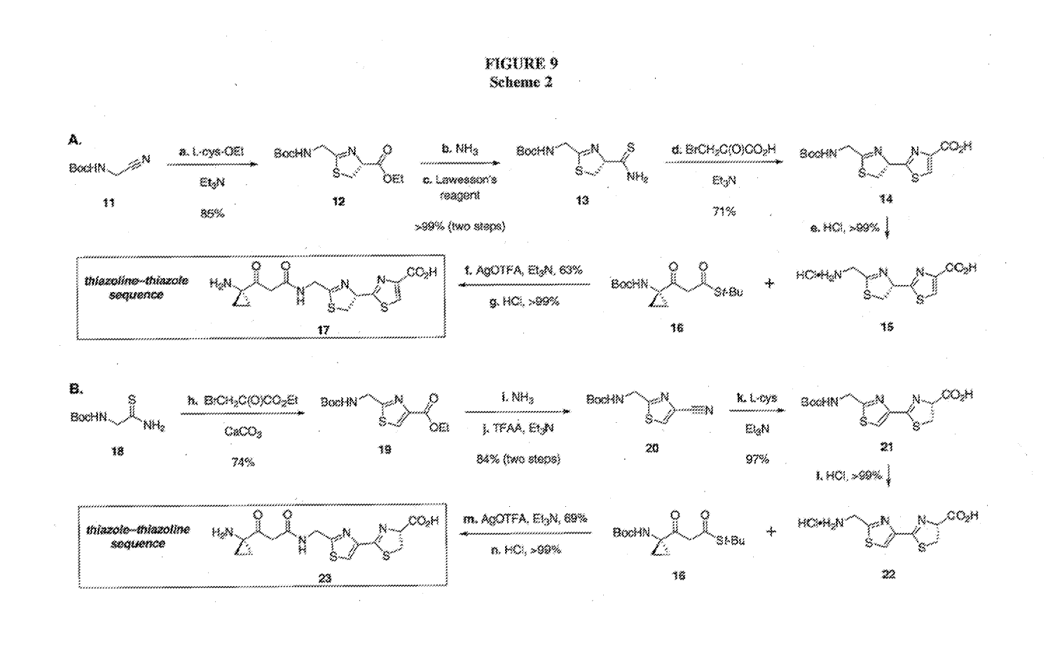

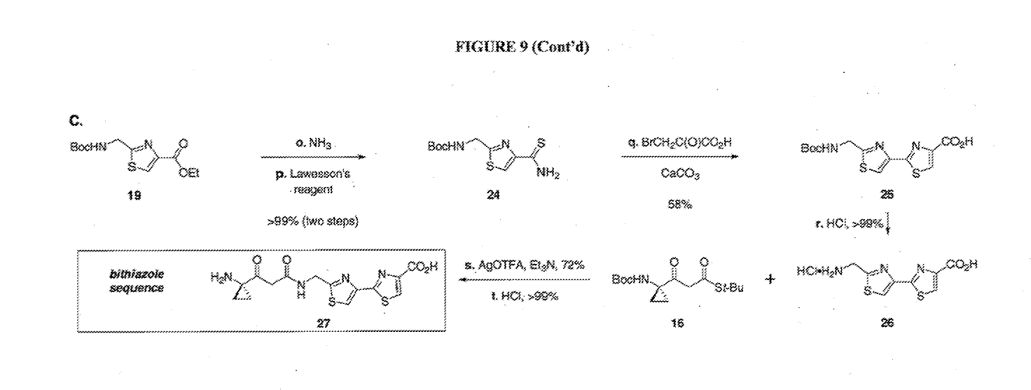

Compound 22 is converted to compound 23 by reacting compound 16 of FIG. 9 with compound 22 in a silver catalyst such as AgCF₃CO₂ in a weak base (e.g., triethylamine) and solvent (e.g. DMF) at reduced temperature (e.g. 0° C.) to produce the compound 23G in 63% yield.

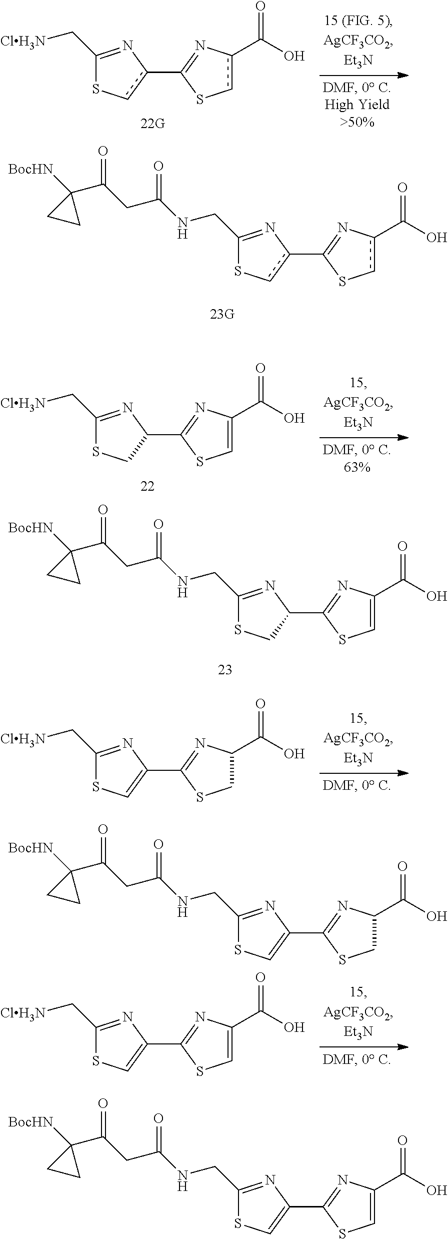

Compound 23, 23G or a related compound, produced above, is converted to compound 24/24G or a related compound in acid (e.g. HCl) in solvent (methylene chlorine-dioxane mixture) at about room temperature (e.g. 23° C.) under conditions to remove the Boc amine protecting group. The reaction proceeds in quantitative or close to quantitative yield.

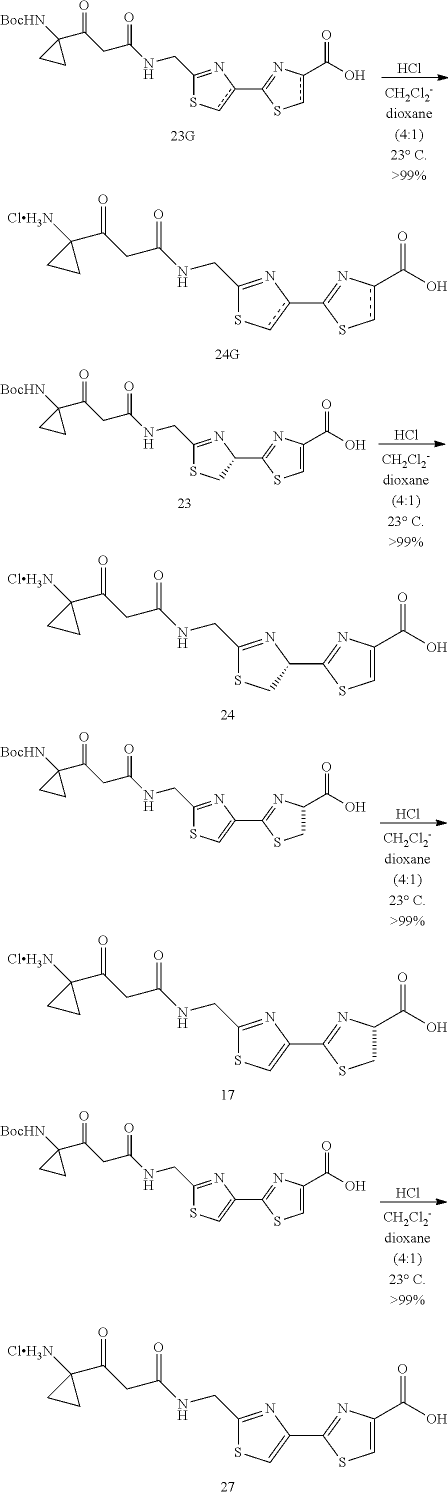

Compounds 9 and 24G, 9 and 17, G, 9 and 24 and 9 and 27 (above) are reacted together in the presence of a silver catalyst (e.g., AgCF₃CO₂) in a weak base (e.g., triethylamine) and solvent (e.g. DMF) at reduced temperature (e.g. 0° C.) to produce compound 25.

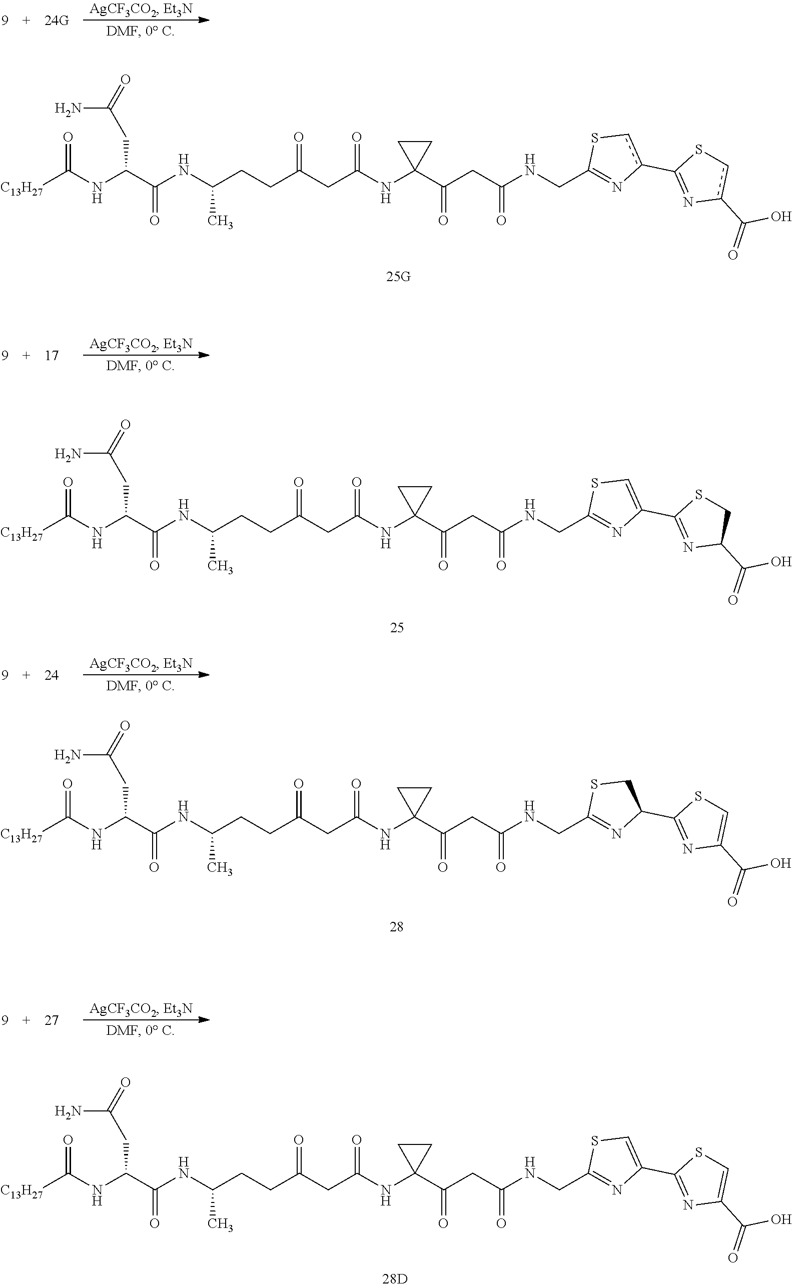

In an alternative embodiment, compound 13 (BL is an amine blocking group, preferably a Boc group), is exposed to propylphosphonic anhydride (T3P) in the presence of an amine to provide a peptide on the carboxylic acid position. The amine may be any amine group containing a primary amine, but preferably is an amine which contains at least one cationic group after formation of the amide bond, and can include a diamine, triamine, tetramine, preferably a NH₂(CH₂)ₘNR^{1D}R^{2D} where m is an integer from 1-6 (1, 2, 3, 4, 5 or 6) and R^{1D} and R^{2D} are each independently H, C₁-C₆ alkyl which is optionally substituted with one or two hydroxyl groups or is a

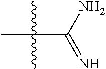 [SMILES: *C(=N)N]

groups which forms a guanidine group with the nitrogen to which it is attached. The protecting group is subsequently removed in trifluoroacetic acid and aqueous bicarbonate to produce the final product 15a.

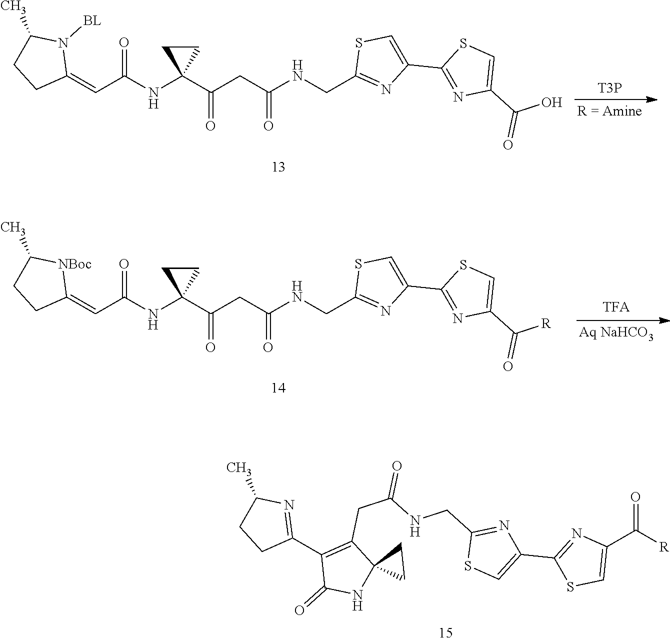

The above embodiments, and additional embodiments of the present invention are readily gleaned from a review of the detailed description of the invention which follows.

### DETAILED DESCRIPTION OF THE INVENTION

The following terms shall be used throughout the specification to describe the present invention. Where a term is not specifically defined herein, that term shall be understood to be used in a manner consistent with its use by those of ordinary skill in the art.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges that may independently be included in the smaller ranges are also encompassed within the invention, subject to any specifically excluded limit in the stated range. Where the stated range includes one or both of the limits, ranges excluding either both of those included limits are also included in the invention. In instances where a substituent is a possibility in one or more Markush groups, it is understood that only those substituents which form stable bonds are to be used.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Furthermore, the following terms shall have the definitions set out below.

The term “patient” or “subject” is used throughout the specification within context to describe an animal, generally a mammal, especially including a domesticated animal and preferably a human, to whom treatment, including prophylactic treatment (prophylaxis), with the compounds or compositions according to the present invention is provided. For treatment of those infections, conditions or disease states which are specific for a specific animal such as a human patient, the term patient refers to that specific animal. In most instances, the patient or subject of the present invention is a human patient of either or both genders.

The term “effective” is used herein, unless otherwise indicated, to describe an amount of a compound or component which, when used within the context of its use, produces or effects an intended result, whether that result relates to the prophylaxis and/or therapy of an infection and/or disease state within the context of its use or as otherwise described herein. The term effective subsumes all other effective amount or effective concentration terms (including the term “therapeutically effective”) which are otherwise described or used in the present application.

The term “compound” is used herein to describe any specific compound or bioactive agent disclosed herein, including any and all stereoisomers (including diastereomers, individual optical isomers/enantiomers or racemic mixtures and geometric isomers), pharmaceutically acceptable salts and prodrug forms. The term compound herein refers to stable compounds. Within its use in context, the term compound may refer to a single compound or a mixture of compounds as otherwise described herein. It is understood that the choice of substituents or bonds within a Markush or other group of substituents or bonds is provided to form a stable compound from those choices within that Markush or other group. The symbol [symbol] signifies that a bond is either a single bond or a double bond. In all compounds, where a variable bond is presented, the variable bond between two atoms is preferably a double bond.

The term “pharmaceutically acceptable” as used herein means that the compound or composition is suitable for administration to a subject to achieve the treatments described herein, without unduly deleterious side effects in light of the severity of the disease and necessity of the treatment.

“Hydrocarbon” or “hydrocarbyl” refers to any monovalent (or divalent in the case of alkylene groups) radical containing carbon and hydrogen, which may be straight, branch-chained or cyclic in nature. Hydrocarbons include linear, branched and cyclic hydrocarbons, including alkyl groups, alkylene groups, saturated and unsaturated hydrocarbon groups including aromatic groups both substituted and unsubstituted, alkene groups (containing double bonds between two carbon atoms) and alkyne groups (containing triple bonds between two carbon atoms). In certain instances, the terms substituted alkyl and alkylene are sometimes used synonymously.

“Alkyl” refers to a fully saturated monovalent radical containing carbon and hydrogen, and which may be cyclic, branched or a straight chain. Examples of alkyl groups are methyl, ethyl, n-butyl, n-hexyl, n-heptyl, n-octyl, n-nonyl, n-decyl, isopropyl, 2-methyl-propyl, cyclopropyl, cyclopropylmethyl, cyclobutyl, cyclopentyl, cyclopentylethyl, cyclohexylethyl and cyclohexyl. Preferred alkyl groups are C₁-C₆ alkyl groups. “Alkylene” refers to a fully saturated hydrocarbon which is divalent (may be linear, branched or cyclic) and which is optionally substituted. Preferred alkylene groups are C₁-C₆ alkylene groups. Other terms used to indicate substitutent groups in compounds according to the present invention are as conventionally used in the art.

The term “aryl” or “aromatic”, in context, refers to a substituted or unsubstituted monovalent aromatic radical having a single ring (e.g., benzene or phenyl). Other examples of aryl groups, in context, may include heterocyclic aromatic ring systems “heteroaryl” groups having one or more nitrogen, oxygen, or sulfur atoms in the ring (5- or 6-membered heterocyclic rings) such as imidazole, furyl, pyrrole, pyridyl, furanyl, thiene, thiazole, pyridine, pyrimidine, pyrazine, triazole, oxazole, among others, which may be substituted or unsubstituted as otherwise described herein.

The term “substituted” shall mean substituted at a carbon or nitrogen position within a molecule or moiety within context, a hydroxyl, carboxyl, cyano (C≡N), nitro (NO₂), halogen (preferably, 1, 2 or 3 halogens, especially on an alkyl, especially a methyl group such as a trifluoromethyl), alkyl group (preferably, C₁-C₁₂, more preferably, C₁-C₆), alkoxy group (preferably, C₁-C₆ alkyl or aryl, including phenyl and substituted phenyl), a C₁-C₆ thioether, ester (both oxycarbonyl esters and carboxy ester, preferably, C₁-C₆ alkyl or aryl esters) including alkylene ester (such that attachment is on the alkylene group, rather than at the ester function which is preferably substituted with a C₁-C₆ alkyl or aryl group), thioester (preferably, C₁-C₆ alkyl or aryl), halogen (preferably, F or Cl), nitro or amine (including a five- or six-membered cyclic alkylene amine, further including a C₁-C₆ alkyl amine or C₁-C₆ dialkyl amine which alkyl groups may be substituted with one or two hydroxyl groups), amido, which is preferably substituted with one or two C₁-C₆ alkyl groups (including a carboxamide which is substituted with one or two C₁-C₆ alkyl groups), alkanol (preferably, C₁-C₆ alkyl or aryl), or alkanoic acid (preferably, C₁-C₆ alkyl or aryl) or a thiol (preferably, C₁-C₆ alkyl or aryl), or thioalkanoic acid (preferably, C₁-C₆ alkyl or aryl). Preferably, the term “substituted” shall mean within its context of use alkyl, alkoxy, halogen, ester, keto, nitro, cyano and amine (especially including mono- or di-C₁-C₆ alkyl substituted amines which may be optionally substituted with one or two hydroxyl groups). Any substitutable position in a compound according to the present invention may be substituted in the present invention, but often no more than 3, more preferably no more than 2 substituents (in some instances only 1 or no substituents) is present on a ring. Preferably, the term “unsubstituted” or within context a bond which is unsubstituted shall mean substituted with one or more H atoms.

The term “tumor” is used to describe a malignant or benign growth or tumefacent.

The term “neoplasia” refers to the uncontrolled and progressive multiplication of tumor cells, under conditions that would not elicit, or would cause cessation of, multiplication of normal cells. Neoplasia results in a “neoplasm”, which is defined herein to mean any new and abnormal growth, particularly a new growth of tissue, in which the growth of cells is uncontrolled and progressive. Thus, neoplasia includes “cancer”, which herein refers to a proliferation of tumor cells having the unique trait of loss of normal controls, resulting in unregulated growth, lack of differentiation, local tissue invasion, and/or metastasis. The cancer may be “naïve”, metastatic or recurrent and includes drug resistant and multiple drug resistant cancers, all of which may be treated using compounds according to the present invention.

As used herein, neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms are distinguished from benign neoplasms in that the former show a greater degree of anaplasia, or loss of differentiation and orientation of cells, and have the properties of invasion and metastasis. Examples of neoplasms or neoplasias from which the target cell of the present invention may be derived include, without limitation, carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine/endometrial cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as WACMs' tumor and teratocarcinomas, which may be treated by one or more compounds according to the present invention. See, (Beers and Berkow (eds.), The Merck Manual of Diagnosis and Therapy, 17.sup.th ed. (Whitehouse Station, N.J.: Merck Research Laboratories, 1999) 973-74, 976, 986, 988, 991.

In certain particular aspects of the present invention, the cancer which is treated is metastatic cancer. Metastatic cancer may be found in virtually all tissues of a cancer patient in late stages of the disease, including the lymph system/nodes (lymphoma), in bones, in bladder tissue, in kidney tissue, liver tissue and in virtually any tissue, including brain (brain cancer/tumor). Thus, the present invention is generally applicable and may be used to treat any cancer in any tissue, regardless of etiology. In other instances, the cancer which is treated, including prophylactically treated, is a recurrent cancer, which often recurs after an initial remission. The present compounds also may be used to reduce the likelihood of a cancer recurring and for treating a cancer which has recurred. In further instances the present compounds may be used to treat cancer stem cells, which often occur in metastatic and recurrent cancers.

The term “targeting element”, “cancer cell targeting element”, “T_{E}”, “CCT_{E}” or “cell targeting element” is used to describe that portion of a chimeric compound according to the present invention which comprises at least one moiety which is capable of selectively binding to a cancer cell. Targeting groups for including in chimeric compounds according to the present invention include small molecules which bind to folate receptors (folate receptor binding moiety), antibody-type CCT_{E}s such as monoclonal antibodies (especially a humanized monoclonal antibody) such as herceptin or antibody fragments (FAB), including single chain variable fragment (scFv) antibodies which bind to cancer cells, a PSMA binding moiety or a YSA peptide (which binds to Ephrin A2 (EphA2), as otherwise described herein. The targeting element T_{E} may also include a peptide (e.g. a low pH insertion peptide), an antibody or antibody fragment, a group according to the chemical structure

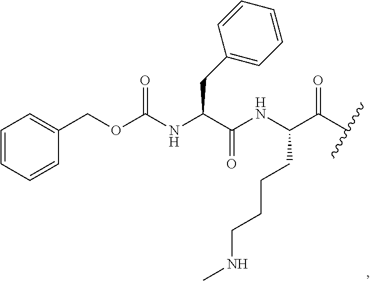 [SMILES: *C(=O)[C@H](CCCCNC)NC(=O)[C@H](Cc1ccccc1)NC(=O)OCc1ccccc1]

or a cysteine-cathepsin moiety.

The term “folate receptor binding moiety” (FRBM) or (FM) is used to describe a folate moiety which binds to cancer cells selectively and is used in the present invention to target folate receptors on cancer cells which are often overexpressed or hyperexpressed on cancer cells compared to normal cells. The folate receptor, given its selective heightened expression on cancer cells compared to normal cells represents an excellent selective target to bind compounds according to the present invention to cancer cells for uptake into cells where the intercalating moiety may exhibit its antiproliferative activity, resulting in cancer cell death. Folate receptor I is often overexpressed in numerous cancer cells including ovarian, breast, uterine, cervical, renal, lung, colorectal and brain cancer cells, thus making it an important targeting site for compounds according to the present invention.

Folate receptor binding moieties for use in the present invention include the following chemical structures:

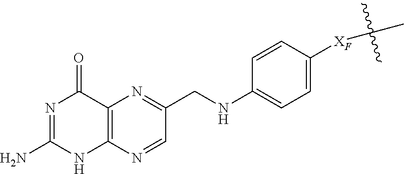

where X_{F} is C(O), S(O), S(O)₂, CR_{F}R_{F}, O, S or N—R_{F},
where R_{F} is H or a C₁-C₃ alkyl (preferably H).

The term “prostate specific membrane antigen” or “PSMA” according to the chemical structure is directed to a cancer cell targeting moiety that binds to prostate specific membrane antigen (PSMA) which is frequently overexpressed or hyperexpressed in cancer cells. PSMA, although found on prostate cancer cells, including metastatic prostate cancer cells, are also found on virtually all other cancer cells and may be used to selectively target compounds according to the present invention to cancer cells. A number of metastatic and recurrent cancers also hyperexpress PSMA compared to naïve cancers and PSMA may represent a particularly useful binding site for metastatic and/or recurrent cancers.

PSMA binding moieties include moieties according to the chemical structure:

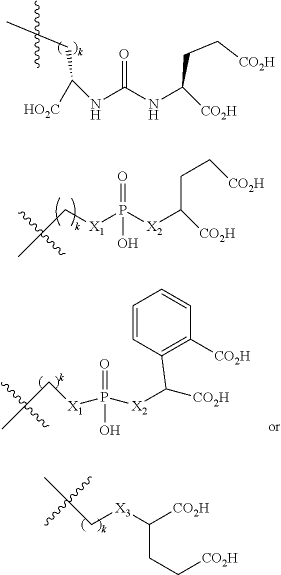

Where X₁ and X₂ are each independently CH₂, O, NH or S;
X₃ is O, CH₂, NR¹, S(O), S(O)₂, —S(O)₂O, —OS(O)₂, or OS(O)₂O;
R¹ is H, a C₁-C₃ alkyl group, or a —C(O)(C₁-C₃) group;
k is an integer from 0 to 20, 8 to 12, 1 to 15, 1 to 10, 1 to 8, 1 to 6, 1, 2, 3, 4, 5 or 6;
or a salt or enantiomer thereof.

A preferred PSMA binding group (CCT_{E}) for use in the present invention is the group

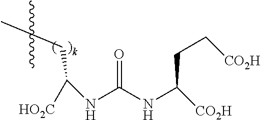

Where k is 2, 3 or 4, preferably 3 or 4. This CCT_{E} group, as well as the others, optionally has an amine group or other functional group at the distill end of the alkylene group (k) such that k is formed from, for example, a lysine amino acid, such that the amine group or other functional group may participate in further reactions to form a linker, a connector group [CON], a multifunctional group [MULTICON] or may be linked directly to an (ACM) as otherwise described herein.

The term “blocking group” refers to a group which is introduced into a molecule by chemical modification of a function group to obtain chemoselectivity in a subsequent chemical reaction. It plays an important role in providing precursors to chemical components which provide compounds according to the present invention. Blocking groups may be used to protect functional groups on ACM groups, CCT_{E} groups, connector molecules and/or linker molecules in order to assemble compounds according to the present invention. Typical blocking groups are used on alcohol groups, amine groups, carbonyl groups, carboxylic acid groups, phosphate groups and alkyne groups among others.

Exemplary alcohol/hydroxyl protecting groups include acetyl (removed by acid or base), benzoyl (removed by acid or base), benzyl (removed by hydrogenolysis, β-methoxyethoxymethyl ether (MEM, removed by acid), dimethoxytrityl [bis-(4-methoxyphenyl)phenylmethyl] (DMT, removed by weak acid), methoxymethyl ether (MOM, removed by acid), methoxytrityl [(4-methoxyphenyl)diphenylmethyl], (MMT, Removed by acid and hydrogenolysis), p-methoxylbenzyl ether (PMB, removed by acid, hydrogenolysis, or oxidation), methylthiomethyl ether (removed by acid), pivaloyl (Piv, removed by acid, base or reductant agents. More stable than other acyl protecting groups, tetrahydropyranyl (THP, removed by acid), tetrahydrofuran (THF, removed by acid), trityl (triphenyl methyl, (Tr, removed by acid), silyl ether (e.g. trimethylsilyl or TMS, tert-butyldimethylsilyl or TBDMS, tri-iso-propylsilyloxymethyl or TOM, and triisopropylsilyl or TIPS, all removed by acid or fluoride ion such as such as NaF, TBAF (tetra-n-butylammonium fluoride, HF-Py, or HF-NEt₃); methyl ethers (removed by TMSI in DCM, MeCN or chloroform or by BBr₃ in DCM) or ethoxyethyl ethers (removed by strong acid).

Exemplary amine-protecting groups include carbobenzyloxy (Cbz group, removed by hydrogenolysis), p-Methoxylbenzyl carbon (Moz or MeOZ group, removed by hydrogenolysis), tert-butyloxycarbonyl (BOC group, removed by concentrated strong acid or by heating at elevated temperatures), 9-Fluorenylmethyloxycarbonyl (FMOC group, removed by weak base, such as piperidine or pyridine), acyl group (acetyl, benzoyl, pivaloyl, by treatment with base), benzyl (Bn groups, removed by hydrogenolysis), carbamate, removed by acid and mild heating, p-methoxybenzyl (PMB, removed by hydrogenolysis), 3,4-dimethoxybenzyl (DMPM, removed by hydrogenolysis), p-methoxyphenyl (PMP group, removed by ammonium cerium IV nitrate or CAN); tosyl (Ts group removed by concentrated acid and reducing agents, other sulfonamides, Mesyl, Nosyl & Nps groups, removed by samarium iodide, tributyl tin hydride.

Exemplary carbonyl protecting groups include acyclical and cyclical acetals and ketals (removed by acid), acylals (removed by Lewis acids) and dithianes (removed by metal salts or oxidizing agents).

Exemplary carboxylic acid protecting groups include methyl esters (removed by acid or base), benzyl esters (removed by hydrogenolysis), tert-butyl esters (removed by acid, base and reductants), esters of 2,6-disubstituted phenols (e.g. 2,6-dimethylphenol, 2,6-diisopropylphenol, 2,6-di-tert-butylphenol, removed at room temperature by DBU-catalyzed methanolysis under high-pressure conditions, silyl esters (removed by acid, base and organometallic reagents), orthoesters (removed by mild aqueous acid), oxazoline (removed by strong hot acid (pH<1, T>100° C.) or strong hot alkali (pH>12, T>100° C.)).

Exemplary phosphate group protecting groups including cyanoethyl (removed by weak base) and methyl (removed by strong nucleophiles, e.g. thiophenol/TEA).

Exemplary terminal alkyne protecting groups include propargyl alcohols and silyl groups.

The term “antibody”, also referred to an immunoglobulin (Ig), is a protein, which is Y-shaped and produced by B-cells that the immune system uses to identify and neutralize foreign objects in the body, such as pathogens, including viruses, bacteria and cancer cells, which the immune system recognizes as objects to the immune system. As used herein, antibody includes, but is not limited to, monoclonal antibodies. The following disclosure from U.S. Patent Application Document No. 20100284921, the entire contents of which are hereby incorporated by reference, exemplifies techniques that are useful in making antibodies which may be modified and employed in chimeric compounds of the instant invention.

Pursuant to its use in the present invention, the antibody is preferably a chimeric antibody. For human use, the antibody is preferably a humanized chimeric antibody. [A]n anti-target-structure antibody . . . may be monovalent, divalent or polyvalent in order to achieve target structure binding. Monovalent immunoglobulins are dimers (HL) formed of a hybrid heavy chain associated through disulfide bridges with a hybrid light chain. Divalent immunoglobulins are tetramers (H2L2) formed of two dimers associated through at least one disulfide bridge.

As discussed above, the term antibody for use in the present invention includes compounds which exhibit binding characteristics comparable to those of the antibodies, and include, for example, hybridized and single chain antibodies, as well as fragments thereof. Methods of producing such compounds are disclosed in PCT Application Nos. WO 1993/21319 and WO 1989/09622. These compounds include polypeptides with amino acid sequences substantially the same as the amino acid sequence of the variable or hypervariable regions of the antibodies raised against targets on cancer cells pursuant to the practice of the present invention. These may be readily modified to link these CCTMs to the (ACM), thus forming chimeric compounds hereunder.

Compounds according to the present invention which serve to bind to target cancer cells include fragments of antibodies (FAB) that have the same, or substantially the same, binding characteristics to those of the whole antibody. Such fragments may contain one or both Fab fragments or the F(ab′)₂ fragment. Preferably the antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Preferred constant regions are gamma 1 (IgG1), gamma 2 (IgG2 and IgG), gamma 3 (IgG3) and gamma 4 (IgG4). The light chain constant region can be of the kappa or lambda type.

In another approach, the monoclonal antibodies may be advantageously cleaved by proteolytic enzymes to generate fragments retaining the target structure binding site. For example, proteolytic treatment of IgG antibodies with papain at neutral pH generates two identical so-called “Fab” fragments, each containing one intact light chain disulfide-bonded to a fragment of the heavy chain (Fc). Each Fab fragment contains one antigen-combining site. The remaining portion of the IgG molecule is a dimer known as “Fc”. Similarly, pepsin cleavage at pH 4 results in the so-called F(ab′)2 fragment.

Single chain antibodies or Fv fragments are polypeptides that consist of the variable region of the heavy chain of the antibody linked to the variable region of the light chain, with or without an interconnecting linker. Thus, the Fv comprises an antibody combining site. Hybrid antibodies also may be employed as CMTs in the chimeric compounds according to the present invention. Hybrid antibodies have constant regions derived substantially or exclusively from human antibody constant regions and variable regions derived substantially or exclusively from the sequence of the variable region of a monoclonal antibody from each stable hybridoma.

Methods for preparation of fragments of antibodies (e.g. for preparing an antibody or an antigen binding fragment thereof having specific binding affinity for a cancer cell target are readily known to those skilled in the art. See, for example, Goding, “Monoclonal Antibodies Principles and Practice”, Academic Press (1983), p. 119-123. Fragments of the monoclonal antibodies containing the antigen binding site, such as Fab and F(ab′)2 fragments, may be preferred in therapeutic applications, owing to their reduced immunogenicity. Such fragments are less immunogenic than the intact antibody, which contains the immunogenic Fc portion. Hence, as used herein, the term “antibody” includes intact antibody molecules and fragments thereof that retain antigen binding ability.

When the antibody used in the methods used in the practice of the invention is a monoclonal antibody, the antibody is generated using any known monoclonal antibody preparation procedures such as those described, for example, in Harlow et al. (supra) and in Tuszynski et al. (Blood 1988, 72:109-115). Generally, monoclonal antibodies directed against a desired antigen are generated from mice immunized with the antigen using standard procedures as referenced herein. Monoclonal antibodies directed against full length or fragments of target structure may be prepared using the techniques described in Harlow et al. (supra).

Chimeric animal-human monoclonal antibodies may be prepared by conventional recombinant DNA and gene transfection techniques well known in the art. The variable region genes of a mouse antibody-producing myeloma cell line of known antigen-binding specificity are joined with human immunoglobulin constant region genes. When such gene constructs are transfected into mouse myeloma cells, the antibodies produced are largely human but contain antigen-binding specificities generated in mice. As demonstrated by Morrison et al., 1984, Proc. Natl. Acad. Sci. USA 81:6851-6855, both chimeric heavy chain V region exon (VH)-human heavy chain C region genes and chimeric mouse light chain V region exon (VK)-human K light chain gene constructs may be expressed when transfected into mouse myeloma cell lines. When both chimeric heavy and light chain genes are transfected into the same myeloma cell, an intact H2L2 chimeric antibody is produced. The methodology for producing such chimeric antibodies by combining genomic clones of V and C region genes is described in the above-mentioned paper of Morrison et al., and by Boulianne et al. (Nature 1984, 312:642-646). Also see Tan et al. (J. Immunol. 1985, 135:3564-3567) for a description of high level expression from a human heavy chain promotor of a human-mouse chimeric K chain after transfection of mouse myeloma cells. As an alternative to combining genomic DNA, cDNA clones of the relevant V and C regions may be combined for production of chimeric antibodies, as described by Whitte et al. (Protein Eng. 1987, 1:499-505) and Liu et al. (Proc. Natl. Acad. Sci. USA 1987, 84:3439-3443). For examples of the preparation of chimeric antibodies, see the following U.S. Pat. Nos. 5,292,867; 5,091,313; 5,204,244; 5,202,238; and 5,169,939. The entire disclosures of these patents, and the publications mentioned in the preceding paragraph, are incorporated herein by reference. Any of these recombinant techniques are available for production of rodent/human chimeric monoclonal antibodies against target structures.

When antibodies other than human antibodies are modified for incorporation into chimeric compounds pursuant to the present invention, it may be necessary to reduce the immunogenicity of the murine antibody. To further reduce the immunogenicity of murine antibodies, “humanized” antibodies have been constructed in which only the minimum necessary parts of the mouse antibody, the complementarity-determining regions (CDRs), are combined with human V region frameworks and human C regions (Jones et al., 1986, Nature 321:522-525; Verhoeyen et al., 1988, Science 239:1534-1536; Hale et al., 1988, Lancet 2:1394-1399; Queen et al., 1989, Proc. Natl. Acad. Sci. USA 86:10029-10033). The entire disclosures of the aforementioned papers are incorporated herein by reference. This technique results in the reduction of the xenogeneic elements in the humanized antibody to a minimum. Rodent antigen binding sites are built directly into human antibodies by transplanting only the antigen binding site, rather than the entire variable domain, from a rodent antibody. This technique is available for production of chimeric rodent/human anti-target structure antibodies of reduced human immunogenicity.”

The term antibody fragment or “FAB” is used to describe a fragment of an antibody which substantially maintains the same binding characteristics of the whole antibody, but eliminates other chemical features of the antibody which may complicate administration and produce untoward immunogenic responses in a patient.

The term “single-chain antibody variable fragment” or “scFv” is used to describe an artificial construct that links the sequences encoding the V_{H} and V_{L} domains of an antibody into a single polypeptide chain and lacks the rest of the antibody molecule. Because the antigen-binding site of an antibody is formed in a cavity at the interface between V_{H} and V_{L} domains, the scFv preserves the antigen binding activity of the intact antibody molecule. Normally the V_{H} and V_{L} domains are parts of different polypeptide chains (the heavy and light chains, respectively), but in the scFv they are joined into a single polypeptide that can be fused genetically to other proteins, for example, proteins on cancer cells to be targeted. These scFvs may form the basis of effective CCTMs on chimeric compounds according to the present invention.

The term “linker” (designated as “L”, “(L)”, “L_{C}” or (L_{C})” in compounds according to the present invention) is used to describe a chemical moiety which, when present in chimeric molecules according to the present invention, covalently binds a (ACM) group to a (CCT_{E}) group, optionally through one or more [CON] groups and/or one or more alternative linker groups. The linker group may be cleavable or noncleavable depending on the function of the CCT_{E} group or the compound itself (in the case of dimeric compounds according to the present invention). In general, antibody or antibody related (CCT_{E}) groups described above are generally, but not exclusively linked to a (ACM) group through a cleavable linker group. Other CCT_{E}s often are linked to (ACM) groups through a non-cleavable linker group.

Typical cleavable linker groups (L), which may be represented as (L_{CL}), for use in the present invention are represented by any chemical structure which is compatible with the chemistry of the chimeric compounds and their administration to a patient and readily cleave in or on a cell in which the chimeric molecule is introduced. In general, the cleavable linker for use in compounds according to the present invention is at least one chemical moiety, more often at least two chemical moieties in length to upwards of 100 or more moieties in length. These linkers are presented in detail hereinbelow. Often, one or more linkers, especially cleavable linker groups may be linked to one or more non-cleavable (non-labile) linker groups either directly or through a connector group (CON) or multiconnector group (MULTICON) as otherwise described herein. These form a more complex linker group.

Cleavable or labile linkers (L_{CL}) allow the [ACM] moiety to be cleaved from the (CCTM) in compounds according to the present invention order to provide a maximal effect in the cell, by allowing the ACM to be cleaved from the CCTM after the compound targets the cancer cell, facilitating entry of the ACM into the cell which causes cleavage/breakage and/or intercalation of the cell's DNA, causing cytotoxicity and cell death. These labile linkers include hydrolytically labile (acid labile) linkers, reductively labile linkers (principally disulfide linkers which are reductively cleaved by intracellular glutathione or other disulfide reducing agent) and enzymatically labile linkers (protease substrates).

In certain embodiments according to the present invention, the cleavable linker L_{CL} is a disulfide wherein one of the sulfurs in the disulfide group is provided by a cysteinyl residue alone or as an oligopeptide ranging from about 1 to about 10 amino acid units in length, often 1, 2 or 3 amino acid units in length. In certain embodiments the oligopeptide is represented by a glutamyl cysteinyl dipeptide (with the amide formed between the sidechain carboxylic acid of the glutamic acid and the amine of the cysteinyl residue), a glycinyl cysteinyl dipeptide, an alaninyl cysteinyl dipeptide or a lysinyl cystinyl dipeptide. The dipeptide may be linked (mated) with another dipeptide of similar or different structure each having a cysteinyl residue linked to the cysteinyl residue of the other dipeptide, or the dipeptide may be linked with a mercaptide such as an alkyl mercaptide (which is further substituted with a group which can further link the cleavable linker to another group, such as a non-cleavable (non-labile) linker an (ACM) group or a (CCT_{E}) group or a connector group, etc.

In other embodiments the cleavable linker group (L_{CL}) is an oligopeptide (containing a disulfide group as described above) or other linker which contains an ester group which may readily cleaved. For example, a linker may consist of a dipeptide such as a glutamyl cysteinyl group which provides a disulfide link to a linker (such as a alkylene group or polyethylene glycol group) which can form an integral connector molecule (such as a difunctional triazole CON group or a MULTICON group) as otherwise described herein, or alternatively bind directly to an ACM group or a CCTM group.

Cleavable or labile linkers (L_{C}) may comprise a group represented by the chemical structures:

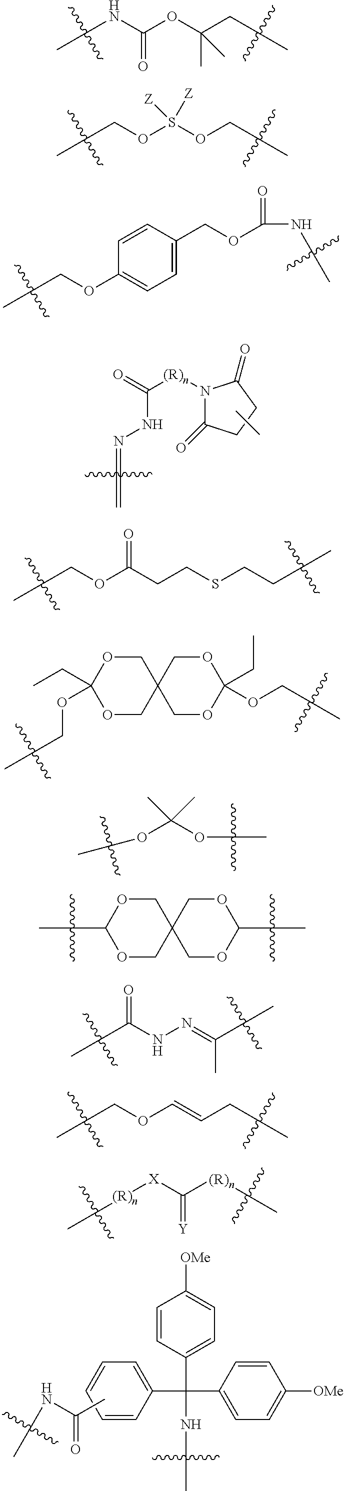

where R is an ethylene glycol group, a methylene group or an amino acid, preferably an ethylene glycol group or an amino acid and n in this labile linker is from 0 to 10, often from 1 to 6, or 1 to 3 and where points of attachment (as indicated) are to other portions of the cleavable or labile linker (L_{C}), a difunctional connector moiety (CON), a non-cleavable (non-labile) linker (L_{N}), or a multifunctional connector molecule [MULTICON], through which an [ACM] functional group and a [CCTM] functional group are linked as otherwise described herein;

### X is O, N—R^{AL} or S;

R^{AL} is H or a C₁-C₃ alkyl group (often H or Me, most often H);

### Y is O or S and

Z=Me, Et, iPr, tBu, Ph, each of which may be optionally substituted with one or more halogen groups (especially from three up to five Fs, preferably no more than three Fs) and where said Ph group may be further optionally substituted with a C₁-C₃ alkyl group (which itself may be substituted with up to three halogens, preferably F) or OMe.

Exemplary reductively cleaved moieties (by glutathione, other reductive species within the cell) include moieties according to the chemical formula:

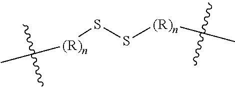

Where R is independently an ethylene glycol group, a methylene group or an amino acid where at least one amino acid (that which provides one of the sulfurs in the disulfide group) is a cysteinyl group (often, (R)n is a glutamyl cysteinyl or lysinyl cysteinyl dipeptide) and n in this labile linker is from 0 to 10, often from 1 to 6, or 1, 2 or 3 and where points of attachment (as indicated) are to other portions of the cleavable/labile linker [L_{CL}], a difunctional connector molecule or group (CON), a non-labile linker (NLL) or a multifunctional connector group molecule [MULTICON] as otherwise described herein.

Another reductively cleaved linker (L_{CL}) which is often used in compounds according to the present invention is represented by the following structure:

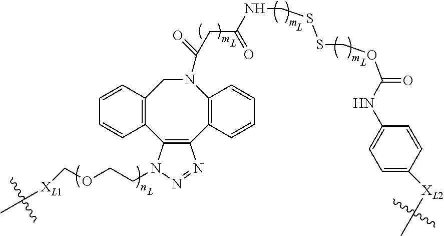

Where X_{L1} is —(CH₂)_{mL′}(C═O)—, —(CH₂)_{mL″}, NR_{1L}, NR_{1L}(C═O), S, S═O or S(O)₂, or a nucleophilic or electrophilic functional group (which can be further reacted to form a covalent link);
X_{L2} is —(CH₂)_{mL′}(C═O)—, —(CH₂)_{mL″}, —(CH₂)_{mL′}NR_{1L}—(CH₂)_{mL″}, NR_{1L}(C═O), S═O or S(O)₂, or a nucleophilic or electrophilic functional group (which can be further reacted to form a covalent link);
R_{1L} is H or a C₁-C₃ alkyl group;
Each m_{L} is independently 1, 2, 3, or 4 (often, each m_{L} is 2);
mL′ is 0, 1, 2, 3, 4, or 5 (preferably 0);
mL″ is 1, 2, 3, 4 or 5; and
n_{L} is 0-20, 1-15, 2-10, 1-6, 1, 2, 3, 4, 5, 6, 7, or 8.

In certain embodiments of the above compounds as described above. X_{L1} and X_{L2} are optionally functional groups on the linker moiety (pre-linker molecules), for example, nucleophilic and/or electrophilic groups which are reactive with a corresponding electrophilic and/or nucleophilic group on the ACM, CCT_{E}, [CON] group or another linker molecule so that the ACM group, CCT_{E}, [CON] group or another linker molecule can be covalent linked or coupled to the linker group. In certain embodiments, X_{L1} and/or X_{L2} groups are nucleophilic groups such as amine groups, hydroxyl groups, sulfhydryl groups or nucleophilic carbon groups (e.g., carbanions) which couple and form covalent bonds with a corresponding electrophilic group such as an ester groups (which may be activated), acyl groups (activated), or other electrophilic groups such as trichloromethylmethyliminoester (—O—C═NH(CCl₃)), among others, on the ACM, CCT_{E}, [CON] moiety or alternative linker molecule. Alternatively, X_{L1} and/or X_{L2} may be nucleophilic groups such as an amine group, a hydroxyl group or a sulfhydryl group which are reactive with a corresponding electrophilic groups as described above. In these pre-linker molecules, each of X_{L1} and X_{L2} may be a nucleophilic and an electrophilic group. This approach applies to all linkers provided herein which may be presented as prelinker compounds capable of coupling with functional groups on the ACM, CCT_{E}, [CON] or alternative linker components of the present compounds.

In certain embodiments, the reductively cleaved linker (L_{CL}) is a moiety according to the chemical structure:

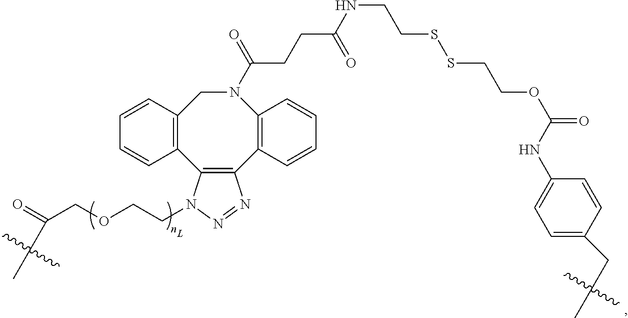

### Where n_{L} is 0-20, 1-15, 2-10, 1-6, 1, 2, 3, 4, 5, 6, 7, or 8.

In certain alternative embodiments, a partial cleavage linker containing an alkynyl containing functional group (which ultimately forms a connector group) is according to the chemical structure:

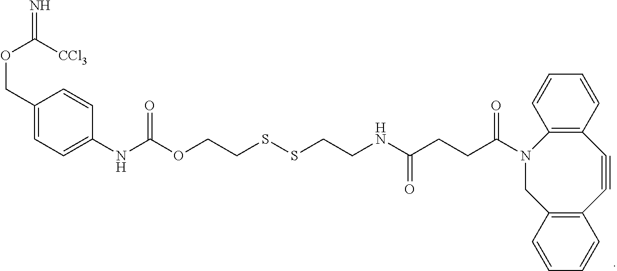

This linker may be reacted with other components (which may include CCT_{E} groups, ACM groups, [CON] groups or alternative linkers containing a hydroxyl group to react with the trichloromethylmethyliminoester (—O—C═NH(CCl₃) functional group at one end of the molecule and an azide group at the alkynyl functionality according to the present invention to form exemplary compounds according to the present invention.

In alternative versions of this approach, the linker molecule is according to the chemical structure:

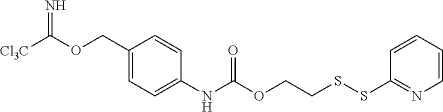

Where the trichloromethylmethyliminoester (—O—C═NH(CCl₃) functional group may be used to covalently link a hydroxyl group to form an ether and the disulfide group is used to bind to a cysteinyl group of an antibody or other oligo- or polypeptide.

Exemplary enzymatically cleaved labile linkers include those according to the chemical structure:

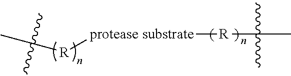

Where the protease (cathepsin) substrate is a peptide containing from 2 to 50 amino acid units or more, often 2 to 25 amino acid units, 2 to 15 amino acid units, 2 to 10 amino acid units, 2 to 6 amino acids, 2 to 4 amino acids, 2, 3 or 4. Often, the protease substrate, above contains, comprises, consists essentially of or consists of the following peptides the point of attachment being at the distal ends of the peptide:
Gly-Phe-Leu-Gly-;
Ala-Leu-Ala-Leu;
Phe-Arg-;
Phe-Lys-;
Val-Cit- (valine-citrillune)
Val-Lys-
Val-Ala- and
   where R (above) is an ethylene glycol group, or a methylene group and n is from 0 to 10, often from 1 to 6, or 1 to 3 and where points of attachment (as indicated) are joined to other portions of the labile linker, a difunctional connector group or molecule (CON), a non-labile linker (NLL) or other moiety as described herein.

Other enzyme labile linkers are the beta-glucosidase labile linkers according to the chemical structure:

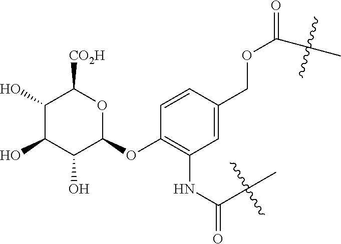 [SMILES: *C(=O)Nc1cc(COC(*)=O)ccc1O[C@@H]1O[C@H](C(=O)O)[C@@H](O)[C@H](O)[C@H]1O]

Where the points of attachment are joined to other portions of the labile linker, a difunctional connector moiety (CON), a non-labile linker (NLL) or a multifunctional connector group or molecule [MULTICON] as otherwise described herein.

In each of the above labile linkers, at the point of attachment in each group, the labile linker may be further linked to a non-labile linker as otherwise described herein, preferably a (poly)ethylene glycol group of from 1 to 12 glycol units (often 2 to 8 glycol units or 4 to 6 units) or an alkylene chain from 1 to 20 methylene units, often 1 to 10 methylene units, often 1 to 8 methylene units, more often 1 to 6 methylene unit, often 2 to 4 methylene units.

Preferred non-labile linkers include, for example, (poly)ethylene glycol linkers ranging in length from 2 to about 100 ethylene glycol units, preferably about 2 to 10 ethylene glycol units, about 2 to about 25, about 2 to about 15, about 2 to about 14, about 4 to about 10 units. In other preferred embodiments, the non-cleavable linker (L_{N}) is a polyethylene-co-polypropylene (PEG/PPG block copolymer) linker ranging from 2 to about 100, about 2 to about 25, about 2 to about 15, about 2 to about 14, about 2 to about 10, about 4 to about 10, combined ethylene glycol and propylene glycol units.

(Poly)alkylene chains as otherwise described herein are also preferred L_{N} for use in the present invention. When present, these have 1 to about 100 units, often about 2 to 10 units, about 2 to about 25, about 2 to about 15, about 2 to about 14, about 4 to about 10 units. L_{N} for use in the present invention may also contain one or more connector CON moieties as otherwise described herein which chemically connect separate (two or more) L_{N} portions, the entire portion being labeled L_{N}. In addition, a non-cleavable linker L_{N} may be linked through at least one connector moiety CON (as described in greater detail herein) to a cleavable linker L_{C} in order to provide a linker moiety.

In certain preferred embodiments, the non-labile linker (NLL) is represented by the following exemplary structures (note that the NLL may contain one ore more CON moieties as discussed above):

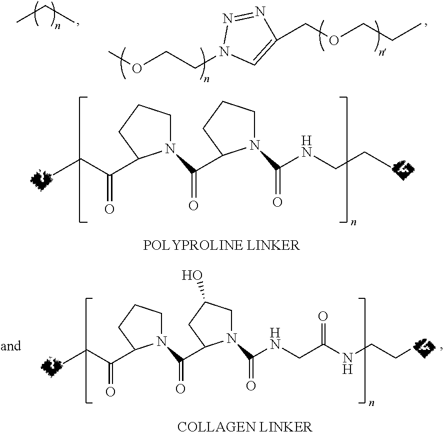

among numerous others, as described herein.
where n and n′ are each independently 0 to 100, preferably 1 to 100, more preferably about 2 to about 20, about 2 to about 10, about 4 to about 10, about 4 to about 8.

The non-labile linker group NLL may also be a linker according to the chemical formula:

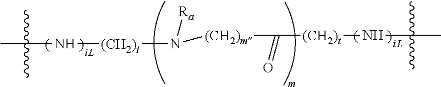

where Rₐ is H or a C₁-C₃ alkyl, preferably CH₃, most often H;
m is an integer from 1 to 12, often 1, 2, 3, 4, 5, or 6;
m″ is an integer 1, 2, 3, 4, 5, or 6, often 6;
t is 0, 1, 2, 3, 4, 5, or 6; and
iL is 0 or 1, often 1; or
a linker according to the structure:

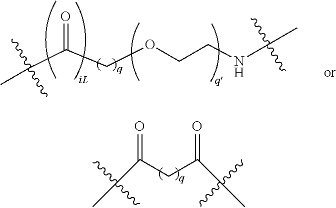

Where q is an integer from 0-12, preferably 1, 2, 3, 4, 5 or 6;
q′ is 1 to 12, often 1, 2, 3, 4, 5 or 6 and
iL is 0 or 1, preferably 1.

The two above linkers may be linked together to provide further linkers which are often used in compounds according to the present invention:

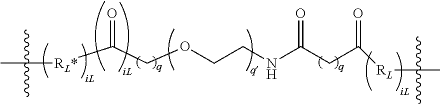

Where q is an integer from 0-12, preferably 0, 1, 2, 3, 4, 5 or 6;
q′ is 1 to 12, often 1, 2, 3, 4, 5 or 6;
iL is 0 or 1; and
R_{L} is an amino acid or an oligopeptide (which term includes a dipeptide) as otherwise described herein, especially including lysine, dilysine, or glycinelysine.

Another linker according to the present invention includes a linker based upon succinimide according to the chemical formula:

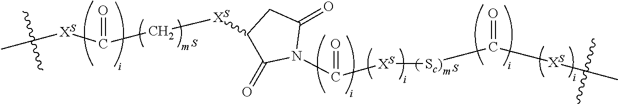

where each X^{S} is independently a bond, S, O or N—R^{S}, preferably S;
R^{S} is H or C₁₋₃ alkyl, preferably H;
S_{c} is CH₂, CH₂O; or CH₂CH₂O;
i is 0 or 1; and
m^{S} is 0, 1, 2, 3, 4, 5, 6, 7, 8, 9 or 10 (preferably 1-5).

In certain additional embodiments, the linker group is an amino acid, a dipeptide or an oligopeptide containing from 1 to 12, preferably 1 to 6 amino acid monomers or more. In certain embodiments, the oligopeptide is a dipeptide and the dipeptide is a dilysine or a glycinelysine dipeptide. When lysine is used as an amino acid in an oligopeptide linker, the sidechain alkylene amine may be used to link other linker groups or other components in the molecule. The dipeptide or oligopeptide may be considered a cleavable linker or non-cleavable depending upon the nature of the peptide.

In certain additional embodiments, as discussed above, the linker group NLL is a group

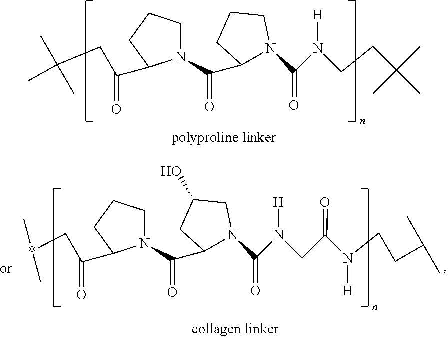

a group

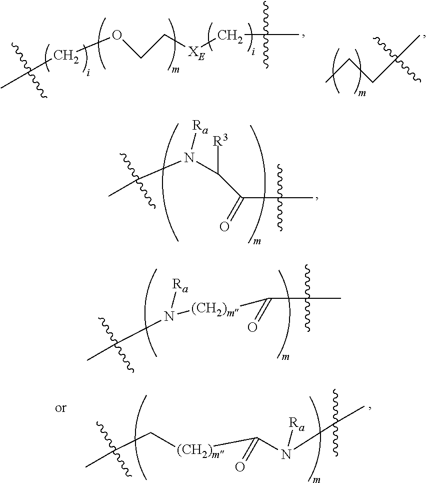

or a polypropylene glycol or polypropylene-co-polyethylene glycol linker having between 1 and 100 glycol units (1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 52 and 50, 3 and 45);
Where Rₐ is H, C₁-C₃ alkyl or alkanol or forms a cyclic ring with R³ (proline) and R³ is a side chain derived of an amino acid preferably selected from the group consisting of alanine (methyl), arginine (propyleneguanidine), asparagine (methylenecarboxyamide), aspartic acid (ethanoic acid), cysteine (thiol, reduced or oxidized di-thiol), glutamine (ethylcarboxyamide), glutamic acid (propanoic acid), glycine (H), histidine (methyleneimidazole), isoleucine (1-methylpropane), leucine (2-methylpropane), lysine (butyleneamine), methionine (ethylmethylthioether), phenylalanine (benzyl), proline (R³ forms a cyclic ring with Rₐ and the adjacent nitrogen group to form a pyrrolidine group), serine (methanol), threonine (ethanol, 1-hydroxyethane), tryptophan (methyleneindole), tyrosine (methylene phenol) or valine (isopropyl);
X_{E} is a bond, 0, N—R_{NA}, or S;
R_{NA} is H or C₁-C₃ alkyl, preferably H;
i is an integer from 0 to 6 (0, 1, 2, 3, 4, 5, or 6);
m″ is an integer from 0 to 25, preferably 1 to 10, 1 to 8, 1, 2, 3, 4, 5, or 6;
m is an integer from 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5; and
n is an integer from 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5; or
L may also be a linker according to the chemical formula:

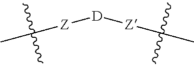

Where Z and Z′ are each independently a bond, —(CH₂)ᵢ—O, —(CH₂)ᵢ—S, —(CH₂)ᵢ—N—R,

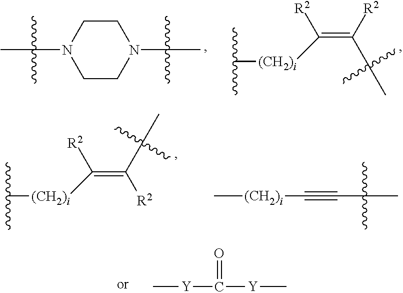

wherein said —(CH₂)ᵢ group, if present in Z or Z′, is bonded to [ACM], [CCT_{E}], or an optional difunctional connector group [CON], if present;
Each R is independently H, or a C₁-C₃ alkyl or alkanol group;
Each R² is independently H or a C₁-C₃ alkyl group;
Each Y is independently a bond, O, S or N—R;
Each i is independently 0 to 100, 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5;

### D is

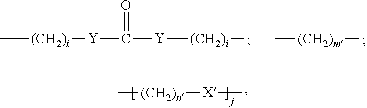

or a bond, or D may be

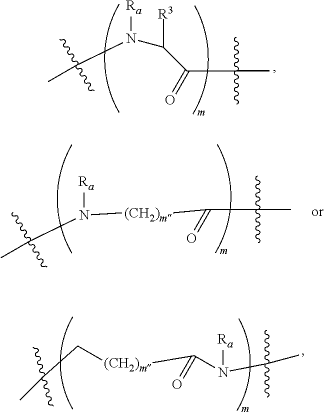

or a polypropylene glycol or polypropylene-co-polyethylene glycol linker having between 1 and 100 glycol units (1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 52 and 50, 3 and 45);
with the proviso that Z, Z′ and D are not each simultaneously bonds;
j is 1 to 100, 1 to 75, 1 to 60, 1 to 55, 1 to 50, 1 to 45, 1 to 40, 2 to 35, 3 to 30, 1 to 15, 1 to 10, 1 to 8, to 6, 1, 2, 3, 4 or 5;
m (within this context) is an integer from 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5; and
n (within this context) is an integer from about 1 to 100, about 1 to 75, about 1 to 60, about 1 to 50, about 1 to 45, about 1 to 35, about 1 to 25, about 1 to 20, about 1 to 15, 2 to 10, about 4 to 12, about 5 to 10, about 4 to 6, about 1 to 8, about 1 to 6, about 1 to 5, about 1 to 4, about 1 to 3, etc.).
m′ is 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5;
m″ is an integer between 0 to 25, preferably 1 to 10, 1 to 8, 0, 1, 2, 3, 4, 5, or 6;
n′ is 1 to 100, 1 to 75, 1 to 60, 1 to 55, 1 to 50, 1 to 45, to 40, 2 to 35, 3 to 30, 1 to 15, 1 to 10, 1 to 8, 1 to 6, 1, 2, 3, 4 or 5;

### X¹ is O, S or N—R;

R is as described above;
Rₐ is H, C₁-C₃ alkyl or alkanol or forms a cyclic ring with R³ (proline) and R³ is a side chain derived of an amino acid preferably selected from the group consisting of alanine (methyl), arginine (propyleneguanidine), asparagine (methylenecarboxyamide), aspartic acid (ethanoic acid), cysteine (thiol, reduced or oxidized di-thiol), glutamine (ethylcarboxyamide), glutamic acid (propanoic acid), glycine (H), histidine (methyleneimidazole), isoleucine (1-methylpropane), leucine (2-methylpropane), lysine (butyleneamine), methionine (ethylmethylthioether), phenylalanine (benzyl), proline (R³ forms a cyclic ring with Rₐ and the adjacent nitrogen group to form a pyrrolidine group), serine (methanol), threonine (ethanol, I-hydroxyethane), tryptophan (methyleneindole), tyrosine (methylene phenol) or valine (isopropyl).

It is noted that each of the linkers (both cleavable and non-cleavable linkers) identified in the present application may be further linked with connector molecules/moieties [CON] molecules/moieties, [ACM] groups and [CCT_{E}] groups through amide groups (which include alkylene groups on either or both sides of the amide group containing one to five methylene units), keto groups (which include alkylene keto groups containing one to five methylene units on either or both sides of the keto group), amine groups (which include alkylene amine groups containing one to five methylene units on either or both sides of the amine group), urethane groups (which include alkylene groups containing one to five methylene units on either or both sides of the urethane moiety), alkylene groups (containing from 1 to 5 methylene units), urea groups (which include alkylene groups containing one to five methylene units on either or both sides of the urethane moiety) amino acids, succinimide groups or other moieties compatible with the linker chemistry in order to link components of the molecules. It is noted that in the case of polyethylene glycol and polypeptide linkers, the use of an additional group (eg, alkylene amine or other group as described above) or a second linker group may be useful for joining the linker to another component of the molecule, including a [CON] group.

Additionally, more than one linker group identified herein may be linked together to form a linker group as otherwise used in the present compounds, consistent with the stability of the linker chemistries. These extended linkers are often, though not exclusively, linked through [CON] connecting groups as otherwise described herein.

In certain embodiments according to the present invention, linker molecules are provided which contain at least one and preferably at least two functional groups through which ACM, CCT_{E}, connector [CON] groups or even additional linker groups may be covalently linked to provide compounds according to the present invention. The functional groups are generally at or are proximally located at the distil ends of a linker molecule and may be electrophilic and/or nucleophilic groups or may be readily functionalized to functional groups (electrophilic and/or nucleophilic groups) which may be used to covalently link other molecules ACM moieties, CCT_{E} moieties, [CON] molecules or even additional linker molecules) to the linker molecule.

In certain embodiments, functional groups on the linker moiety, include, for example, nucleophilic and/or electrophilic groups which are reactive with a corresponding electrophilic and/or nucleophilic group on the ACM, CCT_{E}, group so that the ACM group or the CCT_{E} group can be covalent linked or coupled to the linker group. In certain embodiments, X_{L1} and/or X_{L2} groups are nucleophilic groups such as amine groups, hydroxyl groups, sulfhydryl groups, azide groups (for reaction with an alkynyl group to form triazole connector molecules) or nucleophilic carbon groups (e.g., carbanions) which couple and form covalent bonds with a corresponding electrophilic groups such as ester groups (activated), acyl groups (activated), or other electrophilic groups such as trichloromethylmethyliminoester (—O—C═NH(CCl₃)), or alkynyl groups (reactive with azide groups) among others, on the ACM or CCT_{E}. Alternatively, these functional groups may be used to link additional linker molecules, ACM and/or CCT_{E} groups through connector [CON] molecules.

Another difunctional linker molecule for use in the present invention is

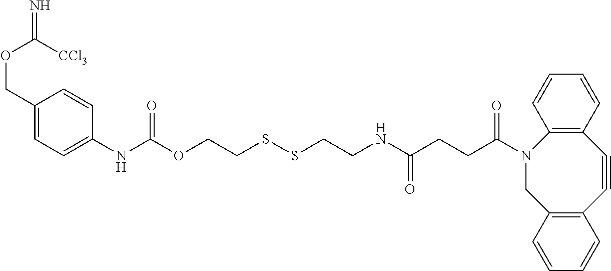

Where the trichloromethylmethyliminoester (—O—C═NH(CCl₃) functional group is reactive with a free hydroxyl group and the alkynyl group is reactive with an azido group to form a triazole connector [CON] moiety.

The term “difunctional connector group” or [CON] is used to describe a difunctional group which connects two (or more) portions of a linker group to extend the length of the linker group. In certain embodiments, a linker group is reacted with or forms a [CON] group with another linker group to form an extended linker group or with another moiety such as a ACM moiety or CCT_{E} moiety to link the linker to that moiety. The reaction product of these groups results in an identifiable connector group [CON] which may be distinguishable from the linker group as otherwise described herein, but is integral to same and essentially forms a portion of the linker group. It is further noted that there is often overlap between the description of the difunctional connector group and the linker group, especially with respect to more common connector groups such as amide groups, oxygen (ether), sulfur (thioether), carbonyl or amine linkages, urea or carbonate —OC(O)O— groups, etc. as otherwise described herein. It is noted that a difunctional connector molecule [CON] used hereunder is often connected to one or two parts of a linker group which binds [ACM] to [CCT_{E}]. Alternatively, a [CON] group may be directly linked to a [ACM] group or more often, a [CCT_{E}] group, as well as a [MULTICON] group as described herein. L CCT_{E}, CCT_{E} and/or ACM groups optionally include [CON] groups to facilitate the binding of a linker group to the CCT_{E} group and/or the ACM group.

Common difunctional connector groups [CON] which are used in the present invention, principally to link one end of a linker to another end of a linker to provide a longer linker or to connect a linker (and essentially become integral to the linker) to a ACM or CCT_{E}r group and include the following chemical groups or to:

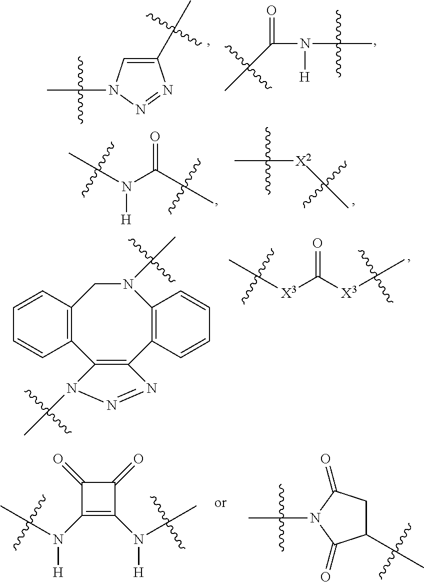

Where X² is CH₂, O, S, NR⁴, S(O), S(O)₂, —S(O)₂O, —OS(O)₂, or OS(O)₂O;
X¹ is absent, CH₂, O, S, NR⁴; and
R⁴ is H, a C₁-C₃ alkyl or alkanol group, or a —C(O)(C₁-C₃) group.

In certain embodiments, [CON] is a

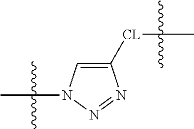

group;
where CL is

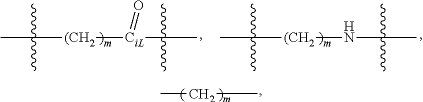

group, urethane or urea;
m in CL is an integer from 0 to 12, often 0, 1, 2, 3, 4, 5 or 6;
and iL is 0 or 1, often 1;

In other embodiments [CON] is a

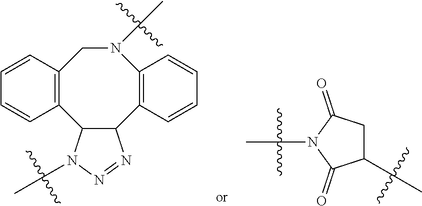 [SMILES: *N1Cc2ccccc2C2C(N=NN2*)c2ccccc21 ; *C1CC(=O)N(*)C1=O]

group.

In certain embodiments, the [CON] group is often linked through the amine of the triazole or the succinimide moiety to a cleavable or non-cleavable linker or to an ACM group or CCT_{E} group.

The term “multifunctional connector”, symbolized by [MULTICON], is used to describe a chemical group or molecule which is optionally included in chimeric compounds according to the present invention which link at least one or more linker groups (which may be cleavable or non-cleavable), difunctional connector groups (CON), (ACM) groups or (CCT_{E}) groups as otherwise described herein. The connector group is the resulting moiety which forms from the facile condensation of at least three separate chemical fragments which contain reactive groups which can provide connector groups as otherwise described to produce chimeric compounds according to the present invention. It is noted that a multifunctional connector moiety or molecule [MULTICON] is readily distinguishable from a linker in that the multifunctional connector is the result of a specific chemistry which is used to provide chimeric compounds according to the present invention.

Connecting moieties in the present invention include at least one multifunctional moiety or molecule [MULTICON] which contains three or more functional groups which may be used to covalently bind (preferably, through a linker) to at least one [ACM] group (preferably more than one) and at least one [CCT_{E}] group (preferably more than one), thus linking each of these functional groups into a single compound. Multifunctional connector groups for use in the present invention include moities which have at least three or more functional groups which can bind to linkers to which are bound [ACM] and/or [CCT_{E}] groups in order to provide compounds which contain at least one [ACM] and [CCT_{E}] groups, but preferably more than one of each of these groups pursuant to the present invention. These multifunctional connector moieties may also bind to other multifunctional connector molecules in order to create compounds containing a number of [ACM] and [CCT_{E}] groups as defined herein.

Multifunctional connector molecules [MULTICON] comprise any molecule or moiety which contains at least three groups which may be linked to [ACM], [CCT_{E}] and/or linkers (non-labile linkers or labile linkers) and/or other connector groups (including difunctional and multifunctional connector groups) and often comprise five or six-membered aryl or heteroaryl groups (especially six-membered ring groups) exemplified by multifunctional, especially trifunctional or tetrafunctional aryl or heteroaryl groups, including phenyl, pyridyl, pyrimidinyl, 1,3,5-triazinyl, 1,2,3-triazinyl, 1,2,4-triazinyl groups, each of which is substituted with at least 3 and up to 6 functional groups. These functional groups may be derived from nucleophilic or electrophilic groups on the multifunctional connector molecule precursor (the multifunctional connector molecule which forms the [MULTICON] moiety in final compounds according to the present invention) which are condensed onto linker groups (each of which contains, for example an [ACM] group or a [CCT_{E}] group) which contains a group which can be linked to the [MULTICON] moiety. [MULTICON] groups which are used in the present invention preferably include substituted phenyl, pyridyl, pyrimidinyl and 1,3,5-triazinyl, 1,2,3-triazinyl, 1,2,4-triazinyl groups, and other groups of multifunctionality especially including groups according to the chemical structure:

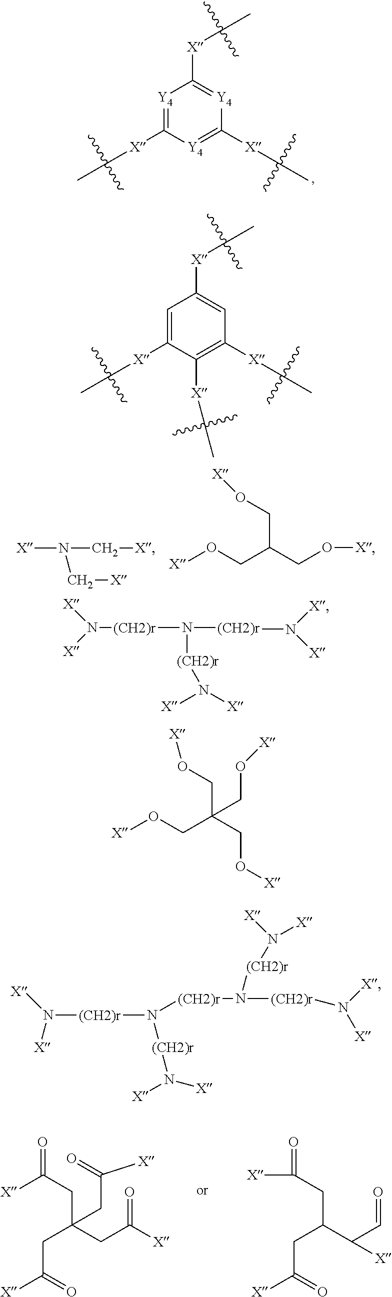

where Y₄ is C—H or N; and
Each X″ is independently derived from an electrophilic or nucleophilic group, preferably (CH₂)ₙ—O, (CH₂)_{n″}NR_{CON}, (CH₂)ₙ—S,
(CH₂)n— or (CH₂)_{n″}C═O;
the substitutent R^{CON} is H or a C₁-C₃ alkyl, preferably H or CH₃,
n″ is 0, 1, 2 or 3 and
r is an integer from 1-12, often 1, 2, 3, 4, 5 or 6.

The term “pharmaceutically acceptable salt” or “salt” is used throughout the specification to describe a salt form of one or more of the compositions herein which are presented to increase the solubility of the compound in saline for parenteral delivery or in the gastric juices of the patient's gastrointestinal tract in order to promote dissolution and the bioavailability of the compounds. Pharmaceutically acceptable salts include those derived from pharmaceutically acceptable inorganic or organic bases and acids. Suitable salts include those derived from alkali metals such as potassium and sodium, alkaline earth metals such as calcium, magnesium and ammonium salts, among numerous other acids well known in the pharmaceutical art. Sodium and potassium salts may be preferred as neutralization salts of carboxylic acids and free acid phosphate containing compositions according to the present invention. The term “salt” shall mean any salt consistent with the use of the compounds according to the present invention. In the case where the compounds are used in pharmaceutical indications, including the treatment of prostate cancer, including metastatic prostate cancer, the term “salt” shall mean a pharmaceutically acceptable salt, consistent with the use of the compounds as pharmaceutical agents.

The term “coadministration” shall mean that at least two compounds or compositions are administered to the patient at the same time, such that effective amounts or concentrations of each of the two or more compounds may be found in the patient at a given point in time. Although compounds according to the present invention may be co-administered to a patient at the same time, the term embraces both administration of two or more agents at the same time or at different times, provided that effective concentrations of all coadministered compounds or compositions are found in the subject at a given time. Chimeric antibody-recruiting compounds according to the present invention may be administered with one or more additional anti-cancer agents or other agents which are used to treat or ameliorate the symptoms of cancer, especially prostate cancer, including metastatic prostate cancer.

The term “anticancer agent” or “additional anticancer agent” refers to a compound other than the chimeric compounds according to the present invention which may be used in combination with a compound according to the present invention for the treatment of cancer. Exemplary anticancer agents which may be coadministered in combination with one or more chimeric compounds according to the present invention include, for example, antimetabolites, inhibitors of topoisomerase I and II, alkylating agents and microtubule inhibitors (e.g., taxol), among others. Exemplary anticancer compounds for use in the present invention may include everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab (Arzerra), zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, irinotecan, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258); 3-[5-(methylsulfonylpiperadinemethyl)-indolylj-quinolone, vatalanib, AG-013736, AVE-0005, the acetate salt of [D-Ser(Bu t) 6, Azgly 10] (pyro-Glu-His-Trp-Ser-Tyr-D-Ser(Bu t)-Leu-Arg-Pro-Azgly-NH₂ acetate [C₅₉H₈₄N₁₈Oi₄-(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, etbitux, EKB-569, PKI-166, GW-572016, lonafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, irinotecan, topotecan, doxorubicin, docetaxel, vinorelbine, bevacizumab (monoclonal antibody) and erbitux, cremophor-free paclitaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa, among others.

In addition to anticancer agents, a number of other agents may be coadministered with chimeric compounds according to the present invention in the treatment of cancer. These include active agents, minerals, vitamins and nutritional supplements which have shown some efficacy in inhibiting cancer tissue or its growth or are otherwise useful in the treatment of cancer. For example, one or more of dietary selenium, vitamin E, lycopene, soy foods, curcumin (turmeric), vitamin D, green tea, omega-3 fatty acids and phytoestrogens, including beta-sitosterol, may be utilized in combination with the present compounds to treat cancer.

Without not being limited by way of theory, anticancer compounds according to the present invention which contain a cancer cell targeting element (CCT_{E}) and an anticancer moiety (ACM) selectively bind to cancer cells and through that binding, facilitate the introduction of the (ACM) moiety into the cancer cell selectively, where, the compound, inside the cell or during transport into the cancer cell, the cleavable linker is cleaved from the cancer cell targeting moiety, providing an agent for intercalating and/or damaging through breakage the cancer cell's DNA and causing cell death.

Pharmaceutical compositions comprising combinations of an effective amount of at least one compound disclosed herein, often a difunctional chimeric compound (containing at least one ACM and at least one CCT_{E}) according to the present invention, and one or more of the compounds as otherwise described herein, all in effective amounts, in combination with a pharmaceutically effective amount of a carrier, additive or excipient, represents a further aspect of the present invention. These may be used in combination with at least one additional, optional anticancer agent as otherwise disclosed herein.

The compositions of the present invention may be formulated in a conventional manner using one or more pharmaceutically acceptable carriers and may also be administered in controlled-release formulations. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions of the present invention may be administered orally, parenterally, by inhalation spray, topically, rectally, nasally, buccally, vaginally or via an implanted reservoir, among others. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally (including via intubation through the mouth or nose into the stomach), intraperitoneally or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions of this invention may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically, especially to treat skin cancers, psoriasis or other diseases which occur in or on the skin. Suitable topical formulations are readily prepared for each of these areas or organs. Topical application for the lower intestinal tract can be effected in a rectal suppository formulation (see above) or in a suitable enema formulation. Topically-acceptable transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water.

Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of compound in a pharmaceutical composition of the instant invention that may be combined with the carrier materials to produce a single dosage form will vary depending upon the host and disease treated, the particular mode of administration. Preferably, the compositions should be formulated to contain between about 0.05 milligram to about 750 milligrams or more, more preferably about 1 milligram to about 600 milligrams, and even more preferably about 10 milligrams to about 500 milligrams of active ingredient, alone or in combination with at least one additional compound which may be used to treat cancer, prostate cancer or metastatic prostate cancer or a secondary effect or condition thereof.

Methods of treating patients or subjects in need for a particular disease state or condition as otherwise described herein, especially cancer, comprise administration of an effective amount of a pharmaceutical composition comprising therapeutic amounts of one or more of the novel compounds described herein and optionally at least one additional bioactive (e.g. anti-cancer) agent according to the present invention. The amount of active ingredient(s) used in the methods of treatment of the instant invention that may be combined with the carrier materials to produce a single dosage form will vary depending upon the host treated, the particular mode of administration. For example, the compositions could be formulated so that a therapeutically effective dose of between about 0.01, 0.1, 1, 5, 10, 15, 20, 25, 30, 35, 40, 45, 50, 55, 60, 65, 70, 75, 80, 85, 90 or 100 mg/kg of patient/day or in some embodiments, greater than 100, 110, 120, 130, 140, 150, 160, 170, 180, 190 or 200 mg/kg of the novel compounds can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

A patient or subject (e.g. a male human) suffering from cancer can be treated by administering to the patient (subject) an effective amount of a chimeric compound according to the present invention including pharmaceutically acceptable salts, solvates or polymorphs, thereof optionally in a pharmaceutically acceptable carrier or diluent, either alone, or in combination with other known anticancer or pharmaceutical agents, preferably agents which can assist in treating cancer, including metastatic cancer or ameliorate the secondary effects and conditions associated with cancer. This treatment can also be administered in conjunction with other conventional cancer therapies, such as radiation treatment or surgery.

The present compounds, alone or in combination with other agents as described herein, can be administered by any appropriate route, for example, orally, parenterally, intravenously, intradermally, subcutaneously, or topically, in liquid, cream, gel, or solid form, or by aerosol form.

The active compound is included in the pharmaceutically acceptable carrier or diluent in an amount sufficient to deliver to a patient a therapeutically effective amount for the desired indication, without causing serious toxic effects in the patient treated. A preferred dose of the active compound for all of the herein-mentioned conditions is in the range from about 10 ng/kg to 300 mg/kg, preferably 0.1 to 100 mg/kg per day, more generally 0.5 to about 25 mg per kilogram body weight of the recipient/patient per day. A typical topical dosage will range from about 0.01-3% wt/wt in a suitable carrier.

The compound is conveniently administered in any suitable unit dosage form, including but not limited to one containing less than 1 mg, 1 mg to 3000 mg, preferably 5 to 500 mg of active ingredient per unit dosage form. An oral dosage of about 25-250 mg is often convenient.

The active ingredient is preferably administered to achieve peak plasma concentrations of the active compound of about 0.00001-30 mM, preferably about 0.1-30 μM. This may be achieved, for example, by the intravenous injection of a solution or formulation of the active ingredient, optionally in saline, or an aqueous medium or administered as a bolus of the active ingredient. Oral administration is also appropriate to generate effective plasma concentrations of active agent.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Oral compositions will generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or enteric agents.

The active compound or pharmaceutically acceptable salt thereof can be administered as a component of an elixir, suspension, syrup, wafer, chewing gum or the like. A syrup may contain, in addition to the active compounds, sucrose as a sweetening agent and certain preservatives, dyes and colorings and flavors.

The active compound or pharmaceutically acceptable salts thereof can also be mixed with other active materials that do not impair the desired action, or with materials that supplement the desired action, such as other anticancer agents, antibiotics, antifungals, antiinflammatories, or antiviral compounds. In certain preferred aspects of the invention, one or more chimeric antibody-recruiting compound according to the present invention is coadministered with another anticancer agent and/or another bioactive agent, as otherwise described herein.

Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

In one embodiment, the active compounds are prepared with carriers that will protect the compound against rapid elimination from the body, such as a controlled and/or sustained release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions or cholestosomes may also be pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin fACM of dried lipid on the surface of the container. An aqueous solution of the active compound are then introduced into the container. The container is then swirled by hand to free lipid material from the sides of the container and to disperse lipid aggregates, thereby forming the liposomal suspension.

### General Chemistry Overview

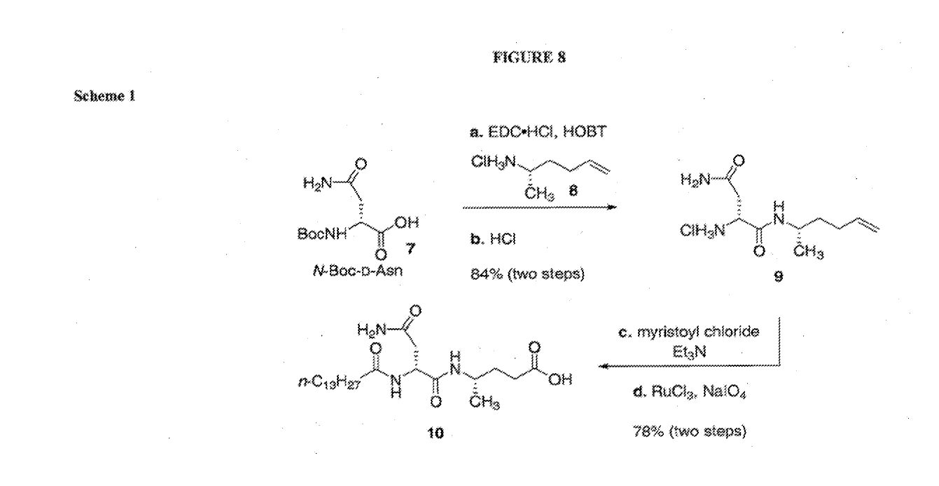

The synthetic route begins with N-myristoyl-D-asparagine (7), which was prepared in one step and high yield by acylation of D-asparagine with myristoyl chloride (FIG. 8, Scheme 1).¹⁵ EDCI-mediated coupling with (S)-hex-5-en-2-amine (8, prepared in three steps, from pent-4-en-al) provided the amide 9 (84%). Oxidative cleavage of the terminal alkene (ruthenium chloride, sodium periodate), then formed the carboxylic acid 10 (78%).

The thiazolinyl-thiazole side chain was prepared by the sequence shown in FIG. 9, Scheme 2A. Treatment of N-(tert-butoxycarbonyl)aminoacetonitrile (11) with L-cysteine methyl ester provided the thiazoline 12 (85%). Conversion of the ester to a primary amide (ammonia), followed by treatment with Lawesson's reagent, generated the thioamide 13 (>99%). Exposure of 13 to bromopyruvic acid in the presence of triethylamine formed the thiazolinyl-thiazole 14 (71%). Cleavage of the tert-butoxycarbonyl protective group (hydrochloric acid, dioxane) generated the amine 15 (>99%). Silver-mediated coupling (silver trifluoroacetate, triethylamine, 63%) of the amine 15 with the β-ketothioester 16 (prepared in one step from N-(tert-butoxycarbonyl)-1-aminocyclopropane-1-carboxylate) followed by carbamate cleavage (hydrochloric acid, dioxane) furnished the thiazoline-thiazole fragment 17.

The isomeric thiazole-thiazolinyl side chain was prepared by a modified sequence (FIG. 9, Scheme 2B). Treatment of N-(tert-butoxycarbonyl)-2-aminoethanthioamide (18) with ethyl bromopyruvate formed the thiazole 19 (74%). Aminolysis of 19 (ammonia) followed by dehydration of the resulting primary amide (trifluoracetic anhydride, triethylamine, 69%) generated the nitrile 20. Coupling of 20 with L-cysteine formed the bicycle 21 (97%). Removal of the carbamate protecting group (hydrochloric acid, dioxane) formed the amine 22 (>99%). Finally, coupling of 22 with the β-ketothioester 16 (silver trifluoroacetate, triethylamine, 69%), followed by carbamate cleavage (hydrochloric acid, dioxane) provided the thiazole-thiazoline 23.

To complete the synthesis of the precolibactin A skeleton, the carboxylic acid 10 was first converted to the β-ketothioester 24 by activation with carbon diimidazole followed by treatment with 3-(tert-butylthio)-3-oxopropanoic acid in the present magnesium ethoxide. Silver-mediated coupling of 24 with the thiazoline-thiazole 17 or the thiazole-thiazoline 23 formed the cyclization precursors 25 and 26, respectively (high yield from 10 for 25 and 26, respectively). Alternatively, the approach to 25 and 26 proceed through compound 19 as per Scheme 2C of FIG. 9.

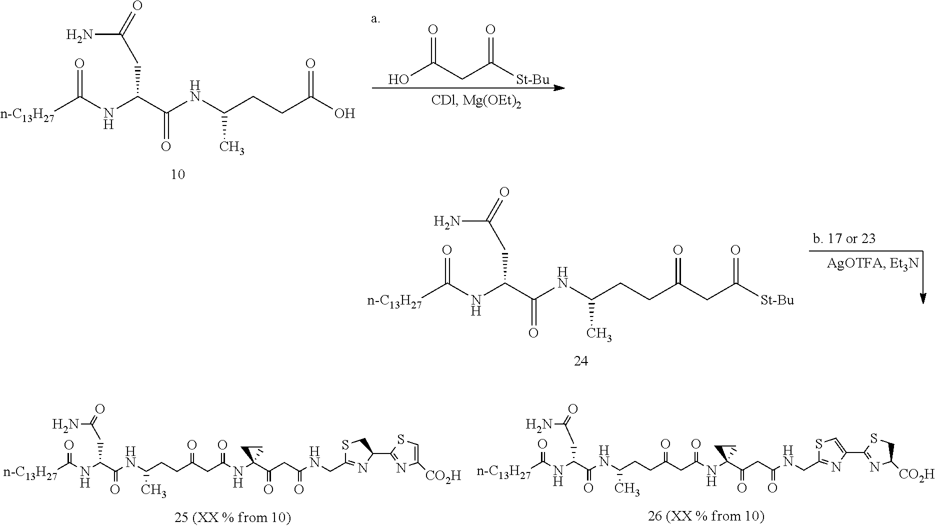

Assembly of the acyclic precolibactin A precursors 25 and 26 proceeded as per FIG. 9, Scheme 9C. The inventors then studied the cyclization of the thiazole-thiazoline intermediate 26. Under a variety of conditions, 26 was found to rapidly cyclize to generate the pyridone 28. We speculated that 28 is formed.

TABLE 1
Cyclization of the linear precursor 26.

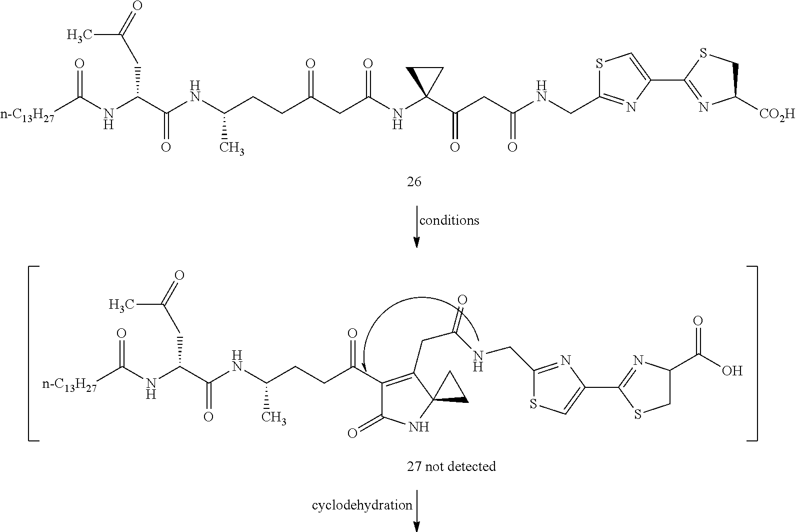

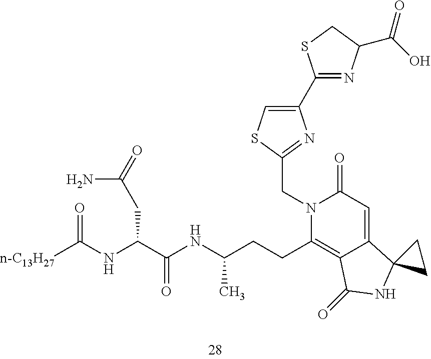

| Entry | Conditions | yield |
|---|---|---|
| 1 | 10% NaOH (aq.), ° C. | |
| 2 | NH4OAc, EtOH, ° C. | |
| 3 | K₂CO₃, EtOH, ° C. | |

A more detailed description of the synthetic chemistry and related experiments associated with the present invention (e.g. alkylation of DNA, etc.) appears herein below.

### Chemical Synthesis and Related Experiments

The colibactins are hybrid polyketide-nonribosomal peptide natural products produced by certain strains of commensal and extraintestinal pathogenic E. coli. The metabolites are encoded by the clb gene cluster as pro-drugs termed precolibactins. clb⁺ E. coli induce DNA double-strand breaks (DSBs) in mammalian cells in vitro and in vivo and are found in 55-67% of colorectal cancer patients, suggesting that mature colibactins could initiate tumorigenesis. However, elucidation of their structures has been an arduous task as the metabolites are obtained in vanishingly small quantities (μg/L) from bacterial cultures and are believed to be unstable. In the present invention, the inventors describe a flexible and convergent synthetic route to prepare advanced precolibactins and derivatives. The synthesis proceeds by late-stage union of two complex precursors (e.g., 28+17→29a, 90%) followed by a base-induced double dehydrative cascade reaction to form two rings of the targets in high yield (e.g., 29a→30a, 79%). The sequence has provided quantities of advanced candidate precolibactins that exceed those obtained by fermentation, and is envisioned to be readily-scaled. These studies have guided a structural revision of the predicted metabolite precolibactin A (from 5a or 5b to 7) and have confirmed the structures of the isolated metabolites precolibactins B (3) and C (6). Synthetic precolibactin C (6) was converted to N-myristoyl-D-Asn and its corresponding colibactin by colibactin peptidase ClbP. The synthetic strategy outlined herein will facilitate mechanism of action and structure-function studies of these fascinating metabolites, and is envisioned to accommodate the synthesis of additional (pre)colibactins as they are isolated.

Bacteria residing in and on humans (the human microbiota) play an integral role in regulating physiology and disease.¹ The intestinal tract has been estimated to contain 500-1000 species of bacteria constituting −1.5 kg of biomass.² Certain strains of gut commensal and extraintestinal pathogenic E. coli harbour a gene cluster (clb or “pks”) that encodes a group of molecules termed precolibactins.³ Precolibactins are substrates for colibactin peptidase ClpP, a protease encoded within the clb gene cluster. ClbP is anchored within the inner periplasmic membrane of the bacteria⁴ and removes an N-acyl-D-asparagine side chain from the precolibactins. This cleavage step converts precolibactins to cytotoxic colibactins and likely constitutes a prodrug resistance mechanism in the bacteria.⁵ clb⁺ E. coli induce DNA double-strand breaks (DSBs) in mammalian cells in viiro^{3a} and in vivo.⁶ Host inflammation promotes proliferation of E. coli⁷ and expression of clb,⁸ the clb pathway promotes colorectal cancer in colitis-susceptible mice treated with azoxymethane,⁷ and two studies revealed the presence of clb⁺ E. coli in 55-67% of colorectal cancer patients.^{7,9} Collectively, these data suggest that colibactins initiate tumorigenesis by a mechanism involving induction of DNA DSBs.

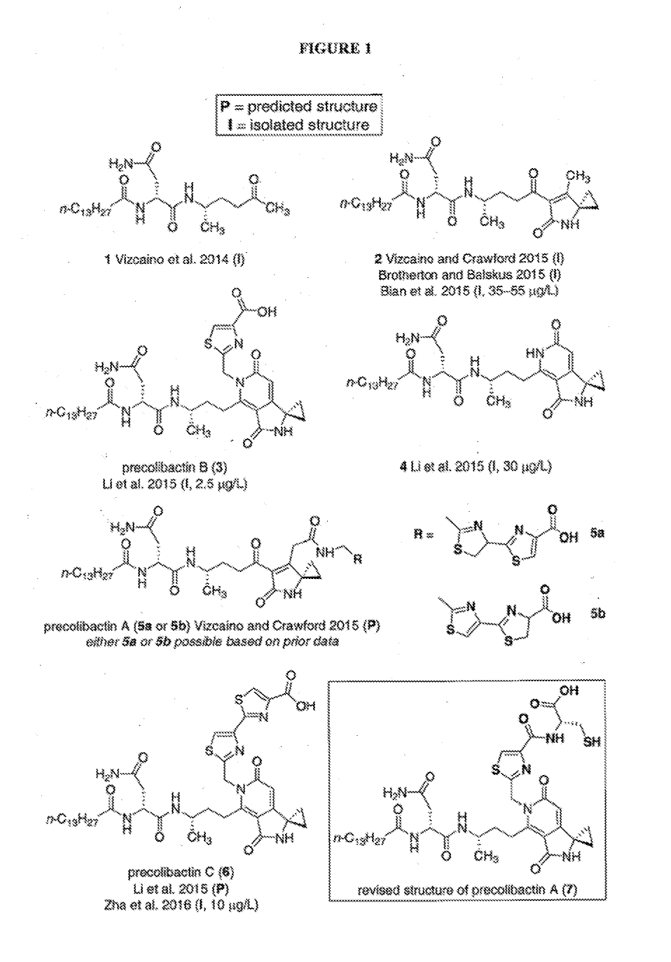

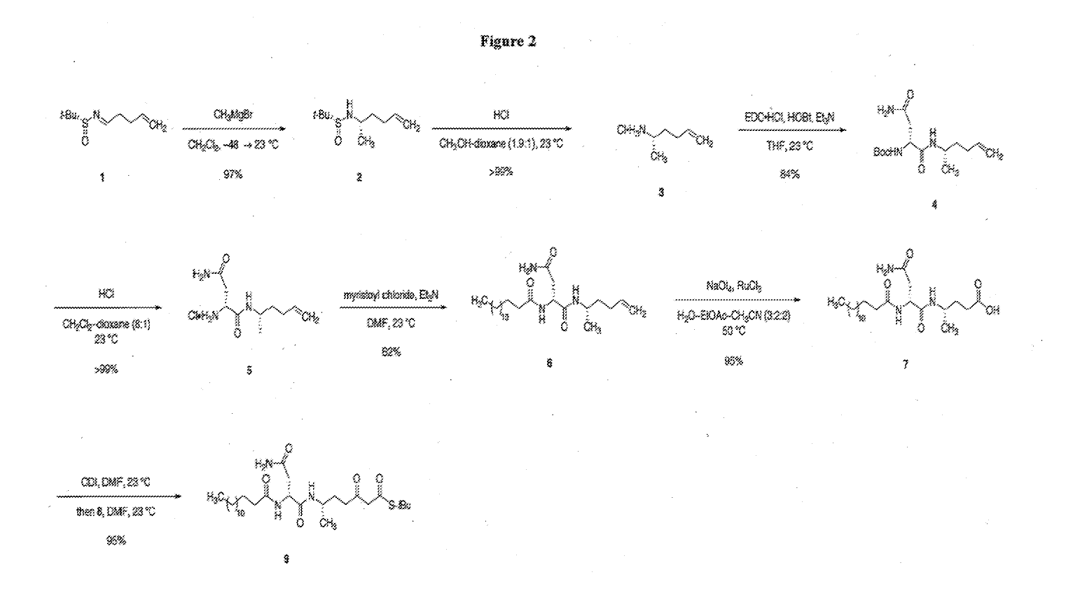

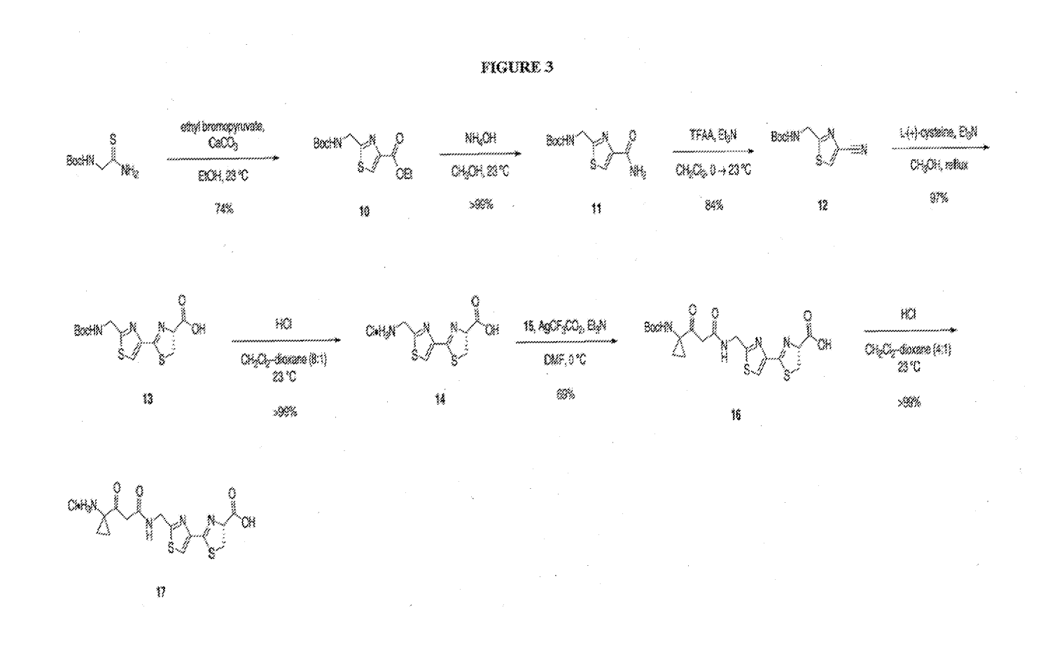

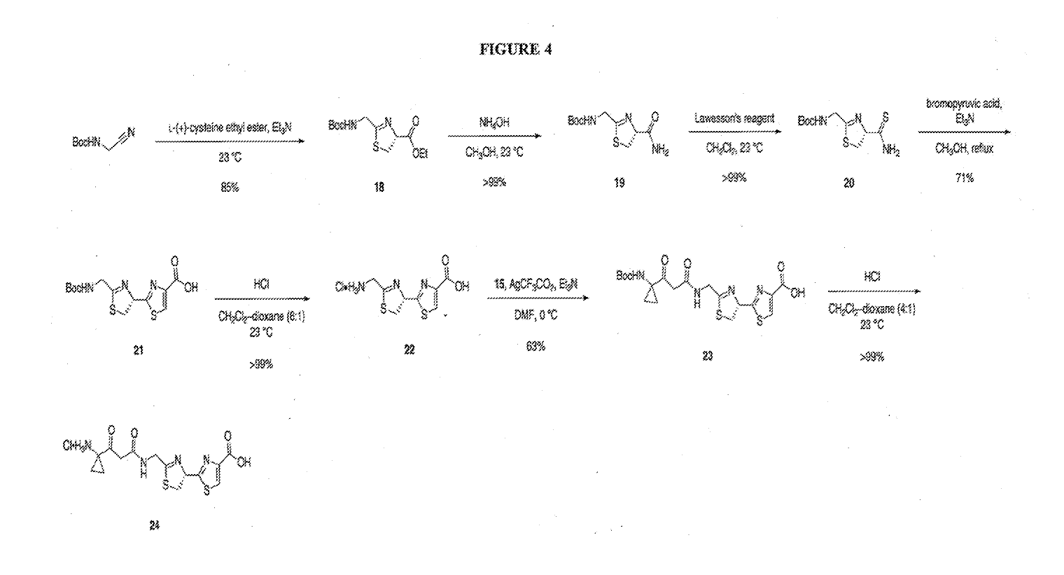

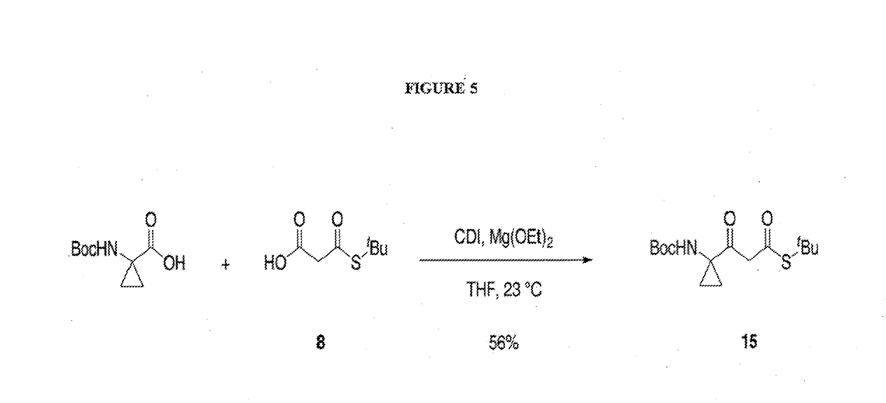

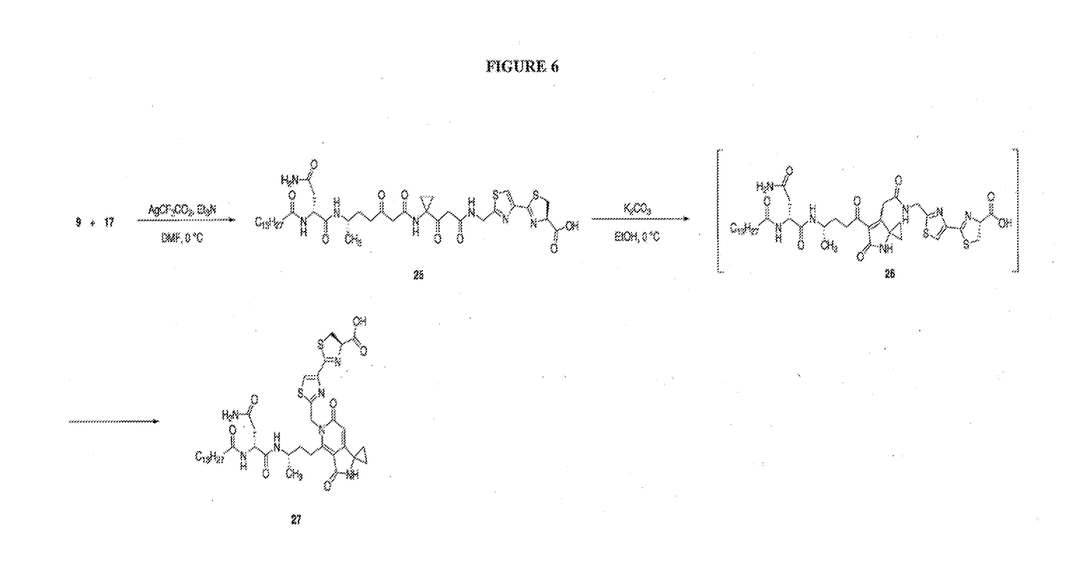

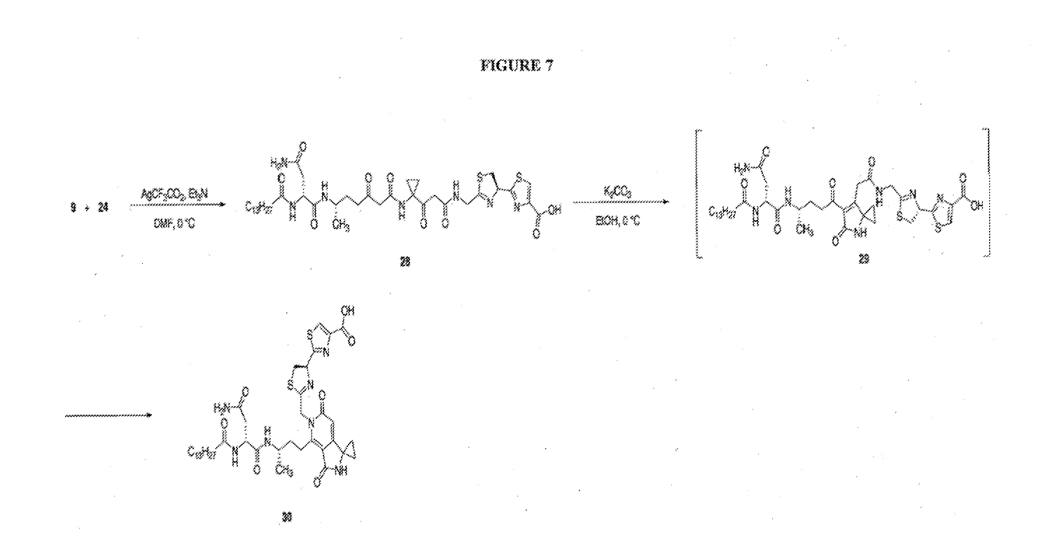

Fully elaborated (pre)colibactins have been difficult to isolate in homogenous form, and the definitive structures of the most active metabolite(s) are not known. This has been attributed to the low levels of natural production of the metabolites, their instability under fermentation conditions, and the inflammation-dependant up-regulation of the native clb gene cluster. The metabolites 1,^{5c} 2,¹⁰ 3 (referred to hereafter as “precolibactin B”),¹⁰ and 4¹⁰ were obtained in vanishingly small quantities (2.5-55 μg/L for 2-4) from the fermentation broth of genetically-engineered clb⁺ E. coli and implicated as shunt metabolites and/or degradation products in the colibactin biosynthetic pathway (FIG. 1). Using the isolation of 2, as well as HRMS analysis, isotope labelling, and bioinformatics based on established biosynthetic logic, the structure of precolibactin A was predicted as 5a or 5b.^{10a} Key elements within the proposed structures include a hydrophobic N-terminal fragment, a spirocyclic aminocyclopropane, and (read from left to right) a thiazoline-thiazole chain. As the presence of the thiazoline-thiazole fragment was inferred by bioinformatic analysis,^{10a} 5a and 5b could not be unequivocally distinguished at that time, and the absolute stereochemistry of the putative thiazoline ring was not determined. A compound with an exact mass corresponding to 5a was observed in unpurified extracts, but all efforts to isolate this structure were hampered by its low levels of production and instability.^{10a} The pyridone structure 6 (referred to hereafter as “precolibactin C”) was recently proposed as a candidate precolibactin based on biosynthetic considerations, isolation of precolibactin B (3), and HRMS analysis,¹¹ and during the preparation of this manuscript, Balskus and co-workers reported the isolation of precolibactin C (6) from a mutant strain (0.5 mg of 6 was obtained from an optimized 48-L fermentation).¹² Although one can envision cyclodehydration of 5a or 5b to form pyridones resembling 6, the biosynthetic relationship between these structures had not been established. 2 was shown to weakly cross-link DNA in vitro,^{10a} suggesting that the colibactins may damage DNA by induction of replication-dependant DSBs.¹³ Detailed structure-function analyses of the colibactins have been impossible to conduct owing to their low yields of natural production and the absence of a synthetic route to the targets. However, the aminocyclopropane fragments within 2-6 are reminiscent of yatakemycin, CC-1065, and the duocarmycins, which have been shown to alkylate DNA via nucleophilic ring-opening,¹⁴ and the biheterocyclic fragment may serve as a DNA intercalation motif.¹⁵

In light of the immense difficulties associated with isolating natural precolibactins, chemical synthesis provides an attractive avenue to resolve the ambiguities surrounding the composition of the active metabolite(s) and to enable mechanism of action and structure-function studies. Studies indicate the presence of an aminomalonyl unit in the biosynthetic pathway,^{12,16} suggesting additional colibactins are formed, but no evidence relevant to the structures of these metabolites exists, to our knowledge. Consequently, the inventors initially focused on the synthesis of the predicted structures of precolibactin A (5a and 5b) and precolibactin C (6), as these represent the most advanced precolibactins for which structural data had been presented. In the present application, the inventors report a convergent and high-yielding synthesis of structures 5a and 5b by cyclization of a fully linear precursor, establish that these materials are distinct from natural precolibactin A, propose and validate by synthesis a revised structure for precolibactin A (as 7), demonstrate that acyclic precolibactins undergo cyclodehydration to form the pyridone residues found in precolibactin B (3), 4, and precolibactin C (6) under mild conditions, and confirm the structures of precolibactins B (3) and C (6) by total synthesis. The inventors anticipate that this route will be amenable to synthesis of more advanced structures, including those containing aminomalonyl units, as they are proposed.

### Results

As shown in FIG. 1, at the time the inventors began their studies, the structure of precolibactin A had been predicted as 5a or 5b. Consequently, the inventors designed their synthetic route to accommodate either heterocyclic sequence and to provide access to the bithiazole found in precolibactin C (6). The synthesis of the common left-hand fragment began with Nₐ-(tert-butoxycarbonyl)-D-asparagine, which was coupled with (S)-hex-5-en-2-amine (8, prepared in three steps, 96% yield, and 88% ee from pent-4-en-al)¹⁷ using N-(3-dimethylaminopropyl)-N′-ethylcarbodiimide hydrochloride (EDC.HCl). Cleavage of the tert-butoxycarbonyl protective group (hydrochloride acid) provided the amine hydrochloride 9 (84%, two steps). Acylation of the amine 9 with myristoyl chloride, followed by oxidative cleavage of the alkene (ruthenium chloride, sodium periodate) generated the carboxylic acid 10 (78%, two steps).

The isomeric thiazoline-thiazole and the related bithiazole fragments were prepared by the sequences shown in FIG. 9, Scheme 2. Deuterated cysteine labelling experiments¹⁸ supported preservation of the L-amino acid configuration in precolibactin A^{10a} so we selected L-cysteine as the building block for the thiazoline ring. Treatment of N-(tert-butoxycarbonyl)aminoacetonitrile (11) with L-cysteine ethyl ester provided the thiazoline 12 (85%, FIG. 9, Scheme 2A). Aminolysis of the ester, followed by heating with Lawesson's reagent, generated the thioamide 13 (>99%, two steps). Exposure of 13 to bromopyruvic acid in the presence of triethylamine formed the thiazoline-thiazole 14 (71%). The thiazoline-thiazole and subsequent intermediates were found to be exceedingly unstable toward hydrolytic ring-opening and, to a lesser and variable extent, oxidation to a bithiazole. Accordingly, the identification of conditions to isolate and purify these intermediates without exposure to water was essential to the success of the route. Cleavage of the tert-butoxycarbonyl protective group (hydrochloric acid, >99%) generated the amine 15. Coupling (silver trifluoroacetate, triethylamine) of the amine 15 with the β-ketothioester 16 (prepared in one step and 56% yield from N-(tert-butoxycarbonyl)-1-aminocyclopropane-1-carboxylate)¹⁷ followed by carbamate cleavage (hydrochloric acid, >99%) furnished the thiazoline-thiazole fragment 17.

The isomeric thiazole-thiazoline fragment was prepared by a modified sequence (FIG. 9, Scheme 2B). Treatment of N-(tert-butoxycarbonyl)-2-aminoethanthioamide (18) with ethyl bromopyruvate formed the thiazole 19 (74%). Aminolysis of 19 followed by dehydration of the resulting primary amide (trifluoroacetic anhydride, triethylamine) generated the nitrile 20 (84%, two steps). Coupling of 20 with L-cysteine formed the thiazole-thiazoline 21 (97%). In contrast to the isomeric intermediate 14, 21 was found to be stable toward aqueous workup and atmospheric oxygen. A three-step sequence analogous to that described above provided the thiazole-thiazoline 23 (69% overall).

The bithiazole fragment was prepared by the sequence shown in Scheme 2C. Aminolysis of the ester 19 followed by heating with Lawesson's reagent formed the thioamide 24 (>99%, two steps). Treatment of the thioamide 24 with bromopyruvic acid in the presence of calcium carbonate formed the bithiazole 25 (58%). Prior efforts to prepare and isolate 25 were impeded by its instability;¹⁹ we found that rigorous exclusion of water during work-up and purification facilitated the isolation of 25 and subsequent intermediates in homogenous form. A three-step sequence analogous to that used to prepare 17 and 23 then generated the bithiazole fragment 27 in high yield (72% overall).

To complete the synthesis of the precolibactin A skeleton, the carboxylic acid 10 was first converted to the β-ketothioester 28 by activation with carbonyl diimidazole followed by the addition of 3-(tert-butylthio)-3-oxopropanoic acid and magnesium ethoxide (Scheme 3).²⁰ Silver-mediated coupling of 28 with the heterocyclic fragments 17, 23, or 27 then formed the penultimate intermediates 29a-c (90%, 87%, and 86% for 29a, 29b, and 29c, respectively). The stabilities of the fully linear precursors 29a-c paralleled those of 17, 23, or 27; the thiazole-thiazoline 29b was stable toward aqueous work-up, while the thiazoline-thiazole 29a and the bithiazole 29e were unstable toward aqueous conditions.

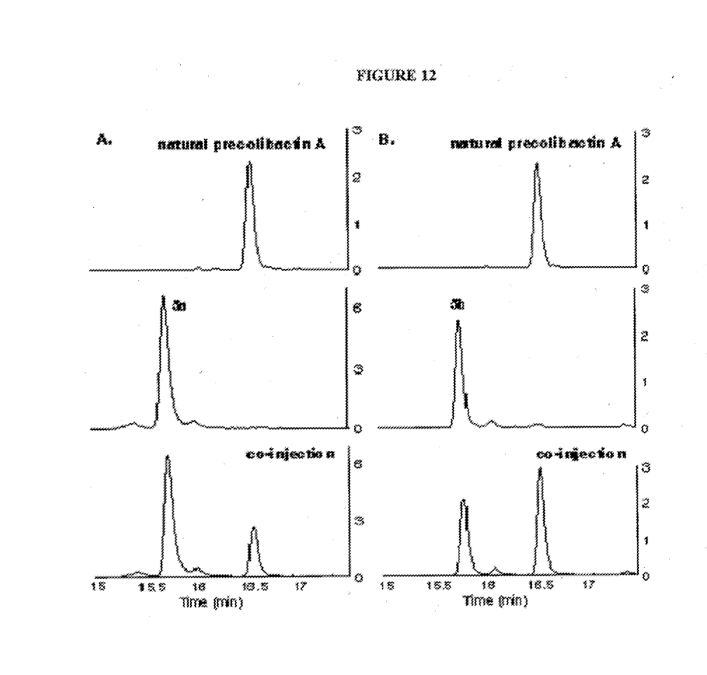

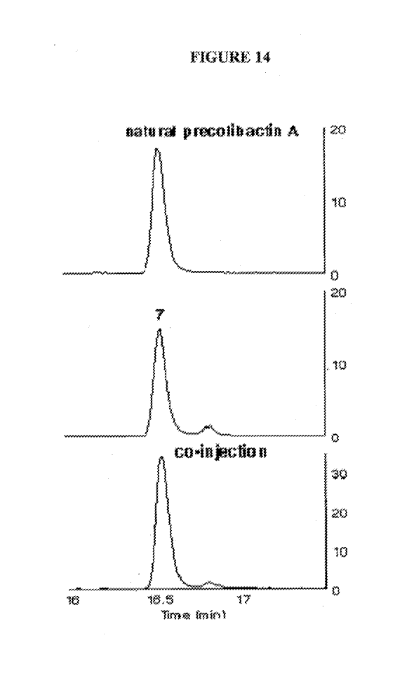

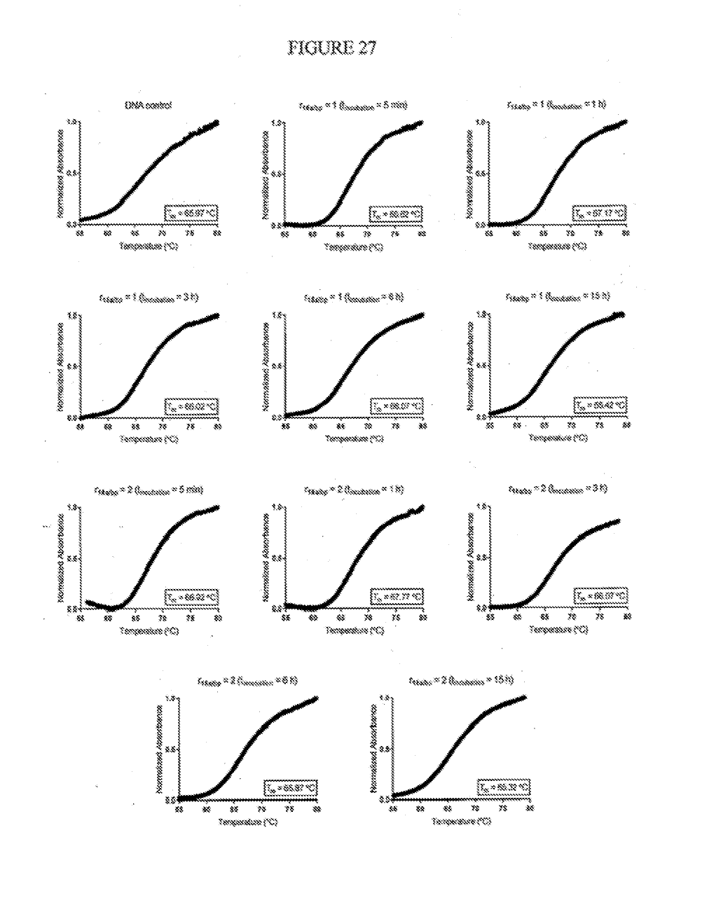

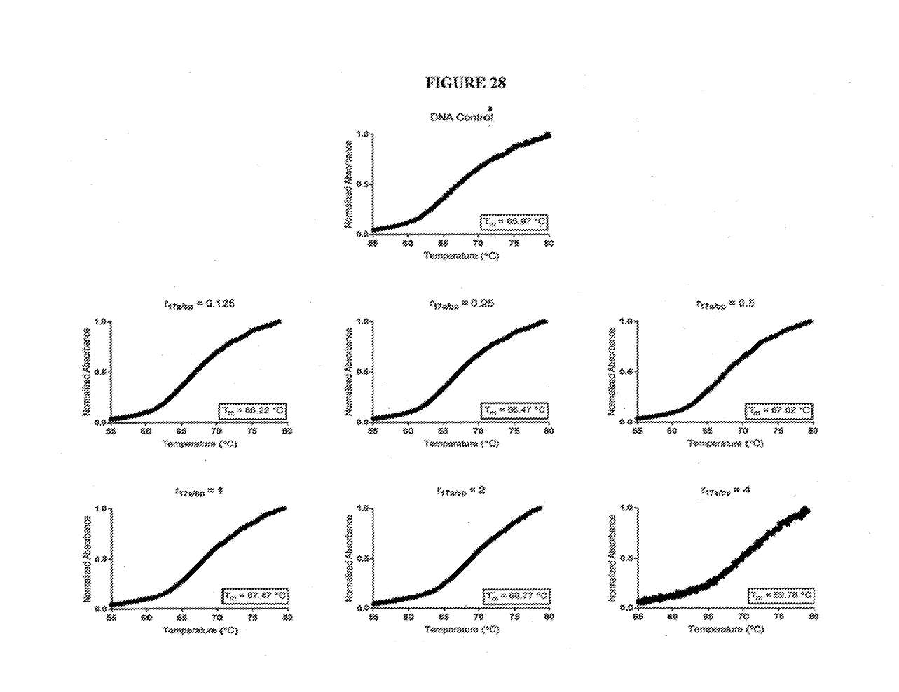

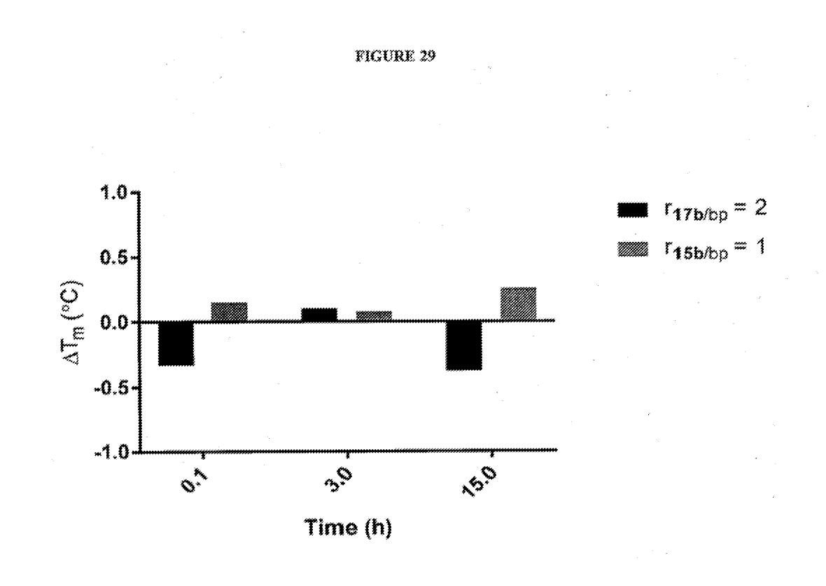

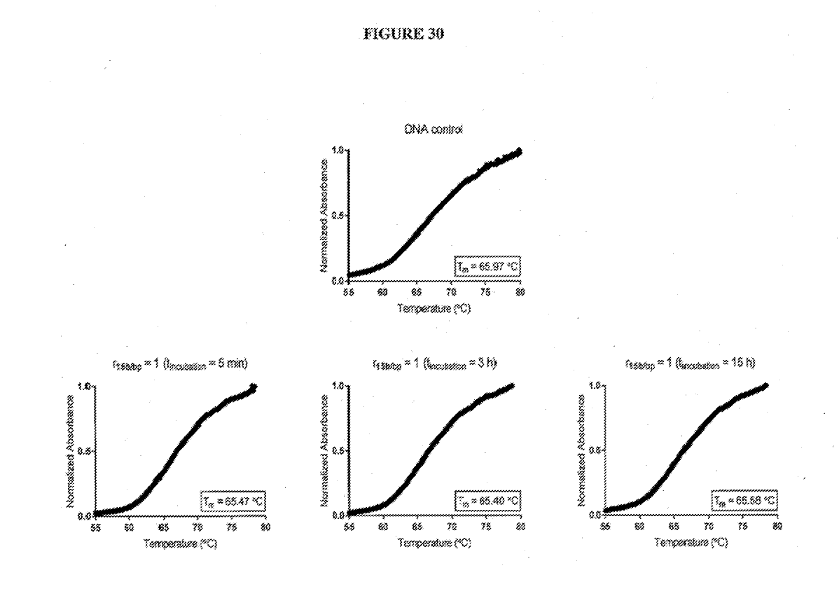

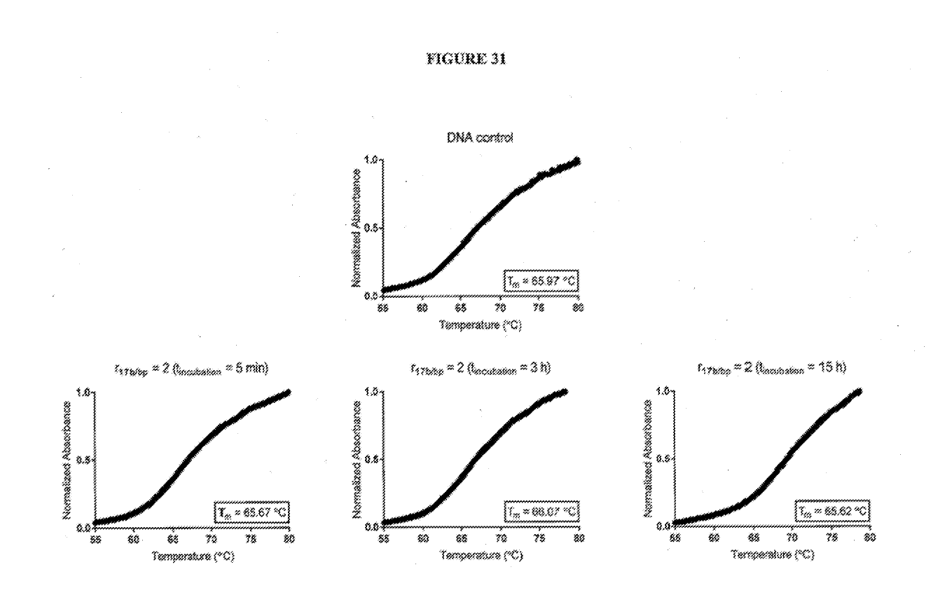

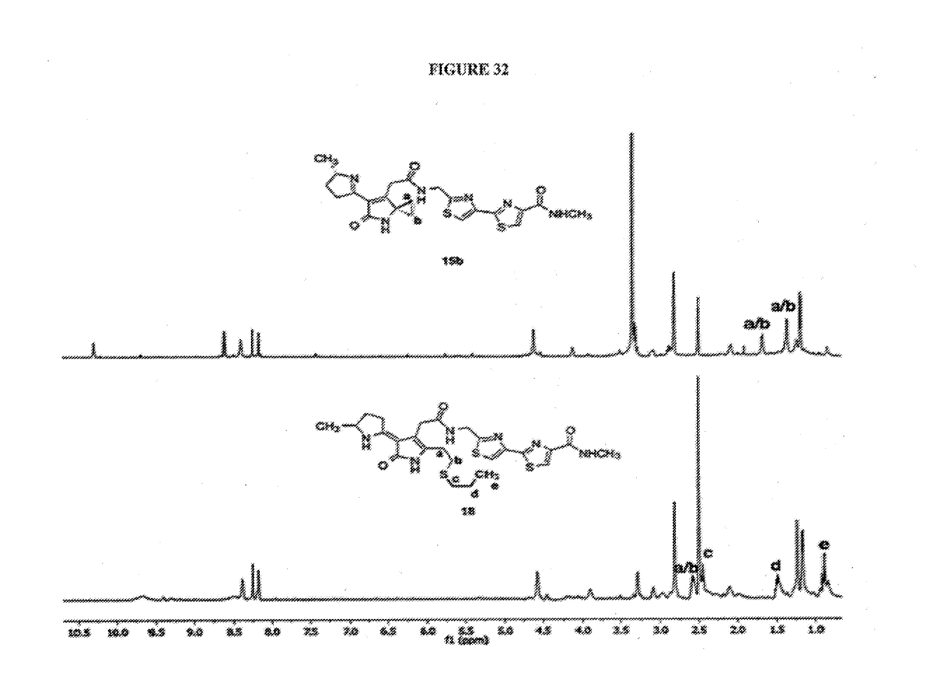

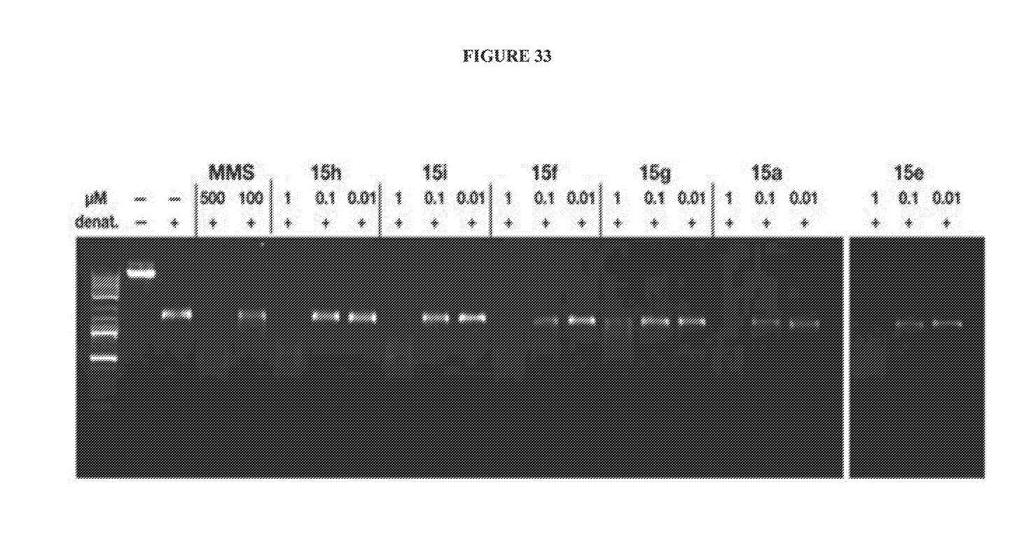

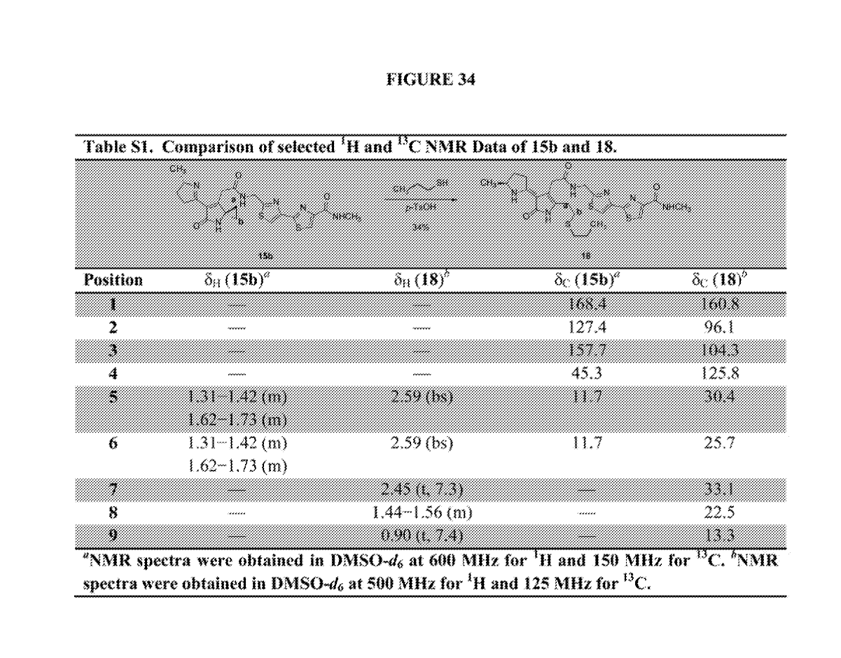

Given the higher stability of the thiazole-thiazoline 29b, this compound was used to develop conditions to effect the key cyclization reaction (to 5b). Surprisingly, the inventors found that in preparative experiments treatment of 29b with potassium carbonate in methanol at 0° C. resulted in formation of the pyridone 30b (80%, Scheme 4A). Similar outcomes were obtained on exposure of 29b to ammonium carbonate in ethanol or aqueous sodium hydroxide. Under these conditions, accumulation of the putative monocyclized intermediate 5b was not observed (LC/MS analysis). The pyridone 30b was fully characterized and spectroscopic data for this compound were in good agreement with the isolated metabolite precolibactin B (3; Table 1, infra). In particular, H-2 and H-4 of 30b resonated at 6.16 and 5.59/5.50 ppm, respectively; these values are nearly identical to those recorded for natural precolibactin B (3; 6.16 and 5.61/5.48 ppm, respectively).¹¹ A plausible mechanism for the formation of the pyridone 30b involves cyclodehydration to 5b, 1,2-addition of the primary amide to the adjacent carbonyl, and aromatization. The facile formation of 30b from 29b provides evidence that the putative and isolated colibactin metabolites 3, 4, and 6 may derive from related acyclic precursors, although the timing of cyclizations in the modular biosynthetic pathway remains unknown. The cyclization of the thiazoline-thiazole derivative 29a and the bithiazole derivative 29c proceeded in a strictly analogous manner to provide the fully cyclized derivatives 30a or precolibactin C (6), respectively.¹⁷ The mass spectroscopic fragmentation data and ¹H NMR data for synthetic precolibactin C (6), as well as LC/MS co-injection with metabolite extracts, matched those of natural material (FIG. 27, FIGS. 12A and B and FIG. 14).^{10,12} In addition, synthetic precolibactin C (6) was converted to N-myristoyl-D-Asn and its corresponding colibactin in a ClbP-dependent manner, indicating that precolibactin C (6) represents a suitable substrate for ClbP (FIG. 29). This sequence provided multimilligram quantities of 30a, 30b, and precolibactin C (6), and is envisioned to be easily scalable.

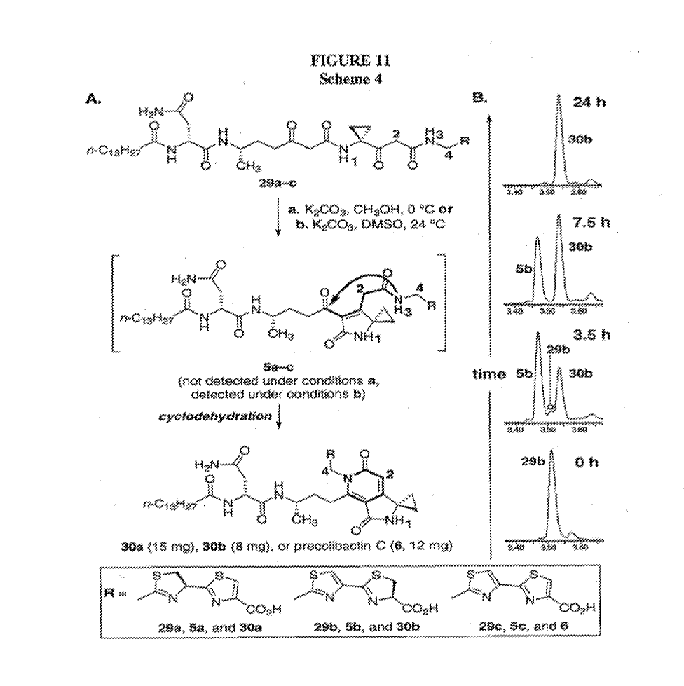

The inventors ultimately found that treatment of 29b with potassium carbonate (˜3.0 equiv) in dimethyl sulfoxide at 24° C. proceeded more slowly and allowed for detection of 5b in the reaction mixture (LC/MS analysis, FIG. 11, Scheme 4B). By conducting the reaction in dimethyl sulfoxide-d₆, signals consistent with the monocyclized intermediate 5b could also be observed by ¹H NMR analysis (Table 1, below).

TABLE 1
Selected ¹H chemical shifts and LC/MS data
for 29b, 5b, 30b, and precolibactin B (3).^{a}

| Position | 1 | 2 | 3 | 4 | tᵣ (min) |
|---|---|---|---|---|---|
| 29b | 8.83 | 3.60 | 8.94 | 4.56 | 3.52 |
| 5b | 8.78 | —^{b} | 8.65 | —^{b} | 3.48 |
| 30b | 8.46 | 6.16 | | 5.59, 5.50 | 3.55 |
| precolibactin B (3)¹⁰ | 8.44 | 6.16 | | 5.61, 5.48 | |

^{a1}H spectroscopic data recorded in DMSO-d₆ (500 MHz, 23° C.).
Complete ¹H NMR spectra (Figure S2) and LC/MS conditions are presented in the Supporting Information.
^{b}Not resolved.

Mass-selective LC/HRMS-QTOF analysis was conducted to determine if either 5a or 5b corresponded to the structure of natural precolibactin A. As shown in FIG. 12, the concentrated ethyl acetate extracts of clb⁺ E. coli ΔclbP^{10a} displayed a single prominent peak of m/z=816.3788, which corresponds to [M+H]⁺ for the proposed structure of precolibactin A. However, the retention times of synthetic 5a and 5b were distinct and the signals did not coalesce upon co-injection with the natural sample (FIGS. 12A and 12B, respectively). The retention times of 5a and 5b were nearly identical (tᵣ=15.70 and 15.80 min, respectively), as expected, and their differences with respect to natural precolibactin A (tᵣ=16.56 min) suggested a significant discrepancy in structure.

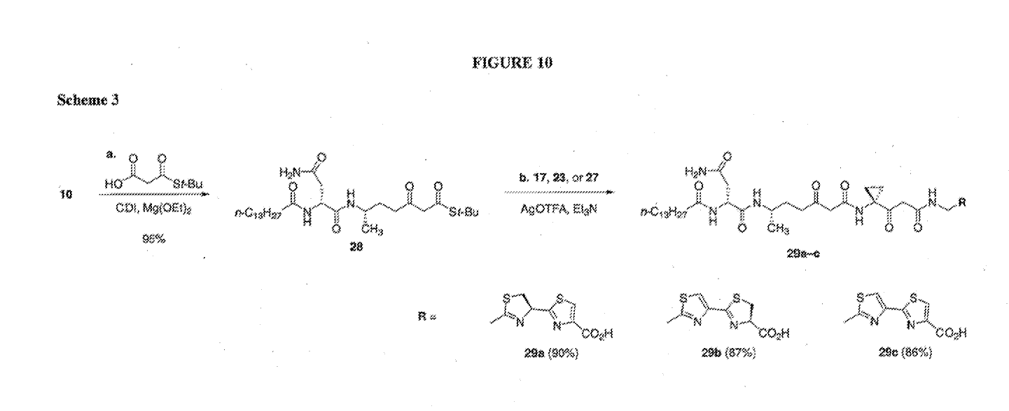

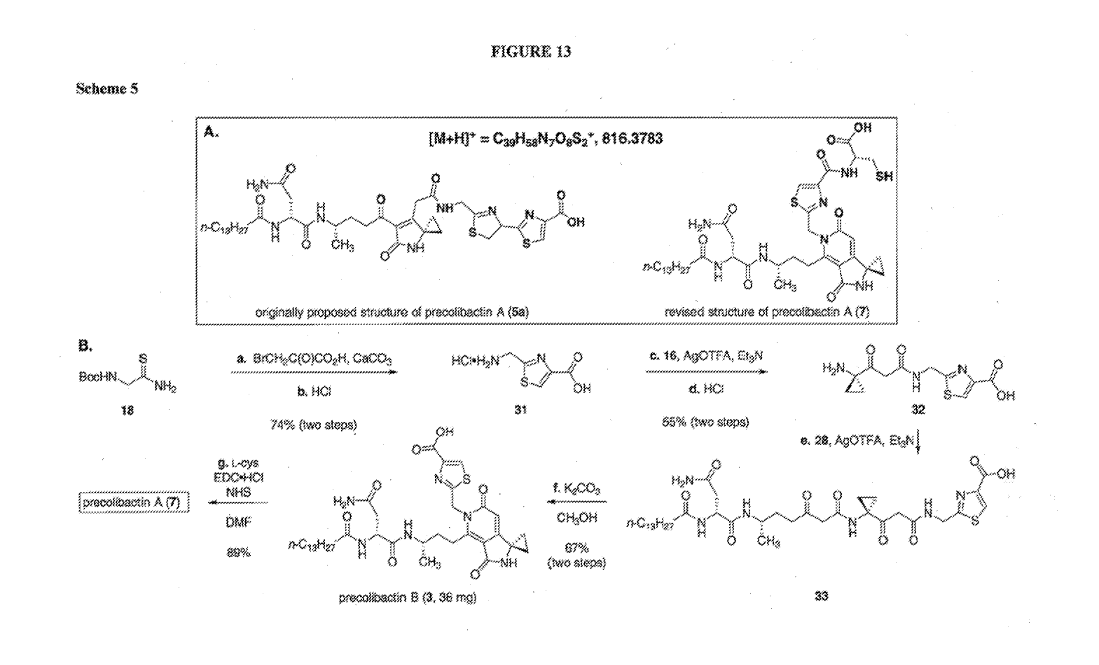

In light of the facile cyclization of our synthetic intermediates to the pyridone residues found in precolibactins B (3) and C (6), the inventors reasoned that precolibactin A may also incorporate this substructure. The original isotope labeling, HRMS, and MSMS data for natural precolibactin A^{10a} could not exclude this assignment. In addition, careful inspection of the initial report revealed that precolibactin A production was optimized by increasing the concentration of L-cysteine in the media 5-fold (to 1 g/L). Walsh and co-workers have previously reported the formation of cysteine derailment products in the biosynthesis of yersiniabactin.²¹ Accordingly, we hypothesized that the structure of natural precolibactin A may comprise the pyridone found in precolibactins B (3) and C (6) and a terminal cysteine residue appended to a single thiazole ring (FIG. 13, Scheme 5A). This structure (7) possesses an exact mass that is identical to the originally predicted structures 5a or 5b and would similarly match the reported amino acid isotope labeling studies. Such a change in the terminal heterocyclic fragment would also be consistent with the large differences in retention times between 5a or 5b and natural precolibactin A. The synthesis of the revised precolibactin A structure 7 was readily-accomplished using our synthetic strategy (FIG. 13, Scheme 5B). Treatment of N-(tert-butoxycarbonyl)-2-aminoethanthioamide (18) with bromopyruvic acid²² followed by removal of the tert-butoxycarbonyl protective group generated the thiazole 31 (74%, two steps). Silver trifluoroacetate-mediated coupling of 31 and the thioester 16, followed by carbamate cleavage, formed the amine 32 (55%, two steps). Coupling of the amine 32 with the thioester 28 (FIG. 10, Scheme 3; silver trifluoroacetate, triethylamine), followed by double-cyclization (potassium carbonate, methanol) generated precolibactin B (3; 67% over two steps, 36 mg). NMR spectroscopic data for synthetic precolibactin B (3) and LC/MS co-injection with metabolite extracts matched those of natural material,^{11,12} thereby confirming the structure of the natural product.¹⁷ Finally, coupling of precolibactin B (3) with L-cysteine mediated by N-hydroxysuccinimide (NHS) and EDC.HCl generated 7 (89%). Mass-selective LC/HRMS-QTOF analysis against the concentrated ethyl acetate extracts of clb⁺ E. coli ΔclbP^{10a} revealed that 7 corresponded exactly to natural material (FIG. 14). In addition, both synthetic 7 and natural precolibactin A displayed identical mass spectral fragmentation patterns, providing further confirmation of structure (Table S2). As natural precolibactin A is not isolable in amounts sufficient for NMR analysis,^{10a} a direct comparison of the NMR spectra of synthetic and natural precolibactin A is not possible at this time.²³

### Discussion and Conclusion

The colibactins are a fascinating family of natural products that are produced by certain strains of commensal and extraintestinal E. coli, and the pathway has been implicated in the progression of colorectal cancer.^{7,8} Despite over a decade of intensive research, their complete structures and mechanism of action have remained unresolved. As highlighted in FIG. 1, many of these compounds have been isolated in astoundingly low yields (μg/L) by painstaking fermentation experiments. By bringing the power of modern bioinformatics, enzymology, and mass spectrometry to bear on this problem, the structures of additional precolibactins, which are recalcitrant to isolation, have been predicted.

At the time the inventors began their work, 3-6 represented the most complex precolibactin structures in the literature. Precolibactin B (3) and 4 were fully characterized by isolation,¹⁰ while 5a^{10a} and precolibactin C (6)¹¹ were predicted. While this manuscript was in preparation, Balskus and co-workers¹² reported the isolation of precolibactin C (6; 0.5 mg from a 48 L fermentation) from a mutant strain. Biosynthetic studies now suggest that additional precolibactins incorporating an aminomalonyl unit exist,^{12,16} but no evidence for their structures has been presented, to our knowledge.

The inventors have developed a high-yielding and modular synthetic route to the most advanced known precolibactin structures. The left hand fragment 10, which is common to all of the precolibactins, is prepared in six steps and 63% overall yield from pent-4-en-al (FIG. 8, Scheme 1). The inventors have executed the synthesis of four distinct heterocyclic side chain fragments, in 4-7 steps and 37-41% overall yield (FIG. 9, Schemes 2 and FIG. 13, Scheme 5). Finally, these intermediates are elaborated to advanced precolibactins in three steps and −50% overall yield (FIG. 10, Scheme 3, FIG. 11, Scheme 4, and FIG. 13, Scheme 5). The inventors have confirmed the structures of the isolated precolibactins B (3) and C (6) by chemical synthesis, and revised the structure of precolibactin A, from 5a or 5b to 7. This structural revision also supports an unexpected biosynthetic route to colibactin bithiazole formation, in which biosynthesis of the first thiazole ring may precede heterocyclization and oxidation of the C-terminal L-cysteine moiety. This is in contrast to bioinformatic proposals for bleomycin bithiazole biosynthesis.²⁴ These synthetic studies also provide insights into the reactivities of these structures, and the facile cyclization of the linear precursors 29a-c to pyridones suggests this element as a common substructure. The inventors envision that the synthetic strategy we have presented will be amenable to the synthesis of precolibactins incorporating an aminomalonyl substituent or other modifications, as their structures are proposed.

The synthetic route outlined herein provides a means to procure sufficient quantities of material to study the cellular responses to isolated colibactins and elucidate their mechanism of action for the first time. As noted in the introduction, mammalian cells were shown to accumulate DNA DSBs when co-cultured with pks⁺ E coli cells,^{3a} but no essential follow-up studies employing single metabolites derived from the clb pathway have been reported, to our knowledge. The inventors expect that their modular synthetic strategy will finally open the door to examining these types of questions regarding colibactin's mode of action with molecular-level resolution.²⁵

### REFERENCES FOR THIS SECTION

1. For a review, see: Donia, M. S.; Fischbach, M. A. Science 2015, 349, 395.
2. Xu, 1; Gordon, J. I. Proc. Nad. Acad. Sci. U.S.A. 2003, 100, 10452.
3. (a) Nougayrède, J.-P.; Homburg, S.; Taieb, F.; Boury, M.; Brzuszkiewicz, E.; Gottschalk, G.; Buchrieser, C.; Hacker, J.; Dobrindt, U.; Oswald, E. Science 2006, 313, 848. For recent reviews, see: (b) Balskus, E. P. Nat. Prod. Rep. 2015, 32, 1534. (c) Trautman, E. P.; Crawford, J. M. Curr. Top. Med. Chem. 2015, 16, 1.
4. Dubois, D.; Baron, O.; Cougnoux, A.; Delmas, J.; Pradel, N.; Boury, M.; Bouchon, B.; Bringer, M. A.; Nougayrede, J. P.; Oswald, E.; Bonnet, R. J. Biol. Chem. 2011, 286, 35562.
5. (a) Brotherton, C. A.; Balskus, E. P. J. Am. Chem. Soc. 2013, 135, 3359. (b) Bian, X.; Fu, J.; Plaza, A.; Herrmann, J.; Pistorius, D.; Stewart, A. F.; Zhang, Y.; Muller, R. Chembiochem 2013, 14, 1194. (c) Vizcaino, M. I.; Engel, P.; Trautman, E.; Crawford, J. M. J. Am. Chem. Soc. 2014, 136, 9244.
6. Cuevas-Ramos, G.; Petit, C. R.; Marcq, I.; Boury, M.; Oswald, E.; Nougayrède, J.-P. Proc. Nad. Acad. Sci. U.S.A 2010, 107, 11537.
7. Arthur, J. C.; Perez-Chanona, E.; Mühlbauer, M.; Tomkovich, S.; Uronis, J. M.; Fan, T.-J.; Campbell, B. J.; Abujamel, T.; Dogan, B.; Rogers, A. B.; Rhodes, J. M.; Stintzi, A.; Simpson, K. W.; Hansen, J. J.; Keku, T. O.; Fodor, A. A.; Jobin, C. Science 2012, 338, 120.
8. Arthur, J. C.; Gharaibeh, R. Z.; Mühlbauer, M.; Perez-Chanona, E.; Uronis, J. M.; McCafferty, J.; Fodor, A. A.; Jobin, C. Nat. Commun. 2014, 5, 4724.
9. Buc, E.; Dubois, D.; Sauvanet, P.; Raisch, J.; Delmas, J.; Darfeuille-Michaud, A.; Pezet, D.; Bonnet, R. PLoS One 2013, 8, e56964.
10. (a) Vizcaino, M. I.; Crawford, J. M. Nat. Chem. 2015, 7, 411. (b) Bian, X.; Plaza, A.; Zhang, Y.; Müller, R. Chem. Sci. 2015, 6, 3154. (c) Brotherton, C. A.; Wilson, M.; Byrd, G.; Balskus, E. P. Org. Lett. 2015, 17, 1545.
11. Li, Z. R.; Li, Y.; Lai, J. Y.; Tang, J.; Wang, B.; Lu, L.; Zhu, G.; Wu, X.; Xu, Y.; Qian, P. Y. Chembiochem 2015, 16, 1715.
12. Zha, L.; Wilson, M. R.; Brotherton, C. A.; Balskus, E. P. ACS Chem. Biol. 2016, [Online early access]. DOI: 10.1021/acschembio.6b00014. Published Online: Feb. 18, 2016. http://pubs.acs.org/doi/abs/10.1021/acschembio.6b00014 p (accessed Mar. 28, 2016).
13. Zhang, J.; Walter, J. C. DNA Repair 2014, 19, 135.
14. Tichenor, M. S.; Boger, D. L. Nat. Prod. Rep. 2008, 25, 220.
15. Wu, W.; Vanderwall, D. E.; Turner, C. J.; Hoehn, S.; Chen, J.; Kozarich, J. W.; Stubbe, J. Nucleic Acids Res. 2002, 30, 4881.
16. Brachmann, A. O.; Garcie, C.; Wu, V.; Martin, P.; Ueoka, R.; Oswald, E.; Piel, J. Chem. Commun. 2015, 51, 13138.
17. See Supporting Information.
18. Bode, H. B.; Reimer, D.; Fuchs, S. W.; Kirchner, F.; Dauth, C.; Keglcr, C.; Lorenzen, W.; Brachmann, A. O.; Grün, P. Chem.—Eur. J. 2012, 18, 2342.
19. Souto, J. A.; Vaz, E.; Lepore, I.; Pöppler, A.-C.; Franci, G.; Álvarez, R.; Altucci, L.; de Lera, Á. R. J. Med. Chem. 2010, 53, 4654.
20. Sodeoka, M.; Sampe, R.; Kojima, S.; Baba, Y.; Usui, T.; Ueda, K.; Osada, H. J. Med. Chem. 2001, 44, 3216.
21. Gehring, A. M.; Mori, I.; Perry, R. D.; Walsh, C. T. Biochemistry 1998, 37, 11637.
22. Videnov, G.; Kaiser, D.; Kempter, C; Jung, G. Angew. Chem., Int. Ed. 1996, 35, 1503.
23. Synthetic precolibactin A (7) was found to readily undergo disulfide bond formation, which may explain its instability in complex cellular organic extracts.
24. Galm, U.; Wendt-Pienkowski, E.; Wang, L.; Huang, S.-X.; Unsin, C.; Tao, M.; Coughlin, J. M.; Shen, B. J. Nat. Prod. 2011, 74, 526.
25. Our data suggest that prior proposals of the colibactin mechanism of action may require revision. See the Supporting Information for a discussion.

### DNA Alkylation and Related Experiments

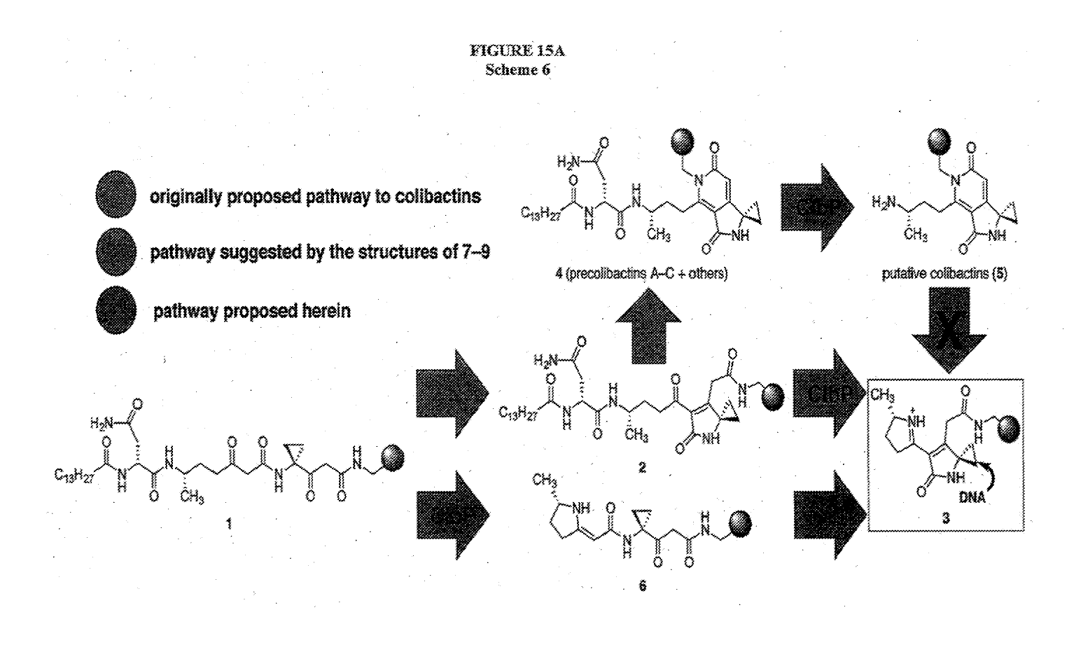

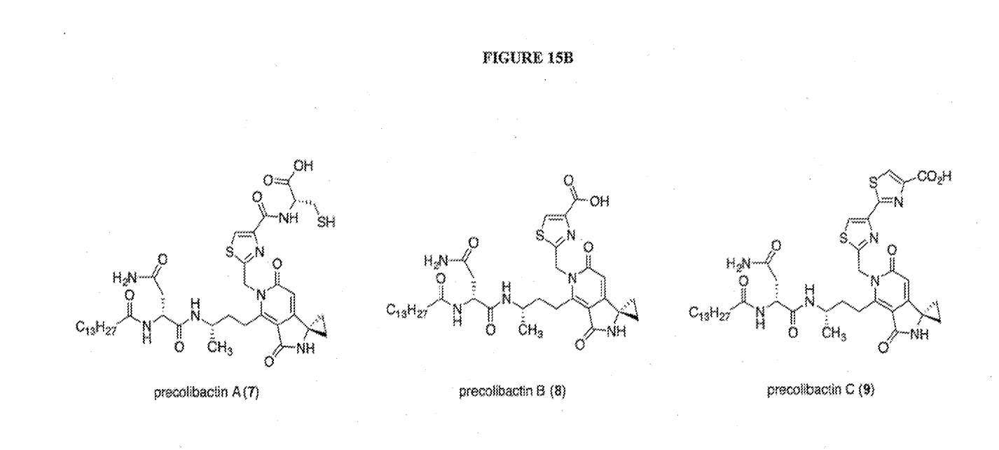

The Precolibactins and colibactins represent a family of natural products that are encoded by the clb (aka pks) gene cluster and are produced by certain commensal, extraintestinal, and probiotic E. coli. clb⁺ E. coli have been shown to induce megalocytosis and DNA double-strand break formation in eukaryotic cells. Paradoxically, this gene cluster is found in the probiotic strain Nissle 1917, which is used for the treatment of gastrointestinal disorders. Evidence suggests that the precolibactins are converted to genotoxic colibactins by colibactin peptidase (ClbP)-mediated cleavage of an N-acyl-D-Asn side chain. It has been hypothesized that colibactins exert genotoxicity by formation of an unsaturated imine and nucleotide alkylation by cyclopropane ring opening (2→3, FIG. 15A, Scheme 6). However, no colibactins have been directly isolated from producing organisms, and this hypothesis has not been tested experimentally. In addition, many advanced precolibactins such as precolibactins A-C (7-9, FIG. 15B) contain a pyridone residue that cannot generate the unsaturated imines that form the basis of this mode of action hypothesis. These examples evaluate the DNA binding and alkylation activity of 13 synthetic pyridone (5) and unsaturated imine (3) colibactin derivatives and show that the unsaturated imines potently alkylate DNA, whereas the corresponding pyridone derivatives do not. The inventors define the imine, unsaturated lactam, and cyclopropane functional groups of these molecules as essential for efficient DNA alkylation. The presence of a cationic side chain enhances DNA alkylation. These studies suggest that precolibactins containing a pyridone residue do not form the basis for the genotoxicity of the clb gene cluster. Instead, as all precolibactins to date have been obtained from clbP mutant strains, the inventors propose that these are off-pathway fermentation products produced by a facile double cyclodehydration route that manifests in the absence of viable ClbP. The structure-function and mechanistic models presented herein will ultimately provide a means to connect metabolite structure with the disparate phenotypes associated with clb⁺ E. coli.

Precolibactins and colibactins are natural products produced by select commensal, extraintestinal, and probiotic E. coli. The metabolites are encoded by a hybrid polyketide synthase-nonribosomal peptide synthetase (PKS-NRPS) gene cluster termed clb or pks.¹ clb⁺ E. coli strains induce DNA damage in eukaryotic cells and are thought to promote colorectal cancer formation,^{1c,2} but the gene cluster is also found in the probiotic strain Nissle 1917, which is used in Europe for the treatment of ulcerative colitis, diarrhea, and other gastrointestinal disorders.³ Mature precolibactins are substrates for the 12-transmembrane multidrug and toxic compound extrusion transporter ClbM, which mediates their transfer to the bacterial periplasm.⁴ There, the colibactin peptidase ClbP converts precolibactins to genotoxic colibactins via removal of an N-acyl-D-asparagine side chain.⁵ Mutation of ClbP abolishes cellular DNA damaging-activity,^{2a,5b} and N-myristoyl-D-asparagine and closely related analogs have been identified in wild type clb⁺ E. coli cultures.^{5d,5e} Whether the differential production of biosynthetically-related but distinct metabolites, or other factors (such as the requirement for cell-to-cell contact to observe cytopathic effects^{2a,6}) underlie the seemingly contradictory phenotypes associated with the clb gene cluster, remains unresolved.

The inventors have focused on understanding the molecular basis of colibactin-induced DNA damage. Advanced precolibactins arise from linear precursors of the generalized structure shown as 1 (Fibure 15, Scheme 6). The linear precursors were suggested to transform to unsaturated lactams 2 that are processed by ClbP to generate unsaturated iminium ions 3 (colibactins), which alkylate DNA by cyclopropane ring-opening (grey pathway).^{7,8} However, this mechanistic hypothesis is ostensibly incompatible with subsequent isolation⁹ and synthesis¹⁰ efforts that lead to the identification and unequivocal structural assignment of precolibactins A (7),¹¹ B (8), and C (9), which contain a pyridone residue (FIG. 15B). Precolibactins A-C (7-9) were obtained from clbP mutant strains; these deletion strains were employed to promote accumulation of the precolibactin metabolites. If 7-9 are the genotoxic precursors, the data outlined above⁵ suggests that amines such as 5 resulting from ClbP-mediated processing in the wild type strains are responsible for the cytopathicity of the clb cluster, as these cannot readily convert to unsaturated iminium ions such as 3. Precolibactin C (9) was demonstrated to be a substrate for ClbP.¹⁰

In earlier synthetic work, we showed that the double dehydrative cyclization of the relatively stable N-acylated linear precursors (1) to pyridones such as 7-9 was facile under mildly acidic or basic conditions (c.f., 1→2→4, Scheme 1).¹⁰ The unsaturated lactam intermediates (2) could be detected by LC/MS analysis, but they were not isolable, arguing against their interception by ClbP in the biosynthesis. We reasoned that the colibactins may instead form by ClbP processing of isolable linear precursors 1 directly. Sequential cyclodehydration reactions proceeding through the vinylogous ureas 6 would then provide 3 (red pathway). It follows from this analysis that precolibactins A-C (7-9) are non-natural cyclization products deriving from the absence of ClbP in the producing organisms, and are unlikely to be genotoxic. To test this hypothesis, the inventors modified their synthetic strategy¹⁰ to allow access to the deacylated pyridone derivatives 5 and the analogous unsaturated iminium ions 3. They show that the iminium ions are potent DNA alkylation agents while the corresponding pyridone structures are not. In addition, we rigorously define the structure-function relationships of 3 that are required for or enhance DNA alkylation activity. Finally, the synthetic studies support the alternative biosynthetic pathway involving the intermediacy of the vinylogous amide 6 en route to 3. Collectively, the data lend further support to the hypothesis that unsaturated iminium ions 3 are responsible for the genotoxic effects of the clb gene cluster and support the conclusion that precolibactins A-C (7-9) (and other pyridone-containing isolates) are off-pathway fermentation products derived from the absence of a functional clbP gene. This work constitutes the first structure-function studies of colibactin metabolites and provides a foundation to begin to connect the disparate phenotypic effects of the clb cluster with metabolite structure.

### Results

### Comparison of DNA Binding and Alkylation Activities of Unsaturated Iminium Ion and Pyridone Structures.

To assess the activities of candidate colibactin scaffolds, we required a route that would provide access to unsaturated imine and pyridone derivatives lacking the natural N-myristoyl-D-Asn prodrug fragment. Synthesis of the unsaturated imine derivatives was a significant challenge since we have previously shown that base- or acid-catalyzed cyclization of the linear precursors proceeded rapidly to the pyridone products.¹⁰ In addition, we targeted synthetic derivatives containing a cationic residue appended to the bithiazole fragment. It has been shown that an α-aminomalonate building block is incorporated into the colibactin structure;^{9b,12} we speculated that this residue could enhance DNA affinity by imparting cationic character to the metabolites, similar to other known DNA-targeting agents.¹³ As the presumed natural C-terminal colibactin cationic substituent remains unknown, we initially targeted an N-(β-dimethylaminoethyl)amide derivative, as this is a common substructure in agents that target DNA. We later prepared a series of other derivatives to further explore this region and optimize activity (vide infra).

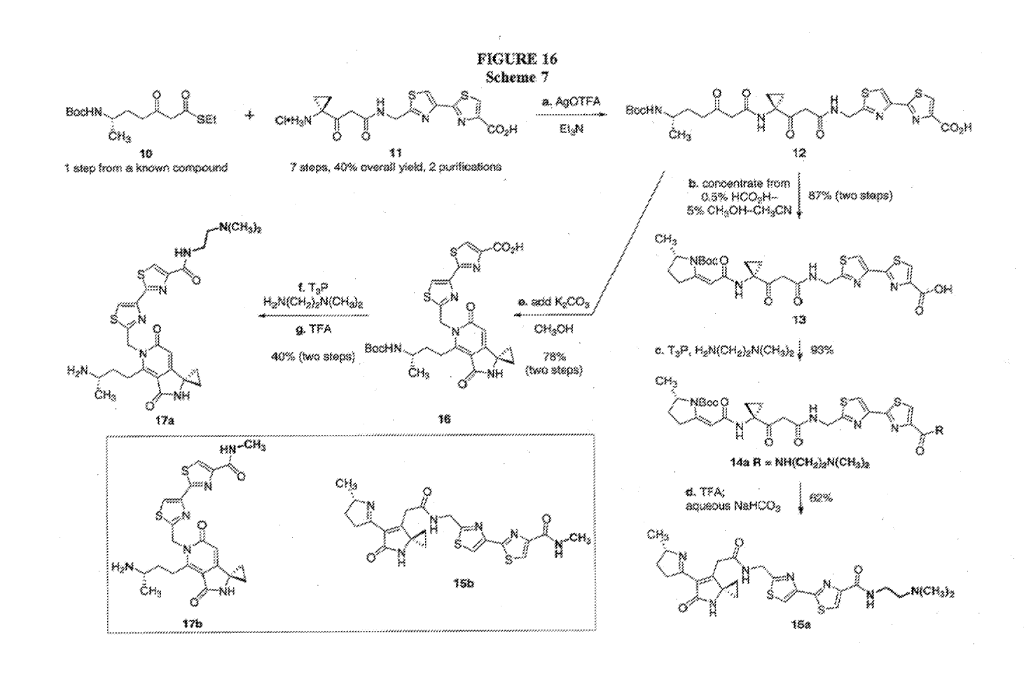

After some experimentation, the inventors identified the linear colibactin derivative 12 as a common intermediate that could be selectively cyclized to provide either unsaturated imine or pyridone products (FIG. 16, Scheme 7). Silver-mediated coupling of the β-ketothioester 10 (prepared in one step and 53% yield by the addition of lithium ethyl thioacetate to tert-butyl (S)-2-methyl-5-oxopyrrolidine-1-carboxylate,¹⁴ see Supporting Information) with the β-ketoamide 11¹⁰ provided the linear precursor 12 (1-3 g scale). The linear precursor 12 cyclized to a variable extent to the vinylogous imide 13 upon purification by anion exchange chromatography. To effect quantitative cyclization, the purified mixture of 12 and 13 was dissolved in 0.5% formic acid-5% methanol-acetonitrile and concentrated (3×). Following this protocol, the vinylogous imide 13 was obtained in 87% isolated yield (over two steps) and >95% purity. Coupling of the carboxylic acid function with N,N-dimethylethylenediamine mediated by propylphosphonic anhydride (T3P)¹⁵ provided the amide 14a (93%). Carbamate cleavage (trifluoroacetic acid) followed by neutralization with saturated aqueous sodium bicarbonate solution (to promote cyclodehydration) then provided the key unsaturated imine 15a (62%).

Alternatively, the direct addition of potassium carbonate (5 equiv) and methanol¹⁰ to the solution of the unpurified coupling product 12 induced double cyclodehydration to provide the pyridone 16 (78% from 10 and 11). T3P-mediated coupling with N,N-dimethylethylenediamine provided an amide (not shown) that was treated with trifluoroacetic acid to yield the amine 17a (40%, two steps). The N-methylamide derivatives 15b and 17b were targeted to directly evaluate the significance of a cationic terminal residue on DNA binding and damaging activity. These were prepared by strictly analogous sequences.

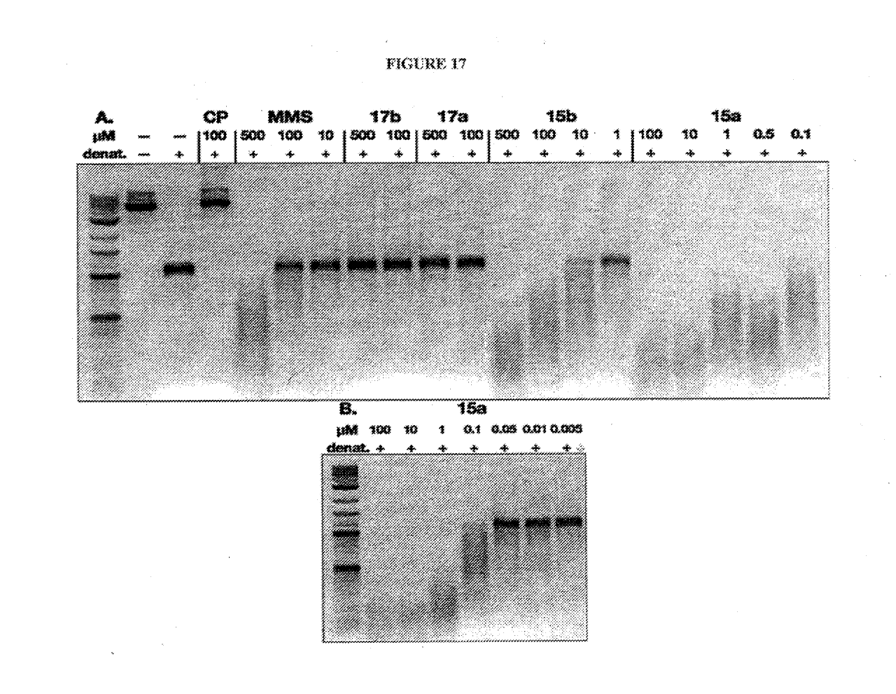

The activity of 15a, 15b, 17a, and 17b was evaluated in a DNA alkylation assay. Plasmid pBR322 DNA was treated with EcoRI-HF in Cutsmart buffer, and the resulting linearized DNA was incubated with 15a, 15b, 17a, or 17b for 15 h at 37° C. The DNA was then denatured, and the sample was eluted on a 1% neutral agarose gel. DNA was visualized with SybrGold. Cisplatin (CP, 100 μM) and methyl methanesulfonate (MMS, 500, 100, or 10 μM) were used as positive controls for DNA cross-linking and alkylation, respectively. As shown in FIG. 17A, neither of the pyridone derivatives 17a or 17b displayed measurable activity in this assay at concentrations up to 500 μM. By comparison, both unsaturated imines 15a and 15b extensively alkylated DNA. The streaking of DNA induced by 15a and 15b parallels that observed with 500 μM of MMS and derives from the presence of shorter DNA strands that are formed by thermal or alkaline denaturation of extensively-alkylated (or in the case of MMS, methylated) DNA.¹⁶ Further evidence for direct alkylation by 15a and 15b is presented in the cross-linking study outlined below. The derivative 15a, which bears a tertiary amine substituent that can engage in an electrostatic interaction with the deoxyribose backbone, was significantly more active than the N-methylamide 15b. These data indicate that the pyridone residue is unlikely to constitute the genotoxic pharmacophore of the colibactins and establish the unsaturated lactam or imine, and cationic terminus as key functional groups in DNA alkylation activity.

A full dose response of the most potent derivative 15a was conducted. As shown in FIG. 17B, extensive DNA alkylation was observed at concentrations as low as 100 nM, and small amounts of DNA damage were detected using 50 or 10 nM of 15a. At 5 nM of 15a the extent of DNA damage was negligible.

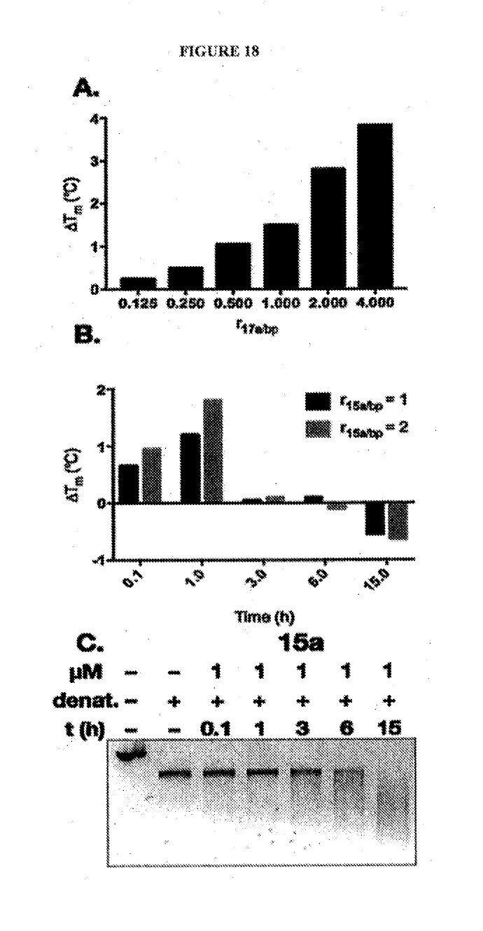

A DNA melting temperature analysis was conducted to determine if the variance in DNA alkylation activity between the pyridones and the unsaturated imines was due to differences in DNA binding affinity. The hyperchromicity of calf thymus DNA (ctDNA) with increasing temperature was measured at 260 nm in the presence of varying concentrations of 15a or 17a. The Tₘ was defined as the temperature at which half of the duplex DNA was unwound and was determined by the maximum of the first derivative of the thermal denaturation profile. As shown in FIG. 18A, treatment of ctDNA with 17a led to a dose-dependent increase in the melting temperature; an increase in Tₘ of 3.8° C. was observed at a ratio of 17a to base pairs (bps) of 4.0. By comparison, the unsaturated imine 15a exhibited time-dependent effects on duplex stability (FIG. 18B). At times less than 1 h, stabilization of the duplex was observed, but upon longer incubations (3-15 h) duplex stability decreased. The inventors hypothesized that this was due to an initial binding of 15a to the duplex followed by slow alkylation and degradation. To probe this, they repeated the plasmid alkylation assay using 15a and evaluated the extent of DNA alkylation as a function of time. As shown in FIG. 18C, alkylation activity correlated approximately with the decrease in duplex stability observed in the melting point experiments (FIG. 18B). Importantly, the affinity of 15a for DNA was less than that of 17a (1.8 and 2.8° C. increase in Tₘ, respectively, at r_{ligand/bp}=2) and thus the variance in DNA alkylation activity between these two compounds cannot be attributed to decreased binding of 17a.

Structure-Function Relationships of Unsaturated Imines.

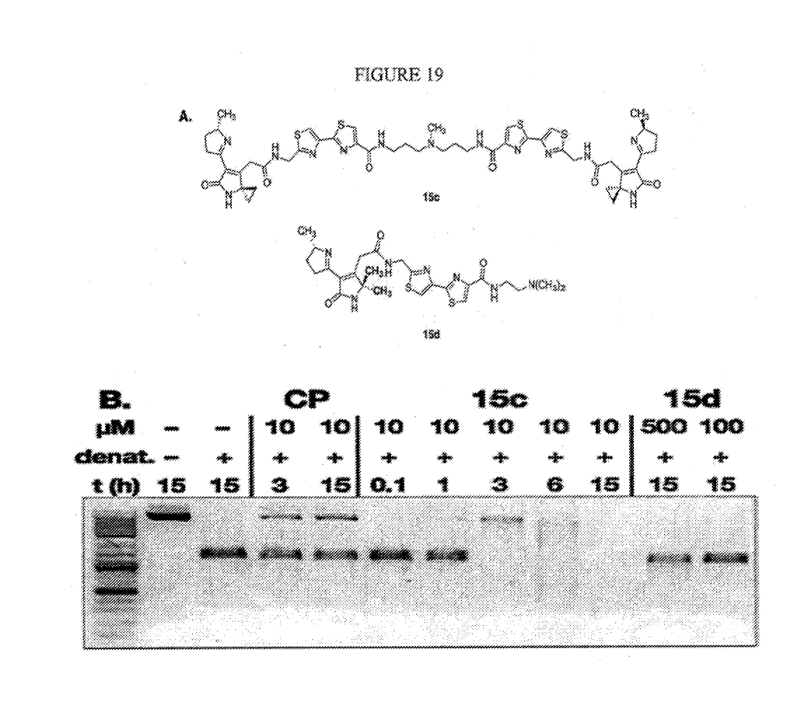

To gain further insights into the functional group requirements for DNA alkylation and to verify the observed activities, we prepared the dimer 15c and the gem-dimethyl derivative 15d (FIG. 19A). As shown in FIG. 19B, incubation of linearized pBR322 DNA with 10 μM of the dimer 15c and monitoring the mixture as a function of time revealed a clear cross-linked band at 3 h. Consistent with the time-dependent alkylation assay shown in FIG. 18C, this cross-linked band diminished at later timepoints (6 or 15 h), suggesting extensive alkylation and degradation of the duplex. The gem-dimethyl derivative 15d did not alkylate DNA at concentrations up to 500 μM. These data further validate the nature of the DNA lesion as alkylation and indicate that the cyclopropane ring is essential for this activity.

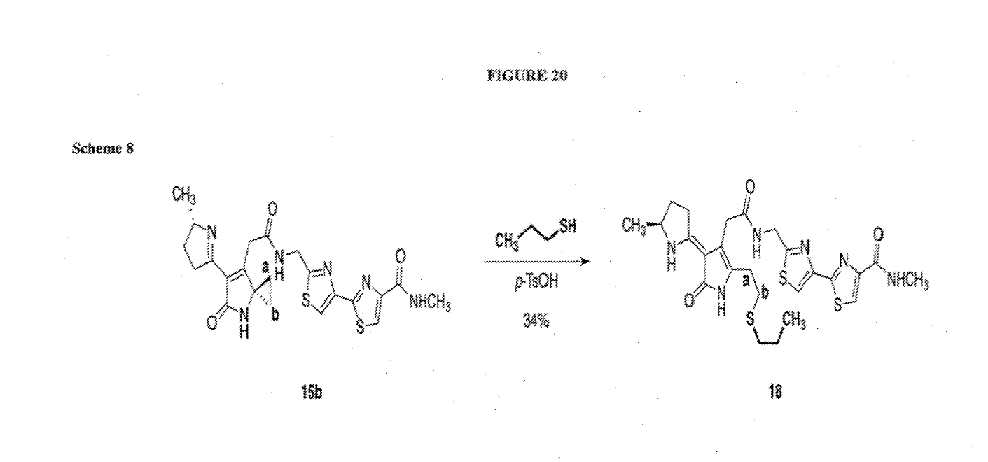

To gain insights into the molecular mechanism of alkylation, we studied the reactivity of 15b in vitro using propanethiol as a model nucleophile (FIG. 20, Scheme 8). The N-methylamide 15b was chosen in preference to the β-(dimethylamino)ethyl amide 15a to simplify isolation. Treatment of the methylamide 15b with excess propanethiol and p-toluenesulfonic acid in 6:1 acetonitrile-N,N-dimethylformamide at 23° C. provided the ring-opened product 18 in 34% isolated yield. The adduct 18 was unstable but was characterized by NMR (¹H, ¹³C, gCOSY, gHSQC, gHMBC) and LC/MS analyses. Cyclopropane ring-opening was evidenced by loss of the resonances at 1.37 and 1.68 ppm (positions a and b in 15b) and generation of a four-proton system centered at 2.57 ppm (positions a, b in 18).

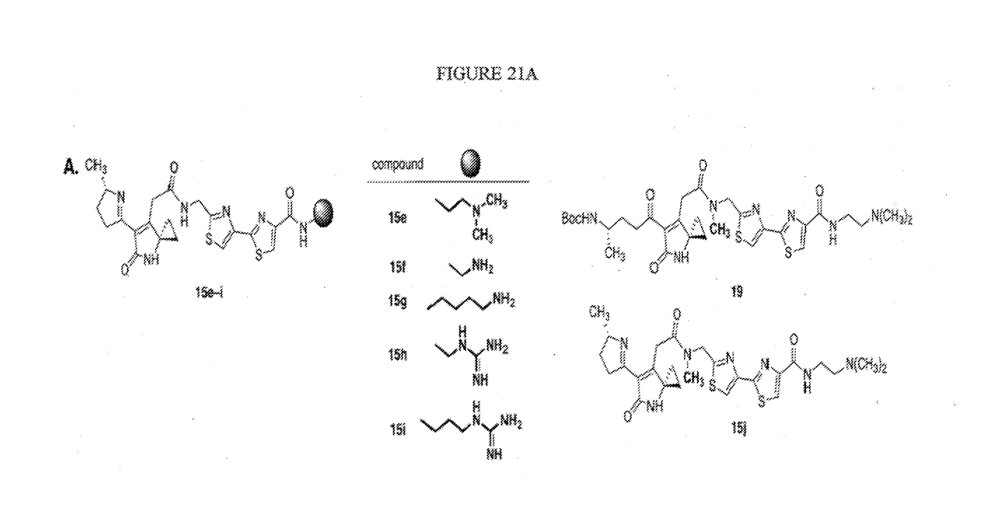

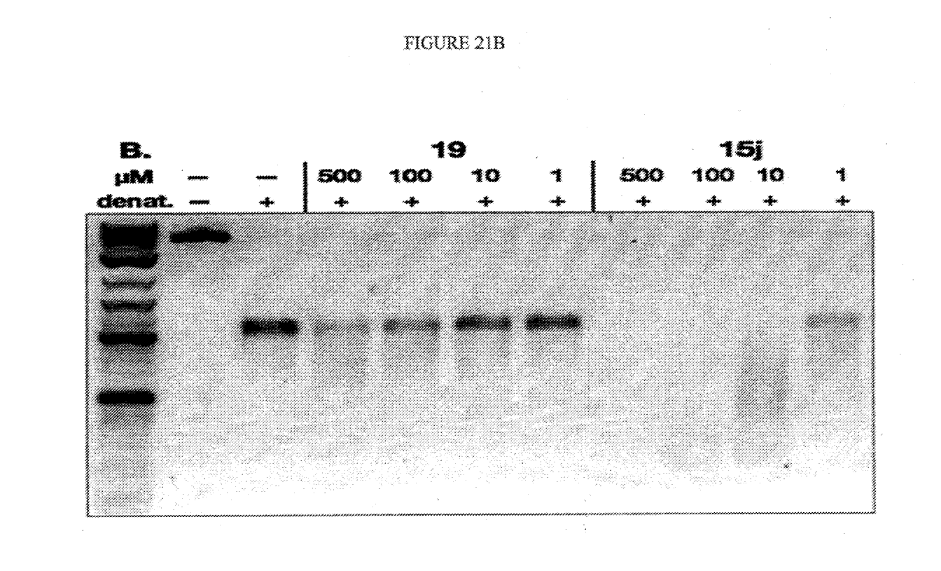

A series of additional compounds were prepared to further interrogate structure-function relationships and optimize DNA alkylation activity (FIG. 21A). The derivatives 15e-15i were synthesized to probe the influence of the nature of the cationic residue on DNA alkylation activity. All compounds were found to potently alkylate DNA although qualitative differences in activity were observed depending on the nature of the cationic residue (guanidine<primary amine<tertiary amine). Finally, to rigorously determine if iminium ion formation is necessary for efficient alkylation, the inventors prepared the unsaturated lactam 19 and the unsaturated imine 15j. Both compounds contain an N-methyl substituent at the central amide residue. This was installed to prevent competitive cyclization of 19 to the corresponding pyridone under the assay conditions; 15j was prepared to confirm that this modification does not influence DNA alkylation activity (as compared to 15a). As shown in FIG. 21B, the lactam 19 showed weak DNA alkylation activity at 500 μM, while the imine derivative 15j was significantly more potent, leading to extensive decomposition of the DNA at low (10 or 1 μM) concentrations, as expected. These final experiments show that the monocyclized derivatives 2 do display some (albeit weak) DNA alkylation activity at high concentrations, but that the unsaturated iminium ion 3 is a significantly more potent electrophile.

### Discussion.

The colibactins are PKS-NRPS-derived natural products and have been implicated in the genotoxic effects of commensal and extraintestinal E. coli.^{2a-c} They are formed from the precolibactins upon removal of an N-acyl-D-Asn side chain by the colibactin peptidase ClbP.⁵ It was proposed that colibactins may generate unsaturated iminium ions that alkylate DNA (see 2, FIG. 15, Scheme 6).⁷ However, this hypothesis has been impossible to evaluate because no colibactins have been obtained from the producing organisms, as researchers have employed clbP mutants to facilitate isolation efforts. This modification is advantageous inasmuch as it allows for the accumulation of candidate precolibactins, determination of their structures, and elucidation of colibactin structures (by inference). However, direct isolation of colibactins is not possible using this strategy.

Many of the advanced isolates reported to date, such as precolibactins A-C (7-9), contain a pyridone nucleus, and the conversion of this aromatic substructure to an unsaturated iminium ion (5→3, FIG. 15, Scheme 6) seems unlikely. The inventors reasoned that removal of ClbP and persistence of an N-acyl-D-Asn side chain engenders unnatural cyclization events leading to the production of the pyridones. This hypothesis was based on our previous synthetic studies, which established a facile double dehydrative cyclization of N-acylated linear precursors 1 to pyridones under mildly acidic or basic conditions.¹⁰ It follows from this line of reasoning that pyridone-containing structures are unlikely to be genotoxic. To test this hypothesis, the inventors prepared the linear precursor 12 (FIG. 16, Scheme 7) and elaborated it to 13 pyridone and unsaturated imine derivatives, in which the substituents throughout the molecules were systematically modified. The successful synthesis of these unsaturated imines allowed us to evaluate their DNA alkylation activity for the first time.

The alkylation assay shown in FIG. 17 demonstrates that the unsaturated imines, but not the corresponding pyridones, are potent DNA alkylation agents. The presence of a terminal cationic substituent enhances DNA alkylation activity (c.f., 15a and 15b, FIG. 17A), as was expected based on literature precedent.¹³ These results are consistent with earlier observations that an α-aminomalonate-derived residue is present in fully-functionalized colibactins and is required for genotoxic effects.^{9b,12} Presumably this residue forms the basis for a cationic substituent that serves the same role as the non-natural cationic functional groups employed herein. It may also be essential for cellular efflux and trafficking to eukaryotic cells.

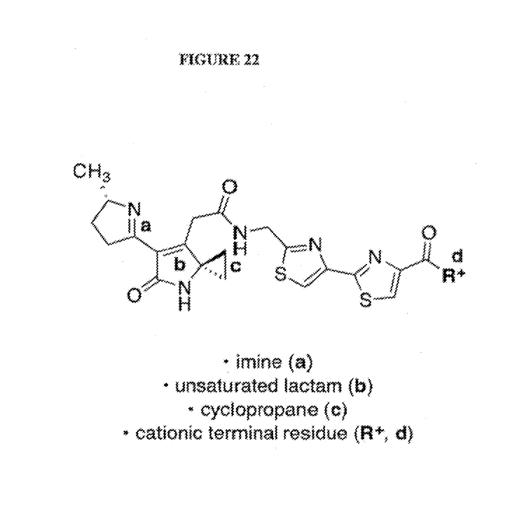

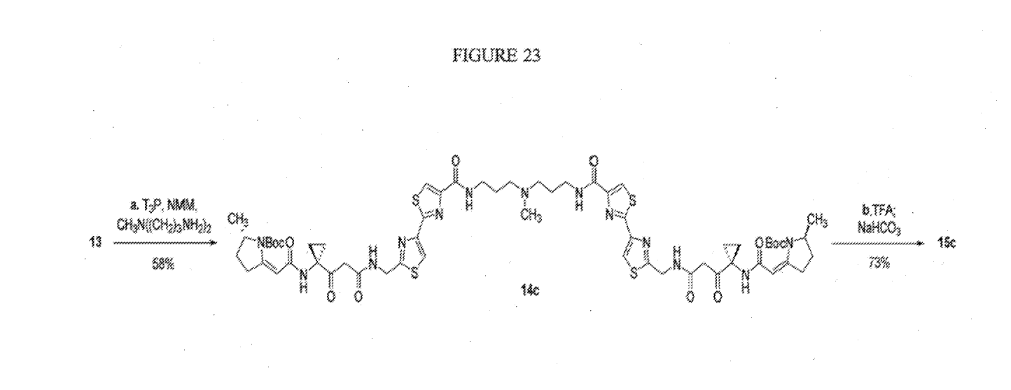

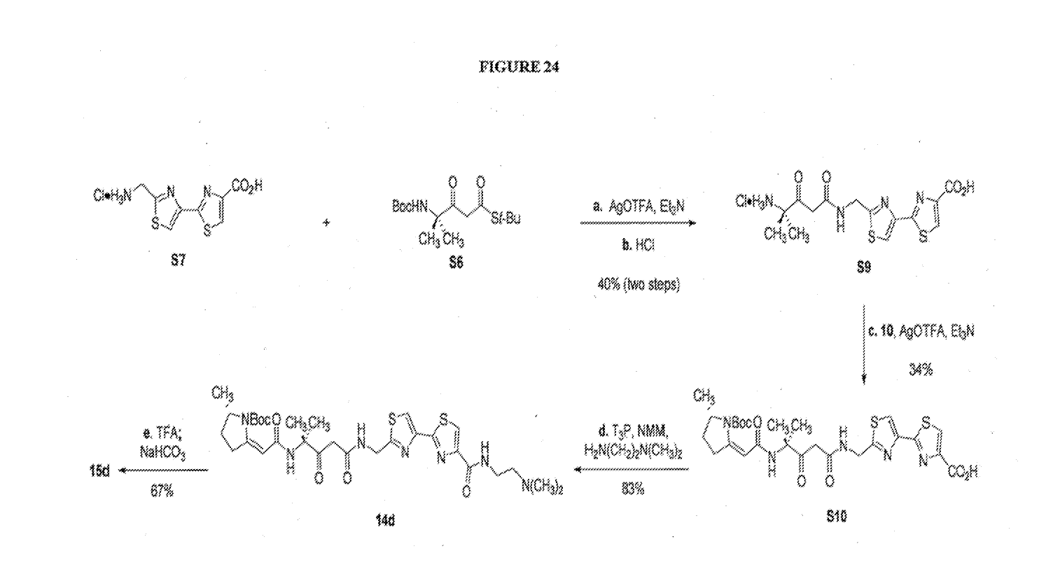

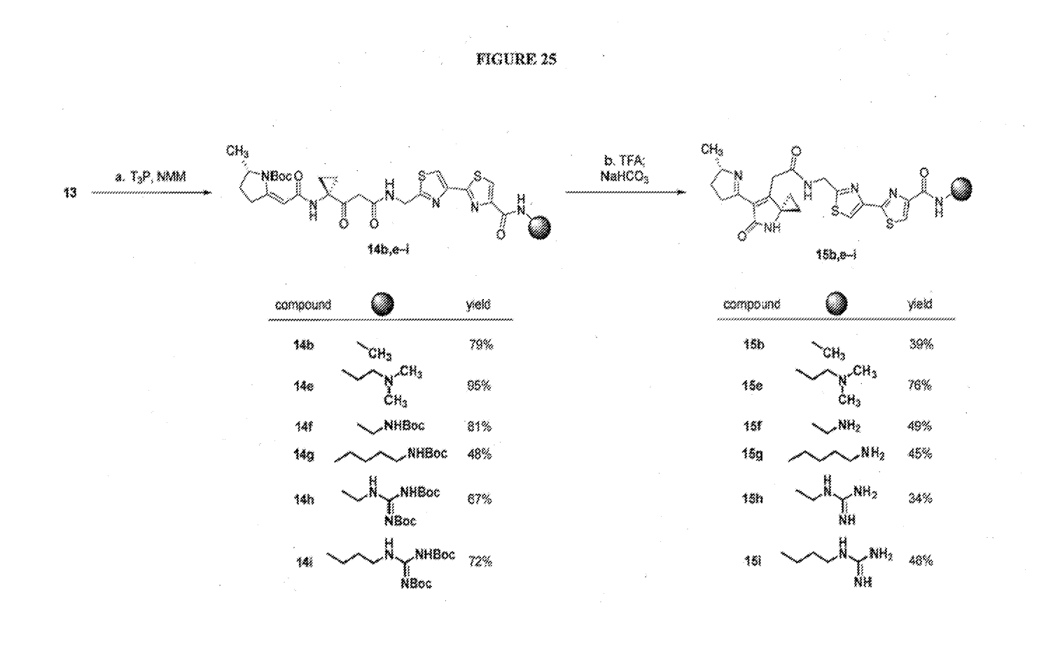

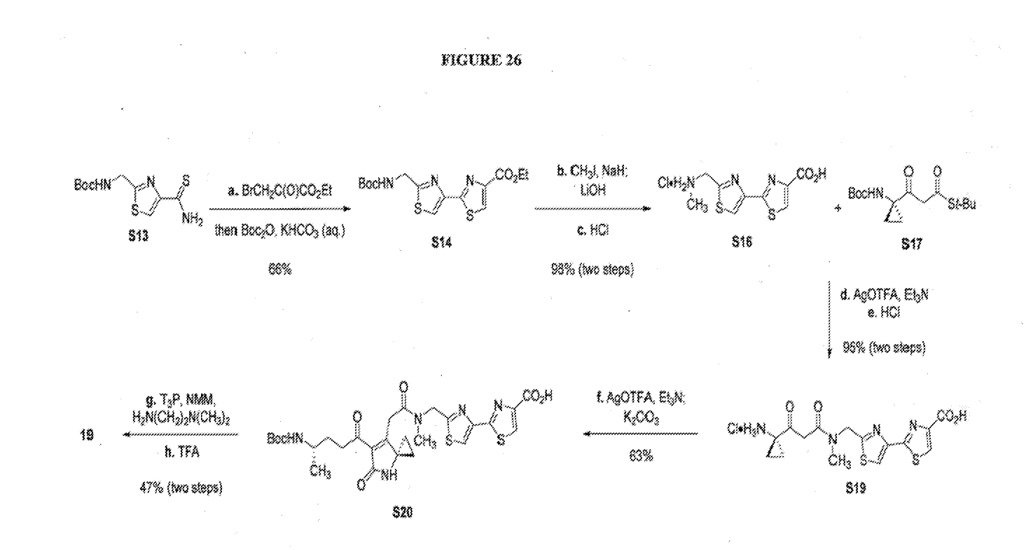

The data support ClbP-mediated deacylation as a key step to prevent formation of pyridone products and facilitate cyclization to unsaturated imines such as 3 (FIG. 15, Scheme 6). While these were originally proposed to form from unsaturated lactams 2, our synthetic studies (FIG. 16, Scheme 7) suggest they may be generated from vinylogous ureas 6 instead. Regardless of the precise mechanism of formation, our structure-function studies show that the unsaturated lactam and cyclopropane are both necessary for DNA alkylation activity (FIG. 22). Formation of an unsaturated imine significantly enhances DNA alkylation activity (c.f., 19 and 15j, FIG. 21 and a cationic terminal residue further increases the extent of DNA alkylation. The observed ring-opening of 15b by propanethiol (FIG. 20, Scheme 8) is consistent with DNA alkylation by cyclopropane ring-opening.

These experiments constitute the first molecular-level analysis of candidate colibactin structure reactivity and provide support for the hypothesis previously proposed to involve DNA alkylation by an unsaturated iminium ion intermediate.⁷ They also suggest that the biosynthetic relevance of precolibactins produced by ΔclbP strains should be interpreted with caution, as it is now clear that the molecules are susceptible to several different modes of reactivity, which vary as a function of substituents within their structures. Indeed, our data indicate that precolibactins A-C (7-9) are unlikely to form the basis of the genotoxicity of the clb gene cluster, a macrocyclic precolibactin recently isolated from a clbP mutant may also similarly derive from unnatural macrocyclization routes.^{12b}

### REFERENCES

1. For reviews, see: (a) Trautman, E. P.; Crawford, J. M. Curr. Top. Med. Chem. 2015, 16, 1. (b) Balskus, E. P. Nat. Prod. Rep. 2015, 32, 1534. (c) Taieb, F.; Petit, C.; Nougayrede, J. P.; Oswald, E. EcoSal Plus 2016, doi.10.1128/ecosalplus.ESP-0008-2016.
2. (a) Nougayrède, J.-P.; Homburg, S.; Taieb, F.; Boury, M.; Brzuszkiewicz, E.; Gottschalk, G.; Buchrieser, C.; Hacker, J.; Dobrindt, U.; Oswald, E. Science 2006, 313, 848. (b) Cuevas-Ramos, G.; Petit, C. R.; Marcq, I.; Boury, M.; Oswald, E.; Nougayrède, J.-P. Proc. Nad. Acad. Sci. U.S.A. 2010, 107, 11537. (c) Arthur, J. C.; Perez-Chanona, E.; Mühlbauer, M.; Tomkovich, S.; Uronis, J. M.; Fan, T.-J.; Campbell, B. J.; Abujamel, T.; Dogan, B.; Rogers, A. B.; Rhodes, J. M.; Stintzi, A.; Simpson, K. W.; Hansen, J. J.; Keku, T. O.; Fodor, A. A.; Jobin, C. Science 2012, 338, 120. (d) Dalmasso, G.; Cougnoux, A.; Delmas, J.; Darfeuille-Michaud, A.; Bonnet, R. Gut Microbes 2014, 5, 675. (e) Grasso, F.; Frisan, T. Biomolecules 2015, 5, 1762. (f) Fais, T.; Delmas, J.; Cougnoux, A.; Dalmasso, G.; Bonnet, R. Gut Microbes 2016, 7, 329. For a review on relationships between gut microbiota composition and cancer, see: (g) Brennan, C. A.; Garrett, W. S. Annu. Rev. Microbiol. 2016, 70, 395.
3. (a) Olier, M.; Marcq, I.; Salvador-Cartier, C.; Secher, T.; Dobrindt, U.; Boury, M.; Bacquié, V.; Penary, M.; Gaultier, E.; Nougayrède, J.-P.; Fioramonti, J.; Oswald, E. Gut Microbes 2012, 3, 501. (b) Scaldaferri, F.; Gerardi, V.; Mangiola, F.; Lopetuso, L. R.; Pizzoferrato, M.; Petito, V.; Papa, A.; Stojanovic, J.; Poscia, A.; Cammarota, G.; Gasbarrini, A. World J. Gastroenterol. 2016, 22, 5505.
4. Mousa, J. J.; Yang, Y.; Tomkovich, S.; Shima, A.; Newsome, R. C.; Tripathi, P.; Oswald, E.; Bruner, S. D.; Jobin, C. Nat. Microbiol 2016, 1, 15009.
5. (a) Dubois, D.; Baron, O.; Cougnoux, A.; Delmas, J.; Pradel, N.; Boury, M.; Bouchon, B.; Bringer, M. A.; Nougayrede, J. P.; Oswald, E.; Bonnet, R. J. Biol. Chem. 2011, 286, 35562. (b) Cougnoux, A.; Gibold, L.; Robin, F.; Dubois, D.; Pradel, N.; Darfeuille-Michaud, A.; Dalmasso, G.; Delmas, J.; Bonnet, R. J. Mol. Biol. 2012, 424, 203. (c) Brotherton, C. A.; Balskus, E. P. J. Am. Chem. Soc. 2013, 135, 3359. (d) Bian, X.; Fu, J.; Plaza, A.; Herrmann, J.; Pistorius, D.; Stewart, A. F.; Zhang, Y.; Muller, R. Chembiochem 2013, 14, 1194. (e) Vizcaino, M. I.; Engel, P.; Trautman, E.; Crawford, J. M. J. Am. Chem. Soc. 2014, 136, 9244.
6. Outer membrane vesicles from Nissle 1917 undergo clathrin-mediated endocytosis in several human epithelial cell lines, suggesting the probiotic effects of Nissle 1917 may not require cell-to-cell contact See: Canas, M. A.; Gimenez, R.; Fabrega, M. J.; Toloza, L.; Baldoma, L.; Badia, J. PLoS One 2016, 11, e0160374.
7. (a) Vizcaino, M. I.; Crawford, J. M. Nat. Chem. 2015, 7, 411. (b) Brotherton, C. A.; Wilson, M.; Byrd, G.; Balskos, E. P. Org. Lett. 2015, 17, 1545.
8. This mode of reactivity bears parallels to DNA alkylation by duocarmycin, CC-1065, and yatakemycin. For reviews, see: (a) Boger, D. L.; Garbaccio, R. M. Acc. Chem. Res. 1999, 32, 1043. (b) Tichenor, M. S.; Boger, D. L. Nat. Prod. Rep. 2008, 25, 220.
9. (a) Li, Z. R.; Li, Y.; Lai, J. Y.; Tang, J.; Wang, B.; Lu, L.; Zhu, G.; Wu, X.; Xu, Y.; Qian, P. Y. Chembiochem 2015, 16, 1715. (b) Zha, L.; Wilson, M. R.; Brotherton, C. A.; Balskus, E. P. ACS Chem. Biol. 2016, 11, 1287.
10. Healy, A. R.; Vizcaino, M. I.; Crawford, J. M.; Herzon, S. B. J. Am. Chem. Soc. 2016, 138, 5426.
11. The structure of precolibactin A was originally predicted in ref. 7a and was later revised to that shown as 7 in ref. 10.
12. (a) Brachmann, A. O.; Garcie, C; Wu, V.; Martin, P.; Ueoka, R.; Oswald, E.; Piel, J. Chem. Commun. 2015, 51, 13138. (b) Li, Z. R.; Li, J.; Gu, J. P.; Lai, J. Y.; Duggan, B. M.; Zhang, W. P.; Li, Z. L.; Li, Y. X.; Tong, R. B.; Xu, Y.; Un, D. H.; Moore, B. S.; Qian, P. Y. Nat. Chem. Biol. 2016, 12, 773.
13. For reviews, see: (a) Tse, W. C.; Boger, D. L. Chem. Biol 2004, 11, 1607. (b) Paul, A.; Bhattacharya, S. Curr. Sci. 2012, 102, 212. (c) Sirajuddin, M.; Ali, S.; Badshah, A. J. Photochem. Photobiol., B 2013, 124, 1.
14. Tanaka, A.; Usuki, T. Tetrahedron Lett. 2011, 52, 5036.
15. Basavaprabhu; Vishwanatha, T. M.; Panguluri, N. R.; Sureshbabu, V. V. Synthesis 2013, 45, 1569.
16. Lundin, C.; North, M.; Erixon, K.; Walters, K.; Jenssen, D.; Goldman, A. S. H.; Helleday, T. Nucleic Acids Res. 2005, 33, 3799.

### EXAMPLES—FIRST SET (FIRST SET OF REFERENCES APPLIES)

### General Experimental Methods.

### Cysteine Incorporation Based on Carbon and Deuterium Labeling.

Nonlabeled control culture conditions, L-[U-¹³C]-Cys isotope culture conditions, and L-[2,3,3-D]-Cys isotope culture conditions were employed as previously reported.¹

### LC-HRMS Analysis for the in Vivo Cleavage of Synthetic 6 by ClbP.

E. coli DH10B pClbP (pPEB018) and the bacteria harboring the empty vector pBAD18 (as previously reported³) were grown in LB medium supplemented with 100 μg/mL ampicillin. The next morning, 50 μL of these saturated cultures were used to inoculate 2.5 mL of LB medium containing 100 μg/mL ampicillin. Cultures were incubated at 37° C. with shaking at 250 rpm. At an OD₆₀₀ of 0.4-0.5, cultures were induced with L-arabinose and left to grow for another 30 min. Then, the synthetic 6 substrate (10 mM stock solution in DMSO) or DMSO (vector control) was added to each culture to a final concentration of 50 μM, and incubated at 37° C. for 24 h. At the designated time point, the cultures were extracted with organic solvent and analyzed by LC-HRMS on a Phenomenex C18-A column (150×4.6 mm, 180 Å, 5 μm particle size, Agilent) with a water:acetonitrile (ACN) gradient containing 0.1% formic acid 0.7 mL/min: 1-2 min, 5% ACN; ramp to 98% ACN over 18 min; hold for 5 min at 100% ACN.

### Extraction of Naturally-Produced Advanced Precolibactins.

Escherichia coli DH10B pBAC ΔclbP, generated by nonpolar gene deletion of clbP,³ was used in co-injection experiments as natural compound comparison with synthetic compounds. A single colony of the ΔclbP strain was grown in M9 media as previously reported.¹ At the designated time point, the culture was extracted with ethyl acetate (EtOAc), and the re-constituted organic extract was utilized in comparison studies with synthetic precolibactins.

### HRMS, and MS/MS Data Acquisition.

All liquid chromatography high-resolution mass spectrometry (LC-HRMS) data described for this paper was collected on an Agilent iFunnel 6550 Quadrupole time-of-flight (QTOF) mass spectrometer equipped with an electrospray ionization (ESI) source coupled to an Agilent Infinity 1290 UHPLC scanning from m/z 50-1200. Data was acquired using MassHunter Workstation Software LC/MS Data Acquisition (Version B.05.01, Agilent Technologies) and processed with Qualitative Analysis (Version B.06.00). Co-injection experiments were analyzed on a C18 Kinetex column (2.5×100 mm, 1.7 μm) using water (0.1% formic acid) as mobile phase A and acetonitrile (0.1% formic acid) as mobile phase B. Gradient conditions were as follow: 0-2 min, 5% B; ramp to 75% B over 20 min; wash at 98% B for 6 min, and equilibrate at 5% B for 8 min. Flow rate was set at 0.3 mL/min, and injection volume at 5 μL. MS data was collected in ESI+ mode with source gas temp at 225° C., drying gas at 15 l/min, nebulizer at 35 psig, Vcap set at 4000V, Nozzle Voltage at 1000 V. Acquisition rate was 1 spectra/s. MSMS fragmentation was acquired with three collision energies (40, 60, 90) with an unbiased isotope model.

### Dft Calculations.

Lowest-energy conformations of 40 and 41 were obtained by molecular mechanics optimization (500 starting conformers) using BOSS.⁴ The corresponding lowest-energy conformers were then subjected to DFT optimization in Gaussian 09 [B3LYP 6-31G(d,p+] in a water environment.⁵

### General Experimental Procedures.

All reactions were performed in single-neck, flame-dried, round-bottomed flasks fitted with rubber septa under a positive pressure of nitrogen unless otherwise noted. Air- and moisture-sensitive liquids were transferred via syringe or stainless steel cannula, or were handled in a nitrogen-filled drybox (working oxygen level <10 ppm). Organic solutions were concentrated by rotary evaporation at 28-32° C. Flash-column chromatography was performed as described by Still et al.,⁶ employing silica gel (60 Å, 40-63 μm particle size) purchased from Sorbent Technologies (Atlanta, Ga.). Anion-exchange chromatography was performed as described by Bland et al.,⁷ employing trimethylamine acetate-functionalized silica gel (SiliaBond® TMA Acetate). Analytical thin-layered chromatography (TLC) was performed using glass plates pre-coated with silica gel (0.25 mm, 60 Å pore size) impregnated with a fluorescent indicator (254 nm). TLC plates were visualized by exposure to ultraviolet light (UV).

### Materials.

Commercial solvents and reagents were used as received with the following exceptions. Dichloromethane, diethyl ether and N,N-dimethylformamide were purified according to the method of Pangborn et al.⁸ Triethylamine was distilled from calcium hydride under an atmosphere of argon immediately before use. Di-iso-propylamine was distilled from calcium hydride and was stored under nitrogen. Methanol was distilled from magnesium turnings under an atmosphere of nitrogen immediately before use. Tetrahydrofuran was distilled from sodium-benzophenone under an atmosphere of nitrogen immediately before use. Trifluoroacetic anhydride was fractionally-distilled before use. Molecular sieves were activated by heating to 200° C. under vacuum (<1 Torr) for 12 h, and were stored in an oven at >160° C. Propylsulfonic acid-functionalized silica gel (SiliaBond® SCX-2) and trimethylamine acetate-functionalized silica gel (SiliaBond® TMA Acetate) were purchased from SiliCycle (Quebec City, Calif.). (−)-(R_{S})-2-methyl-N-(pent-4-en-1-ylidene)propane-2-sulfinamide (S1)⁹, 3-(tert-butylthio)-3-oxopropanoic acid (S7)¹⁰ and 2-(((tert-butoxycarbonyl)amino)methyl)thiazole-4-carboxylic acid (S12)¹¹ were prepared according to published procedures.

### Instrumentation.

Proton nuclear magnetic resonance spectra (¹H NMR) were recorded at 400, 500, or 600 MHz at 24° C., unless otherwise noted. Chemical shifts are expressed in parts per million (ppm, δ scale) downfield from tetramethylsilane and are referenced to residual protium in the NMR solvent (CDCl₃, δ 7.26; CD₃OD, δ 3.31; C₂D₆OS, δ 2.50). Data are represented as follows: chemical shift, multiplicity (s=singlet, d=doublet, t=triplet, q=quarter, m=multiplet and/or multiple resonances, br=broad, app=apparent), coupling constant in Hertz, integration, and assignment. Proton-decoupled carbon nuclear magnetic resonance spectra (¹³C NMR) were recorded at 100, 125 or 150 MHz at 24° C., unless otherwise noted. Chemical shifts are expressed in parts per million (ppm, δ scale) downfield from tetramethylsilane and are referenced to the carbon resonances of the solvent (CDCl₃, δ 77.0; CD₃OD, δ 49.0; C₂D₆OS, δ 39.5). Signals of protons and carbons were assigned, as far as possible, by using the following two dimensional NMR spectroscopy techniques: [1H, 1H] COSY (Correlation Spectroscopy), [1H, 13C] HSQC (Heteronuclear Single Quantum Coherence) and long range [1H, 13C] HMBC (Heteronuclear Multiple Bond Connectivity). Attenuated total reflectance Fourier transform infrared (ATR-FTIR) spectra were obtained using a Thermo Electron Corporation Nicolet 6700 FTIR spectrometer referenced to a polystyrene standard. Data are represented as follows: frequency of absorption (cm⁻¹), intensity of absorption (s=strong, m=medium, w=weak, br=broad). Analytical ultra high-performance liquid chromatography/mass spectrometry (UPLC/MS) was performed on a Waters UPLC/MS instrument equipped with a reverse-phase C₁₈ column (1.7 μm particle size, 2.1×50 mm), dual atmospheric pressure chemical ionization (API)/electrospray (ESI) mass spectrometry detector, and photodiode array detector. Samples were eluted with a linear gradient of 5% acetonitrile-water containing 0.1% formic acid→100% acetonitrile containing 0.1% formic acid over 0.75 min, followed by 100% acetonitrile containing 0.1% formic acid for 0.75 min, at a flow rate of 800 μL/min. High-resolution mass spectrometry (HRMS) were obtained on a Waters UPLC/HRMS instrument equipped with a dual AP/ESI high-resolution mass spectrometry detector and photodiode array detector. Unless otherwise noted, samples were eluted over a reverse-phase C₁₈ column (1.7 μm particle size, 2.1×50 mm) with a linear gradient of 5% acetonitrile-water containing 0.1% formic acid→95% acetonitrile-water containing 0.1% formic acid for 1 min, at a flow rate of 600 μL/min. Optical rotations were measured on a Perkin Elmer polarimeter equipped with a sodium (589 nm, D) lamp. Optical rotation data are represented as follows: specific rotation ([α]_{δ}^{T}), concentration (g/100 mL), and solvent.

### Synthetic Procedures.

### Synthesis of the Sulfinamine S2:

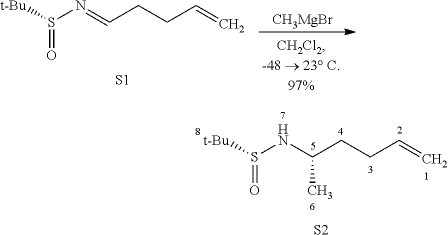

A solution of methylmagnesium bromide in ether (3.0 N, 3.56 mL, 10.7 mmol, 2.00 equiv) was added dropwise via syringe pump over 30 min to a solution of the sulfinimine S1 (1.00 g, 5.34 mmol, 1 equiv) in dichloromethane (35 mL) at −48° C. The resulting mixture was allowed to warm over 30 min to −30° C. The reaction mixture was stirred for 4 h at −30° C. The reaction mixture was then allowed to warm over 30 min to 23° C. The product mixture was diluted sequentially with saturated aqueous ammonium chloride solution (20 mL) and ethyl acetate (20 mL). The diluted product mixture was transferred to a separatory funnel and the layers that formed were separated. The aqueous layer was extracted with ethyl acetate (2×30 mL). The organic layers were combined and the combined organic layers were washed with saturated aqueous sodium chloride solution (30 mL). The washed organic layer was dried over sodium sulfate. The dried solution was filtered and the filtrate was concentrated to provide the unpurified sulfinamine S2 as a yellow oil (1.05 g, 97%).

The product S2 obtained in this way was estimated to be of >95% purity and 88% de by ¹H NMR analysis (see accompanying spectrum) and was used without further purification. The configuration of the newly-formed stereocenter was assigned by analogy to related addition products.^{12,13}

¹H NMR (400 MHz MHz, CDCl₃): δ 5.79 (ddt, J=16.9, 10.1, 6.5 Hz, 1H, H₂), 5.08-4.87 (m, 2H, H₁), 3.37 (app hept, J=6.8 Hz, 1H, H₅), 2.86 (d, J=7.2 Hz, 1H, H₇), 2.20-2.04 (m, 2H, H₃), 1.66-1.42 (m, 2H, H₄), 1.28 (d, J=6.5 Hz, 3H, H₆), 1.20 (s, 9H, H₈). ¹³C NMR (151 MHz, CDCl₃) δ 138.2 (CH), 115.1 (CH₂), 55.8 (C), 52.3 (CH), 37.5 (CH₂), 30.2 (CH₂), 23.4 (CH₃), 22.8 (CH). IR (ATR-FTIR), cm⁻¹: 2972 (w), 1642 (s), 1457 (m), 1364 (s), 1050 (s), 910 (s). HRMS-CI (m/z): [M+Na]⁺ calcd for C₁₀H₂₁NNaOS, 226.1242; found, 226.1294. [α]_{D}²⁰ −21.2 (c 1.0, CH₃OH).

### Synthesis of the Amine 8:

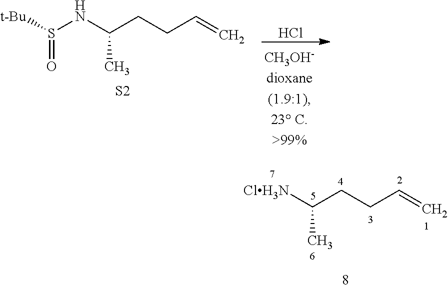

A solution of hydrogen chloride in 1,4-dioxane (4.0 N, 2.58 mL, 10.3 mmol, 2.00 equiv) was added dropwise via syringe pump over 30 min to a solution of the sulfinamine S2 (1.05 g, 5.16 mmol, 1 equiv) in methanol (5.0 mL) at 23° C. The resulting mixture was stirred for 1 h at 23° C. The product mixture was concentrated to dryness. The residue obtained was suspended in ether (10 mL) and the resulting suspension was concentrated to dryness. This process was repeated to provide the amine 8 as white solid (700 mg, >99%, CAUTION: hygroscopic).

The product 8 obtained in this way was estimated to be of >95% purity by ¹H and ¹³C NMR analysis (see accompanying spectra) and was used without further purification.

¹H NMR (600 MHz, CDCl₃) δ 8.36 (bs, 3H, H₇), 5.75 (ddt, J=17.0, 10.3, 6.6 Hz, 1H, H₂), 5.11 (dd, J=17.0, 1.9 Hz, 1H, H₁), 5.02 (dd, J=10.3, 1.9 Hz, 1H, H₁), 3.46-3.03 (m, 1H, H₅), 2.34-2.09 (m, 2H, H₃), 1.98-1.87 (m, 1H, H₄), 1.76-1.67 (m, 1H, H₄), 1.42 (d, J=6.3 Hz, 1H). ¹³C NMR (151 MHz, CDCl₃) δ 136.4 (CH), 116.5 (CH₂), 48.0 (CH), 34.1 (CH₂), 29.7 (CH₂), 18.8 (CH₃). IR (ATR-FTIR), cm⁻¹: 2915 (w), 1645 (s), 1612 (s), 1510 (s), 1454 (s), 1390 (s), 1198 (s), 996 (s), 910 (s). [α]_{D}²⁰ +3.5 (c 2.0, CH₃OH).

### Synthesis of the Alkene S3:

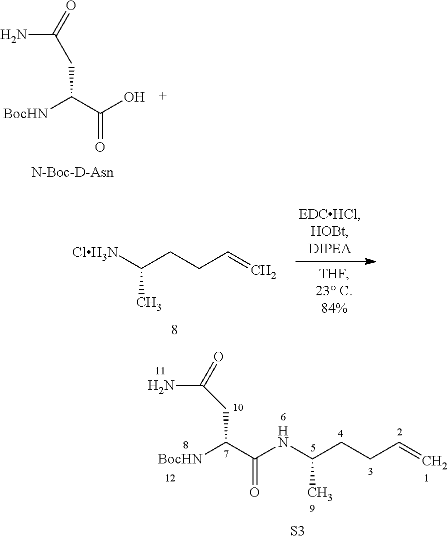

N_{α}-(tert-butoxycarbonyl)-D-asparagine (880 mg, 3.79 mmol, 1 equiv), 1-hydroxybenzotriazole hydrate (HOBt, 638 mg, 4.17 mmol, 1.10 equiv), N,N-diisopropylethylamine (1.45 mL, 8.34 mmol, 2.20 equiv), and N-(3-dimethylaminopropyl)-N′-ethylcarbodiimide hydrogenchloride (EDC.HCl, 726 mg, 3.79 mmol, 1.00 equiv) were added in sequence to a solution of the amine 8 (514 mg, 3.79 mmol, 1.00 equiv) in tetrahydrofuran (40 mL) at 23° C. The reaction mixture was stirred for 4 h at 23° C. The heterogeneous product mixture was concentrated and the residue obtained was diluted with saturated aqueous ammonium chloride solution (60 mL). The resulting mixture was extracted with ethyl acetate (5×30 mL) and the organic layers were combined. The combined organic layers were washed sequentially with water (30 mL) and saturated aqueous sodium chloride solution (30 mL). The washed organic layer was dried over magnesium sulfate and the dried solution was filtered. The filtrate was concentrated to provide the alkene S3 as a white solid (995 mg, 84%).

The product S3 obtained in this way was estimated to be of >95% purity by ¹H and ¹³C NMR analysis (see accompanying spectra) and was used without further purification.

¹H NMR (400 MHz, DMSO-d₆) δ 7.46 (d, J=8.5 Hz, 1H, H₆), 7.25 (bs, 1H, H₁₁), 6.87 (bs, 1H, H₁₁), 6.84 (d, J=8.1 Hz, 1H, H₈), 5.77 (ddt, J=16.9, 10.2, 6.6 Hz, 1H, H₂), 4.98 (dd, J=17.2, 2.0 Hz, 1H, H₁), 4.92 (d, J=10.2 Hz, 1H, H₁), 4.14 (td, J=8.0, 5.6 Hz, 1H, H₇), 3.83-3.54 (m, 1H, H₅), 2.43-2.27 (m, 2H, H₁₀), 2.10-1.90 (m, 2H, H₃), 1.53-1.37 (m, 2H, H₄), 1.37 (s, 9H, H₁₂), 1.00 (d, J=6.5 Hz, 3H, H₉). ¹³C NMR (151 MHz, DMSO-d₆) δ 171.5 (C), 170.5 (C), 155.1 (C), 138.5 (CH), 114.7 (CH₂), 78.1 (C), 51.5 (CH), 43.8 (CH), 37.3 (CH₂), 35.2 (CH₂), 29.8 (CH₂), 28.2 (CH₃), 20.7 (CH₃). IR (ATR-FTIR), cm⁻¹: 3393 (br), 3298 (br), 2977 (w), 2930 (w), 1688 (m), 1635 (s), 1552 (m), 1519 (m), 1171 (s), 1054 (m), 610 (br). HRMS-CI (m/z): [M+H]⁺ calcd for C₁₅H₂₇N₃O₄, 314.2080; found, 314.2001. [α]_{D}²⁰ +15.4 (c 0.9, CH₃OH).

### Synthesis of the Amine 9:

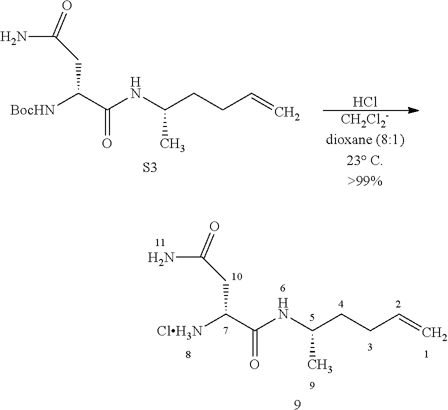

A solution of hydrogen chloride in 1,4-dioxane (4.0 N, 4.31 mL, 17.2 mmol, 6.00 equiv) was added dropwise via syringe pump over 20 min to a solution of the alkene S3 (900 mg, 2.87 mmol, 1 equiv) in dichloromethane (30 mL) at 23° C. The resulting mixture was stirred for 1 h at 23° C. The product mixture was concentrated to provide the amine 9 as a white solid (717 mg, >99%).

The product 9 obtained in this way was estimated to be of >95% purity by ¹H and ¹³C NMR analysis (see accompanying spectra) and was used without further purification.

¹H NMR (600 MHz, DMSO-d₆) δ 8.34 (d, J=8.0 Hz, 1H, H₆), 8.19 (bs, 3H, H₈), 7.72 (bs, 1H, H₁₁), 7.22 (bs, 1H, H₁₁), 5.79 (ddt, J=16.9, 10.2, 6.6 Hz, 1H, H₂), 5.02 (dd, J=17.2, 1.9 Hz, 1H, H₁), 4.95 (dt, J=10.2, 1.7 Hz, 1H, H₁), 4.05-3.85 (m, 1H, H₇), 3.84-3.64 (m, 1H, H₅), 2.67 (dd, J=16.5, 5.1 Hz, 1H, H₁₀), 2.60 (dd, J=16.5, 8.1 Hz, 1H, H₁₀), 2.13-1.89 (m, 2H, H₃), 1.59-1.41 (m, 2H, H₄), 1.04 (d, J=6.6 Hz, 3H, H₉). ¹³C NMR (151 MHz, DMSO-d₆) δ 170.6 (C), 167.0 (C), 138.3 (CH), 114.9 (CH₂), 49.3 (CH), 44.5 (CH), 35.6 (CH₂), 34.8 (CH₂), 29.8 (CH₂), 20.4 (CH₃). IR (ATR-FTIR), cm⁻¹: 2932 (br), 1655 (s), 1548 (m), 1452 (m), 1427 (m), 906 (m), 812 (br). HRMS-CI (m/z): [M+Na]⁺ calcd for C₁₀H₁₉N₃NaO₂, 236.1375; found, 236.1361. [α]_{D}²⁰ −3.9 (c 1.0, CH₃OH).

### Synthesis of the Alkene S4:

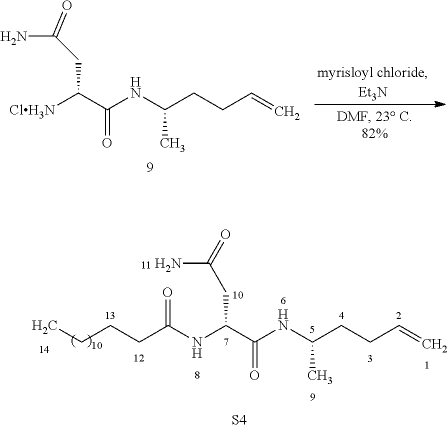

Triethylamine (977 μL, 7.01 mmol, 2.50 equiv) and myristoyl chloride (990 μL, 3.64 mmol, 1.30 equiv) were added in sequence to a solution of the amine 9 (700 mg, 2.80 mmol, 1 equiv) in N,N-dimethylformamide (35 mL) at 23° C. The reaction mixture was stirred for 4 h at 23° C. The heterogeneous product mixture was diluted with aqueous hydrogen chloride solution (1.0 N, 50 mL). The precipitate that formed was isolated by filtration and the isolated precipitate was washed sequentially with aqueous hydrogen chloride solution (1.0 N, 20 mL) and water (20 mL). The resulting solid was triturated with dichloromethane (20 mL) to afford the alkene S4 as a white solid (974 mg, 82%).

The product S4 obtained in this way was estimated to be of >95% purity by ¹H and ¹³C NMR analysis (see accompanying spectra) and was used without further purification.

¹H NMR (600 MHz, DMSO-d₆) δ 7.91 (d, J=8.1 Hz, 1H, H₈), 7.44 (d, J=8.4 Hz, 1H, H₆), 7.24 (bs, 1H, H₁), 6.83 (bs, 1H, H₁), 5.77 (ddt, J=16.9, 10.2, 6.6 Hz, 1H, H₂), 4.98 (dd, J=17.2, 2.0 Hz, 1H, H₁), 4.92 (dd, J=10.2, 2.0 Hz, 1H, H₁), 4.52-4.40 (m, 1H, H₇), 3.80-3.59 (m, 1H, H₅), 2.44 (dd, J=15.2, 6.2 Hz, 1H, H₁₀), 2.32 (dd, J=15.2, 7.7 Hz, 1H, H₁₀), 2.08 (t, J=7.5 Hz, 2H, H₁₂), 2.03-1.89 (m, 2H, H₃), 1.51-1.34 (m, 4H, H₄, H₁₃), 1.23 (s, 20H, CH₂), 1.00 (d, J=6.6 Hz, 3H, H₉), 0.85 (t, J=6.9 Hz, 3H, H₁₄). ¹³C NMR (151 MHz, DMSO-d₆) δ 172.1 (C), 171.4 (C), 170.3 (C), 138.5 (CH), 114.8 (CH₂), 49.8 (CH), 43.8 (CH), 37.4 (CH₂), 35.2 (2×CH₂), 31.4 (CH₂), 29.9 (CH₂), 29.1 (3×CH₂), 29.0 (2×CH₂), 28.9 (CH₂), 28.7 (CH₂), 28.6 (CH₂), 25.3 (CH₂), 22.2 (CH₂), 20.6 (CH₃), 14.1 (CH₃). IR (ATR-FTIR), cm⁻¹: 3297 (w), 2916 (w), 2850 (s), 1662 (s), 1638 (s), 1540 (s), 1394 (m), 909 (s). HRMS-CI (m/z): [M+H]⁺ calcd for C₂₄H₄₆N₃O₃, 424.3539; found, 424.3516. [α]_{D}²⁰ +23.7 (c 0.5, CH₃OH-DMF (1:1)).

### Synthesis of the Carboxylic Acid 10:

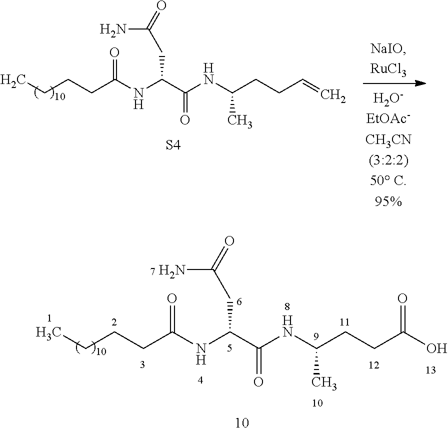

Water (12 mL), sodium periodate (611 mg, 2.86 mmol, 4.10 equiv), and ruthenium chloride (3.9 mg, 17.0 μmol, 0.025 equiv) were added in sequence to a suspension of the alkene S4 (295 mg, 696 μmol, 1 equiv) in ethyl acetate (8.0 mL) and acetonitrile (8.0 mL) at 23° C. The reaction vessel was placed in an oil bath that had been preheated to 50° C. The reaction mixture was stirred vigorously for 2 h at 50° C. The heterogeneous product mixture was partially concentrated to remove ethyl acetate and acetonitrile. The partially concentrated solution was diluted with aqueous hydrogen chloride solution (1.0 N, 50 mL). The precipitate that formed was isolated by filtration and the isolated precipitate was dissolved in ethyl acetate-methanol (5:1 v/v, 60 mL). Activated charcoal (4.5 g) was added and the resulting heterogeneous mixture was stirred for 3 h at 23° C. The stirred heterogeneous mixture was filtered and the filtrate was concentrated to provide the carboxylic acid 10 as a white solid (292 mg, 95%).

The product 10 obtained in this way was estimated to be of >95% purity by ¹H and ¹³C NMR analysis (see accompanying spectra) and was used without further purification.

¹H NMR (600 MHz, DMSO-d₆) δ 7.90 (d, J=8.0 Hz, 1H, H₄), 7.48 (d, J=8.4 Hz, 1H, H₈), 7.25 (bs, 1H, H₇), 6.84 (bs, 1H, H₇), 4.46 (td, J=7.8, 6.0 Hz, 1H, H₅), 3.77-3.64 (m, 1H, H₉), 2.43 (dd, J=15.2, 6.0 Hz, 1H, H₆), 2.32 (dd, J=15.2, 7.7 Hz, 1H, H₆), 2.24-2.09 (m, 2H, H₂), 2.08 (t, J=7.5 Hz, 2H, H₃), 1.65-1.51 (m, 2H, H₁₁), 1.51-1.36 (m, 2H, H₂), 1.23 (bs, 20H, CH₂), 1.00 (d, J=6.6 Hz, 3H, H₁₀), 0.85 (t, J=7.0 Hz, 3H, H₁). ¹³C NMR (151 MHz, DMSO-d₆) δ 174.4 (C), 172.1 (C), 171.4 (C), 170.4 (C), 49.8 (CH), 43.9 (CH), 37.4 (CH₂), 35.2 (CH₂), 31.3 (CH₂), 31.2 (CH₂), 30.5 (CH₂), 29.09 (CH₂), 29.07 (2×CH₂), 29.04 (CH₂), 28.97 (CH₂), 28.86 (CH₂), 28.73 (CH₂), 28.65 (CH₂), 25.2 (CH₂), 22.1 (CH₂), 20.5 (CH₃), 14.0 (CH₃). IR (ATR-FTIR), cm⁻¹: 3295 (w), 2920 (w), 2851 (s), 1666 (s), 1631 (s), 1541 (s), 1340 (m). HRMS-CI (m/z): [M+H]⁺ calcd for C₂₃H₄₄N₃O₅, 442.3281; found, 442.3251. [α]_{D}²⁰ +16.6 (c 0.9, CH₃OH).

### Synthesis of the Thiazoline 12:

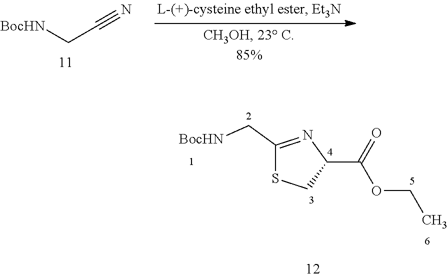 [SMILES: CC(C)(C)OC(=O)NCC#N ; CCOC(=O)[C@@H]1CSC(CNC(=O)OC(C)(C)C)=N1]

Triethylamine (540 μL, 3.84 mmol, 0.20 equiv) was added to a deoxygenated solution of N-(tert-butoxycarbonyl)-2-aminoacetonitrile (11, 3.00 g, 19.2 mmol, 1 equiv) and L-(+)-cysteine ethyl ester hydrogenchloride (5.35 g, 28.8 mmol, 1.50 equiv) in methanol (35 mL) at 23° C. The resulting mixture was stirred for 14 h at 23° C. The product mixture was concentrated and the residue obtained was purified by flash-column chromatography (eluting with 20% ethyl acetate-hexanes initially, grading to 40% ethyl acetate-hexanes, linear gradient) to provide the thiazoline 12 as a colorless oil (4.71 g, 85%).

The product 12 obtained in this way was estimated to be of >95% purity by ¹H and ¹³C NMR analysis (see accompanying spectra) and was used without further purification.

¹H and ¹³C NMR data for the thiazoline 12 prepared in this way were in agreement with the literature.¹⁴

¹H NMR (600 MHz, DMSO-d₆) δ 7.46 (t, J=6.3 Hz, 1H), 5.12 (app t, J=9.3 Hz, 1H, H₄), 4.23-4.06 (m, 2H, H₅), 3.94-3.90 (m, 2H, H₂), 3.53 (app t, J=10.5 Hz, 1H, H₃), 3.39 (dd, J=11.3, 8.9 Hz, 1H, H₃), 1.39 (s, 9H, H₁), 1.22 (t, J=7.1 Hz, 3H, H₆). ¹³C NMR (151 MHz, DMSO-d₆) δ 173.8 (C), 170.4 (C), 155.6 (C), 78.4 (C), 78.0 (CH), 61.0 (CH₂), 42.3 (CH₂), 33.9 (CH₂), 28.2 (CH₃), 14.0 (CH₂).

### Synthesis of the Amide S5:

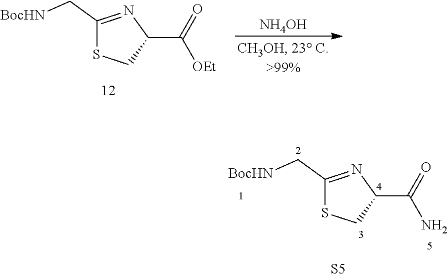 [SMILES: CCOC(=O)[C@@H]1CSC(CNC(=O)OC(C)(C)C)=N1 ; "NH4OH" ; CC(C)(C)OC(=O)NCC1=N[C@H](C(N)=O)CS1 ; "S5"]

Aqueous ammonium hydroxide solution (28% w/v, 50 mL) was added to a solution of the ester 12 (3.86 g, 13.5 mmol, 1 equiv) in methanol (100 mL) at 23° C. The resulting mixture was stirred for 16 h at 23° C. The product mixture was concentrated and the residue obtained was dried by azeotropic distillation from toluene (2×30 mL) to provide the amide S5 as a white solid (3.49 g, >99/%).

The product S5 obtained in this way was estimated to be of >95% purity by ¹H and ¹³C NMR analysis (see accompanying spectra) and was used without further purification.

¹H NMR (600 MHz, DMSO-d₆) δ 7.45 (t, J=6.1 Hz, 1H), 7.33 (bs, 1H, H₅), 7.14 (bs, 1H, H₅), 4.99-4.91 (m, 1H, H₄), 4.02-3.82 (m, 2H, H₂), 3.54-3.39 (m, 2H, H₃), 1.39 (s, 9H, H₁). ¹³C NMR (151 MHz, DMSO-d₆) δ 172.5 (C), 172.3 (C), 155.7 (C), 78.5 (CH), 78.4 (C), 42.5 (CH₂), 34.6 (CH₂), 28.2 (CH₃). IR (ATR-FTIR), cm⁻¹: 3321 (w), 1684 (s), 1615 (m), 1503 (s), 1251 (s), 1156 (w). m/z (ES+) 260.15 ([M+H]+, 100%). [α]_{D}²⁰ +6.5 (c 0.8, CH₃OH).

### Synthesis of the Thioamide 13:

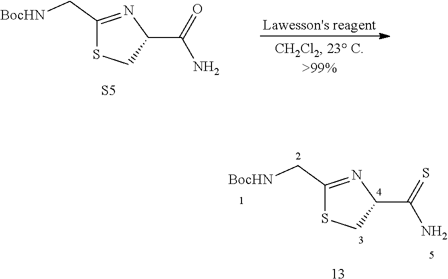 [SMILES: CC(C)(C)OC(=O)NCC1=N[C@H](C(N)=O)CS1 ; "Lawesson's reagent" ; "S5" ; CC(C)(C)OC(=O)NCC1=N[C@H](C(N)=S)CS1 ; "13"]

Lawesson's reagent (4.03 g, 9.96 mmol, 0.75 equiv) was added to a solution of the amide S5 (3.44 g, 13.3 mmol, 1 equiv) in dichloromethane (80 mL) at 23° C. The resulting mixture was stirred for 16 h at 23° C. The product mixture was filtered through a pad of celite (2.5×4.5 cm). The filter cake was washed with dichloromethane (20 mL). The filtrates were combined and the combined filtrates were concentrated. The residue obtained was dissolved in ethyl acetate (50 mL) and the resulting solution was washed sequentially with saturated aqueous sodium bicarbonate solution (2×30 mL) and saturated aqueous sodium chloride solution (30 mL). The washed organic layer was dried over sodium sulfate. The dried solution was filtered and the filtrate was concentrated to provide the thioamide 13 as a pale yellow solid (3.66 g, >99%).

The thioamide 13 obtained in this way was estimated to be of >95% purity by ¹H and ¹³C NMR analysis (see accompanying spectra) and was used without further purification.

¹H NMR (600 MHz, CDCl₃) δ 8.37 (bs, 1H, H₅), 7.74 (bs, 1H, H₅), 5.35 (t, J=9.1 Hz, 1H, H₄), 5.29-5.12 (m, 1H), 4.18-4.05 (m, 2H, H₂), 3.90-3.83 (m, 1H, H₃), 3.90-3.83 (m, 1H, H₃), 1.45 (s, 9H, H₁). ¹³C NMR (151 MHz, CDCl₃) δ 206.7 (C), 174.5 (C), 156.2 (C), 84.3 (CH), 80.7 (C), 43.6 (CH₂), 39.3 (CH₂), 28.4 (CH₃). IR (ATR-FTIR), cm⁻¹: 3300 (w), 1688 (s), 1596 (s), 1501 (s), 1248 (s), 1157 (w). HRMS-CI (m/z): [M+Na]⁺ calcd for C₁₀H₁₇N₃NaO₂S₂, 298.0660; found, 298.0621. [α]_{D}²⁰ −1.4 (c 1.0, CH₃OH).

### Synthesis of the Thiazoline-Thiazole 14:

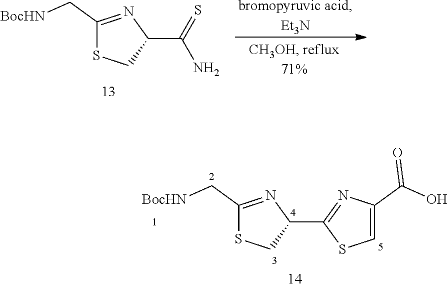

Triethylamine (759 μL, 5.48 mmol, 3.00 equiv) was added dropwise via syringe to a solution of the thioamide 13 (500 mg, 1.82 mmol, 1 equiv) and bromopyruvic acid (364 mg, 2.18 mmol, 1.20 equiv) in methanol (13 mL) at 23° C. The reaction vessel was fitted with a reflux condenser and then was placed in an oil bath that had been preheated to 72° C. The reaction mixture was stirred and heated for 3 h at 72° C. The product mixture was concentrated and the residue obtained was purified using trimethylamine acetate-functionalized silica gel (Si-TMA acetate; eluting with 2% acetic acid-methanol) to provide the thiazoline-thiazole 14 as a white solid (440 mg, 71%).

¹H NMR (600 MHz, CDCl₃) δ 8.20 (s, 1H, H₄), 5.89 (app t, J=8.6 Hz, 1H, H₄), 5.35 (t, J=5.7 Hz, 1H), 4.30-4.16 (m, 2H, H₂), 3.89 (dd, J=11.4, 9.2 Hz, 1H, H₃), 3.64 (dd, J=11.4, 7.6 Hz, 1H, H₃), 1.46 (s, 9H, H₁). ¹³C NMR (151 MHz, CDCl₃) δ 175.7 (C), 172.4 (C), 164.2 (C), 155.7 (C), 147.0 (C), 129.0 (CH), 80.5 (C), 77.0 (CH), 43.3 (CH₂), 39.1 (CH₂), 28.5 (CH₃). IR (ATR-FTIR), cm⁻¹: 1696 (w), 1507 (s), 1248 (s), 1156 (s). HRMS-CI (m/z): [M+Na]⁺ calcd for C₁₃H₁₇N₃NaO₄S₂, 366.0558; found, 366.0508. [α]_{D}²⁰ +6.6 (c 2.7, CH₃OH).

### Synthesis of the Amine 15:

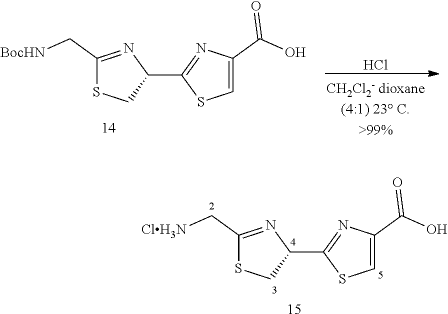

A solution of hydrogen chloride in 1,4-dioxane (4.0 N, 3.50 mL, 14.0 mmol, 16.7 equiv) was added dropwise via syringe pump over 20 min to a solution of the thiazoline-thiazole 14 (287 mg, 836 μmol, 1 equiv) in dichloromethane (14 mL) at 23° C. The resulting mixture was stirred for 1 h at 23° C. The product mixture was concentrated to provide the amine 15 as a white solid (234 mg, >99%).

The product 15 obtained in this way was used directly in the following step without further purification.

¹H NMR (600 MHz, DMSO-d₆) δ 8.65-8.54 (bs, 3H), 8.47 (s, 1H, H₅), 6.02-5.83 (m, 1H, H₄), 4.05 (s, 2H, H₂), 4.04-4.00 (m, 1H, H₃), 3.70 (dd, J=11.3, 8.5 Hz, 1H, H₃). ¹³C NMR (151 MHz, DMSO-d₆) δ 170.3 (C), 167.9 (C), 162.0 (C), 147.1 (C), 129.3 (CH), 76.3 (CH), 40.2 (CH₂), 38.9 (CH₂).

### Synthesis of the β-Ketothioester 16:

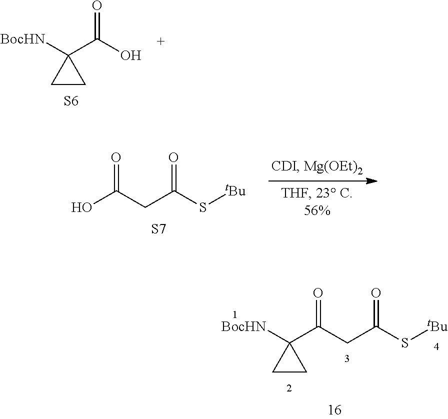

1,1′-Carbonyldiimidazole (846 mg, 5.22 mmol, 1.50 equiv) was added to a solution of N-(tert-butoxycarbonyl)-1-amino-1-cyclopropane carboxylic acid (S6, 700 mg, 3.48 mmol, 1 equiv) in tetrahydrofuran (18 mL) at 23° C. The resulting mixture was stirred for 6 h at 23° C. In a second round-bottomed flask, magnesium ethoxide (299 mg, 2.61 mmol, 0.75 equiv) was added to a solution of 3-(tert-butylthio)-3-oxopropanoic acid (S7, 920 mg, 5.22 mmol, 1.50 equiv) in tetrahydrofuran (9.0 mL) at 23° C. The resulting mixture was stirred for 6 h at 23° C., and then was concentrated to dryness. The activated carboxylic acid prepared in the first flask was transferred via cannula to the dried magnesium salt prepared in the second flask. The resulting mixture was stirred for 14 h at 23° C. The product mixture was diluted sequentially with saturated aqueous ammonium chloride solution (20 mL) and ethyl acetate (30 mL). The diluted product mixture was transferred to a separatory funnel and the layers that formed were separated. The aqueous layer was extracted with ethyl acetate (2×30 mL). The organic layers were combined and the combined organic layers were washed with saturated aqueous sodium chloride solution (30 mL). The washed organic layer was dried over sodium sulfate. The dried solution was filtered and the filtrate was concentrated. The residue obtained was purified by flash-column chromatography (eluting with hexanes initially, grading to 10% ethyl acetate-hexanes, linear gradient) to provide the I-ketothioester 16 as a colorless oil (614 mg, 56%).

R_{f}=0.36 (20% ethyl acetate-hexanes; UV). ¹H NMR (600 MHz, CDCl₃) δ 5.25 (bs, 1H), 3.80 (s, 2H, H₃), 1.62 (q, J=4.5 Hz, 2H, H₂), 1.49 (s, 9H, H₄), 1.47 (s, 9H, H₁), 1.22-1.15 (m, 2H, H₂). ¹³C NMR (151 MHz, CDCl₃) δ 203.7 (C), 193.9 (C), 155.8 (C), 80.7 (C), 55.3 (CH₂), 49.1 (C), 41.6 (C), 29.8 (CH₃), 28.5 (CH₃), 21.8 (CH₂). IR (ATR-FTIR), cm⁻¹: 3382 (m), 3333 (m), 2969 (br), 1699 (s), 1658 (m), 1597 (m), 1487 (s), 1249 (s), 1161 (s), 1065 (s). HRMS-CI (m/z): [M+Na]⁺ calcd for C₁₅H₂₅NNaO₄S, 338.1402; found, 338.1384.

### Synthesis of the β-Ketoamide S8:

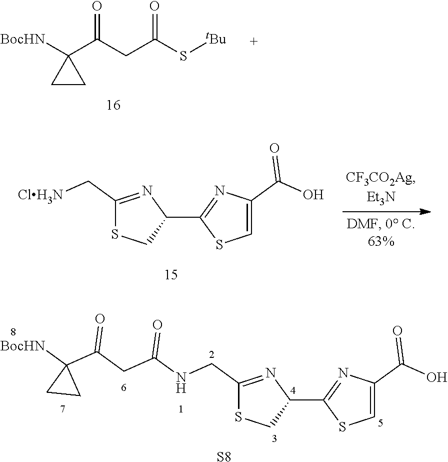

Three equal portions of silver trifluoroacetate (26.8 mg, 122 μmol, 0.40 equiv each) were added over 1 h to a solution of triethylamine (170 μt, 1.22 mmol, 4.00 equiv), 15 (85.0 mg, 304 μmol, 1 equiv), and 16 (115 mg, 365 μmol, 1.20 equiv) in N,N-dimethylformamide (3.5 mL) at 0° C. The reaction mixture was stirred for 1 h at 0° C. The product mixture was directly applied to a column containing trimethylamine acetate-functionalized silica gel (Si-TMA acetate; eluting with 2% acetic acid-methanol). The fractions containing product were collected, combined, and concentrated. The residue obtained was further purified by automated flash-column chromatography (eluting with 2% acetic acid-dichloromethane initially, grading to 2% acetic acid-6% methanol-dichloromethane, linear gradient) to afford the β-ketoamide S8 as a white solid (90.0 mg, 63%).

¹H NMR (600 MHz, DMSO-d₆) δ 12.99 (bs, 1H), 8.67 (t, J=6.0 Hz, 1H), 8.40 (s, 1H, H₅), 7.73 (bs, 1H), 5.91 (t, J=8.7 Hz, 1H, H₄), 4.31-4.08 (m, 2H, H₂), 3.86 (dd, J=11.3, 9.3 Hz, 1H, H₃), 3.55-3.49 (m, 3H, H₆, H₃), 1.40 (s, 9H, H₈), 1.35 (d, J=3.1 Hz, 2H, H₇), 1.06 (d, J=3.6 Hz, 2H, H₇). ¹³C NMR (151 MHz, DMSO-d₆) δ 204.6 (C), 173.9 (C), 171.5 (C), 166.6 (C), 162.2 (C), 156.3 (C), 147.1 (C), 128.9 (CH), 78.6 (C), 76.9 (CH), 45.9 (CH₂), 41.2 (C), 41.1 (CH₂), 37.7 (CH₂), 28.2 (CH₃), 19.5 (CH₂). IR (ATR-FTIR), cm⁻¹: 3000 (br), 1702 (s), 1507 (m), 1249 (m), 1164 (3), 1068 (m). HRMS-CI (m/z): [M+H]⁺ calcd for C₁₉H₂₅N₄O₆S₂, 469.1216; found, 469.1257. [α]_{D}²⁰ +3.0 (c 2.0, CH₃OH).

### Synthesis of the Amine 17:

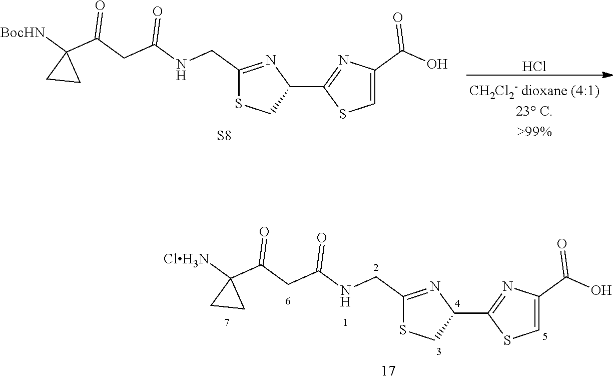

A solution of hydrogen chloride in 1,4-dioxane (4.0 N, 1.00 mL, 4.00 mmol, 22.6 equiv) was added dropwise via syringe pump over 20 min to a solution of the β-ketoamide S5 (83.0 mg, 177 μmol, 1 equiv) in dichloromethane (4.0 mL) at 23° C. The resulting mixture was stirred for 1 h at 23° C. The reaction mixture was concentrated to provide the amine 17 as a white solid (71.7 mg, >99%).

The product 17 obtained in this way was used directly in the following step.

¹H NMR (600 MHz, DMSO-d₆) δ 8.87 (t, J=6.0 Hz, 1H, H₁), 8.81 (s, 3H), 8.41 (s, 1H, H₅), 5.96-5.86 (m, 1H, H₄), 4.25-4.01 (min, 2H, H₂), 3.87 (dd, J=11.3, 9.2 Hz, 1H, H₃), 3.54 (dd, J=11.8, 8.2 Hz, 1H, H₃), 3.35 (s, 2H, H₆), 1.86-1.67 (m, 2H, H₇), 1.56-1.37 (m, 2H, H₇). ¹³C NMR (151 MHz, DMSO-d₆) δ 199.2 (C), 173.4 (C), 171.3 (C), 165.7 (C), 162.0 (C), 147.1 (C), 129.0 (CH), 76.8 (CH), 42.3 (CH₂), 42.0 (C), 41.2 (CH₂), 37.8 (CH₂), 13.1 (CH_{Z}).

### Synthesis of the Thiazole 19:

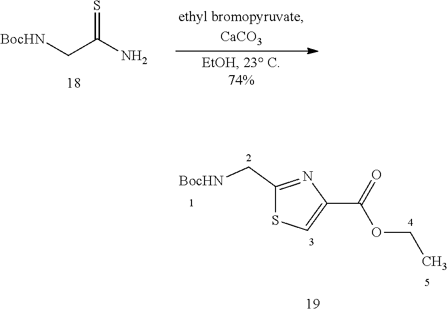 [SMILES: CC(C)(C)OC(=O)NCC(N)=S ; CCOC(=O)c1csc(CNC(=O)OC(C)(C)C)n1]

Ethyl bromopyruvate (2.37 mL, 18.9 mmol, 1.20 equiv) and calcium carbonate (1.58 g, 15.8 mmol, 1.00 equiv) were added in sequence to a solution of tert-butyl 2-amino-2-thioxoethylcarbamate (18, 3.00 g, 15.8 mmol, 1 equiv) in ethanol (60 mL) at 23° C. The reaction mixture was stirred for 6 h at 23° C. The product mixture was concentrated and the residue obtained was purified by flash-column chromatography (eluting with 10% ethyl acetate-hexanes initially, grading to 30% ethyl acetate-hexanes, linear gradient) to furnish the thiazole 19 as a white solid (3.34 g, 74%).

¹H and ¹³C NMR data for thiazole 19 prepared in this way were in agreement with the literature.¹⁵

R_{f}=0.39 (40% ethyl acetate-hexanes; UV). ¹H NMR (600 MHz, CDCl₃) δ 8.12 (s, 1H, H₃), 4.65 (d, J=6.4 Hz, 2H, H₂), 4.42 (q, J=7.1 Hz, 2H, H₄), 1.46 (s, 9H, H₁), 1.40 (t, J=7.1 Hz, 3H, H₅). ¹³C NMR (151 MHz, CDCl₃) δ 170.1 (C), 161.4 (C), 155.8 (C), 147.1 (C), 128.1 (CH), 80.7 (C), 61.7 (CH₂), 42.5 (CH₂), 28.5 (CH₃), 14.5 (CH₃).

### Synthesis of the Amide S9:

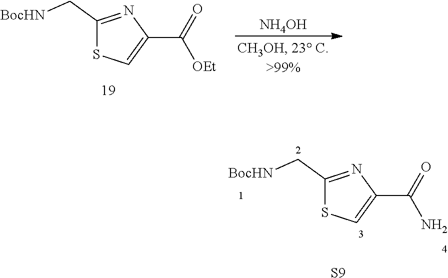 [SMILES: CCOC(=O)c1csc(CNC(=O)OC(C)(C)C)n1 ; "NH4OH" ; CC(C)(C)OC(=O)NCc1nc(C(N)=O)cs1 ; "S9"]

A solution of aqueous ammonia (28% w/v, 42 mL) was added to a solution of the thiazole 19 (2.38 g, 8.29 mmol, 1 equiv) in anhydrous methanol (84 mL) at 23° C. The resulting mixture was stirred for 16 h at 23° C. The product mixture was concentrated and the residue obtained was dried by azeotropic distillation from toluene (2×30 mL) to afford the product S9 as a yellow solid (2.13 g, >99%).

The amide S9 obtained in this way was estimated to be of >95% purity by ¹H and ¹³C NMR analysis (see accompanying spectra) and was used without further purification.

¹H and ¹³C NMR data for amide S9 prepared in this way were in agreement with the literature.¹⁶

¹H NMR (600 MHz, CDCl₃) δ 8.08 (s, 1H, H₃), 7.12 (bs, 1H, H₄), 5.83 (bs, 1H, H₄), 5.30 (t, J=6.2 Hz, 1H), 4.60 (d, J=6.2 Hz, 2H, H₂), 1.47 (s, 9H, H₁). ¹³C NMR (151 MHz, CDCl₃) δ 169.5 (C), 163.0 (C), 155.7 (C), 149.3 (C), 124.8 (CH), 80.7 (C), 42.5 (CH₂), 28.5 (CH₃).

### Synthesis of the Nitrile 20:

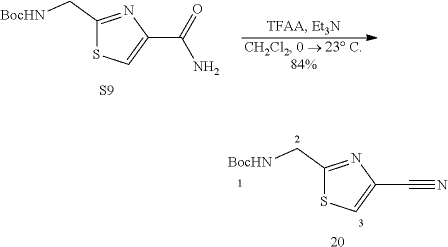

Trifluoroacetic anhydride (1.24 mL, 8.94 mmol, 1.10 equiv) was added dropwise via syringe pump over 20 min to a solution of the amide S9 (2.09 g, 8.12 mmol, 1 equiv) and triethylamine (2.49 mL, 17.9 mmol, 2.20 equiv) in dichloromethane (120 mL) at 0° C. The resulting mixture was stirred for 30 min at 0° C. The reaction mixture was then allowed to warm over 30 min to 23° C. The warmed reaction mixture was stirred for 2 h at 23° C. The product mixture was concentrated and the residue obtained was purified by flash-column chromatography (eluting with hexanes initially, grading to 20% ethyl acetate-hexanes, linear gradient) to furnish the nitrile 20 as a white solid (1.63 g, 84%).

¹H and ¹³C NMR data for nitrile 20 prepared in this way were in agreement with the literature.¹⁶

R_{f}=0.51 (40% ethyl acetate-hexanes; UV). ¹H NMR (600 MHz, CDCl₃) δ 7.95 (s, 1H, H₃), 5.31 (t, J=6.4 Hz, 1H), 4.62 (d, J=6.4 Hz, 2H, H₂), 1.47 (s, 9H, H₁). ¹³C NMR (151 MHz, CDCl₃) δ 171.5 (C), 155.8 (C), 131.0 (CH), 126.6 (C), 113.9 (C), 81.0 (C), 42.5 (CH₂), 28.4 (CH₃). IR (ATR-FTIR), cm⁻¹: 3334 (w), 3073 (w), 1684 (s), 1520 (s), 1297 (s), 1161 (m), 618 (m).

### Synthesis of the Thiazole-Thiazoline 21:

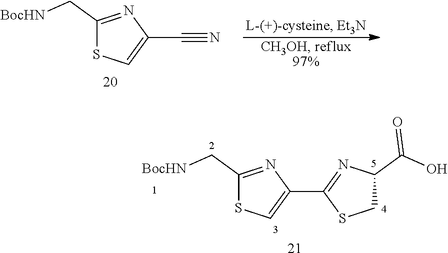

Triethylamine (1.01 mL, 7.21 mmol, 1.10 equiv) was added dropwise via syringe to a solution of the nitrile 20 (1.57 g, 6.55 mmol, 1 equiv) and L-cysteine (870 mg, 7.21 mmol, 1.10 equiv) in methanol (60 mL) at 23° C. The reaction vessel was fitted with a reflux condenser and then placed in an oil bath that had been preheated to 73° C. The reaction mixture was stirred and heated for 3 h at reflux. The product mixture was cooled to 23° C. and the cooled product mixture was concentrated. The residue obtained was dissolved in saturated aqueous sodium bicarbonate solution (40 mL) and the resulting solution was washed with ether (30 mL). The aqueous layer was acidified to pH ˜3-4 by the dropwise addition of 3.0 N aqueous hydrochloric acid solution. The resulting mixture was extracted with ethyl acetate (3×30 mL) and the organic layers were combined. The combined organic layers were dried over sodium sulfate and the dried solution was filtered. The filtrate was concentrated to provide the thiazole-thiazoline 21 as a white solid (2.18 g, 97%).

The thiazole-thiazoline 21 obtained in this way was estimated to be of >95% purity by ¹H and ¹³C NMR analysis (see accompanying spectra) and was used without further purification.

¹H and ¹³C NMR data for thiazole-thiazoline 21 prepared in this way were in agreement with the literature.¹⁷

¹H NMR (600 MHz, CD₃OD) δ 8.16 (s, 1H, H₃), 5.30 (t, J=9.1 Hz, 1H, H₅), 4.52 (s, 2H, H₂), 3.85-3.51 (m, 2H, H₄), 1.47 (s, 9H, H₁). ¹³C NMR (151 MHz, CD₃OD) δ 173.8 (C), 173.3 (C), 167.9 (C), 158.3 (C), 149.3 (C), 123.0 (CH), 81.0 (C), 79.1 (CH), 43.1 (CH₂), 35.6 (CH₂), 28.7 (CH₃). IR (ATR-FTIR), cm⁻¹: 3351 (w), 2978 (w), 1701 (w), 1519 (w), 1250 (s), 1165 (s).

### Synthesis of the Amine 22:

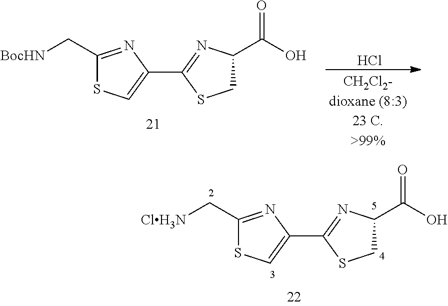

A solution of hydrogen chloride in 1,4-dioxane (4.0 N, 1.5 mL, 6.00 mmol, 6.90 equiv) was added dropwise via syringe pump over 20 min to a solution of the thiazole-thiazoline 21 (300 mg, 870 μmol, 1 equiv) in dichloromethane (12 mL) at 23° C. The resulting mixture was stirred for 1 h at 23° C. The reaction mixture was concentrated to provide the amine 22 as a white solid (244 mg, >99%).

The product 22 obtained in this way was used directly in the following step.

¹H NMR (600 MHz, CD₃OD) δ 9.02 (s, 1H, H₃), 5.66 (dd, J=10.5, 5.8 Hz, 1H, H₅), 4.64 (s, 2H, H₂), 4.17 (dd, J=12.1, 10.5 Hz, 1H, H₄), 4.10 (dd, J=12.1, 5.8 Hz, 1H, H₄). ¹³C NMR (151 MHz, CD₃OD) δ 180.3 (C), 170.3 (C), 166.0 (C), 143.5 (C), 134.2 (CH), 69.9 (CH), 40.9 (CH₂), 35.7 (CH₂).

### Synthesis of the β-Ketoamide S10:

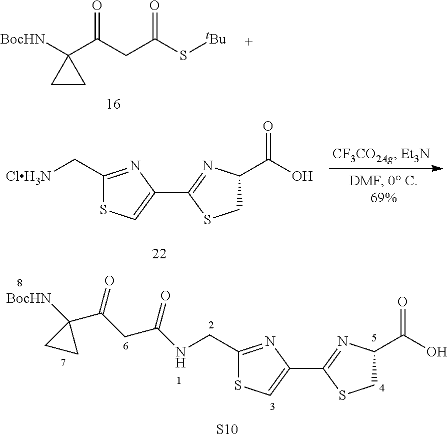

Three equal portions of silver trifluoroacetate (31.6 mg, 14.3 μmol, 0.40 equiv each) were added over 1 h to a solution of triethylamine (199 μl, 1.43 mmol, 4.00 equiv), the β-ketothioester 16 (135 mg, 42.9 μmol, 1.20 equiv), and the amine 22 (100 mg, 35.7 μmol, 1 equiv) in N,N-dimethylformamide (3.5 mL) at 0° C. The reaction mixture was stirred for 1 h at 0° C. The product mixture was directly applied to a column containing trimethylamine acetate-functionalized silica gel (Si-TMA acetate; eluting with 2% acetic acid-methanol). The fractions containing product were collected, combined, and concentrated. The residue obtained was further purified by automated flash-column chromatography (eluting with 2% acetic acid-dichloromethane initially, grading to 2% acetic acid-6% methanol-dichloromethane, linear gradient). The fractions containing the product S10 were collected, combined, and concentrated to provide the β-ketoamide S10 as a white solid (115 mg, 69%).

¹H NMR (600 MHz, DMSO-d₆) δ 8.95 (t, J=6.0 Hz, 1H, H₁), 8.23 (s, 1H, H₃), 5.26 (dd, J=9.7, 8.2 Hz, 1H, H₅), 4.55 (d, J=4.5 Hz, 1H, H₂), 3.63 (dd, J=11.3, 9.7 Hz, 1H, H₄), 3.56 (s, 2H, H₆), 3.56-3.51 (m, 1H, H₄), 1.41 (s, 9H, H₈), 1.40-1.31 (m, 2H, H₇), 1.07 (q, J=4.3 Hz, 2H, H₇). ¹³C NMR (151 MHz, DMSO-d₆) δ 204.7 (C), 171.8 (C), 170.1 (C), 166.8 (C), 163.3 (C), 156.0 (C), 147.4 (C), 122.1 (CH), 78.6 (C), 78.3 (CH), 46.1 (CH₂), 41.1 (C), 40.3 (CH₂), 34.4 (CH₂), 28.2 (CH₃), 19.5 (CH₂). IR (ATR-FTIR), cm⁻¹: 3317 (br), 1702 (s), 1506 (m), 1248 (m), 1162 (s), 1063 (m). HRMS-CI (m/z): [M+H]⁺ calcd for C₁₉H₂₅N₄O₆S₂, 469.1216; found, 469.1257. [α]_{D}²⁰ +5.6 (c 2.2, CH₃OH).

### Synthesis of the Amine 23:

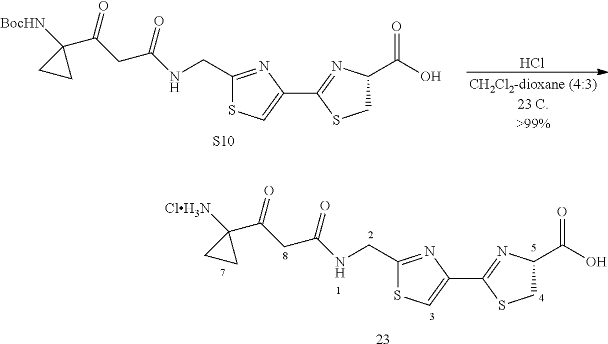

A solution of hydrogen chloride in 1,4-dioxane (4.0 N, 1.0 mL, 4.00 mmol, 21.5 equiv) was added dropwise via syringe pump over 20 min to a solution of the β-ketoamide S10 (87.0 mg, 186 μmol, 1 equiv) in dichloromethane (4.0 mL) at 23° C. The resulting mixture was stirred for 1 h at 23° C. The reaction mixture was concentrated to provide the amine 23 as a white solid (75.2 mg, >99%).

The product 23 obtained in this way was used directly in the following step.

¹H NMR (600 MHz, DMSO-d₆) δ 9.17 (t, J=6.0 Hz, 1H, H₁), 8.83 (bs, 3H), 8.30 (s, 1H, H₃), 5.28 (dd, J=9.7, 8.1 Hz, 1H, H₅), 4.58 (d, J=5.9 Hz, 2H, H₂), 3.66 (dd, J=11.2, 9.9 Hz, 1H, H₄), 3.60-3.51 (m, 1H, H₄), 3.38 (s, 2H, H₆), 1.81-1.72 (m, 2H, H₇), 1.57-1.49 (m, 2H, H₇). ¹³C NMR (151 MHz, DMSO-d₆) δ 199.39 (C), 171.66 (C), 169.76 (C), 165.92 (C), 164.04 (C), 147.09 (C), 122.66 (CH), 77.72 (CH), 42.38 (CH₂), 42.00 (C), 40.43 (CH₂), 34.40 (CH₂), 13.12 (CH₂).

### Synthesis of the Thioamide 24:

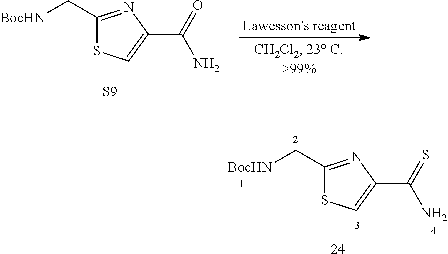

Lawesson's reagent (1.51 g, 7.73 mmol, 0.75 equiv) was added to a solution of the amide S9 (1.28 g, 4.98 mmol, 1 equiv) in dichloromethane (30 mL) at 23° C. The resulting mixture was stirred for 16 h at 23° C. The product mixture was filtered through a pad of celite (2.5×4.5 cm). The filter cake was washed with dichloromethane (10 mL). The filtrates were combined and the combined filtrates were concentrated. The residue obtained was dissolved in ethyl acetate (40 mL) and the resulting solution was washed sequentially with saturated aqueous sodium bicarbonate solution (2×20 mL) and saturated aqueous sodium chloride solution (20 mL). The washed organic layer was dried over sodium sulfate. The dried solution was filtered and the filtrate was concentrated to provide the thioamide 24 as a pale yellow solid (1.36 g, >99%).

The thioamide 24 obtained in this way was estimated to be of >95% purity by ¹H and ¹³C NMR analysis (see accompanying spectra) and was used without further purification.

¹H and ¹³C NMR data for the thioamide 24 prepared in this way were in agreement with the literature.¹⁷

R_{f}=0.37 (2% methanol-CH₂Cl₂; UV). ¹H NMR (600 MHz, (CD₃)CO₂) δ 9.04 (bs, 1H, H₄), 8.38 (s, 1H, H₃), 6.96 (t, J=6.3 Hz, 1H), 4.55 (d, J=6.1 Hz, 2H, H₂), 1.44 (s, 9H, H₁). ¹³C NMR (151 MHz, (CD₃)CO₂) δ 191.3 (C), 172.0 (C), 156.7 (C), 154.6 (C), 127.5 (CH), 79.8 (C), 43.2 (CH₂), 28.5 (CH₃).

### Synthesis of the Bithiazole 25:

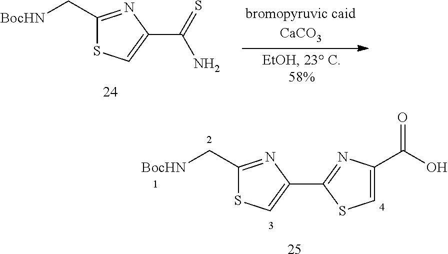

Bromopyruvic acid (170 mg, 1.02 mmol, 1.50 equiv) and calcium carbonate (136 mg, 1.36 mmol, 2.00 equiv) were added in sequence to a solution of the thioamide 24 (186 mg, 680 μmol, 1 equiv) in ethanol (6.0 mL) at 23° C. The reaction mixture was stirred for 16 h at 23° C. The product mixture was concentrated and the residue obtained was applied to a trimethylamine acetate-functionalized silica column (S Si-TMA acetate; eluting with 2% acetic acid-methanol) to provide the bithiazole 25 as a white solid (135 mg, 58%).

¹H NMR (600 MHz, DMSO-d₆) δ 8.42 (s, 1H, H₄), 8.22 (s, 1H, H₃), 7.87 (t, J=6.1 Hz, 1H), 4.45 (d, J=6.1 Hz, 2H, H₂), 1.42 (s, 9H, H₁). ¹³C NMR (151 MHz, DMSO-d₆) δ 172.82 (C), 162.25 (C), 161.92 (C), 155.80 (C), 149.09 (C), 147.34 (C), 128.29 (CH), 117.78 (CH), 78.72 (C), 41.93 (CH₂), 28.16 (CH₃). IR (ATR-FTIR), cm⁻¹: 3322 (w), 3128 (w), 1685 (s), 1534 (m), 1290 (m), 1233 (m), 1168 (m), 772 (m), 751 (m). HRMS-CI (m/z): [M+H]⁺ calcd for C₁₃H₁₆N₃O₄S₂, 342.0582; found, 342.0577.

### Synthesis of the Amine 26:

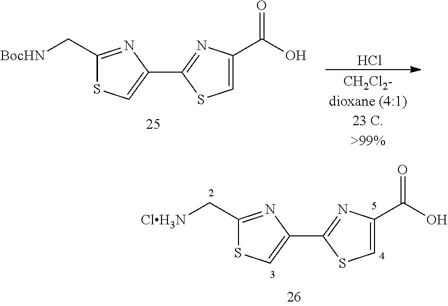

A solution of hydrogen chloride in 1,4-dioxane (4.0 N, 1.5 mL, 6.00 mmol, 16.8 equiv) was added dropwise via syringe pump over 20 min to a solution of the bithiazole 25 (122 mg, 357 μmol, 1 equiv) in dichloromethane (6.0 mL) at 23° C. The resulting mixture was stirred for 1 h at 23° C. The reaction mixture was concentrated to provide the amine 26 as a white solid (99.3 mg, >99%).

The product 26 obtained in this way was used directly in the following step.

¹H NMR (600 MHz, DMSO-d₆) δ 9.57 (bs, 3H), 9.33 (s, 1H, H₄), 9.24 (s, 1H, H₃), 5.32 (s, 2H, H₂). ¹³C NMR (151 MHz, DMSO-d₆) δ 164.2 (C), 162.4 (C), 162.1 (C), 148.6 (C), 147.7 (C), 129.7 (CH), 120.7 (CH), 39.7 (CH₂).

### Synthesis of the β-Ketoamide S11:

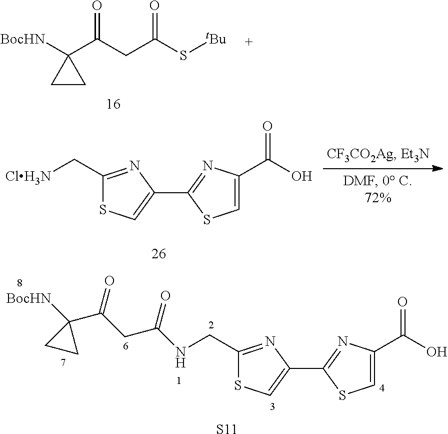

Three equal portions of silver trifluoroacetate (27.0 mg, 12.2 μmol, 0.40 equiv) were added over 1 h to a solution of triethylamine (171 μL, 1.22 mmol, 4.00 equiv), the β-ketothioester 16 (116 mg, 36.7 μmol, 1.20 equiv), and the amine 26 (85.0 mg, 30.6 μmol, 1 equiv) in N,N-dimethylformamide (3.50 mL) at 0° C. The reaction mixture was stirred for 1 h at 0° C. The product mixture was directly applied to a column containing trimethylamine acetate-functionalized silica gel (Si-TMA acetate; eluting with 2% acetic acid-methanol). The fractions containing product were collected, combined, and concentrated. The residue obtained was further purified by automated flash-column chromatography (eluting with 2% acetic acid-dichloromethane initially, grading to 2% acetic acid-6% dichloromethane-methanol, linear gradient). The fractions containing the product S11 were collected, combined, and concentrated to provide the β-ketoamide S11 as a white solid (102 mg, 72%).

¹H NMR (600 MHz, DMSO-d₆) δ 13.14 (bs, 1H), 8.98 (t, J=6.0 Hz, 1H, H₁), 8.47 (s, 1H, H₄), 8.25 (s, 1H, H₃), 7.77 (s, 1H), 4.60 (d, J=6.0 Hz, 2H, H₂), 3.57 (s, 2H, H₆), 1.41 (s, 9H, H₈), 1.40-1.36 (m, 2H, H₇), 1.08 (q, J=4.6 Hz, 2H, H₇). ¹³C NMR (151 MHz, DMSO-d₆) δ 204.7 (C), 171.3 (C), 166.9 (C), 162.1 (C), 162.0 (C), 156.1 (C), 148.2 (C), 147.1 (C), 128.9 (CH), 118.4 (CH), 78.6 (C), 46.1 (CH₂), 41.2 (C), 40.5 (CH₂), 28.2 (CH₃), 19.6 (CH₂). IR (ATR-FTIR), cm⁻¹: 3322 (m), 2931 (br), 1709 (s), 1686 (s), 1511 (m), 1282 (m), 1237 (m), 1164 (m), 758 (m). HRMS-CI (m/z): [M+Na]⁺ calcd for Cl₉H₂₂N₄NaO₆S₂, 489.0878; found, 489.0794.

### Synthesis of the Amine 27:

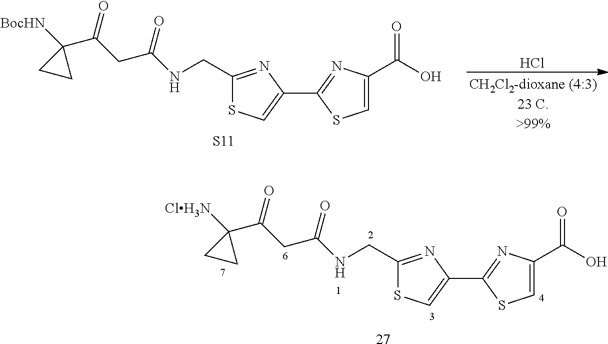

A solution of hydrogen chloride in 1,4-dioxane (4.0 N, 0.5 mL, 2.00 mmol, 33.3 equiv) was added dropwise via syringe pump over 20 min to a solution of the β-ketoamide S11 (28.0 mg, 60.0 μmol, 1 equiv) in dichloromethane (2.0 mL) at 23° C. The resulting mixture was stirred for 1 h at 23° C. The reaction mixture was concentrated to provide the amine 27 as a white solid (24.2 mg, >99%).

The product 27 obtained in this way was used directly in the following step.

¹H NMR (600 MHz, DMSO-d₆) δ 13.15 (bs, 1H), 9.19 (t, J=6.0 Hz, 1H, H₁), 8.79 (bs, 3H), 8.49 (s, 1H, H₄), 8.28 (s, 1H, H₃), 4.63 (d, J=5.9 Hz, 2H, H₂), 3.40 (s, 2H, H₆), 1.78 (q, J=5.9 Hz, 2H, H₇), 1.53 (q, J=6.0 Hz, 2H, H₇). ¹³C NMR (151 MHz, DMSO-d₆) δ 199.4 (C), 170.7 (C), 166.0 (C), 162.1 (C), 162.0 (C), 148.1 (C), 147.2 (C), 129.0 (CH), 118.4 (CH), 42.4 (CH₂), 42.0 (C), 40.5 (CH₂), 13.1 (CH₂).

### Synthesis of the Thioester 28:

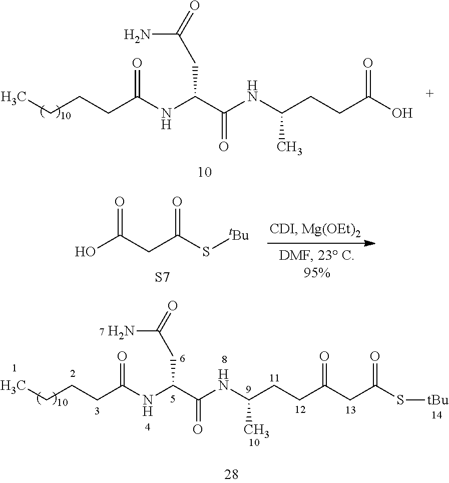

1,1′-Carbonyldiimidazole (CDI; 165 mg, 1.02 mmol, 1.50 equiv) was added to a solution of the carboxylic acid 10 (300 mg, 679 μmol, 1 equiv) in N,N-dimethylformamide (7.0 mL). The resulting solution was stirred for 8 h at 23° C. In a second round-bottomed flask, magnesium ethoxide (77.7 mg, 679 μmol, 1.00 equiv) was added to a solution of 3-(tert-butylthio)-3-oxopropanoic acid (S7, 238 mg, 1.36 mmol, 2.00 equiv) in tetrahydrofuran (3.0 mL). The resulting mixture was stirred for 10 h at 23° C. The reaction mixture was concentrated to provide the magnesium salt of the β=ketothioester S7 (271 mg, >99%) as a colorless solid. A solution of the magnesium salt of the β-ketothioester S7 in N,N-dimethylformamide (1.0 mL) was transferred via cannula to the activated carboxylic acid and the resulting mixture was stirred for 16 h at 23° C. The product mixture was diluted with aqueous hydrogen chloride solution (1.0 N, 20 mL). The resulting solid precipitate was isolated by filtration and the isolated filtrate was applied to a trimethylamine acetate-functionalized silica column (Si-TMA acetate; eluting with methanol) to provide the β-ketothioester 28 as a white solid (359 mg, 95%).

¹H NMR (400 MHz, DMSO-d₆) δ 7.88 (d, J=7.8 Hz, 1H, H₄), 7.50 (d, J=8.6 Hz, 1H, H₈), 7.25 (bs, 1H, H₇), 6.85 (bs, 1H, H₇), 4.41 (q, J=7.4 Hz, 1H, H₅), 3.74-3.59 (m, 3H, H₅, H₁₃), 2.50-2.42 (m, 2H, H₁₂), 2.41 (dd, J=14.1, 5.0 Hz, 1H, H₆), 2.33 (dd, J=15.1, 7.7 Hz, 1H, H₆), 2.09 (dd, J=9.4, 6.7 Hz, 2H, H₃), 1.66-1.52 (m, 1H, H₁₁), 1.54-1.42 (m, 3H, H₁₁, H₂), 1.41 (s, 9H, H₁₄), 1.23 (bs, 20H, 10×CH₂), 0.98 (d, J=6.6 Hz, 3H, H₁₀), 0.85 (t, J=6.5 Hz, 3H, H₁). ¹³C NMR (126 MHz, DMSO-d₆) δ 202.5 (C), 192.8 (C), 172.1 (C), 171.4 (C), 170.5 (C), 57.5 (CH₂), 49.9 (CH), 48.1 (C), 43.5 (CH), 39.0 (CH₂), 37.3 (CH₂), 35.2 (CH₂), 31.3 (CH₂), 29.6 (CH₂), 29.2 (CH), 29.06 (CH₃), 29.04 (2×CH₂), 29.01 (CH₂), 28.95 (CH₂), 28.87 (CH₂), 28.84 (CH₂), 28.7 (CH₂), 25.2 (CH₂), 22.1 (CH₂), 20.5 (CH₃), 14.0 (CH₃). IR (ATR-FTIR), cm⁻¹: 3297 (m), 2955 (m), 1660 (s), 1633 (s), 1542 (m), 1121 (br), 1029 (m), 587 (m). HRMS-CI (m/z): [M+H]⁺ calcd for C₂₉H₅₄N₃O₅S, 556.3784; found, 556.3759. [α]_{D}²⁰ +4.8 (c 1.7, CH₃OH).

### Synthesis of the Linear Precursor 29a:

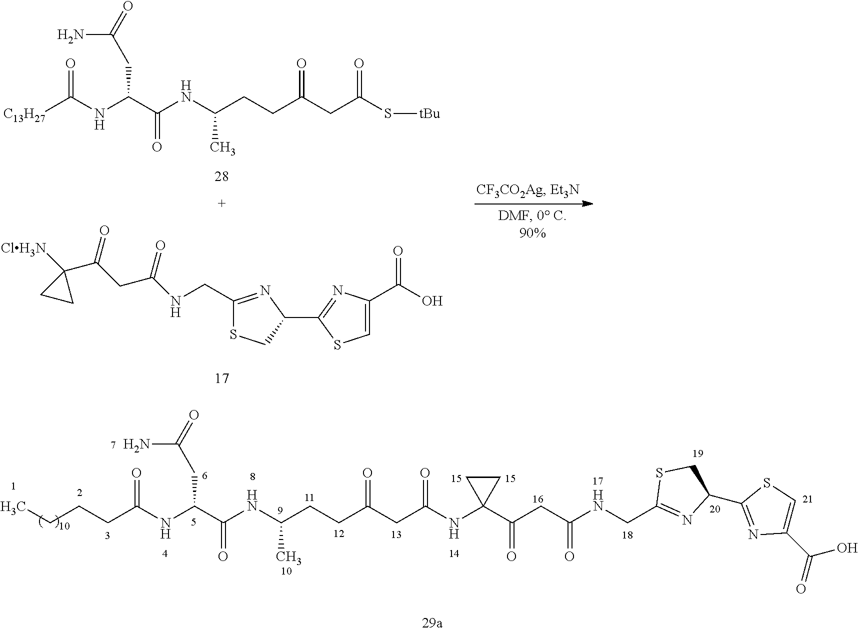

Silver trifluoroacetate (18.3 mg, 83.0 μmol, 2.00 equiv) was added to a solution of triethylamine (23.0 μL, 166 μmol, 4.00 equiv), the β-ketothioester 28 (23.0 mg, 41.0 μmol, 1 equiv), and the amine 17 (20.1 mg, 50.0 μmol, 1.20 equiv) in N,N-dimethylformamide (0.8 mL) at 0° C. The reaction mixture was stirred for 1 h at 0° C. The heterogeneous product mixture was diluted with aqueous citric acid solution (5%, 4.0 mL). The resulting precipitate was isolated by filtration. The solid was dried in vacuo to provide the linear precursor 29a as a white solid (31.0 mg, 90%).

¹H NMR (600 MHz, DMSO-d₆) δ 8.80 (s, 1H, H₁₄), 8.67 (t, J=6.1 Hz, 1H, H₁₇), 8.37 (s, 1H, H₂₁), 7.90 (bs, 1H, H₄), 7.52 (d, J=8.5 Hz, 1H, H₈), 7.28 (bs, 1H, H₇), 6.86 (bs, 1H, H₇), 5.91 (t, J=8.6 Hz, 1H, H₂₀), 4.43 (q, J=7.2 Hz, 1H, H₅), 4.17 (d, J=5.5 Hz, 2H, H₁₈), 3.86 (t, J=10.2 Hz, 1H, H₁₉), 3.75-3.63 (m, 1H, H₉), 3.55 (s, 2H, H₁₆), 3.52 (dd, J=11.5, 8.4 Hz, 1H, H₁₉), 3.34 (s, 1H, H₁₃), 2.55-2.43 (m, 2H, H₁₂), 2.42 (dd, J=15.2, 6.1 Hz, 1H, H₆), 2.36 (dd, J=15.5, 7.9 Hz, 1H, H₆), 2.13-2.03 (m, 2H, H₃), 1.67-1.53 (m, 1H, H₁₁), 1.54-1.47 (m, 1H, H₁₁), 1.50-1.40 (m, 2H, H₂), 1.38 (d, J=4.4 Hz, 2H, H₁₅), 1.22 (bs, 20H, 10×CH₂), 1.05-1.01 (m, 2H, H₁₅), 0.99 (d, J=6.6 Hz, 3H, H₁₀), 0.85 (t, J=6.9 Hz, 3H, H₁). ¹³C NMR (151 MHz, DMSO-d₆) δ 204.8 (C), 204.0 (C), 173.8 (C), 172.2 (C), 172.1 (C), 171.4 (C), 171.3 (C), 170.5 (C), 168.0 (C), 166.7 (C), 162.2 (C), 128.4 (CH), 76.9 (CH), 50.2 (CH₂), 50.0 (CH), 46.4 (CH₂), 43.6 (CH), 41.1 (CH₂), 40.5 (C), 39.1 (CH₂), 37.7 (CH₂), 37.4 (CH₂), 35.2 (CH₂), 31.3 (CH₂), 29.7 (CH₂), 29.09 (CH₂), 29.07 (2×CH₂), 29.03 (CH₂), 28.97 (CH₂), 28.87 (CH₂), 28.73 (CH₂), 28.67 (CH₂), 25.2 (CH₂), 22.1 (CH₂), 20.6 (CH₃), 19.4 (CH₂), 14.0 (CH₃). HRMS-CI (m/z): [M+H]⁺ calcd for C₃₉H₆₀N₇O₉S₂, 834.3894; found, 834.3833. [α]_{D}²⁰ −17.0 (c 0.8, DMSO).

### Synthesis of the Linear Precursor 29b:

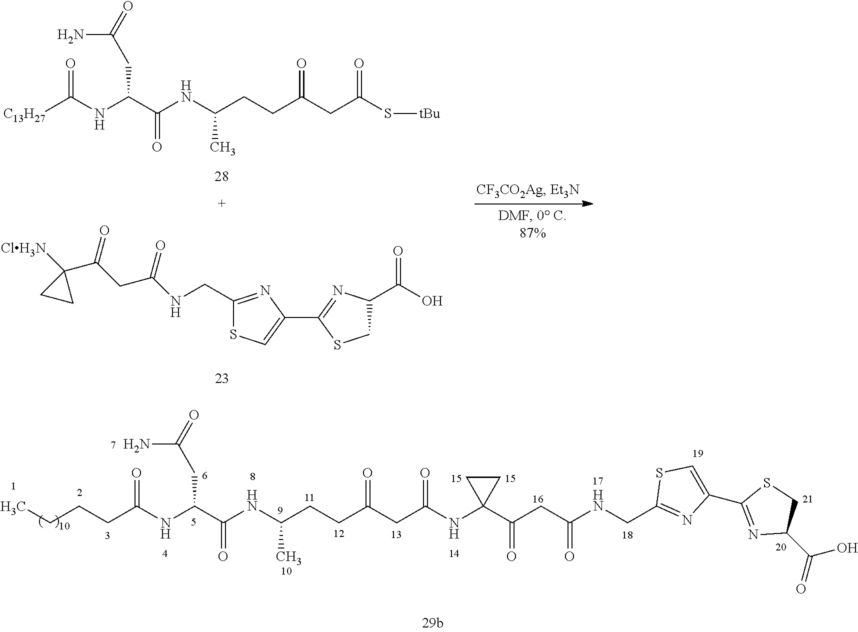

Silver trifluoroacetate (23.8 mg, 0.11 mmol, 2.00 equiv) was added to a solution of triethylamine (30.0 μL, 0.22 mmol, 4.00 equiv), the β-ketothioester 28 (30.0 mg, 54.0 μmol, 1 equiv), and the amine 23 (26.2 mg, 65.0 μmol, 1.20 equiv) in N,N-dimethylformamide (2.0 mL) at 0° C. The reaction mixture was stirred for 1 h at 0° C. The heterogeneous product mixture was diluted with aqueous citric acid solution (5%, 10 mL). The resulting precipitate was isolated by filtration. The solid was dried in vacuo to provide the linear precursor 29b as a white solid (39.0 mg, 87%).

¹H NMR (600 MHz, DMSO-d₆) δ 88.94 (t, J=6.0 Hz, 1H, H₁₇), 8.83 (s, 1H, H₁₄), 8.22 (s, 1H, H₁₉), 7.89 (d, J=7.7 Hz, 1H, H₄), 7.52 (d, J=8.5 Hz, 1H, H₈), 7.26 (bs, 1H, H₇), 6.87 (bs, 1H, H₇), 5.26 (t, J=9.0 Hz, 1H, H₂₀), 4.56 (d, J=6.0 Hz, 2H, H₁₈), 4.43 (q, J=7.2 Hz, 1H, H₅), 3.70 (td, J=8.6, 4.4 Hz, 1H, H₉), 3.67-3.60 (m, 1H, H₂₁), 3.60 (s, 2H, H₁₆), 3.54 (dd, J=11.3, 8.2 Hz, 1H, H₂₁), 3.34 (s, 2H, H₁₃), 2.56-2.44 (m, 2H, H₁₂), 2.42 (dd, 0.1=15.4, 6.0 Hz, 1H, H₆), 2.35 (dd, J=15.4, 7.7 Hz, 1H, H₆), 2.09 (t, J=7.5 Hz, 2H, H₃), 1.68-1.53 (m, 1H, H₁₁), 1.55-1.47 (m, 1H, H₁₁), 1.49-1.41 (m, 2H, H₂) 1.42-1.31 (m, 2H, H₁₅), 1.23 (bs, 20H, 10×CH₂), 1.05 (q, J=3.3 Hz, 2H, H₁₅), 0.99 (d, J=6.7 Hz, 3H, H₁₀), 0.85 (t, J=6.9 Hz, 3H, H₁). ¹³C NMR (151 MHz, DMSO-d₆) δ 204.8 (C), 204.1 (C), 172.2 (C), 171.8 (C), 171.3 (C), 170.5 (C), 170.2 (C), 168.1 (C), 167.0 (C), 163.3 (C), 147.3 (C), 122.2 (CH), 78.2 (CH), 50.2 (CH₂), 49.9 (CH), 46.5 (CH₂), 43.6 (CH), 40.5 (CH₂), 40.5 (C), 39.1 (CH₂), 37.4 (CH₂), 35.2 (CH₂), 34.4 (CH₂), 31.3 (CH₂), 29.7 (CH₂), 29.09 (CH₂), 29.07 (2×CH₂), 29.03 (CH₂), 28.97 (CH₂), 28.87 (CH₂), 28.73 (CH₂), 28.67 (CH₂), 25.2 (CH₂), 22.1 (CH₂), 20.6 (CH₃), 19.4 (CH₂), 14.0 (CH₃). HRMS-CI (m/z): [M+H]⁺ calcd for C₃₉H₆₀N₇O₉S₂, 834.3894; found, 834.3835. [α]_{D}²⁰ −10.0 (c 0.8, DMSO).

### Synthesis of the Linear Precursor 29c:

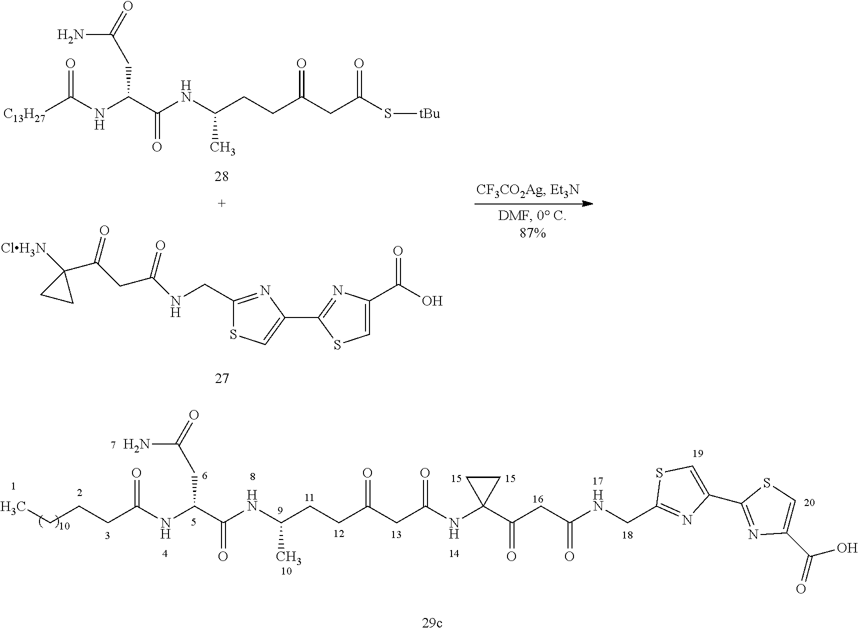

Silver trifluoroacetate (19.1 mg, 86.0 μmol, 2.00 equiv) was added to a solution of triethylamine (24.0 μL, 0.17 mmol, 4.00 equiv), the β-ketothioester 28 (24.0 mg, 43.0 μmol, 1 equiv), and the amine 27 (21.0 mg, 52.0 μmol, 1.20 equiv) in N,N-dimethylformamide (0.8 mL) at 0° C. The reaction mixture was stirred for 1 h at 0° C. The heterogeneous product mixture was diluted with aqueous citric acid solution (5%, 8.0 mL). The resulting precipitate was isolated by filtration. The solid was dried in vacuo to provide the linear precursor 29c as a white solid (31.0 mg, 86%).

¹H NMR (600 MHz, DMSO-d₆) δ 88.97 (t, J=6.0 Hz, 1H, H₁₇), 8.84 (s, 1H, H₁₄), 8.47 (s, 1H, H₂₀), 8.25 (s, 1H, H₁₉), 7.90 (d, J=7.7 Hz, 1H, H₄), 7.52 (d, J=8.6 Hz, 1H, H₈), 7.27 (bs, 1H, H₇), 6.87 (bs, 1H, H₇), 4.60 (d, J=6.0 Hz, 2H, H₁₈), 4.43 (q, J=7.2 Hz, 1H, H₅), 3.76-3.64 (m, 1H, H₉), 3.62 (s, 2H, H₁₆), 3.35 (s, 2H, H₁₃), 2.55-2.44 (m, 2H, H₁₂), 2.42 (dd, J=15.3, 5.8 Hz, 1H, H₆), 2.35 (dd, J=15.3, 7.8 Hz, 1H, H₆), 2.14-1.94 (m, 2H, H₃), 1.67-1.53 (m, 1H, H₁₁), 1.54-1.46 (m, 1H, H₁₁), 1.48-1.42 (m, 2H, H₂), 1.40 (q, J=3.2 Hz, 2H, H₁₅), 1.31-1.13 (m, 20H, 10×CH₂), 1.06 (q, J=3.5 Hz, 2H, H₁₅), 0.99 (d, J=6.6 Hz, 3H, H₁₀), 0.84 (t, J=6.9 Hz, 3H, H₁). ¹³C NMR (151 MHz, Chloroform-d) δ 204.8 (C), 204.1 (C), 172.2 (C), 171.4 (C), 171.3 (C), 170.5 (C), 168.1 (C), 167.0 (C), 162.1 (C), 162.0 (C), 148.1 (C), 147.1 (C), 129.0 (CH), 118.3 (CH), 50.2 (CH₂), 50.0 (CH), 46.5 (CH₂), 43.6 (CH), 40.6 (CH₂), 40.5 (C), 39.1 (CH₂), 37.4 (CH₂), 35.2 (CH₂), 31.3 (CH₂), 29.7 (CH₂), 29.08 (CH₂), 29.07 (2×CH₂), 29.03 (CH₂), 28.97 (CH₂), 28.87 (CH₂), 28.73 (CH₂), 28.67 (CH₂), 25.2 (CH₂), 22.1 (CH₂), 20.6 (CH₃), 19.4 (CH₂), 14.0 (CH₃). HRMS-CI (m/z): [M+H]⁺ calcd for C₃₉H₅₈N₇O₉S₂, 832.3737; found, 832.3693. [α]_{D}²⁰ +28.0 (c 0.8, DMSO).

### Synthesis of the Pyridone 30a:

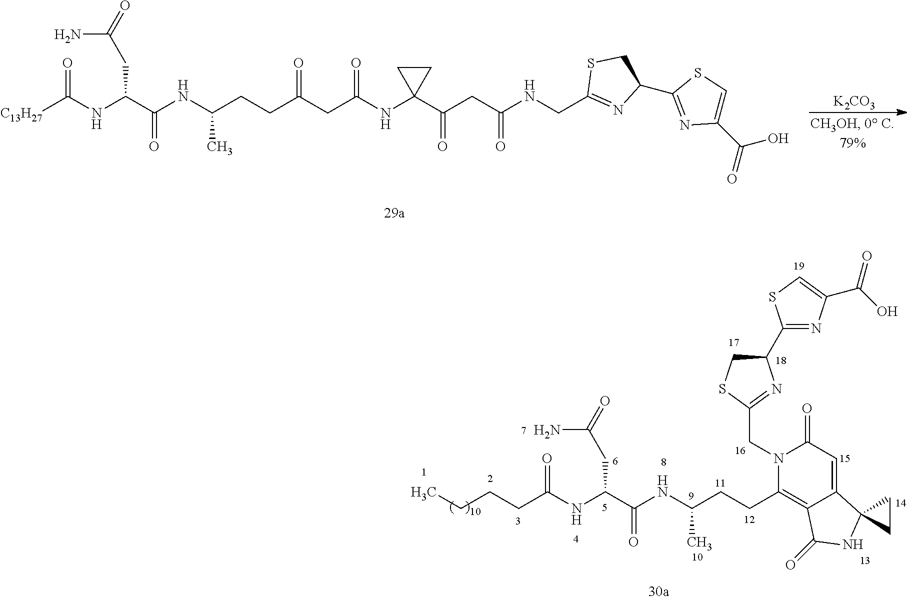

Potassium carbonate (9.94 mg, 72.0 μmol, 3.00 equiv) was added to a solution of the linear precursor 29a (20.0 mg, 24.0 μmol, 1 equiv) in methanol (1.5 mL) at 0° C. The reaction mixture was stirred for 3 h at 0° C. The heterogeneous product mixture was filtered through a plug of propylsulfonic acid functionalized silica gel. The filter cake was washed with methanol (10 mL). The filtrates were combined and the combined filtrates were concentrated. The residue obtained was applied to a trimethylamine acetate-functionalized silica column (Si-TMA acetate; eluting with 0.5% formic acid-acetonitrile). The fractions containing the product 30a were collected, combined, and concentrated to provide the pyridone 30a as a white solid (15.1 mg, 79%).

¹H NMR (500 MHz, DMSO-d₆-CD₃OD (3:1)) δ 8.19* (bs, 1H, H₁₃), 8.17 (s, 1H, H₁₉), 7.83* (app t, J=7.7 Hz, 1H, H₄), 7.64* (app dd, J=8.0, 5.2 Hz, 1H, H₈), 7.29* (bs, 1H, H₇), 6.79* (bs, 1H, H₇), 6.07 (s, 1H, H₁₅), 5.86 (app t, J=8.4 Hz, 1H, H₁₈), 5.24-5.06 (m, 2H, H₁₆), 4.58-4.46 (m, 1H, H₅), 3.92 (app t, J=10.3 Hz, 1H, H₁₇), 3.89-3.77 (m, 1H, H₉), 3.68-3.57 (m, 1H, H₁₇), 3.34-3.16 (m, 2H, H₁₂), 2.49-2.37 (m, 2H, H₆), 2.16-2.02 (m, 2H, H₃), 1.80-1.68 (m, 1H, H₃), 1.68-1.57 (m, 1H, H₃), 1.51-1.41 (m, 2H, H₂), 1.39 (q, J=4.8, 3.9 Hz, 2H, H₁₄), 1.32 (app t, J=3.5 Hz, 2H, H₁₄), 1.28-1.12 (m, 20H, 10×CH₂), 1.05 (app dd, J=9.0, 6.5 Hz, 3H, H₁₀), 0.83 (t, J=6.9 Hz, 3H, H₁). ¹³C NMR (151 MHz, DMSO-d₆-CD₃OD (3:1)) δ 172.7 (C), 172.1 (C), 171.1 (C), 170.8 (C), 167.2 (C), 164.1 (C), 163.1 (C), 162.1 (C), 160.0 (C), 153.6 (C), 149.7 (C), 127.4 (CH), 109.5 (C), 103.2 (CH), 76.7 (CH), 50.2 (CH), 45.1 (CH), 45.0 (CH₂), 40.1 (C), 39.0 (CH₂), 37.6 (CH₂), 35.6 (CH₂), 35.5 (CH₂), 31.7 (CH₂), 29.45 (CH₂), 29.43 (2×CH₂), 29.41 (CH₂), 29.34 (CH₂), 29.25 (CH₂), 29.1 (CH₂), 29.0 (CH₂), 25.6 (CH₂), 24.8 (CH₂), 22.5 (CH₂), 20.3 (CH₃), 15.4 (CH₂), 14.1 (CH₃). HRMS-CI (m/z): [M+H]⁺ calcd for C₃₉H₅₆N₇O₇S₂, 798.3683; found, 798.3623. [α]_{D}²⁰ −5.0 (c 0.8, DMSO).

*H-D exchange occurred slowly in solution.

### Synthesis of the Pyridone 30b:

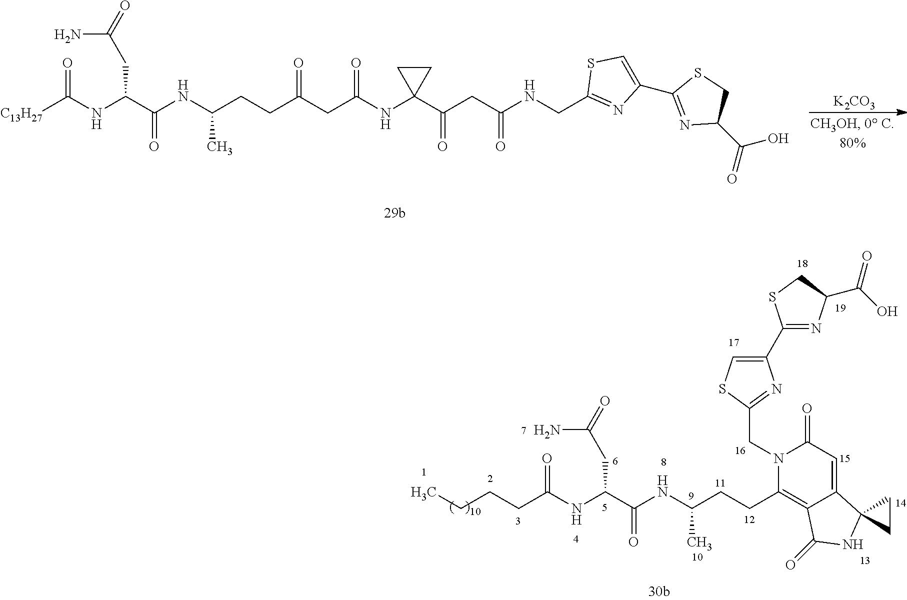

Potassium carbonate (4.97 mg, 36.0 μmol, 3.00 equiv) was added to a solution of the linear precursor 29b (10.0 mg, 12.0 μmol, 1 equiv) in methanol (800 μL) at 0° C. The reaction mixture was stirred for 3 h at 0° C. The heterogeneous product mixture was filtered through a plug of propylsulfonic acid functionalized silica gel. The filter cake was washed with methanol (8.0 mL). The filtrates were combined and the combined filtrates were concentrated. The residue obtained was applied to a trimethylamine acetate-functionalized silica column (Si-TMA acetate; eluting with 0.5% formic acid-acetonitrile). The fractions containing the product 30b were collected, combined, and concentrated to provide the pyridone 30b as a white solid (7.7 mg, 80%).

¹H NMR (400 MHz, DMSO-d₆-CD₃OD (3:1)) δ 8.17 (bs, 2H, H₁₃*, H₁₇), 7.89* (d, J=7.9 Hz, 1H, H₄), 7.65* (d, J=7.9 Hz, 1H, H₈), 7.30* (bs, 1H, H₇), 6.77* (bs, 1H, H₇), 6.11 (s, 1H, H₁₅), 5.70-5.39 (m, 2H, H₁₆), 5.24-4.98 (m, 1H, H₁₉), 4.53 (app q, J=7.3 Hz, 1H, H₅), 3.97-3.77 (m, 1H, H₉), 3.64-3.45 (m, 2H, H₁₈), 3.44-3.26 (m, 2H, H₁₂), 2.53 (dd, J=15.5, 6.3 Hz, 1H, H₆), 2.43 (dd, J=15.2, 7.4 Hz, 1H, H₆), 2.23-1.98 (m, 2H, H₃), 1.82-1.66 (m, 1H, H₁₁), 1.66-1.55 (m, 1H, H₁₁), 1.50-1.41 (m, 2H, H₂), 1.39 (q, J=5.1, 4.0 Hz, 2H, H₁₄), 1.32 (q, J=6.1, 5.0 Hz, 2H, H₁₄), 1.28-1.11 (m, 20H, 10×CH₂), 1.07 (d, J=6.5 Hz, 3H, H₁₀), 0.82 (t, J=6.7 Hz, 3H, H₁). ¹³C NMR (DMSO-d₆-CD₃OD (3:1)) δ 173.2 (C), 172.5 (C), 172.4 (C), 171.1 (C), 167.3 (C), 166.1 (C), 163.4 (C), 162.5 (C), 160.5 (C), 153.7 (C), 147.8 (C), 123.7 (CH), 110.2 (C), 103.6 (CH), 79.6 (CH), 50.5 (CH), 45.2 (CH), 44.8 (CH₂), 40.1 (C), 37.5 (CH₂), 35.9 (CH₂), 35.8 (CH₂), 35.2 (CH₂), 31.9 (CH₂), 29.61 (2×CH₂), 29.58 (CH₂), 29.56 (CH₂), 29.50 (CH₂), 29.4 (CH₂), 29.3 (CH₂), 29.1 (CH₂), 25.7 (CH₂), 24.7 (CH₂), 22.6 (CH₂), 20.5 (CH₃), 15.5 (CH₂), 14.1 (CH₃). HRMS-CI (m/z): [M+H]⁺ calcd for C₃₉H₅₆N₇O₇S₂, 798.3683; found, 798.3625. [α]_{D}²⁰ +39.0 (c 0.8, DMSO).

*H-D exchange occurred slowly in solution.

### Synthesis of Precolibactin C (6):

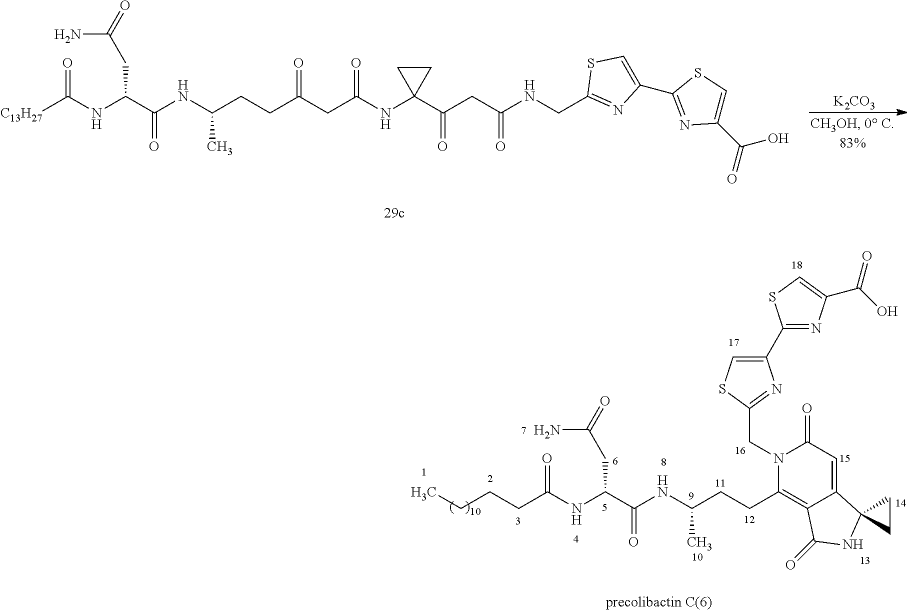

Potassium carbonate (7.48 mg, 54.0 μmol, 3.00 equiv) was added to a solution of the linear precursor 29c (15.0 mg, 18.0 μmol, 1 equiv) in methanol (1.0 mL) at 0° C. The reaction mixture was stirred for 3 h at 0° C. The heterogeneous product mixture was filtered through a plug of propylsulfonic acid functionalized silica gel (1.0×0.5 cm). The filter cake was washed with methanol (10 mL). The filtrates were combined and the combined filtrates were concentrated. The residue obtained was applied to a trimethylamine acetate-functionalized silica column (Si-TMA acetate; eluting with 0.5% formic acid-acetonitrile). The fractions containing the product 6 were collected, combined, and concentrated to provide precolibactin C (6) as a white solid (11.9 mg, 83%).

¹H NMR (600 MHz, DMSO-d₆) δ 8.51 (s, 1H, H₁₃), 8.24 (s, 1H, H₁₇), 8.18 (s, 1H, H₁₈), 7.99 (d, J=7.8 Hz, 1H, H₄), 7.72 (d, J=8.0 Hz, 1H, H₈), 7.29 (bs, 1H, H₇), 6.82 (bs, 1H, H₇), 6.16 (s, 1H, H₁₅), 5.57 (q, J=15.9 Hz, 2H, H₁₆), 4.48 (q, J=7.2 Hz, 1H, H₅), 3.88 (dq, J=13.8, 6.8 Hz, 1H, H₉), 3.50-3.31 (m, 2H, H₁₂), 2.47 (dd, J=15.2, 6.2 Hz, 1H, H₆), 2.37 (dd, J=15.0, 7.4 Hz, 1H, H₆), 2.11-1.98 (m, 2H, H₃), 1.78-1.64 (m, 2H, H₁₁), 1.44-1.35 (m, 4H, H₂, H₁₄), 1.36-1.31 (m, 2H, H₁₄), 1.26-1.11 (m, 20H, 10×CH₂), 1.06 (d, J=6.5 Hz, 3H, H₁₀), 0.83 (t, J=7.0 Hz, 3H, H₁). ¹³C NMR (151 MHz, DMSO-d₆) δ 172.2 (C), 171.6 (C), 170.5 (C), 166.7 (C), 166.5 (C), 164.0 (C), 163.2 (C), 161.8 (C), 161.0 (C), 159.8 (C), 153.1 (C), 147.2 (C), 126.1 (CH), 118.6 (CH), 109.6 (C), 103.3 (CH), 50.1 (CH), 44.7 (CH), 44.4 (CH₂), 39.7 (C), 37.4 (CH₂), 35.4 (CH₂), 35.2 (CH₂), 31.3 (CH₂), 29.07 (CH₂), 29.04 (2×CH₂), 29.02 (CH₂), 28.96 (CH₂), 28.87 (CH₂), 28.7 (CH₂), 28.6 (CH₂), 25.2 (CH₂), 24.1 (CH₂), 22.1 (CH₂), 20.3 (CH₃), 15.2 (CH₂), 14.0 (CH₃).

¹H NMR (500 MHz, CD₃OD) δ 8.28 (s, 1H, H₁₇), 8.16 (s, 1H, H₁₈), 6.16 (s, 1H, H₁₅), 5.75 (d, J=15.7 Hz, 1H, H₁₆), 5.70 (d, J:=15.7 Hz, 1H, H₁₆), 4.73 (t, J=6.5 Hz, 1H, H₅), 4.12-3.99 (m, 1H, H₉), 3.71-3.57 (m, 1H, H₁₂), 3.55-3.42 (m, 1H, H₁₂), 2.71 (t, J=6.1 Hz, 2H, H₆), 2.23 (t, J=7.4 Hz, 2H, H₃), 1.98-1.86 (m, 1H, H₁₁), 1.81-1.68 (m, 1H, H₁₁), 1.66-1.53 (m, 2H, H₂), 1.54-1.50 (m, 2H, H₁₄), 1.47-1.38 (m, 2H, H₁₄), 1.32-1.20 (m, 20H, 10×CH₂), 1.18 (d, J=6.6 Hz, 3H, H₁₀), 0.89 (t, J=7.0 Hz, 3H, H₁). ¹³C NMR (126 MHz, Methanol-d4) δ 176.3 (C), 175.0 (C), 172.9 (C), 169.3 (C), 167.2 (C), 164.9 (C), 163.6 (C), 163.4 (C), 162.5 (C), 155.2 (C), 149.3 (C), 148.1 (C), 126.9 (CH), 120.2 (CH), 112.2 (C), 104.2 (CH), 52.0 (CH), 47.0 (CH), 46.0 (CH₂), 41.5 (C), 38.0 (CH₂), 37.0 (CH₂), 36.8 (CH₂), 33.1 (CH₂), 30.80 (CH₂), 30.77 (CH₂), 30.76 (2×CH₂), 30.65 (CH₂), 30.51 (CH₂), 30.48 (CH₂), 30.3 (CH₂), 26.8 (CH₂), 26.0 (CH₂), 23.7 (CH₂), 20.8 (CH₃), 16.3 (CH₂), 14.4 (CH₃). HRMS-CI (m/z): [M+H]⁺ calcd for C₃₉H₅₄N₇O₇S₂, 796.3526; found, 796.3466. [α]_{D}²⁰ −21.0 (c 0.8, DMSO).

### Synthesis of the Amine 31:

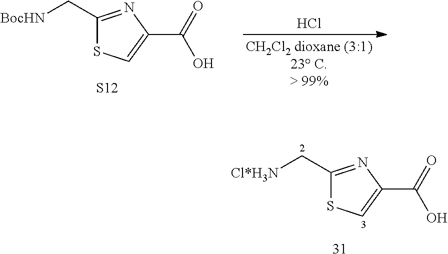

A solution of hydrogen chloride in 1,4-dioxane (4.0 N, 6.0 mL, 24.0 mmol, 13.3 equiv) was added dropwise via syringe pump over 20 min to a solution of the thiazole S12 (467 mg, 1.81 mmol, 1 equiv) in dichloromethane (18.0 mL) at 23° C. The resulting mixture was stirred for 1 h at 23° C. The reaction mixture was concentrated to provide the amine 31 as a white solid (352 mg, >99%).

The product 31 obtained in this way was used directly in the following step.

¹H NMR (600 MHz, DMSO-d₆) δ 8.80 (bs, 3H), 8.52 (s, 1H, H₃), 4.44 (q, J=5.8 Hz, 2H, H₂). ¹³C NMR (151 MHz, DMSO-d₆) δ 162.8 (C), 161.8 (C), 146.8 (C), 130.6 (CH), 39.5 (CH₂).

### Synthesis of the β-Ketoamide S13:

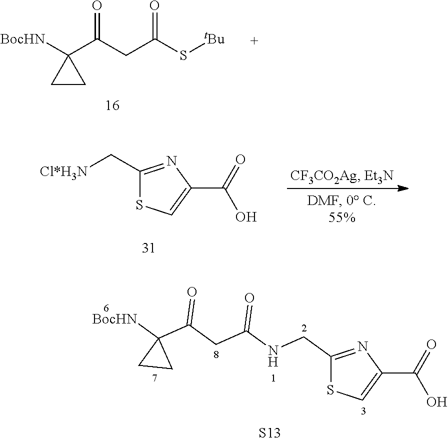

Three equal portions of silver trifluoroacetate (72.6 mg, 328 μmol, 0.40 equiv) were added over 1 h to a solution of triethylamine (459 μL, 3.29 mmol, 4.00 equiv), the 0-ketothioester 16 (311 mg, 986 μmol, 1.20 equiv), and the amine 31 (160 mg, 822 μmol, 1 equiv) in N,N-dimethylformamide (9.0 mL) at 0° C. The reaction mixture was stirred for 1 h at 0° C. The product mixture was directly applied to a column containing trimethylamine acetate-functionalized silica gel (Si-TMA acetate; eluting with 2% acetic acid-methanol).

The fractions containing product were collected, combined, and concentrated. The residue obtained was further purified by automated flash-column chromatography (eluting with 2% acetic acid-dichloromethane initially, grading to 2% acetic acid-10% dichloromethane-methanol, linear gradient). The fractions containing the product S13 were collected, combined, and concentrated to provide the β-ketoamide S13 as a white solid (173 mg, 55%).

¹H NMR (600 MHz, DMSO-d₆) δ 12.95 (bs, 1H), 8.93 (t, J=6.0 Hz, 1H, H₁), 8.36 (s, 1H, H₃), 7.76 (s, 1H), 4.54 (d, J=6.0 Hz, 2H, H₂), 3.55 (s, 2H, H₆), 1.40 (s, 9H, H₈), 1.38-1.33 (m, 2H, H₁), 1.07 (q, J=4.2 Hz, 2H, H₁). ¹³C NMR (151 MHz, DMSO-d₆) δ 204.7 (C), 169.8 (C), 166.8 (C), 162.0 (C), 156.0 (C), 146.6 (C), 128.9 (CH), 78.6 (C), 46.1 (CH₂), 41.2 (C), 40.5 (CH₂), 28.2 (CH₃), 19.5 (CH₂). IR (ATR-FTIR), cm⁻¹: 3316 (br), 2977 (w), 1701 (s), 1684 (s), 1509 (s), 1249 (s), 1161 (s), 1069 (s), 751 (m). HRMS-CI (m/z): [M+H]⁺ calcd for C₁₆H₂₂N₃OS, 384.1229; found, 384.1224.

### Synthesis of the Amine 32:

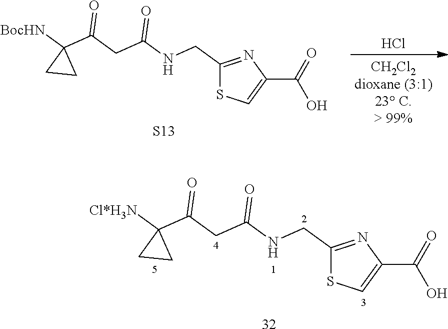

A solution of hydrogen chloride in 1,4-dioxane (4.0 N, 2.0 mL, 8.0 mmol, 55.9 equiv) was added dropwise via syringe pump over 20 min to a solution of the β-ketoamide S13 (55.0 mg, 143 μmol, 1 equiv) in dichloromethane (6.0 mL) at 23° C. The resulting mixture was stirred for 1 h at 23° C. The reaction mixture was concentrated to provide the amine 32 as a white solid (45.9 mg, >99%).

The product 32 obtained in this way was used directly in the following step.

¹H NMR (600 MHz, DMSO-d₆) δ 9.13 (t, J=6.0 Hz, 1H, H₁), 8.79 (bs, 3H), 8.38 (s, 1H, H₃), 4.57 (d, J=5.9 Hz, 2H, H₂), 3.38 (s, 2H, H₄), 1.83-1.68 (m, 2H, H₅), 1.57-1.26 (m, 2H, H₅). ¹³C NMR (151 MHz, DMSO-d₆) δ 199.40 (C), 169.29 (C), 165.86 (C), 161.99 (C), 146.67 (C), 128.94 (CH), 42.35 (CH₂), 42.01 (C), 40.51 (CH₂), 13.10 (CH₂).

### Synthesis of the Linear Precursor 33:

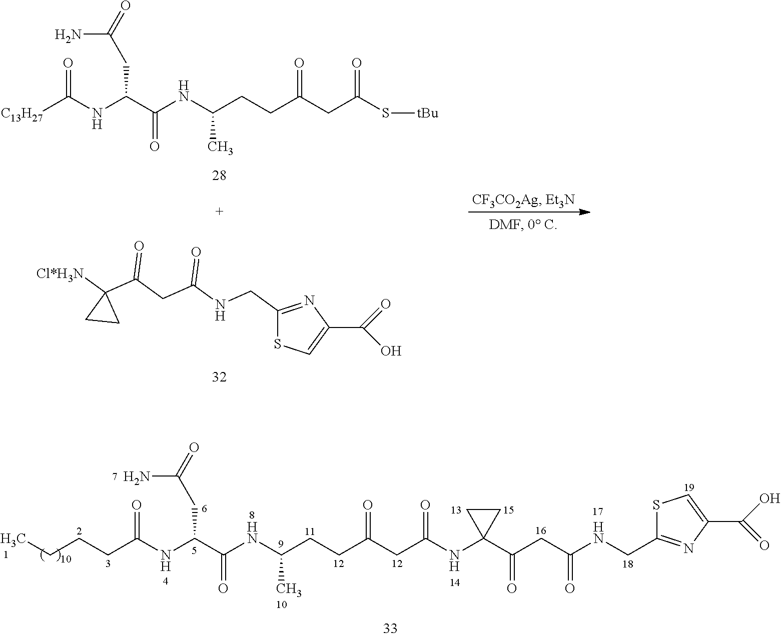

Silver trifluoroacetate (33.4 mg, 151 μmol, 2.00 equiv) was added to a solution of triethylamine (42.1 μL, 302 μmol, 4.00 equiv), the β-ketothioester 28 (42.0 mg, 75.6 μmol, 1 equiv), and the amine 32 (24.2 mg, 75.6 μmol, 1.00 equiv) in N,N-dimethylformamide (1.5 mL) at 0° C. The reaction mixture was stirred for 1 h at 0° C. The heterogeneous product mixture was diluted with aqueous citric acid solution (5%, 12 mL). The resulting precipitate was isolated by filtration and was dried in vacuo to provide 33.

The product 33 obtained in this way was used directly in the following step.

¹H NMR (600 MHz, DMSO-d₆) δ 88.92 (t, J=6.0 Hz, 1H, H₁₇), 8.83 (s, 1H, H₁₄), 8.36 (s, 1H, H₁₉), 7.89 (d, J=8.0 Hz, 1H, H₄), 7.52 (d, J=8.6 Hz, 1H, H₈), 7.29-7.18 (m, 1H, H₇), 6.91-6.78 (m, 1H, H₇), 4.55 (d, J=5.9 Hz, 2H, H₁₈), 4.49-4.36 (m, 1H, H₅), 3.78-3.62 (m, 1H, H₉), 3.59 (s, 2H, H₁₆), 3.34 (s, 2H, H₁), 2.56-2.45 (m, 2H, H₁₂), 2.48-2.37 (m, 1H, H₆), 2.39-2.27 (m, 1H, H₆), 2.08 (q, J=7.7 Hz, 2H, H₃), 1.64-1.53 (m, 1H, H₁₁), 1.51-1.43 (m, 3H, H₁₁, H₂), 1.39 (q, J=3.4 Hz, 2H, H₁₅), 1.29-1.18 (m, 20H, 10×CH₂), 1.05 (q, J=3.6 Hz, 2H, H₁₅), 1.04-0.93 (m, 3H, H₁₀), 0.85 (t, J=7.0 Hz, 3H, H₁). ¹³C NMR (151 MHz, DMSO-d₆) δ 204.8 (C), 204.1 (C), 172.2 (C), 171.4 (C), 171.3 (C), 170.5 (C), 168.1 (C), 166.9 (C), 162.0 (C), 146.6 (C), 128.9 (CH), 50.2 (CH₂), 49.9 (CH), 46.6 (CH₂), 43.6 (CH), 40.53 (CH₂), 40.50 (C), 39.1 (CH₂), 37.4 (CH₂), 35.2 (CH₂), 31.3 (CH₂), 29.7 (CH₂), 29.08 (CH₂), 29.07 (2×CH₂), 29.03 (CH₂), 28.97 (CH₂), 28.87 (CH₂), 28.73 (CH₂), 28.67 (CH₂), 25.2 (CH₂), 22.1 (CH₂), 20.6 (CH₃), 19.4 (CH₂), 14.0 (CH₃).

### Synthesis of Precolibactin B (3):

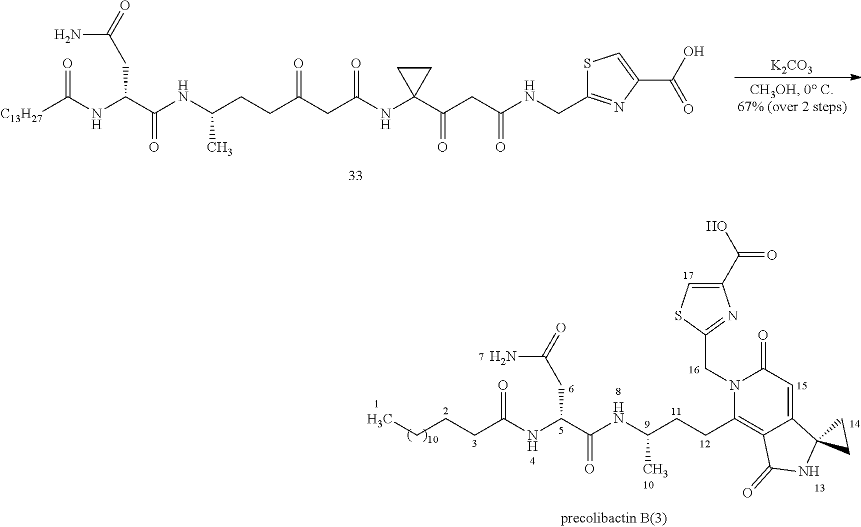

Potassium carbonate (31.3 mg, 227 μmol, 3.00 equiv) was added to a solution of the unpurified linear precursor 33 (nominally 56.6 mg, 75.6 μmol, 1 equiv) in methanol (3.5 mL) at 0° C. The reaction mixture was stirred for 2 h at 0° C. The heterogeneous product mixture was filtered through a pad of propylsulfonic acid functionalized silica gel (1.0×0.5 cm). The filter cake was washed with methanol (10 mL). The filtrates were combined and the combined filtrates were concentrated. The residue obtained was applied to a trimethylamine acetate-functionalized silica column (Si-TMA acetate; eluting with 0.5% formic acid-acetonitrile). The fractions containing the product 3 were collected, combined, and concentrated to provide precolibactin B (3) as a white solid (36.0 mg, 67% over 2 steps).

¹H NMR (600 MHz, DMSO-d₆) δ 13.09 (bs, 1H), 8.47 (s, 1H, H₁₃), 8.39 (s, 1H, H₁₇), 7.83 (d, J=8.1 Hz, 1H, H₄), 7.72 (d, J=8.3 Hz, 1H, H₈), 7.25 (bs, 1H, H₇), 6.83 (bs, 1H, H₇), 6.16 (s, 1H, H₁₅), 5.62 (d, J=15.7 Hz, 1H, H₁₆), 5.51 (d, J=15.8 Hz, 1H, H₁₆), 4.52 (q, J=7.2 Hz, 1H, H₅), 3.93-3.79 (m, 1H, H₉), 3.44-3.35 (m, 1H, H₁₂), 3.33-3.22 (m, 1H, H₁₂), 2.50-2.45 (m, 1H, H₆), 2.41 (dd, J=15.2, 7.4 Hz, 1H, H₆), 2.07-1.97 (m, 2H, H₃), 1.77-1.61 (m, 1H, H₁₁), 1.62-1.51 (m, 1H, H₁₁), 1.48-1.39 (m, 2H, H₂), 1.39-1.30 (m, 4H, H₁₄), 1.27-1.12 (m, 20H, 10×CH₂), 1.03 (d, J=6.6 Hz, 3H, H₁₀), 0.84 (t, J=7.0 Hz, 3H, H₁). ¹³C NMR (151 MHz, DMSO-d₆) δ 172.2 (C), 171.7 (C), 170.6 (C), 166.7 (C), 165.7 (C), 162.0 (C), 161.9 (C), 159.9 (C), 153.2 (C), 146.3 (C), 130.1 (CH), 109.7 (C), 103.3 (CH), 50.0 (CH), 44.6 (CH), 44.3 (CH₂), 39.9 (C), 37.3 (CH₂), 35.5 (CH₂), 35.3 (CH₂), 31.4 (CH₂), 29.4 (CH₂), 29.13 (2×CH₂), 29.11 (CH₂), 29.04 (CH₂), 28.9 (CH₂), 28.81 (CH₂), 28.7 (CH₂), 25.2 (CH₂), 24.2 (CH₂), 22.2 (CH₂), 20.6 (CH₃), 15.3 (CH₂), 14.1 (CH₃).

¹H NMR (500 MHz, CD₃OD) δ 8.17 (s, 1H, H₁₇), 6.17 (s, 1H, H₁₅), 5.82 (d, J=15.3 Hz, 1H, H₁₆), 5.68 (d, J=15.2 Hz, 1H, H₁₆), 4.82 (t, J=6.5 Hz, 1H, H₅), 4.20-4.03 (m, 1H, H₉), 3.71-3.53 (m, 1H, H₁₂), 3.52-3.42 (m, 1H, H₁₂), 2.81-2.72 (m, 2H, H₆), 2.30-2.16 (m, 2H, H₃), 1.99-1.82 (m, 1H, H₁₁), 1.62-1.54 (m, 3H, H₁₁, H₂), 1.56-1.47 (m, 2H, H₁₄), 1.45-1.35 (m, 2H, H₁₄), 1.34-1.19 (m, 20H, 10×CH₂), 1.16 (d, J=6.6 Hz, 3H, H₁₀), 0.90 (t, J=6.9 Hz, 3H, H₁). ¹³C NMR (126 MHz, CD₃OD) δ 176.4 (C), 175.2 (C), 172.9 (C), 169.2 (C), 166.4 (C), 166.0 (C), 164.9 (C), 162.4 (C), 155.2 (C), 151.5 (C), 128.9 (CH), 112.2 (C), 104.1 (CH), 52.3 (CH), 46.7 (CH), 46.0 (CH₂), 41.4 (C), 37.9 (CH₂), 37.0 (CH₂), 36.8 (CH₂), 33.0 (CH₂), 30.74 (CH₂), 30.71 (3×CH₂), 30.6 (CH₂), 30.4 (2×CH₂), 30.3 (CH₂), 26.8 (CH₂), 26.0 (CH₂), 23.7 (CH₂), 21.1 (CH₃), 16.3 (CH₂), 14.4 (CH₃). HRMS-CI (m/z): [M+H]⁺ calcd for C₃₆H₅₃N₆O₇S, 713.3696; found, 713.3689. [α]_{D}²⁰ −15.0 (c 0.7, CH₃OH).

### Synthesis of Precolibactin A (7):

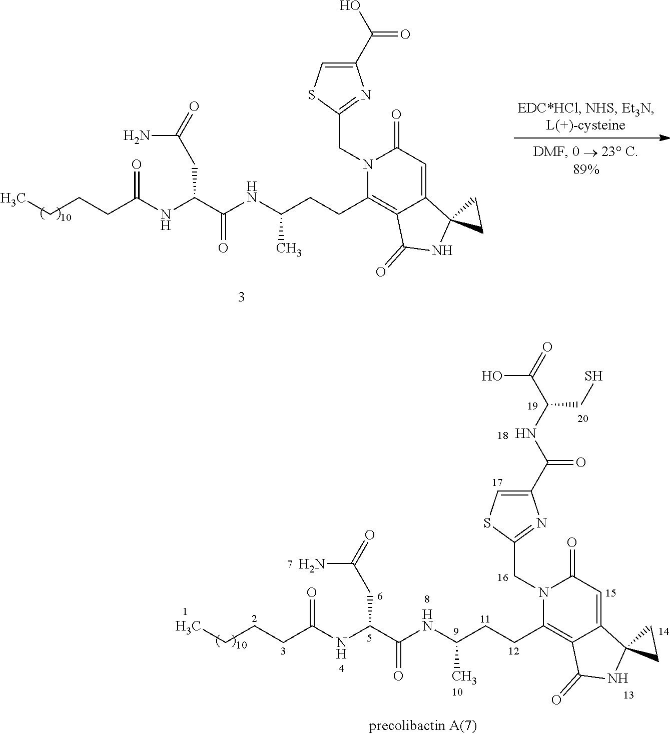

N-(3-dimethylaminopropyl)-N′-ethylcarbodiimide hydrogen chloride (EDC.HCl, 2.6 mg, 13.5 μmol, 1.20 equiv) was added to a degassed solution of the pyridone 3 (8.00 mg, 11.2 μmol, 1 equiv) and N-hydroxysuccinimide (NHS, 1.81 mg, 15.7 μmol, 1.40 equiv) in N,N-dimethylformamide (300 μL) at 0° C. After 30 min the reaction mixture was warmed to 23° C. and stirred for 8 h. L-cysteine (2.72 mg, 22.4 μmol, 2.00 equiv) and triethylamine (9.26 μL, 44.9 μmol, 4.00 equiv) were added to the reaction mixture. The reaction mixture was stirred for a further 14 h at 23° C. The product mixture was filtered through a plug of propylsulfonic acid functionalized silica gel (1.0×0.5 cm) under an atmosphere of dinitrogen. The filter cake was washed with methanol (5.0 mL). The filtrates were combined and the combined filtrates were concentrated. The residue obtained was diluted with water (10 mL) and the resulting precipitate was isolated by filtration through a plug of propylsulfonic acid functionalized silica gel (2.0×1.0 cm) under a N₂ atmosphere. The filter cake was washed with methanol (10 mL). The filtrates were combined and the combined filtrates were concentrated to provide precolibactin A (7) as a white solid (8.2 mg, 89%).

¹H NMR (600 MHz, CD₃OD) δ 8.23 (s, 1H, H₁₇), 6.16 (s, 1H, H₁₅), 5.70 (s, 2H, H₁₆), 4.79 (t, J=5.2 Hz, 1H, H₁₉), 4.73 (t, J=6.5 Hz, 1H, H₅), 4.10-4.02 (m, 1H, H₉), 3.70-3.63 (m, 1H, H₁₂), 3.48-3.41 (m, 1H, H₁₂), 3.11 (d, J=5.3 Hz, 2H, H₂₀), 2.70 (app t, J=6.2 Hz, 2H, H₆), 2.23 (t, J=7.4 Hz, 2H, H₃), 1.99-1.90 (m, 1H, H₁₁), 1.77-1.67 (m, 1H, H₁₁), 1.62-1.54 (m, 2H, H₂), 1.54-1.49 (m, 2H, H₁₄), 1.44-1.38 (m, 2H, H₁₄), 1.36-1.20 (m, 20H, 10×CH₂), 1.21 (d, J=6.9 Hz, 3H, H₁₀), 0.89 (t, J=7.1 Hz, 3H, H₁). ¹³C NMR (151 MHz, CD₃OD) δ 176.3 (C), 175.1 (C), 172.83 (C), 172.81 (C), 169.2 (C), 166.8 (C), 164.9 (C), 162.7 (C), 162.6 (C), 155.2 (C), 149.6 (C), 127.2 (CH), 112.2 (C), 104.2 (CH), 55.6 (CH), 51.9 (CH), 46.9 (CH), 46.0 (CH₂), 41.5 (C), 38.1 (CH₂), 37.0 (CH₂), 36.7 (CH₂), 33.1 (CH₂), 30.81 (CH₂), 30.79 (CH₂), 30.77 (2×CH₂), 30.66 (CH₂), 30.51 (CH₂), 30.49 (CH₂), 30.3 (CH₂), 26.83 (CH₂), 26.79 (CH₂), 25.8 (CH₂), 23.8 (CH₂), 20.7 (CH₃), 16.3 (CH₂), 14.5 (CH₃). HRMS-CI (m/z): [M+H]⁺ calcd for C₃₉H₅₈N₇O₈S₂, 816.3788; found, 816.3787. [α]_{D}²⁰ −1.0 (c 1.0, CH₃OH).

### FURTHER EXAMPLES (SECOND SET OF REFERENCES APPLY)

### General Experimental Methods

UV Spectroscopy.

UV thermal denaturation samples were prepared by mixing calf thymus DNA [32.0 mM base pairs (bps)] in 2.09 mM NaH₂PO₄, 7.13 mM Na₂HPO₄, 928 μM Na₂EDTA, 1.01 mM DMSO, pH 7.18 to a final volume of 1.0 mL. Samples were subjected to sonication (6 h) at 25° C. to effect complete dissolution. After incubation with 15a, 15b, 17a, and 17b for 5 min, 1 h, 3 h, 6 h, or 15 h, the UV thermal denaturation spectra of the samples were recorded at 260 nm as a function of temperature (55→80° C., heating rate: 0.5° C./min). First derivative plots were used to determine the denaturation temperature.

Electrophoretic Gel Assay.

The 4,163 bp plasmid pBR322 was propogated in DH5a, isolated by MaxiPrep (Qiagen), and linearized with 5 U/μg EcoRI (NEB). The cut plasmid was column purified and eluted into 10 mM Tris pH 8.0. For each reaction, 130 ng of DNA (20 μM base pairs) was incubated with compound in a 10 μL total volume. Reactions proceeded for 15 h at 37° C., unless otherwise noted. Compounds were diluted in DMSO such that each reaction consisted of a fixed 5% DMSO concentration. Pure MMS (Alfa Aesar) and cisplatin (Biovision) stock solutions were diluted into DMSO immediately prior to use. After incubation, 35 μL of denaturation buffer (6% sucrose, 1% sodium hydroxide, 0.04% bromophenol blue) was added to each reaction. Non-denatured control samples were diluted with 6% sucrose, 0.04% bromophenol blue. Samples were vortexed for 1 min, left at room temperature for 15 min, and then immediately frozen at −80° C. Thawed samples were then loaded onto a 1% agarose Tris-Borate-EDTA (TBE) gel stained with SybrGold (Molecular Probes) and run in TBE buffer for 1 hour at 120V.

General Experimental Procedures.

All reactions were performed in single-neck, flame-dried, round-bottomed flasks fitted with rubber septa under a positive pressure of nitrogen unless otherwise noted. Air- and moisture-sensitive liquids were transferred via syringe or stainless steel cannula, or were handled in a nitrogen-filled drybox (working oxygen level <10 ppm). Organic solutions were concentrated by rotary evaporation at 28-32° C. Flash-column chromatography was performed as described by Still et al.,¹ employing silica gel (60 Å, 40-63 μm particle size) purchased from Sorbent Technologies (Atlanta, Ga.). Anion-exchange chromatography was performed as described by Béland et al.,² employing trimethylamine acetate-functionalized silica gel (SiliaBond® TMA Acetate). Analytical thin-layered chromatography (TLC) was performed using glass plates pre-coated with silica gel (0.25 mm, 60 Å pore size) impregnated with a fluorescent indicator (254 nm). TLC plates were visualized by exposure to ultraviolet light (UV).

Materials.

Commercial solvents and reagents were used as received with the following exceptions. Dichloromethane, ether and N,N-dimethylformamide were purified according to the method of Pangborn et al.³ Triethylamine was distilled from calcium hydride under an atmosphere of argon immediately before use. Di-iso-propylamine was distilled from calcium hydride and was stored under nitrogen. Methanol was distilled from magnesium turnings under an atmosphere of nitrogen immediately before use. Tetrahydrofuran was distilled from sodium-benzophenone under an atmosphere of nitrogen immediately before use. Deoxyribonucleic acid sodium salt from calf thymus (Type I, fibers) was purchased from Sigma Aldrich. Trimethylamine acetate-functionalized silica gel (SiliaBond® TMA Acetate) was purchased from SiliCycle (Quebec City, Calif.). tert-butyl-(S)-2-methyl-5-oxopyrrolidine-1-carboxylate (S1),⁴ 2′-((3-(1-aminocyclopropyl)-3-oxopropanamido)methyl)-[2,4′-bithiazole]-4-carboxylic acid hydrochloride (11),⁵ 3-(tert-butylthio)-3-oxopropanoic acid (S5),⁶ 2′-(aminomethyl)-[2,4′-bithiazole]-4-carboxylic acid hydrochloride (S7),⁵ N,N′-bis-(tert-butoxycarbonyl)-N″-(2-aminoethyl)-guanidine (S11),⁷ N,N′-bis-(tert-butoxycarbonyl)-N″-(4-aminobutyl)-guanidine (S12),⁵ tert-butyl-((4-carbamothioylthiazol-2-yl)methyl)carbamate (S13),⁵ and S-(tert-butyl)-3-(1-((ter-butoxycarbonyl)amino)cyclopropyl)-3-oxopropanethioate (S17)⁵ were prepared according to published procedures.

Instrumentation.

Proton nuclear magnetic resonance spectra (¹H NMR) were recorded at 500 or 600 MHz at 24° C., unless otherwise noted. Chemical shifts are expressed in parts per million (ppm, δ scale) downfield from tetramethylsilane and are referenced to residual protium in the NMR solvent (CD₂Cl₂, δ 5.32; CD₃OD, δ 3.31; C₂D₆OS, δ 2.50). Data are represented as follows: chemical shift, multiplicity (s=singlet, d=doublet, t=triplet, q=quartet, m=multiplet and/or multiple resonances, br=broad, app=apparent), coupling constant in Hertz, integration, and assignment. Proton-decoupled carbon nuclear magnetic resonance spectra (¹³C NMR) were recorded at 125 MHz at 24° C., unless otherwise noted. Chemical shifts are expressed in parts per million (ppm, δ scale) downfield from tetramethylsilane and are referenced to the carbon resonances of the solvent (CD₂Cl₂, δ 54.0; CD₃OD, δ 49.0; C₂D₆OS, δ 39.5). Signals of protons and carbons were assigned, as far as possible, by using the following two dimensional NMR spectroscopy techniques: [¹H, ¹H] COSY (Correlation Spectroscopy), [¹H, ¹³C] HSQC (Heteronuclear Single Quantum Coherence) and long range [¹H, ¹³C] HMBC (Heteronuclear Multiple Bond Connectivity). Attenuated total reflectance Fourier transform infrared (ATR-FTIR) spectra were obtained using a Thermo Electron Corporation Nicolet 6700 FTIR spectrometer referenced to a polystyrene standard. Data are represented as follows: frequency of absorption (cm⁻¹), intensity of absorption (s=strong, m=medium, w=weak, br=broad). Analytical ultra high-performance liquid chromatography/mass spectrometry (UPLC/MS) was performed on a Waters UPLC/MS instrument equipped with a reverse-phase C₁₈ column (1.7 μm particle size, 2.1×50 mm), dual atmospheric pressure chemical ionization (API)/electrospray (ESI) mass spectrometry detector, and photodiode array detector. Samples were eluted with a linear gradient of 5% acetonitrile-water containing 0.1% formic acid→100% acetonitrile containing 0.1% formic acid over 0.75 min, followed by 100% acetonitrile containing 0.1% formic acid for 0.75 min, at a flow rate of 800 μL/min. High-resolution mass spectrometry (HRMS) were obtained on a Waters UPLC/HRMS instrument equipped with a dual API/ESI high-resolution mass spectrometry detector and photodiode array detector. Unless otherwise noted, samples were eluted over a reverse-phase C₁₈ column (1.7 μm particle size, 2.1×50 mm) with a linear gradient of 5% acetonitrile-water containing 0.1% formic acid→95% acetonitrile-water containing 0.1% formic acid for 1 min, at a flow rate of 600 μL/min. Optical rotations were measured on a Perkin Elmer polarimeter equipped with a sodium (589 nm, D) lamp. Optical rotation data are represented as follows: specific rotation ([α]_{λ}^{T}), concentration (g/100 mL), and solvent. UV spectra were recorded on a Cary 3E UV/Vis spectrophotometer equipped with a thermoelectrically controlled 12-cell holder. High precision quartz SUPRASIL cells with a 1 cm path length were used for all absorbance studies.

### Synthetic Procedures.

### Synthesis of the β-Ketothioester 10:

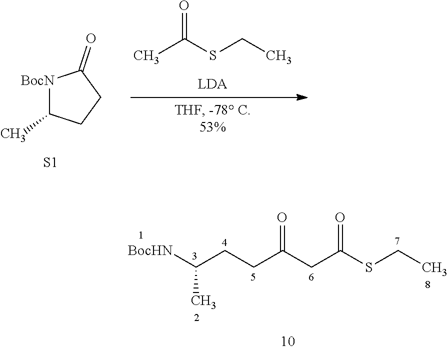

Ethyl thioacetate (2.08 mL, 19.5 mmol, 1.30 equiv) was added dropwise via syringe to a solution of lithium di-iso-propylamide (19.5 mmol, 1.30 equiv) in tetrahydrofuran (75 mL) at −78° C. The reaction mixture was stirred for 30 min at −78° C. A solution of the imide S1 (3.00 g, 15.1 mmol, 1 equiv) in tetrahydrofuran (28 mL) was added dropwise via cannula to the reaction mixture. The resulting mixture was stirred for 3 h at −78° C. The product mixture was diluted sequentially with saturated aqueous ammonium chloride solution (30 mL) and ethyl acetate (50 mL). The diluted product mixture was transferred to a separatory funnel and the layers that formed were separated. The aqueous layer was extracted with ethyl acetate (2×50 mL). The organic layers were combined and the combined organic layers were washed with saturated aqueous sodium chloride solution (30 mL). The washed organic layer was dried over sodium sulfate. The dried solution was filtered and the filtrate was concentrated. The residue obtained was purified by flash-column chromatography (eluting with 5% ethyl acetate-hexanes initially, grading to 20% ethyl acetate-hexanes, linear gradient) to provide the 3-ketothioester 10 as a light pink solid (2.40 g, 53%).

¹H NMR (500 MHz, CD₂Cl₂) δ 4.38 (bs, 1H), 3.66 (s, 2H, H₆), 3.58 (m, 1H, H₃), 2.90 (q, J=6.9 Hz, 2H, H₇), 2.65-2.50 (m, 2H, H₅), 1.77-1.67 (m, 1H, H₄), 1.62-1.52 (m, 1H, H₄), 1.41 (s, 9H, H₁), 1.25 (t, J=7.8 Hz, 3H, H₈), 1.10 (d, J=6.6 Hz, 3H, H₂). ¹³C NMR (126 MHz, CD₂Cl₂) δ 202.4 (C), 192.6 (C), 155.9 (C), 79.3 (C), 58.2 (CH₂), 46.4 (CH), 40.4 (CH₂), 31.2 (CH₂), 28.7 (CH₃), 24.5 (CH₂), 21.8 (CH₃), 14.9 (CH₃). IR (ATR-FTIR), cm⁻¹: 3387 (m), 2797 (w), 2929 (w), 1717 (w), 1683 (s), 1512 (s), 1310 (m), 1170 (m), 1051 (s), 541 (m). HRMS-CI (m/z): [M+Na]⁺ calcd for C₁₄H₂₅NNaO₄S, 326.1397; found, 326.1399. [α]_{D}²⁰ +8.0 (c 1.0, CH₂Cl₂).

### Synthesis of the Acid 13:

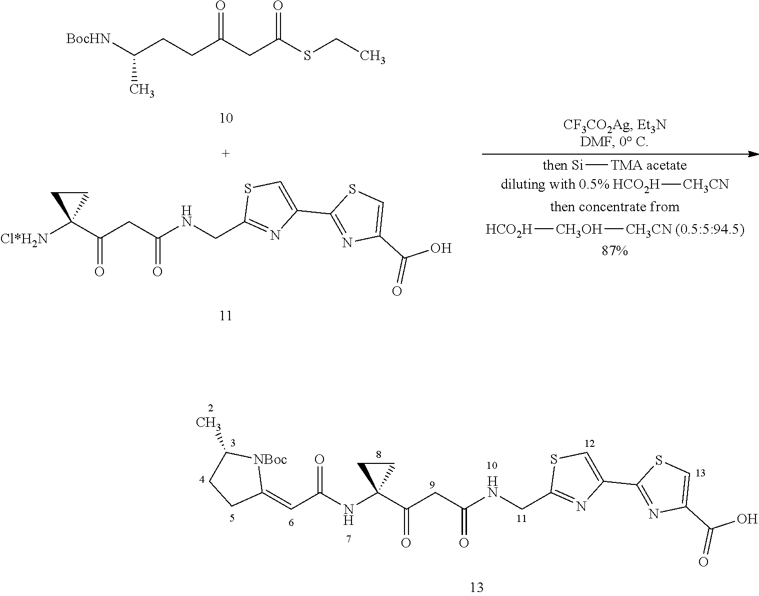

Silver trifluoroacetate (164 mg, 742 μmol, 2.00 equiv) was added to a solution of triethylamine (207 μL, 1.48 mmol, 4.00 equiv) and the amine 11 (149 mg, 371 μmol, 1 equiv) in N,N-dimethylformamide (2.7 mL) at 0° C. A solution of the β-ketothioester 10 (146 mg, 482 μmol, 1.30 equiv) in N,N-dimethylformamide (1.2 mL) was added dropwise via syringe to the reaction mixture. The reaction vessel was covered with foil to exclude light and the reaction mixture was stirred for 1 h at 0° C. The heterogeneous product mixture was filtered through a fritted funnel the filtrate was concentrated. The residue obtained was applied to a column containing trimethylamine acetate-functionalized silica gel (Si-TMA acetate; eluting with 0.5% formic acid-acetonitrile). The fractions containing product were collected, combined, and concentrated. The concentrated product was diluted with a solution containing 0.5% formic acid-5% methanol-acetonitrile (600 mL). The diluted product solution was concentrated. This process was repeated until LC/MS analysis indicated full conversion to the acid 13 (white solid, 185 mg, 87%).

¹H NMR (500 MHz, DMSO-d₆) δ 13.12 (bs, 1H), 8.94 (t, J=6.0 Hz, 1H, H₁₀), 8.47 (bs, 1H, H₇), 8.46 (s, 1H, H₁₃), 8.23 (s, 1H, H₁₂), 6.55 (s, 1H, H₆), 4.59 (d, J=5.9 Hz, 2H, H₁₁), 4.17 (app p, J=6.6 Hz, 1H, H₃), 3.55 (s, 2H, H₉), 3.41 (dd, J=18.2, 8.7 Hz, 1H, H₅), 2.82 (dt, J=18.6, 9.9 Hz, 1H, H₅), 1.89 (ddd, J=20.6, 12.2, 8.6 Hz, 1H, H₄), 1.55 (app t, J=10.4 Hz, 1H, H₄), 1.47 (s, 9H, H₁), 1.39-1.34 (m, 2H, H₈), 1.15 (d, J=6.3 Hz, 3H, H₂), 1.05-0.99 (m, 2H, H₈). ¹³C NMR (126 MHz, DMSO-d₆) δ 204.7 (C), 171.4 (C), 169.0 (C), 167.0 (C), 163.1 (C), 162.1 (C), 153.3 (C), 151.2 (C), 148.3 (C), 147.1 (C), 128.8 (CH), 118.2 (CH), 98.4 (CH), 80.9 (C), 56.0 (CH), 46.3 (CH₂), 40.6 (C), 40.5 (CH₂), 28.9 (CH₂), 27.8 (CH₂), 27.8 (CH₃), 19.5 (CH₂), 19.5 (CH₂), 19.3 (CH₃). IR (ATR-FTIR), cm⁻¹: 3329 (m), 2978 (w), 1722 (w), 1711 (w), 1673 (s), 1641 (w), 1586 (m), 1543 (m), 1518 (m), 1286 (s), 1230 (s), 1180 (s), 1157 (s), 1142 (s), 780 (m). HRMS-CI (m/z): [M+H]⁺ calcd for C₂₆H₃₂N₅O₇S₂, 590.1738; found, 590.1731. [α]_{D}²⁰ +13.0 (c 1.0, DMSO).

### Synthesis of the Amide 14a:

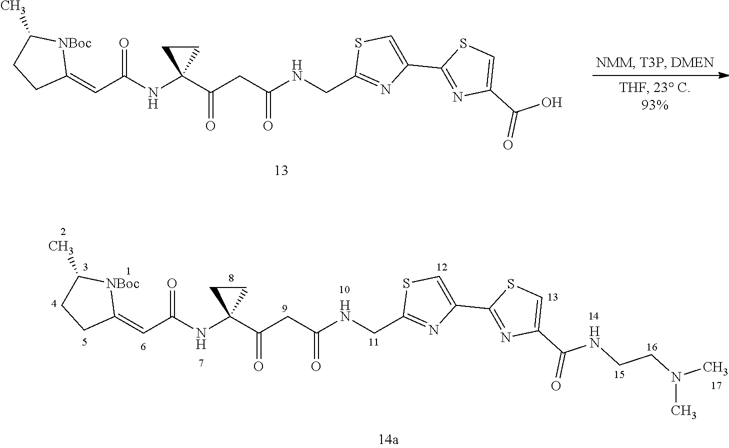

A solution of T3P in ethyl acetate (50 wt %, 10.6 μL, 17.8 μmol, 1.50 equiv) and 4-methylmorpholine (6.5 μL, 59.4 μmol, 5.00 equiv) were added in sequence to a solution of the acid 13 (7.0 mg, 11.9 μmol, 1 equiv) in tetrahydrofuran (240 μL) at 23° C. The reaction mixture was stirred for 20 min at 23° C. A solution of N,N-dimethylethylenediamine (3.2 μL, 29.7 μmol, 2.50 equiv) in tetrahydrofuran (50 μL) was added to the reaction mixture. The resulting mixture was stirred for 7 h at 23° C. The product mixture was concentrated. The concentrated product mixture was diluted with ethyl acetate (10 mL). The diluted product mixture was poured into a separatory funnel that had been charged with saturated aqueous sodium bicarbonate solution (5.0 mL) and the layers that formed were separated. The aqueous layer was extracted with ethyl acetate (2×10 mL). The organic layers were combined and the combined organic layers were dried over sodium sulfate. The dried solution was filtered and the filtrate was concentrated to provide the amide 14a as a white solid (7.3 mg, 93%). The product so obtained was used without further purification.

¹H NMR (400 MHz, DMSO-d₆) δ 8.96 (t, J=5.7 Hz, 1H, H₁₀), 8.48 (bs, 1H, H₇), 8.26 (s, 1H, H₁₃), 8.24 (bs, 1H, H₁₄), 8.19 (s, 1H, H₁₂), 6.55 (s, 1H, H₆), 4.59 (d, J=5.4 Hz, 2H, H₁₁), 4.17 (app p, J=5.7 Hz, 1H, H₃), 3.55 (s, 2H, H₉), 3.47-3.35 (m, 3H, H₅, H₁₅), 2.82 (dt, J=19.0, 9.9 Hz, 1H, H₅), 2.41 (t, J=6.2 Hz, 2H, H₁₆), 2.18 (s, 6H, H₁₇), 1.97-1.81 (m, 1H, H₄), 1.55 (app t, J=10.4 Hz, 1H, H₄), 1.47 (s, 9H, H₁), 1.39-1.33 (m, 2H, H₈), 1.15 (d, J=5.8 Hz, 3H, H₂), 1.06-0.96 (m, 2H, H₈). ¹³C NMR (101 MHz, DMSO-d₆) δ 204.7 (C), 171.6 (C), 169.0 (C), 167.1 (C), 161.9 (C), 160.2 (C), 153.3 (C), 151.2 (C), 150.8 (C), 147.2 (C), 124.1 (CH), 118.2 (CH), 98.4 (CH), 80.9 (C), 58.1 (CH₂), 56.0 (CH), 46.4 (CH₂), 45.2 (CH₃), 40.7 (C), 40.1 (CH₂), 36.7 (CH₂), 28.9 (CH₂), 27.8 (CH₂), 27.8 (CH₃), 19.5 (CH₂), 19.5 (CH₂), 19.3 (CH₃). IR (ATR-FTIR), cm⁻¹: 3278 (br w), 2975 (w), 2931 (w), 1703 (m), 1648 (s), 1603 (m), 1549 (m), 1291 (m), 1158 (s). HRMS-CI (m/z): [M+H]⁺ calcd for C₃₀H₄₂NO₆S₂, 660.2633; found, 660.2631. [α]_{D}²⁰+25.0 (c 1.0, CH₃OH).

### Synthesis of the Lactam 15a:

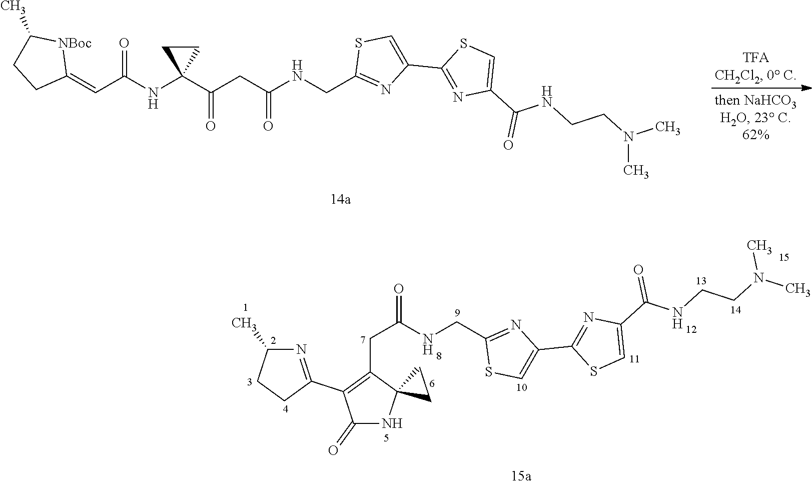

Trifluoroacetic acid (751 μL, 9.82 mmol, 120 equiv) was added dropwise via syringe to a solution of the amide 14a (54.0 mg, 81.8 μmol, 1 equiv) in dichloromethane (1.6 mL) at 0° C. The reaction mixture was stirred for 14 h at 0° C. The reaction mixture was concentrated and the concentrated reaction mixture was diluted with saturated aqueous sodium bicarbonate solution (4.0 mL). The diluted reaction mixture was stirred for 1 h at 23° C. The product mixture was diluted sequentially with water (10 mL) and ethyl acetate (30 mL). The diluted product mixture was transferred to a separatory funnel and the layers that formed were separated. The aqueous layer was extracted with ethyl acetate (5×30 mL). The organic layers were combined and the combined organic layers were dried over sodium sulfate. The dried solution was filtered and the filtrate was concentrated to provide the lactam 15a as a light yellow solid (27.5 mg, 62%). The product so obtained was used without further purification.

¹H NMR (500 MHz, DMSO-d₆) δ 10.28 (t, J=6.0 Hz, 1H, H₈), 8.60 (bs, 1H, H₅), 8.28-8.23 (m, 2H, H₁₁, H₁₂), 8.19 (s, 1H, H₁₀), 4.66-4.57 (m, 2H, H₉), 4.18-4.07 (m, 1H, H₂), 3.39 (app q, J=6.5 Hz, 2H, H₁₃), 3.35-3.30 (m, 2H, H₇), 3.15-3.06 (m, 1H, H₄), 2.87 (dt, J=17.8, 8.8 Hz, 1H, H₄), 2.45 (t, J=6.7 Hz, 2H, H₁₄), 2.21 (s, 6H, H₁₅), 2.12-2.06 (m, 1H, H₃), 1.76-1.61 (m, 2H, H₆), 1.42-1.33 (m, 3H, H₃, H₆), 1.19 (d, J=6.6 Hz, 3H, H₁). ¹³C NMR (126 MHz, DMSO-d₆) δ 171.1 (C), 169.6 (C), 168.3 (C), 168.2 (C), 161.8 (C), 160.2 (C), 157.6 (C), 150.8 (C), 147.4 (C), 127.4 (C), 124.2 (CH), 118.0 (CH), 66.5 (CH), 58.0 (CH₂), 45.3 (C), 45.1 (CH₃), 40.4 (CH₂), 36.6 (CH₂), 36.5 (CH₂), 33.5 (CH₂), 29.7 (CH₂), 21.9 (CH₃), 11.8 (CH₂), 11.7 (CH₂). HRMS-CI (m/z): [M+H]⁺ calcd for C₂₅H₃₂N₇O₃S₂, 542.2003; found, 542.2016. [α]_{D}²⁰ −6.0 (c 1.5, DMSO-d₆).

### Synthesis of the Pyridone 16:

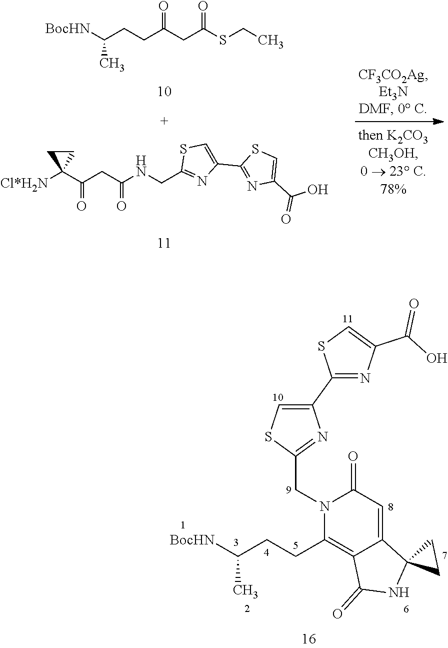

Silver trifluoroacetate (410 mg, 1.86 mmol, 2.00 equiv) was added to a solution of triethylamine (518 μL, 3.71 mmol, 4.00 equiv) and the amine 11 (374 mg, 930 μmol, 1 equiv) in N,N-dimethylformamide (6.0 mL) at 0° C. A solution of the β-ketothioester 10 (366 mg, 1.21 mmol, 1.30 equiv) in N,N-dimethylformamide (2.0 mL) was added dropwise via syringe to the reaction mixture. The reaction vessel was covered with foil to exclude light and the reaction mixture was stirred for 1 h at 0° C. Potassium carbonate (385 mg, 2.79 mmol, 3.00 equiv) and methanol (8.0 mL) were then added in sequence to the reaction mixture at 0° C. The reaction mixture was stirred for 30 min at 0° C. The heterogeneous product mixture was filtered through a fritted funnel. The filter cake was washed with methanol (10 mL). The filtrates were combined and the combined filtrates were concentrated. The residue obtained was applied to a trimethylamine acetate-functionalized silica column (Si-TMA acetate; eluting with 0.5% formic acid-acetonitrile). The fractions containing the product 16 were collected, combined, and concentrated to provide the pyridone 16 as a white solid (414 mg, 78%).

¹H NMR (600 MHz, DMSO-d₆) δ 8.48 (bs, 1H, H₆), 8.39 (s, 1H, H₁₁), 8.25 (s, 1H, H₁₀), 6.80 (d, J=8.0 Hz, 1H), 6.17 (s, 1H, H₈), 5.60 (d, J=16.0 Hz, 1H, H₉), 5.49 (d, J=15.9 Hz, 1H, H₉), 3.63-3.52 (m, 1H, H₃), 3.53-3.45 (m, 1H, H₅), 3.32-3.14 (m, 1H, H₅), 1.75-1.60 (m, 2H, H₄), 1.37 (app t, J=2.7 Hz, 2H, H₇), 1.35 (app t, J=2.8 Hz, 1H, H₇), 1.30 (s, 9H, H₁), 1.06 (d, J=6.6 Hz, 3H, H₂). ¹³C NMR (151 MHz, DMSO-d₆) δ 166.9 (C), 166.7 (C), 162.3 (C), 161.8 (C), 161.6 (C), 159.9 (C), 155.1 (C), 153.0 (C), 149.3 (C), 147.2 (C), 128.3 (CH), 118.7 (CH), 109.7 (C), 103.3 (CH), 77.4 (C), 46.1 (CH), 44.2 (CH₂), 39.7 (C), 35.5 (CH₂), 28.2 (CH₃), 24.1 (CH₂), 20.7 (CH₃), 15.2 (CH₂). IR (ATR-FTIR), cm⁻¹: 3327 (w), 3121 (w), 2971 (w), 2355 (br w), 1720 (w), 1702 (w), 1674 (m), 1649 (s), 1571 (m), 1518 (m), 1171 (m), 578 (s). HRMS-CI (m/z): [M+H]⁺ calcd for C₂₆H₃₀N₅O₆S₂, 572.1632; found, 572.1630. [α]_{D}²⁰ −64.0 (c 0.5, DMSO).

### Synthesis of the Amide S2:

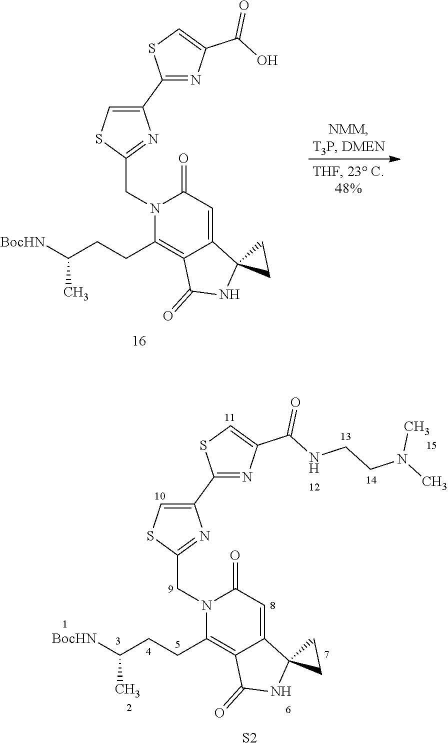

A solution of T3P in ethyl acetate (50 wt %, 54.7 μL, 91.8 μmol, 1.50 equiv) and 4-methylmorpholine (33.7 μL, 306 μmol, 5.00 equiv) were added in sequence to a solution of the acid 16 (35.0 mg, 61.2 μmol, 1 equiv) in tetrahydrofuran (790 μL) at 23° C. N,N-Dimethylethylenediamine (16.7 μL, 153 μmol, 2.50 equiv) was then added to the reaction mixture. The resulting mixture was stirred for 7 h at 23° C. The product mixture was concentrated. The concentrated product mixture was diluted with ethyl acetate (10 mL). The diluted product mixture was poured into a separatory funnel that had been charged with saturated aqueous sodium bicarbonate solution (5.0 mL) and the layers that formed were separated. The aqueous layer was extracted with ethyl acetate (2×10 mL). The organic layers were combined and the combined organic layers were dried over sodium sulfate. The dried solution was filtered and the filtrate was concentrated to provide the amide S2 as a white solid (18.1 mg, 46%). The product so obtained was used without further purification.

¹H NMR (500 MHz, CD₂Cl₂) δ 8.06 (s, 1H, H₁₁), 8.01 (s, 1H, H₁₀), 7.64 (bs, 1H, H₁₂), 6.33 (bs, 1H, H₆), 6.01 (s, 1H, H₈), 5.62 (d, J=15.0 Hz, 1H, H₉), 5.55 (d, J=14.2 Hz, 1H, H₉), 5.38 (d, J=7.4 Hz, 1H), 3.82-3.74 (m, 1H, H₃), 3.66-3.58 (m, 1H, H₅), 3.51 (app q, J=5.9 Hz, 2H, H₁₃), 3.48-3.41 (m, 1H, H₅), 2.51 (t, J=6.1 Hz, 2H, H₁₄), 2.27 (s, 6H, H₁₅), 1.89-1.81 (m, 1H, H₄), 1.80-1.69 (m, 1H, H₄), 1.52-1.45 (m, 2H, H₇), 1.41 (s, 9H, H₁), 1.37-1.32 (m, 2H, H₇), 1.19 (d, J=6.5 Hz, 3H, H₂). ¹³C NMR (126 MHz, CD₂Cl₂) δ 168.4 (C), 166.0 (C), 163.1 (C), 162.7 (C), 161.2 (C), 160.5 (C), 156.1 (C), 154.4 (C), 151.8 (C), 148.5 (C), 123.8 (CH), 119.2 (CH), 110.3 (C), 103.9 (CH), 79.2 (C), 58.7 (CH₂), 47.1 (CH), 45.7 (CH₃), 45.1 (CH₂), 40.6 (C), 37.5 (CH₂), 36.1 (CH₂), 28.7 (CH₃), 25.0 (CH₂), 21.3 (CH₃), 16.2 (CH₂), IR (ATR-FTIR), cm⁻¹: 3327 (br w), 2972 (w), 1694 (w), 1651 (s), 1541 (m), 1250 (m), 1165 (m), 568 (m). HRMS-CI (m/z): [M+H]⁺ calcd for C₃₀H₄₀N₇O₅S₂, 642.2527; found, 642.2532. [α]_{D}²⁰ −125.8 (c 0.93, CH₃OH).

### Synthesis of the Amide 17a:

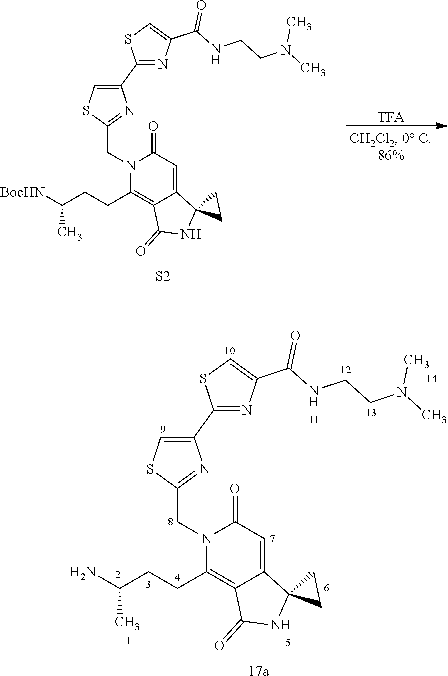

Trifluoroacetic acid (206 μL, 2.69 mmol, 120 equiv) was added dropwise via syringe to a solution of the amide S2 (14.4 mg, 22.4 μmol, 1 equiv) in dichloromethane (560 μL) at 0° C. The reaction mixture was stirred for 14 h at 0° C. The reaction mixture was concentrated. The concentrated product mixture was diluted with chloroform (10 mL). The diluted product mixture was poured into a separatory funnel that had been charged with saturated aqueous sodium bicarbonate solution (5.0 mL) and the layers that formed were separated. The aqueous layer was extracted with chloroform (2×10 mL). The organic layers were combined and the combined organic layers were dried over sodium sulfate. The dried solution was filtered and the filtrate was concentrated to provide the amide 17a as a white solid (10.5 mg, 86%). The product so obtained was used without further purification.

¹H NMR (400 MHz, CD₃OD) δ 8.24 (s, 1H, H₁₀), 8.18 (s, 1H, H₉), 6.19 (s, 1H, H₇), 5.73 (d, J=15.4 Hz, 1H, H₈), 5.68 (d, J=15.5 Hz, 1H, H₈), 3.75-3.62 (m, 1H, H₄), 3.60-3.50 (m, 3H, H₄, H₁₂), 3.10-3.02 (m, 1H, H₂), 2.59 (t, J=6.7 Hz, 2H, H₁₃), 2.32 (s, 6H, H₁₄), 1.87-1.72 (m, 2H, H₃), 1.58-1.50 (m, 2H, H₆), 1.47-1.40 (m, 2H, H₆), 1.18 (d, J=6.3 Hz, 3H, H₁). ¹³C NMR (151 MHz, CD₃OD) δ 169.5 (C), 167.5 (C), 165.0 (C), 163.7 (C), 163.4 (C), 162.5 (C), 154.9 (C), 151.7 (C), 149.1 (C), 125.2 (CH), 120.2 (CH), 112.3 (C), 104.4 (CH), 59.3 (CH₂), 47.7 (CH), 46.1 (CH₂), 45.5 (CH₃), 41.5 (C), 39.0 (CH₂), 38.0 (CH₂), 25.5 (CH₂), 22.5 (CH₃), 16.3 (CH₂). IR (ATR-FTIR), cm⁻¹: 3355 (br w), 2956 (w), 1691 (w), 1648 (s), 1572 (w), 1545 (w), 1288 (m), 568 (m). HRMS-CI (m/z): [M+H]⁺ calcd for C₂₅H₃₂N₇O₃S₂, 542.2003; found, 542.2004. [α]_{D}²⁰ −13.0 (c 1.0, CH₃OH).

### Synthesis of the Amide 14b:

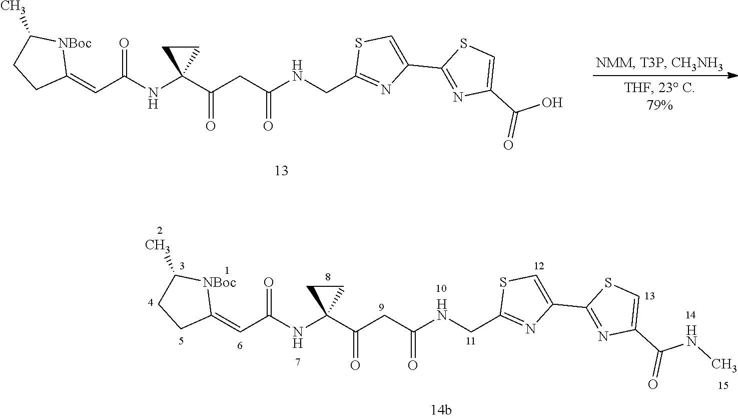

A solution of T3P in ethyl acetate (50 wt %, 22.7 μL, 38.2 μmol, 1.50 equiv) and 4-methylmorpholine (14.0 μL, 127 μmol, 5.00 equiv) were added in sequence to a solution of the acid 13 (15.0 mg, 25.4 μmol, 1 equiv) in tetrahydrofuran (330 μL) at 23° C. The reaction mixture was stirred for 20 min at 23° C. A solution of methylamine in tetrahydrofuran (2.00 M, 23 μL, 63.6 μmol, 2.50 equiv) was then added to the reaction mixture. The resulting mixture was stirred for 7 h at 23° C. The product mixture was concentrated. The residue obtained was purified by flash-column chromatography (eluting with dichloromethane initially, grading to 10% methanol-dichloromethane, linear gradient) to provide the amide 14b as an off-white solid (12.1 mg, 79%).

¹H NMR (500 MHz, CD₂Cl₂) δ 8.05 (s, 1H, H₁₃), 8.04 (bs, 1H, H₁₀), 7.93 (s, 1H, H₁₂), 7.38 (bs, 1H, H₁₄), 6.55 (s, 1H, H₆), 6.23 (bs, 1H, H₇), 4.76 (d, J=6.0 Hz, 2H, H₁₁), 4.23 (app p, J=6.8 Hz, 1H, H₃), 3.66 (s, 2H, H₉), 3.47 (dd, J=18.3, 8.7 Hz, 1H, H₅), 2.98 (d, J=5.1 Hz, 3H, H₁₃), 2.89 (dddd, J=18.3, 11.1, 8.3, 2.2 Hz, 1H, H₅), 1.92 (tt, J=12.1, 8.5 Hz, 1H, H₄), 1.62 (app q, J=4.4 Hz, 2H, H₈), 1.56 (dd, J=12.2, 8.6 Hz, 1H, H₄), 1.18 (d, J=6.5 Hz, 3H, H₂), 1.17-1.13 (m, 2H, H₈). ¹³C NMR (126 MHz, CD₂Cl₂) δ 206.1 (C), 170.6 (C), 170.4 (C), 167.2 (C), 163.0 (C), 161.9 (C), 156.9 (C), 152.4 (C), 151.6 (C), 148.9 (C), 123.5 (CH), 117.6 (CH), 97.2 (CH), 82.2 (C), 57.5 (CH), 45.6 (CH₂), 42.0 (C), 41.7 (CH₂), 30.2 (CH₂), 28.9 (CH₂), 28.5 (CH₃), 26.3 (CH₃), 21.6 (CH₂), 21.6 (CH₂), 19.9 (CH₃). IR (ATR-FTIR), cm⁻¹: 3295 (br w), 2975 (w), 2931 (w), 1706 (m), 1651 (s), 1606 (m), 1547 (m), 1289 (m), 1156 (s), 771 (m). HRMS-CI (m/z): [M+H]⁺ calcd for C₂₇H₃₅N₆O₆S₂, 603.2054; found, 603.2053. [α]_{D}²⁰ +36.1 (c 0.83, CH₂Cl₂).

### Synthesis of the Lactam 15b:

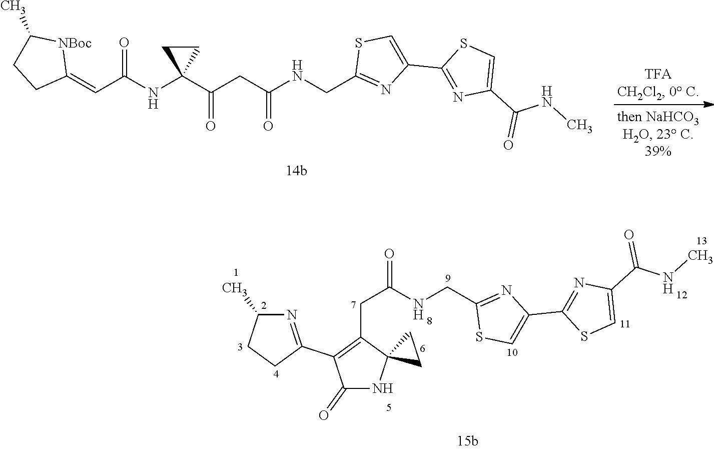

Trifluoroacetic acid (216 μL, 2.83 mmol, 120 equiv) was added dropwise via syringe to a solution of the amide 14b (14.2 mg, 23.6 μmol, 1 equiv) in dichloromethane (590 μL) at 0° C. The reaction mixture was stirred for 14 h at 0° C. The reaction mixture was concentrated. The concentrated reaction mixture was diluted with saturated aqueous sodium bicarbonate solution (400 μL). The diluted reaction mixture was stirred for 1 h at 23° C. The product mixture was diluted sequentially with water (1.0 mL) and ethyl acetate (3.0 mL). The diluted product mixture was transferred to a separatory funnel and the layers that formed were separated. The aqueous layer was extracted with ethyl acetate (5×3.0 mL). The organic layers were combined and the combined organic layers were dried over sodium sulfate. The dried solution was filtered and the filtrate was concentrated to provide the lactam 15b as a light yellow solid (4.5 mg, 39%). The product so obtained was used without further purification.

¹H NMR (500 MHz, DMSO-d₆) δ 10.28 (t, J=6.0 Hz, 1H, H₈), 8.60 (bs, 1H, H₅), 8.41-8.34 (m, 1H, H₁₂), 8.25 (s, 1H, H₁₁), 8.17 (s, 1H, H₁₀), 4.65-4.57 (m, 2H, H₉), 4.17-4.07 (m, 1H, H₂), 3.38-3.26 (m, 2H, H₇), 3.15-3.05 (m, 1H, H₄), 2.91-2.85 (m, 1H, H₄), 2.81 (d, J=4.8 Hz, 3H, H₁₃), 2.14-2.03 (m, 1H, H₃), 1.73-1.61 (m, 2H, H₆), 1.42-1.29 (m, 3H, H₃, H₆), 1.19 (d, J=6.7 Hz, 3H, H₁). ¹³C NMR (126 MHz, DMSO-d₆) δ 171.1 (C), 169.6 (C), 168.4 (C), 168.2 (C), 161.7 (C), 160.9 (C), 157.7 (C), 150.9 (C), 147.5 (C), 127.4 (C), 123.8 (CH), 117.8 (CH), 66.5 (CH), 45.3 (C), 40.4 (CH₂), 36.5 (CH₂), 33.5 (CH₂), 29.7 (CH₂), 25.8 (CH₃), 21.9 (CH₃), 11.8 (CH₂), 11.8 (CH₂). HRMS-CI (m/z): [M+H]⁺ calcd for C₂₂H₂₅N₆O₃S₂, 485.1424; found, 485.1418. [α]_{D}²⁰ −2.3 (c 1.3, DMSO-d₆).

### Synthesis of the Amide S3:

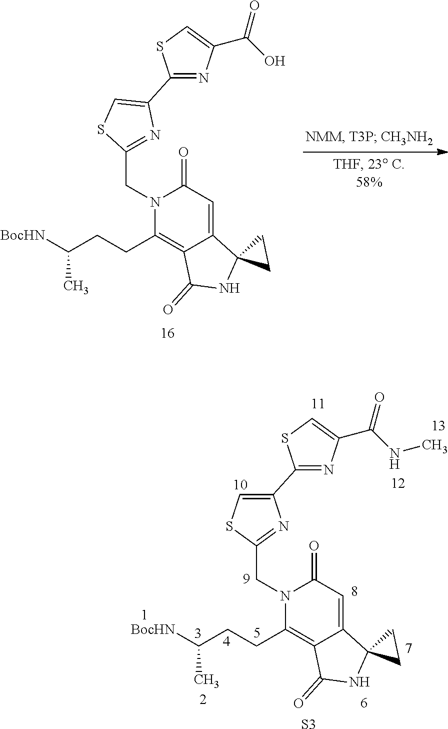

A solution of T3P in ethyl acetate (50 wt %, 54.7 μL, 91.8 μmol, 1.50 equiv) and 4-methylmorpholine (33.7 μL, 306 μmol, 5.00 equiv) were added in sequence to a solution of the acid 16 (35.0 mg, 61.2 μmol, 1 equiv) in tetrahydrofuran (790 μL) at 23° C. A solution of methylamine in tetrahydrofuran (2.00 M, 77 μL, 153 μmol, 2.50 equiv) was then added to the reaction mixture. The resulting mixture was stirred for 7 h at 23° C. The product mixture was concentrated. The concentrated product mixture was diluted with ethyl acetate (10 mL). The diluted product mixture was poured into a separatory funnel that had been charged with saturated aqueous sodium bicarbonate solution (5.0 mL) and the layers that formed were separated. The aqueous layer was extracted with ethyl acetate (2×10 mL). The organic layers were combined and the combined organic layers were dried over sodium sulfate. The dried solution was filtered and the filtrate was concentrated to provide the amide S3 as a white solid (20.8 mg, 58%). The product so obtained was used without further purification.

¹H NMR (600 MHz, CD₃OD) δ 8.22 (s, 1H, H₁₁), 8.16 (s, 1H, H₁₀), 6.16 (s, 1H, H₈), 5.71 (d, J=15.8 Hz, 1H, H₉), 5.65 (d, J=15.8 Hz, 1H, H₉), 3.74 (app h, J=6.4 Hz, 1H, H₃), 3.60-3.50 (m, 2H, H₅), 2.96 (s, 3H, H₁₃), 1.89-1.75 (m, 2H, H₄), 1.54-1.51 (m, 2H, H₇), 1.43-1.40 (m, 2H, H₇), 1.37 (s, 9H, H₁), 1.17 (d, J=6.7 Hz, 3H, H₂). ¹³C NMR (151 MHz, CD₃OD) δ 169.4 (C), 167.4 (C), 164.9 (C), 164.0 (C), 163.7 (C), 162.5 (C), 157.9 (C), 155.1 (C), 151.8 (C), 149.3 (C), 124.9 (CH), 119.8 (CH), 112.2 (C), 104.3 (CH), 79.9 (C), 47.8 (CH), 45.9 (CH₂), 41.5 (C), 36.8 (CH₂), 28.9 (CH₃), 26.3 (CH₃), 25.9 (CH₂), 21.2 (CH₃), 16.3 (CH₂). IR (ATR-FTIR), cm⁻¹: 3284 (br w), 2971 (w), 1694 (m), 1652 (s), 1573 (m), 1550 (m), 1167 (m), 570 (s). HRMS-CI (m/z): [M+H]⁺ calcd for C₂₇H₃₃N₆O₅S₂, 585.1948; found, 585.1948. [α]_{D}²⁰ −101.2 (c 0.85, CH₃OH).

### Synthesis of the Amide 17b:

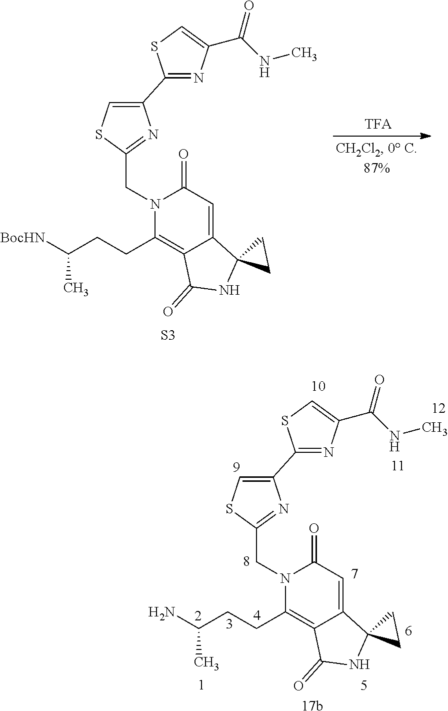

Trifluoroacetic acid (273 μL, 3.57 mmol, 120 equiv) was added dropwise via syringe to a solution of the amide S3 (17.4 mg, 29.8 μmol, 1 equiv) in dichloromethane (740 μL) at 0° C. The reaction mixture was stirred for 14 h at 0° C. The reaction mixture was concentrated. The concentrated product mixture was diluted with chloroform (10 mL). The diluted product mixture was poured into a separatory funnel that had been charged with saturated aqueous sodium bicarbonate solution (5.0 mL) and the layers that formed were separated. The aqueous layer was extracted with chloroform (2×10 mL). The organic layers were combined and the combined organic layers were dried over sodium sulfate. The dried solution was filtered and the filtrate was concentrated to provide the amide 17b as a white solid (12.6 mg, 87%). The product so obtained was used without further purification.

¹H NMR (600 MHz, CD₃OD) δ 8.24 (s, 1H, H₉), 8.16 (s, 1H, H₁₀), 6.21 (s, 1H, H₇), 5.72 (d, J=15.7 Hz, 1H, H₈), 5.67 (d, J=15.7 Hz, 1H, H₈), 3.74-3.65 (m, 1H, H₄), 3.62-3.51 (m, 1H, H₄), 3.31-3.26 (m, 1H, H₂), 2.96 (s, 3H, H₁₂), 2.04-1.86 (m, 2H, H₃), 1.58-1.50 (m, 2H, H₆), 1.48-1.41 (m, 2H, H₆), 1.30 (d, J=6.6 Hz, 3H, H₁). ¹³C NMR (151 MHz, CD₃OD) δ 169.6 (C), 167.3 (C), 164.8 (C), 164.0 (C), 163.7 (C), 162.3 (C), 153.7 (C), 151.8 (C), 149.1 (C), 124.8 (CH), 120.3 (CH), 112.5 (C), 104.8 (CH), 48.1 (CH), 46.0 (CH₂), 41.6 (C), 36.4 (CH₂), 26.4 (CH₃), 25.0 (CH₂), 20.3 (CH₃), 16.4 (CH₂). IR (ATR-FTIR), cm⁻¹: 3419 (br w), 2926 (w), 2857 (w), 1688 (w), 1647 (s), 1556 (m), 1289 (w), 568 (m). HRMS-CI (m/z): [M+H]⁺ calcd for C₂₂H₂₅N₆O₃S₂, 485.1424; found, 485.1425. [α]_{D}²⁰ −29.0 (c 1.0, CH₃OH).

### Synthesis of the Dimeric Amide 14c:

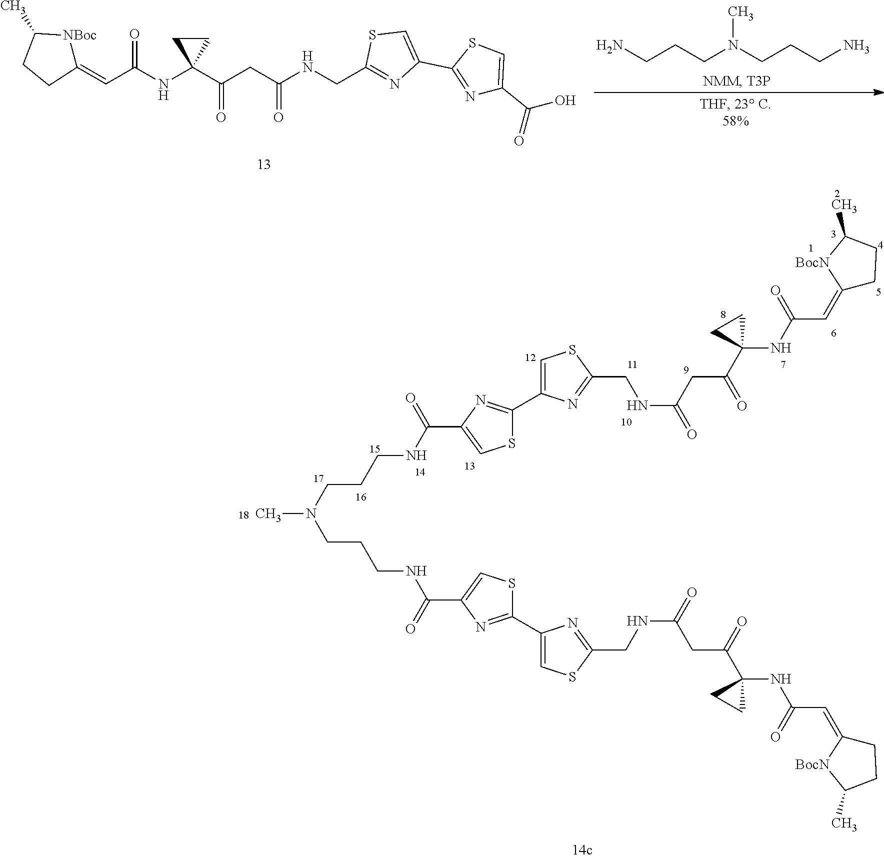

A solution of T3P it ethyl acetate (50 wt %, 50.5 μL, 84.8 μmol, 2.50 equiv) an 4-methylmorpholine (37.3 μL, 339 μmol, 10.00 equiv) were added in sequence to a solution of the acid 13 (20.0 mg, 33.9 μmol, 1 equiv) in tetrahydrofuran (680) at 23° C. The reaction mixture was stirred for 20 min at 23° C. A solution of N,N-bis(3-aminopropyl)methylamine (2.7 μL, 17.0 μmol, 0.50 equiv) in tetrahydrofuran (50 μL) was then added to the reaction mixture. The resulting mixture was stirred for 7 h at 23° C. The product mixture was concentrated. The concentrated product mixture was diluted with ethyl acetate (10 mL). The diluted product mixture was poured into a separatory funnel that had been charged with saturated aqueous sodium bicarbonate solution (5.0 mL) and the layers that formed were separated. The aqueous layer was extracted with ethyl acetate (2×10 mL). The organic layers were combined and the combined organic layers were dried over sodium sulfate. The dried solution was filtered and the filtrate was concentrated to provide the dimeric amide 14c as a white solid (12.5 mg, 58%). The product so obtained was used without further purification.

¹H NMR (500 MHz, CD₂Cl₂) δ 8.21-8.14 (m, 2H, H₁₀), 8.07-8.01 (m, 2H, H₁₄), 8.01 (s, 2H, H₁₃), 7.80 (s, 2H, H₁₂), 6.76 (bs, 2H, H₇), 6.57 (s, 2H, H₆), 4.68 (d, J=5.9 Hz, 4H, H₁₁), 4.28-4.16 (m, 2H, H₃), 3.68 (s, 4H, H₉), 3.55-3.49 (m, 4H, H₁₅), 3.50-3.42 (m, 2H, H₅), 2.95-2.77 (m, 2H, H₅), 2.51 (t, J=6.6 Hz, 4H, H₁₇), 2.28 (s, 3H, H₁₈), 1.98-1.86 (m, 2H, H₄), 1.87-1.80 (m, 4H, H₁₆), 1.64-1.58 (m, 4H, H₈), 1.60-1.52 (m, 2H, H₄), 1.49 (s, 18H, H₁), 1.17 (d, J=6.5 Hz, 6H, H₂), 1.14-1.01 (m, 4H, H₈). ¹³C NMR (126 MHz, CD₂Cl₂) δ 206.2 (C), 170.6 (C), 170.2 (C), 167.4 (C), 162.8 (C), 161.3 (C), 156.6 (C), 152.4 (C), 151.8 (C), 148.7 (C), 123.6 (CH), 117.6 (CH), 97.5 (CH), 82.1 (C), 57.4 (CH), 56.7 (CH₂), 46.0 (CH₂), 42.5 (CH₃), 41.9 (C), 41.6 (CH₂), 38.9 (CH₂), 30.1 (CH₂), 28.9 (CH₂), 28.5 (CH₃), 27.5 (CH₂), 21.6 (CH₂), 21.6 (CH₂), 19.8 (CH₃). IR (ATR-FTIR), cm⁻¹: 3282 (br w), 2974 (w), 2933 (w), 1708 (m), 1651 (s), 1608 (m), 1541 (s), 1315 (m), 1290 (s), 1156 (s), 1026 (m), 770 (m), 755 (m), 620 (w). HRMS-CI (m/z): [M+H]⁺ calcd for C₅₉H₇₈N₁₃O₁₂S₄, 1288.4770; found, 1288.4768. [α]_{D}²⁰ +19.0 (c 1.0, CH₂Cl₂).

### Synthesis of the Dimeric Lactam 15c:

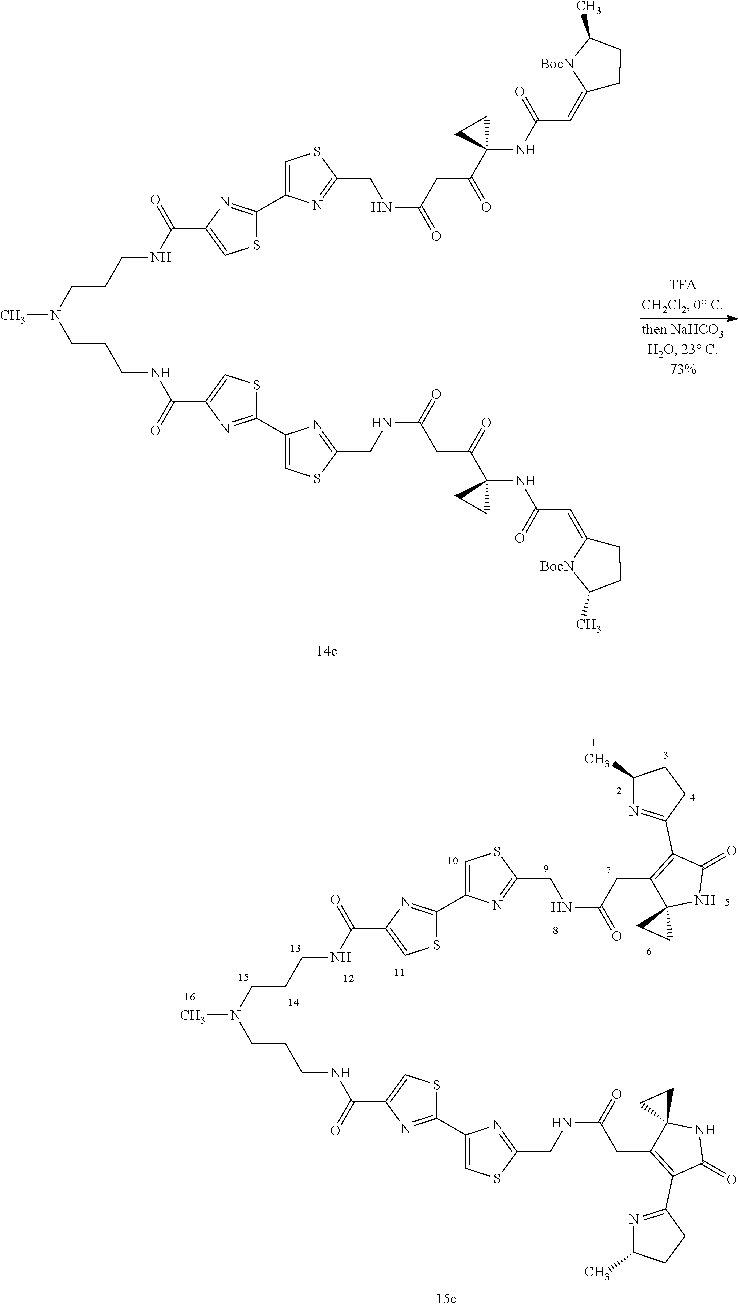

Trifluoroacetic acid (57.0 μL, 745 μmol, 120 equiv) was added dropwise via syringe to a solution of the amide 14c (8.0 mg, 6.21 μmol, 1 equiv) in dichloromethane (200 μL) at 0° C. The reaction mixture was stirred for 14 h at 0° C. The reaction mixture was concentrated. The concentrated reaction mixture was diluted with saturated aqueous sodium bicarbonate solution (650 μL). The diluted reaction mixture was stirred for 1 h at 23° C. The product mixture was diluted sequentially with water (1.0 mL) and ethyl acetate (3.0 mL). The diluted product mixture was transferred to a separatory funnel and the layers that formed were separated. The aqueous layer was extracted with ethyl acetate (5×3.0 mL). The organic layers were combined and the combined organic layers were dried over sodium sulfate. The dried solution was filtered and the filtrate was concentrated to provide the dimeric lactam 15c as a light yellow solid (4.8 mg, 73%). The product so obtained was used without further purification.

¹H NMR (500 MHz, DMSO-d₆) δ 10.28 (t, J=5.9 Hz, 2H, H₈), 8.61 (bs, 2H, H₅), 8.55 (t, J=5.9 Hz, 2H, H₁₂), 8.22 (s, 2H, H₁₁), 8.15 (s, 2H, H₁₀), 4.60 (d, J=6.0 Hz, 4H, H₉), 4.16-4.07 (m, 2H, H₂), 3.38-3.29 (m, 8H, H₇, H₁₃), 3.16-3.05 (m, 2H, H₄), 2.86 (dt, J=18.1, 9.1 Hz, 2H, H₄), 2.39 (t, J=6.8 Hz, 4H, H₁₅), 2.19 (s, 3H, H₁₆), 2.10-2.04 (m, 2H, H₃), 1.77-1.65 (m, 8H, H₆, H₁₄), 1.38-1.34 (m, 6H, H₃, H₆), 1.17 (d, J=7.0 Hz, 6H, H₁). ¹³C NMR (126 MHz, DMSO-d₆) δ 171.0 (C), 169.6 (C), 168.4 (C), 168.2 (C), 161.7 (C), 160.2 (C), 157.7 (C), 151.0 (C), 147.4 (C), 127.4 (C), 123.9 (CH), 117.8 (CH), 66.5 (CH), 55.4 (CH₂), 45.3 (C), 41.8 (CH₃), 40.4 (CH₂), 37.7 (CH₂), 36.5 (CH₂), 33.5 (CH₂), 29.7 (CH₂), 26.7 (CH₂), 21.9 (CH₃), 11.8 (CH₂), 11.8 (CH₂). HRMS-CI (m/z): [M+H]⁺ calcd for C₄₉H₅₈N₁₃O₆S₄, 1052.3510; found, 1052.3514. [α]_{D}²⁰ +1.3 (c 1.5, DMSO-d₆).

### Synthesis of the β-Ketothioester S6:

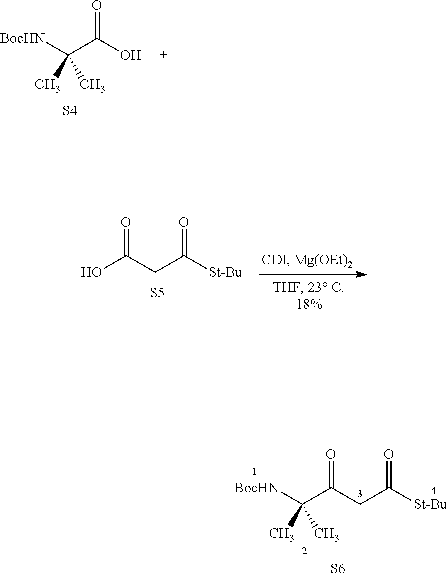

1,1′-Carbonyldiimidazole (1.20 g, 7.38 mmol, 1.50 equiv) was added to a solution of 2-((tert-butoxycarbonyl)amino)-2-methylpropanoic acid (S4, 1.00 g, 4.92 mmol, 1 equiv) in tetrahydrofuran (25 mL) at 23° C. The resulting mixture was stirred for 6 h at 23° C. In a second round-bottomed flask, magnesium ethoxide (845 mg, 7.38 mmol, 1.50 equiv) was added to a solution of 3-(tert-butylthio)-3-oxopropanoic acid (S5, 2.60 g, 14.8 mmol, 3.00 equiv) in tetrahydrofuran (13 mL) at 23° C. The resulting mixture was stirred for 6 h at 23° C., and then was concentrated to dryness. The activated carboxylic acid prepared in the first flask was transferred via cannula to the dried magnesium salt prepared in the second flask. The resulting mixture was stirred for 14 h at 23° C. The product mixture was diluted sequentially with saturated aqueous ammonium chloride solution (20 mL) and ethyl acetate (30 mL). The diluted product mixture was transferred to a separatory funnel and the layers that formed were separated. The aqueous layer was extracted with ethyl acetate (2×30 mL). The organic layers were combined and the combined organic layers were washed with saturated aqueous sodium chloride solution (30 mL). The washed organic layer was dried over sodium sulfate. The dried solution was filtered and the filtrate was concentrated. The residue obtained was purified by flash-column chromatography (eluting with 75% dichloromethane-hexanes initially, grading to dichloromethane, linear gradient) to provide the β-ketothioester S6 as a white solid (275 mg, 18%).

¹H NMR (500 MHz, CD₂Cl₂) δ 5.04 (bs, 1H), 3.72 (s, 2H, H₃), 1.46 (s, 9H, H₄), 1.42 (s, 9H, H₁), 1.34 (s, 6H, H₂). ¹³C NMR (126 MHz, CD₂Cl₂) δ 203.7 (C), 193.7 (C), 155.2 (C), 80.7 (C), 61.6 (C), 51.7 (CH₂), 49.1 (C), 29.9 (CH₃), 28.6 (CH₃), 24.2 (CH₃). IR (ATR-FTIR), cm⁻¹: 3348 (m), 2974 (m), 2942 (w), 1723 (s), 1697 (s), 1668 (s), 1522 (s), 1452 (m), 1366 (m), 1273 (s), 1166 (s), 1080 (s), 1022 (m), 995 (s), 686 (s). HRMS-CI (m/z): [M+Na]⁺ calcd for C₁₅H₂₇NNaO₄S, 340.1553; found, 340.1553.

### Synthesis of the β-Ketoamide S8:

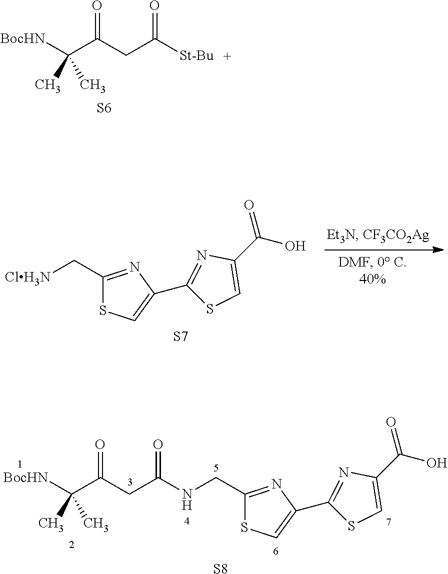

A solution of the thioester S6 (821 mg, 2.59 mmol, 1.30 equiv) in N,N-dimethylformamide (5.4 mL) was added dropwise via syringe over 20 min to a solution of silver trifluoroacetate (879 mg, 3.98 mmol, 2.00 equiv), triethylamine (1.11 mL, 7.96 mmol, 4.00 equiv), and the amine S7 (480 mg, 1.99 mmol, 1 equiv) in N,N-dimethylformamide (21 mL) at 0° C. The reaction mixture was stirred for 1 h at 0° C. The product mixture was filtered through a fritted funnel and the filtrate was concentrated. The concentrated product mixture was applied to a column containing trimethylamine acetate-functionalized silica gel (Si-TMA acetate; eluting with 2% acetic acid-methanol). The fractions containing product were collected, combined, and concentrated. The residue obtained was triturated with dichloromethane (50 mL) to provide the β-ketoamide S8 as a white solid (368 mg, 40%).

¹H NMR (500 MHz, DMSO-d₆) δ 13.01 (bs, 1H), 8.94-8.86 (m, 1H, H₄), 8.47 (s, 1H, H₇), 8.25 (s, 1H, H₆), 7.49 (bs, 1H), 4.60 (d, J=5.9 Hz, 2H, H₅), 3.49 (s, 2H, H₃), 1.39 (s, 9H, H₁), 1.23 (s, 6H, H₂). ¹³C NMR (126 MHz, DMSO-d₆) δ 205.4 (C), 171.3 (C), 167.2 (C), 162.1 (C), 162.0 (C), 155.0 (C), 148.2 (C), 147.1 (C), 128.9 (CH), 118.3 (CH), 78.6 (C), 60.2 (C), 43.0 (CH₂), 40.5 (CH₂), 28.2 (CH₃), 23.2 (CH₃). IR (ATR-FTIR), cm⁻¹: 3308 (br m), 2962 (w), 2933 (w), 1713 (m), 1670 (s), 1650 (s), 1516 (s), 1293 (s), 1163 (s), 1053 (m), 754 (m). HRMS-CI (m/z): [M+H]⁺ calcd for C₁₉H₂₅N₄O₆S₂, 469.1210; found, 469.1212.

### Synthesis of the Hydrochloride Salt S9:

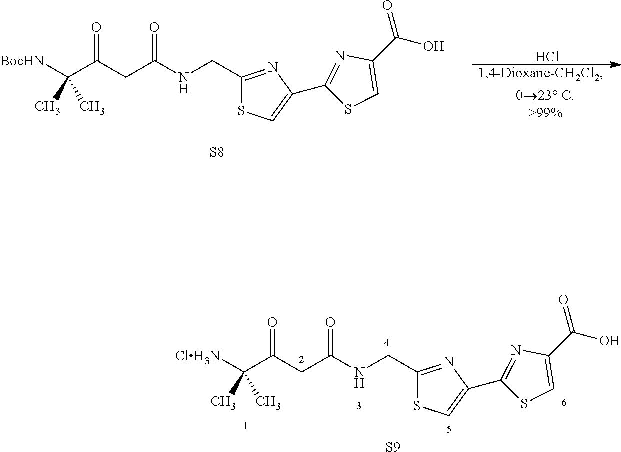

A solution of hydrogen chloride in 1,4-dioxane (4.0 N, 3.74 mL, 14.5 mmol, 70.2 equiv) was added dropwise via syringe to a solution of the β-ketoamide S8 (100 mg, 213 μmol, 1 equiv) in dichloromethane (4.0 mL) at 0° C. The reaction mixture was allowed to warm to 23° C. and stirred at this temperature for 1 h. The product mixture was concentrated to provide the hydrochloride salt S9 as a white solid (86.4 mg, >99%). The product S9 obtained in this way was used directly in the following step.

¹H NMR (400 MHz, DMSO-d₆) δ 9.22 (t, J=6.1 Hz, 1H, H₃), 8.49 (s, 1H, H₆), 8.41 (bs, 3H), 8.29 (s, 1H, H₅), 4.65 (d, J=5.8 Hz, 2H, H₄), 3.77 (s, 2H, H₂), 1.49 (s, 6H, H₁). ¹³C NMR (126 MHz, DMSO-d₆) δ 202.7 (C), 170.7 (C), 165.9 (C), 162.0 (C), 162.0 (C), 148.1 (C), 147.1 (C), 128.9 (CH), 118.4 (CH), 61.5 (C), 43.7 (CH₂), 40.5 (CH₂), 21.9 (CH₃).

### Synthesis of the Acid S10:

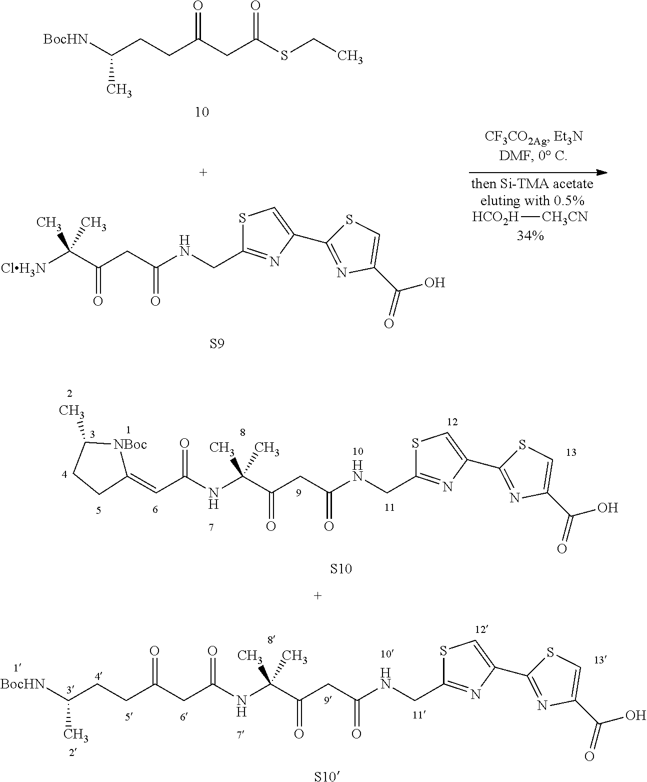

Silver trifluoroacetate (164 ng, 742 μmol, 2.00 equiv) was added to a solution of triethylamine (207 μL, 1.48 mmol, 4.00 equiv) and the amine 9 (149 mg, 371 μmol, 1 equiv) in N,N-dimethylformamide (2.7 mL) at 0° C. A solution of the β-ketothioester 10 (146 mg, 482 μmol, 1.30 equiv) in N,N-dimethylformamide (1.2 mL) was added dropwise via syringe to the reaction mixture. The reaction vessel was covered with foil to exclude light and the reaction mixture was stirred for 1 h at 0° C. The heterogeneous product mixture was filtered through a fritted funnel and the filtrate was concentrated. The residue obtained was applied to a column containing trimethylamine acetate-functionalized silica gel (Si-TMA acetate; eluting with 0.5% formic acid-acetonitrile). The fractions containing product were collected, combined, and concentrated to provide a 2.3:1 mixture of the acids S10 and S10′ as a white solid (146 mg, 34%). The product mixture so obtained was used without further purification.

¹H NMR (S10, 400 MHz, DMSO-d₆) δ 8.81 (t, J=6.0 Hz, 1H, H₁₀), 8.47 (s, 1H, H₁₃), 8.29 (bs, 1H, H₇), 8.25 (s, 1H, H₁₂), 6.59 (s, 1H, H₆), 4.60 (d, J=5.9 Hz, 2H, H₁₁), 4.15 (app p, J=6.5 Hz, 1H, H₃), 3.43 (s, 2H, H₉), 3.40-3.26 (m, 1H, H₅), 2.75 (dt, J=18.9, 10.0 Hz, 1H, H₅), 1.95-1.77 (m, 18, H₄), 1.56-1.47 (m, 1H, H₄), 1.47 (s, 9H, H₁), 1.23 (s, 6H, H₈), 1.13 (d, J=6.4 Hz, 3H, H₂). ¹H NMR (S10′, 400 MHz, DMSO-d₆) δ 8.87 (t, J=6.1 Hz, 1H, H_{10′}), 8.61 (bs, 1H, H_{7′}), 8.47 (s, 1H, H_{13′}), 8.25 (s, 1H, H_{12′}), 6.71-6.64 (m, 1H), 4.60 (d, J=5.9 Hz, 2H, H_{4′}), 3.51 (s, 2H, H_{9′}), 3.39-3.27 (m, 3H, H_{3′}, H_{6′}), 2.54-2.44 (m, 2H, H_{5′}), 1.56-1.45 (m, 2H, H_{4′}), 1.37 (s, 9H, H_{1′}), 1.27 (s, 6H, H_{8′}), 0.98 (d, J=6.5 Hz, 3H, H_{2′}). ¹³C NMR (S10, 101 MHz, DMSO-d₆) δ 204.9 (C), 171.4 (C), 167.6 (C), 167.5 (C), 163.1 (C), 162.0 (C), 153.3 (C), 151.2 (C), 148.1 (C), 147.1 (C), 128.9 (CH), 118.3 (CH), 98.2 (CH), 80.8 (C), 59.9 (C), 56.0 (CH), 43.0 (CH₂), 40.6 (CH₂), 28.8 (CH₂), 27.8 (CH₂), 27.8 (CH₃), 23.4 (CH₃), 23.3 (CH₃), 19.3 (CH₃). ¹³C NMR (S10′, 101 MHz, DMSO-d₆) δ 204.7 (C), 204.6 (C), 171.4 (C), 167.3 (C), 166.5 (C), 163.1 (C), 162.0 (C), 155.1 (C), 148.1 (C), 147.1 (C), 128.9 (CH), 118.3 (CH), 77.4 (C), 60.4 (C), 50.0 (CH₂), 45.2 (CH), 43.4 (CH₂), 40.6 (CH₂), 38.8 (CH₂), 29.9 (CH₂), 28.3 (CH₃), 23.4 (CH₃), 23.3 (CH₃), 20.9 (CH₃). IR (ATR-FTIR), cm⁻¹: 3297 (br w), 2976 (w), 2933 (w), 1711 (s), 1648 (s), 1244 (m), 1158 (s), 754 (m), 635 (w). HRMS-CI (m/z): [M+H]⁺ calcd for C₂₆H₃₄N₅O₇S₂, 592.1894; found, 592.1891. [α]_{D}²⁰ +15.0 (c 1.0, CH₃OH).

### Synthesis of the Amide 14d:

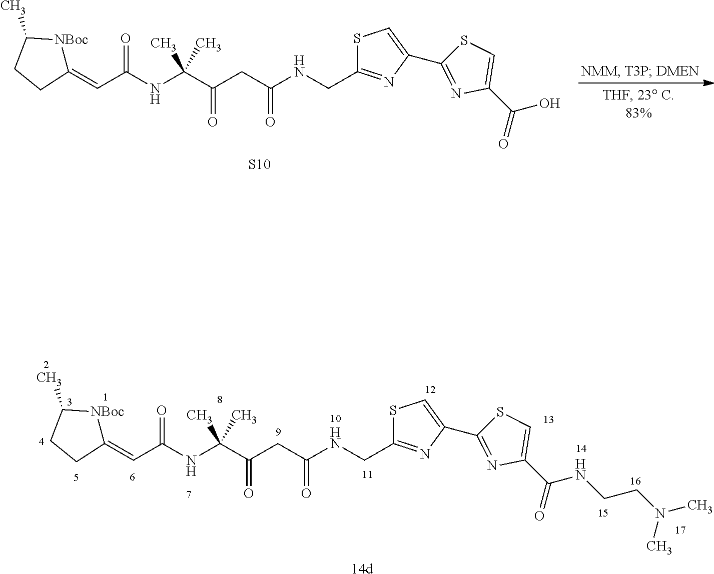

A solution of T3P in ethyl acetate (50 wt %, 154 μL, 259 μmol, 1.50 equiv) and 4-methylmorpholine (94.9 μL, 863 μmol, 5.00 equiv) were added in sequence to a solution of the acid S10 (102.1 mg, 173 μmol, 1 equiv) in tetrahydrofuran (1.2 mL) at 23° C. The reaction mixture was stirred for 20 min at 23° C. N,N-Dimethylethylenediamine (47.1 μL, 431 μmol, 2.50 equiv) was added to the reaction mixture. The resulting mixture was stirred for 7 h at 23° C. The product mixture was concentrated. The concentrated product mixture was diluted with ethyl acetate (10 mL). The diluted product mixture was poured into a separatory funnel that had been charged with saturated aqueous sodium bicarbonate solution (5.0 mL) and the layers that formed were separated. The aqueous layer was extracted with ethyl acetate (2×10 mL). The organic layers were combined and the combined organic layers were dried over sodium sulfate. The dried solution was filtered and the filtrate was concentrated. The residue obtained was purified by flash-column chromatography (eluting with 15% methanol-dichloromethane initially, grading to 40% methanol-dichloromethane, linear gradient) to provide the amide 14d as a white solid (94.7 mg, 83%).

¹H NMR (500 MHz, CD₂Cl₂) δ 8.05 (bs, 2H, H₁₂, H₁₃), 7.92-7.85 (m, 2H, H₁₀, H₁₄), 6.55 (s, 1H, H₆), 5.97 (bs, 1H, H₇), 4.75 (d, J=6.0 Hz, 2H, H₁₁), 4.20 (app p, J=6.7 Hz, 1H, H₃), 3.67-3.60 (m, 2H, H₁₅), 3.54 (s, 2H, H₉), 3.37 (dd, J=18.3, 9.0 Hz, 1H, H₅), 2.86-2.71 (m, 3H, H₅, H₁₆), 2.50 (s, 6H, H₁₇), 1.86 (ddd, J=20.7, 12.1, 8.6 Hz, 1H, H₄), 1.50 (s, 10H, H₁, H₄), 1.34 (s, 6H, H₈), 1.14 (d, J=6.4 Hz, 3H, H₂). ¹³C NMR (126 MHz, CD₂Cl₂) δ 206.1 (C), 170.7 (C), 169.3 (C), 168.0 (C), 163.2 (C), 161.7 (C), 156.9 (C), 152.4 (C), 151.4 (C), 148.8 (C), 123.9 (CH), 118.1 (CH), 96.9 (CH), 82.2 (C), 61.2 (C), 58.7 (CH₂), 57.4 (CH), 45.2 (CH₃), 43.3 (CH₂), 41.7 (CH₂), 36.7 (CH₂), 30.1 (CH₂), 28.8 (CH₂), 28.5 (CH₃), 24.3 (CH₃), 24.3 (CH₃), 19.8 (CH₃). IR (ATR-FTIR), cm⁻¹: 3306 (br w), 2974 (w), 2934 (w), 1712 (m), 1654 (s), 1601 (w), 1540 (m), 1245 (m), 1156 (s), 620 (w). HRMS-CI (m/z): [M+H]⁺ calcd for C₃₀H₄₄N₇O₆S₂, 662.2789; found, 662.2787. [α]_{D}²⁰ −3.0 (c 1.0, CH₂Cl₂).

### Synthesis of the Lactam 15d:

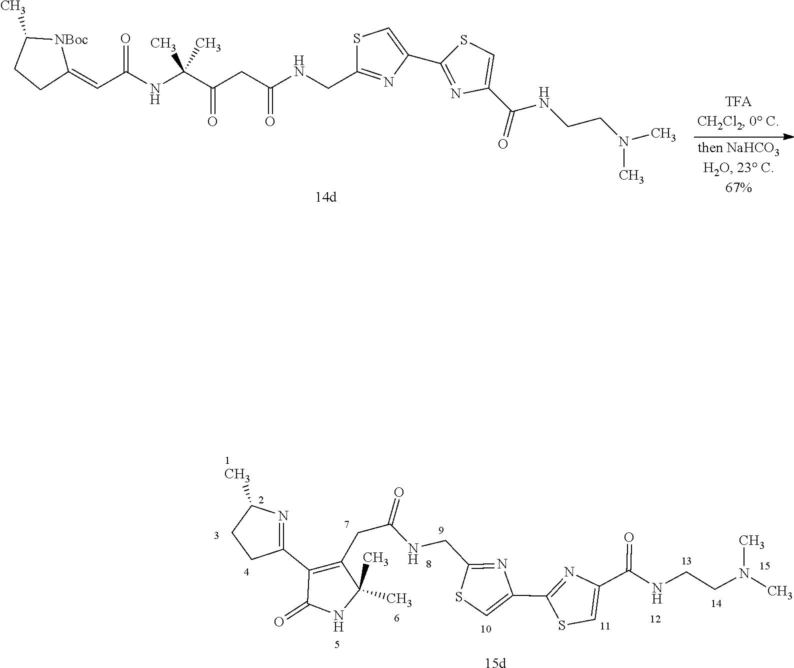

Trifluoroacetic acid (31.9 μL, 417 μmol, 120 equiv) was added dropwise via syringe to a solution of the amide 14d (2.3 mg, 3.48 μmol, 1 equiv) in dichloromethane (200 μL) at 0° C. The reaction mixture was stirred for 14 h at 0° C. The reaction mixture was concentrated. The concentrated reaction mixture was diluted with saturated aqueous sodium bicarbonate solution (200 μL). The diluted reaction mixture was stirred for 1 h at 23° C. The product mixture was diluted sequentially with water (1.0 mL) and ethyl acetate (3.0 mL). The diluted product mixture was transferred to a separatory funnel and the layers that formed were separated. The aqueous layer was extracted with ethyl acetate (5×3.0 mL). The organic layers were combined and the combined organic layers were dried over sodium sulfate. The dried solution was filtered and the filtrate was concentrated. To induce complete cyclization, the residue obtained was diluted with dichloromethane (1.0 mL) and left to stand for 3 h at 23° C. The diluted product mixture was concentrated to provide the lactam 15d as an off-white solid (1.3 mg, 67%).

¹H NMR (500 MHz, CD₂Cl₂) δ 10.84 (t, J=6.0 Hz, 1H, H₈), 8.05 (s, 1H, H₁₁), 7.92 (s, 1H, H₁₀), 7.66 (bs, 1H, H₁₂), 6.23 (bs, 1H, H₅), 4.71-4.65 (m, 2H, H₉), 4.23-4.15 (m, 1H, H₂), 3.59-3.45 (m, 4H, H₇, H₁₃), 3.21-3.11 (m, 1H, H₄), 3.03-2.90 (m, 1H, H₄), 2.54-2.47 (m, 2H, H₁₄), 2.27 (s, 6H, H₁₅), 2.21-2.13 (m, 1H, H₃), 1.44-1.40 (m, 7H, H₃, H₆), 1.25 (d, J=6.8 Hz, 3H, H₁). ¹³C NMR (126 MHz, CD₂Cl₂) δ 171.2 (C), 169.8 (C), 169.8 (C), 169.2 (C), 163.6 (C), 163.0 (C), 161.3 (C), 151.7 (C), 149.0 (C), 127.9 (C), 123.7 (CH), 117.4 (CH), 67.9 (CH), 62.1 (C), 58.8 (CH₂), 45.7 (CH₃), 41.4 (CH₂), 37.5 (CH₂), 37.5 (CH₂), 36.5 (CH₂), 30.8 (CH₂), 25.5 (CH₃), 25.5 (CH₃), 22.4 (CH₃). IR (ATR-FTIR), cm⁻¹: 3277 (br w), 2961 (w), 1655 (s), 1604 (w), 1543 (s), 1187 (m), 619 (m). HRMS-CI (m/z): [M+H]⁺ calcd for C₂₅H₃₄N₇O₃S₂, 544.2159; found, 544.2164. [α]_{D}²⁰ −15.0 (c 1.0, CH₂Cl₂).

### Synthesis of the Thioether 18:

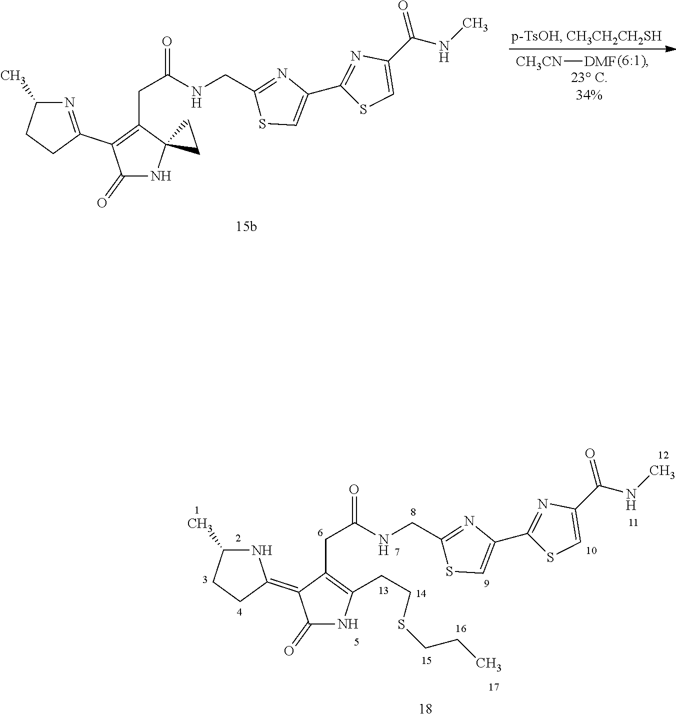

Propanethiol (100 μL) and p-toluenesulfonic acid monohydrate (2.0 mg, 10.3 μmol, 1.00 equiv) were added in sequence to a solution of the amide 15b (5.0 mg, 10.3 μmol, 1 equiv) in acetonitrile (300 μL) at 23° C. The reaction mixture was stirred for 30 min at 23° C. N, Dimethylformamide (50 μL) was added to the reaction mixture at 23° C. The reaction mixture was stirred for 3 h at 23° C. The product mixture was diluted sequentially with toluene (5.0 mL) and hexanes (3.0 mL). The diluted product mixture was filtered and the filtrate was concentrated. The residue obtained was dried by azeotropic distillation with toluene (10 mL) to provide the thioether 18 as a light yellow solid (1.7 mg, 34%).

¹H NMR (500 MHz, DMSO-d₆) δ 9.34-9.26 (m, 1H, H₇), 8.41-8.36 (m, 2H, H₅, H₁₁), 8.25 (s, 1H, H₁₀), 8.18 (s, 1H, H₉), 4.60-4.55 (m, 2H, H₈), 3.93-3.85 (m, 1H, H₂), 3.32 (d, J=17.0 Hz, 1H, H₆), 3.28 (d, J=16.8 Hz, 1H, H₆), 3.02-2.92 (m, 1H, H₄), 2.91-2.84 (m, 1H, H₄), 2.82 (bs, 3H, H₁₂), 2.62-2.54 (m, 4H, H₁₃, H₁₄), 2.49-2.42 (m, 2H, H₁₅), 2.14-2.07 (m, 1H, H₃), 1.55-1.41 (m, 3H, H₃, H₁₆), 1.17 (d, J=6.8 Hz, 3H, H₁), 0.90 (t, J=7.2 Hz, 3H, H₁₇). ¹H NMR (400 MHz, CD₃OD) δ 8.19-8.14 (m, 2H, H₉, H₁₀), 4.71 (d, J=5.2 Hz, 2H, H₈), 4.12-3.99 (m, 1H, H₂), 3.49 (s, 2H, H₆), 3.04-2.89 (m, 5H, H₄, H₁₂), 2.77-2.65 (m, 4H, H₁₃, H₁₄), 2.44 (t, J=7.2 Hz, 2H, H₁₅), 2.32-2.19 (m, 1H, H₃), 1.67-1.56 (m, 1H, H₃), 1.54 (app q, J=7.3 Hz, 2H, H₁₆), 1.34-1.24 (m, 3H, H₁), 0.93 (t, J=7.3 Hz, 3H, H₁₇). ¹³C NMR (126 MHz, DMSO-d₆) δ 171.8 (C), 171.3 (C), 161.7 (C), 160.9 (C), 160.8 (C), 150.9 (C), 147.2 (C), 125.8 (C), 123.7 (CH), 117.9 (CH), 104.3 (C), 96.1 (C), 54.6 (CH), 40.6 (CH₂), 33.1 (CH₂), 32.0 (CH₂), 30.4 (CH₂), 30.2 (CH₂), 29.2 (CH₂), 25.8 (CH₃), 25.7 (CH₂), 22.5 (CH₂), 21.3 (CH₃), 13.3 (CH₃). HRMS-CI (m/z): [M+H]⁺ calcd for C₂₅H₃₃N₆O₃S₃, 561.1771; found, 561.1776. [α]_{D}²⁰ +60.5 (c 0.22, DMSO-d₆).

### Synthesis of the Amide 14e:

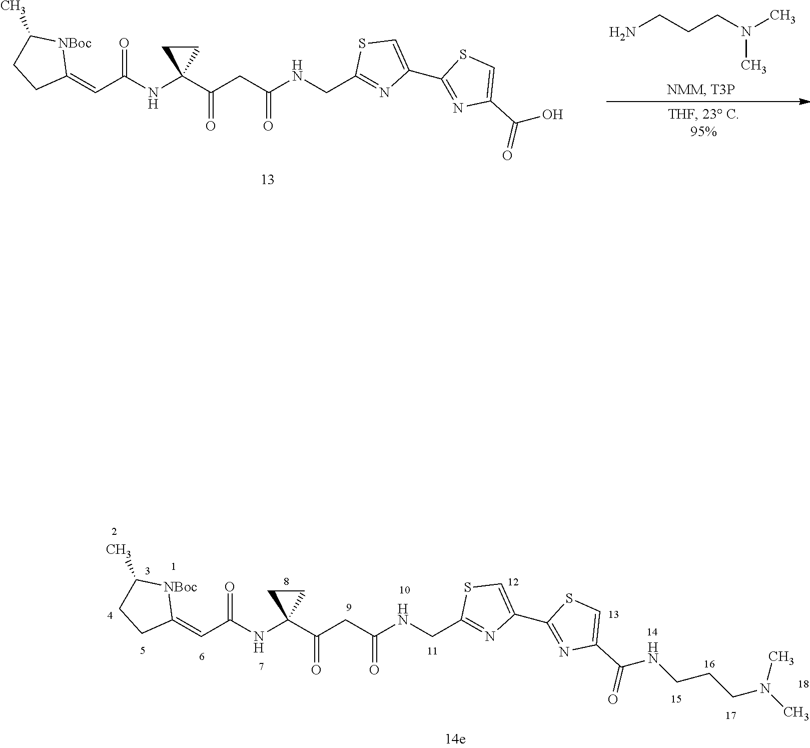

A solution of T3P in ethyl acetate (50 wt %, 30.3 μL, 50.9 μmol, 1.50 equiv) and 4-methylmorpholine (18.6 μL, 170 μmol, 5.00 equiv) were added in sequence to a solution of the acid 13 (20.0 mg, 33.9 μmol, 1 equiv) in tetrahydrofuran (680 μL) at 23° C. The reaction mixture was stirred for 20 min at 23° C. N,N-Dimethyl-1,3-diaminopropane (10.7 μL, 84.8 μmol, 2.50 equiv) was then added to the reaction mixture. The resulting mixture was stirred for 7 h at 23° C. The product mixture was concentrated. The concentrated product mixture was diluted with ethyl acetate (10 mL). The diluted product mixture was poured into a separatory funnel that had been charged with saturated aqueous sodium bicarbonate solution (5.0 mL) and the layers that formed were separated. The aqueous layer was extracted with ethyl acetate (2×10 mL). The organic layers were combined and the combined organic layers were dried over sodium sulfate. The dried solution was filtered and the filtrate was concentrated to provide the amide 14e as a white solid (21.8 mg, 95%). The product so obtained was used without further purification.

¹H NMR (500 MHz, CD₂Cl₂) δ 8.33-8.22 (m, 1H, H₁₄), 8.11-8.05 (m, 1H, H₁₀), 8.03 (s, 1H, H₁₃), 7.90 (s, 1H, H₁₂), 6.55 (s, 1H, H₆), 6.29 (bs, 1H, H₇), 4.76 (d, J=5.9 Hz, 2H, H₁₁), 4.30-4.16 (m, 1H, H₃), 3.66 (s, 2H, H₉), 3.53-3.45 (m, 2H, H₁₅), 3.48-3.42 (m, 1H, H₅), 2.99-2.80 (m, 1H, H₅), 2.41 (t, J=6.5 Hz, 2H, H₁₇), 2.25 (s, 6H, H₁₈), 1.92 (tt, J=11.9, 8.5 Hz, 1H, H₄), 1.83-1.70 (m, 2H, H₁₆), 1.62 (app q, J=4.4 Hz, 2H, H₈), 1.56 (dd, J=12.4, 8.4 Hz, 1H, H₄), 1.49 (s, 9H, H₁), 1.17 (d, J=6.4 Hz, 3H, H₂), 1.18-1.12 (m, 2H, H₈). ¹³C NMR (126 MHz, CD₂Cl₂) δ 206.1 (C), 170.6 (C), 170.4 (C), 167.2 (C), 162.8 (C), 161.3 (C), 156.8 (C), 152.4 (C), 152.1 (C), 149.0 (C), 123.4 (CH), 117.3 (CH), 97.3 (CH), 82.2 (C), 58.7 (CH₂), 57.4 (CH), 45.8 (CH₃), 45.6 (CH₂), 42.0 (C), 41.7 (CH₂), 39.2 (CH₂), 30.2 (CH₂), 28.5 (CH₃), 28.5 (CH₂), 27.3 (CH₂), 21.6 (CH₂), 21.6 (CH₂), 19.8 (CH₃). IR (ATR-FTIR), cm⁻¹: 3282 (w), 2976 (w), 1709 (m), 1650 (s), 1606 (w), 1542 (m), 1290 (m), 1155 (s), 770 (w). HRMS-CI (m/z): [M+H]⁺ calcd for C₃₁H₄₄N₇O₄S₂, 674.2789; found, 674.2795. [α]_{D}²⁰ +33.0 (c 1.0, CH₂Cl₂).

### Synthesis of the Amide 14f:

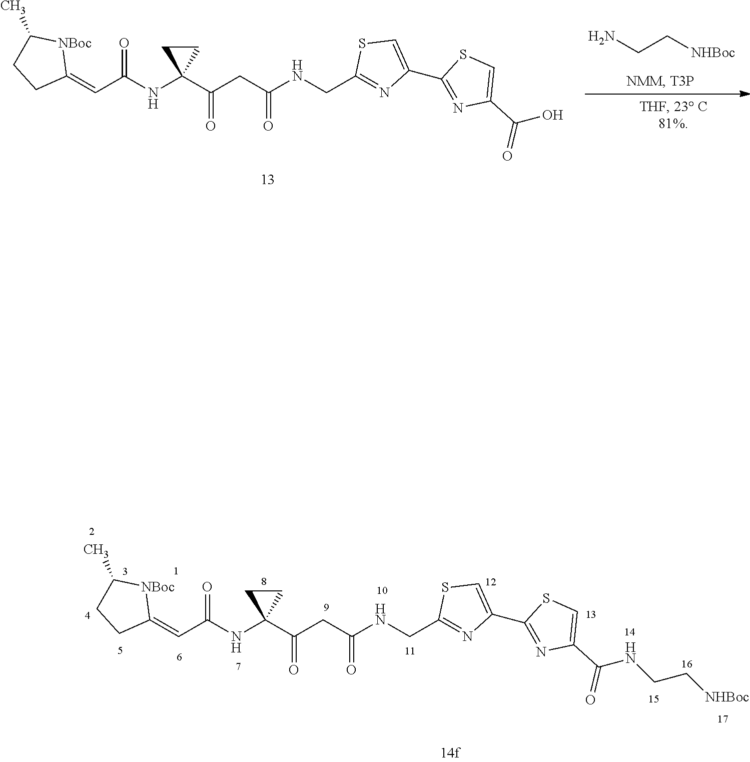

A solution of T3P in ethyl acetate (50 wt %, 22.7 μL, 38.2 μmol, 1.50 equiv) and 4-methylmorpholine (14.0 μL, 127 μmol, 5.00 equiv) were added in sequence to a solution of the acid 13 (15.0 mg, 25.4 μmol, 1 equiv) in tetrahydrofuran (510 μL) at 23° C. The reaction mixture was stirred for 20 min at 23° C. tert-Butyl-N-(2-aminoethyl)carbamate (9.1 μL, 57.2 μmol, 2.25 equiv) was added to the reaction mixture. The resulting mixture was stirred for 7 h at 23° C. The product mixture was concentrated. The residue obtained was purified by flash-column chromatography (eluting with dichloromethane initially, grading to 10% methanol-dichloromethane, linear gradient) to provide the amide 14f as an off-white solid (15.1 mg, 81%).

¹H NMR (600 MHz, CD₂Cl₂) δ 8.10-8.04 (m, 2H, H₁₀, H₁₃), 7.96 (s, 1H, H₁₂), 7.76 (bs, 1H, H₁₄), 6.55 (s, 1H, H₆), 6.24 (bs, 1H, H₇), 5.08 (bs, 1H), 4.76 (d, J=4.1 Hz, 2H, H₁₁), 4.32-4.19 (m, 1H, H₃), 3.67 (s, 2H, H₉), 3.57-3.50 (m, 2H, H₁₅), 3.48 (dd, J=18.3, 8.8 Hz, 1H, H₅), 3.38-3.33 (m, 2H, H₁₆), 2.96-2.81 (m, 1H, H₅), 2.03-1.85 (m, 1H, H₄), 1.65-1.59 (m, 2H, H₈), 1.61-1.52 (m, 1H, H₄), 1.50 (s, 9H, H₁), 1.40 (s, 9H, H₁₇), 1.20-1.13 (m, 5H, H₂, H₈). ¹³C NMR (126 MHz, CD₂Cl₂) δ 206.2 (C), 170.6 (C), 170.4 (C), 167.3 (C), 163.0 (C), 162.1 (C), 156.8 (C), 156.8 (C), 152.4 (C), 151.3 (C), 148.8 (C), 123.9 (CH), 117.8 (CH), 97.3 (CH), 82.2 (C), 79.7 (C), 57.4 (CH), 45.7 (CH₂), 42.0 (C), 41.6 (CH₂), 41.2 (CH₂), 40.5 (CH₂), 30.2 (CH₂), 28.8 (CH₂), 28.6 (CH₃), 28.5 (CH₃), 21.6 (CH₂), 21.6 (CH₂), 19.8 (CH₃). IR (ATR-FTIR), cm⁻¹: 3313 (br w), 2970 (w), 2930 (w), 1704 (m), 1650 (m), 1524 (w), 1154 (s), 1025 (w), 802 (m). HRMS-CI (m/z): [M+H]⁺ calcd for C₃₃H₄₆N₇O₈S₂, 732.2844; found, 732.2852. [α]_{D}²⁰ +4.0 (c 1.0, CH₂Cl₂).

### Synthesis of the Amide 14g:

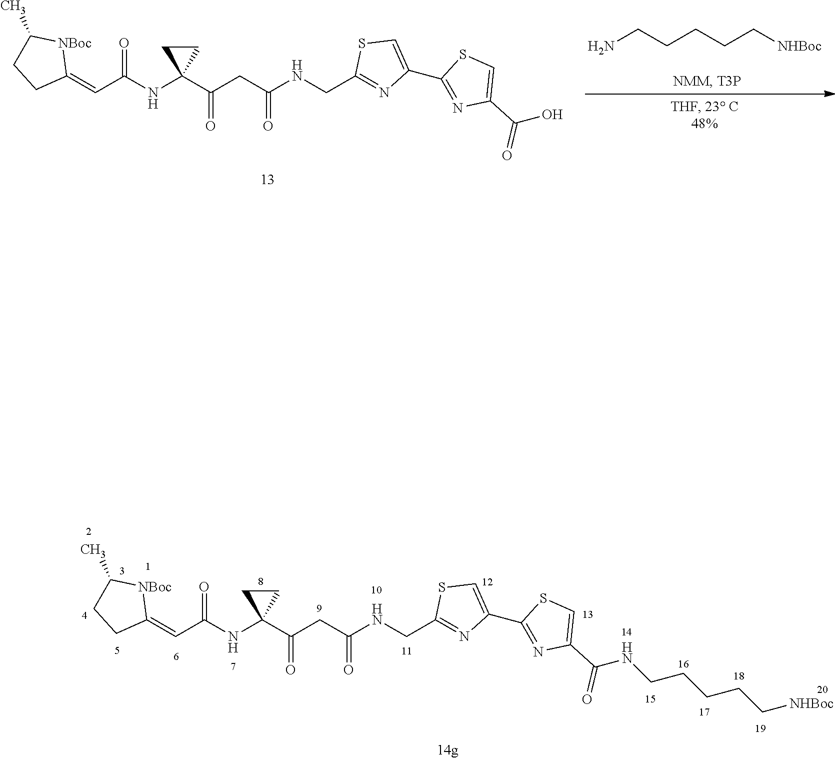

A solution of T3P in ethyl acetate (50 wt %, 30.3 μL, 50.9 μmol, 1.50 equiv) and 4-methylmorpholine (18.6 μL, 170 μmol, 5.00 equiv) were added in sequence to a solution of the acid 13 (20.0 mg, 33.9 μmol, 1 equiv) in tetrahydrofuran (670 μL) at 23° C. The reaction mixture was stirred for 20 min at 23° C. tert-Butyl-N-(2-aminopentyl)carbamate (17.7 μL, 84.8 μmol, 2.50 equiv) was added to the reaction mixture. The resulting mixture was stirred for 7 h at 23° C. The product mixture was concentrated. The residue obtained was purified by flash-column chromatography (eluting with dichloromethane initially, grading to 10% methanol-dichloromethane, linear gradient) to provide the amide 14g as an off-white solid (12.6 mg, 48%).

¹H NMR (500 MHz, CD₂Cl₂) δ 8.09-8.04 (m, 1H, H₁₀), 8.05 (s, 1H, H₁₃), 7.96 (s, 1H, H₁₂), 7.42 (t, J=6.2 Hz, 1H, H₁₄), 6.55 (s, 1H, H₆), 6.27 (bs, 1H, H₇), 4.76 (d, J=6.0 Hz, 2H, H₁₁), 4.64 (bs, 1H), 4.23 (app p, J=6.7 Hz, 1H, H₃), 3.66 (s, 2H, H₉), 3.51-3.44 (m, 1H, H₅), 3.43 (app q, J=6.7 Hz, 2H, H₁₅), 3.13-3.05 (m, 2H, H₁₉), 2.95-2.84 (m, 1H, H₅), 1.92 (tt, J=12.6, 8.6 Hz, 1H, H₄), 1.68-1.60 (m, 4H, H₈, H₆), 1.60-1.53 (m, 1H, H₄), 1.54-1.50 (m, 2H, H₁₈), 1.50 (s, 9H, H₁), 1.43-1.37 (m, 1H, H₁₇, H₂₀), 1.18 (d, J=6.5 Hz, 3H, H₂), 1.18-1.12 (m, 2H, H₈). ¹³C NMR (126 MHz, CD₂Cl₂) δ 206.1 (C), 170.6 (C), 170.4 (C), 167.2 (C), 163.0 (C), 161.3 (C), 156.8 (C), 156.4 (C), 152.4 (C), 151.7 (C), 148.8 (C), 123.6 (CH), 117.7 (CH), 97.3 (CH), 82.2 (C), 79.2 (C), 57.4 (CH), 45.6 (CH₂), 42.0 (C), 41.7 (CH₂), 41.0 (CH₂), 39.7 (CH₂), 30.3 (CH₂), 30.2 (CH₂), 30.1 (CH₂), 28.9 (CH₂), 28.7 (CH₃), 28.5 (CH₃), 24.7 (CH₂), 21.6 (CH₂), 21.6 (CH₂), 19.9 (CH₃). IR (ATR-FTIR), cm⁻¹: 3305 (br m), 2970 (w), 2932 (w), 1699 (s), 1653 (s), 1541 (m), 1242 (m), 1156 (s), 1026 (m), 621 (w). HRMS-CI (m/z): [M+H]⁺ calcd for C₃₆H₅₂N₇O₈S₂, 774.3313; found, 774.3309. [α]_{D}²⁰ +30.0 (c 0.9, CH₂Cl₂).

### Synthesis of the Amide 14h:

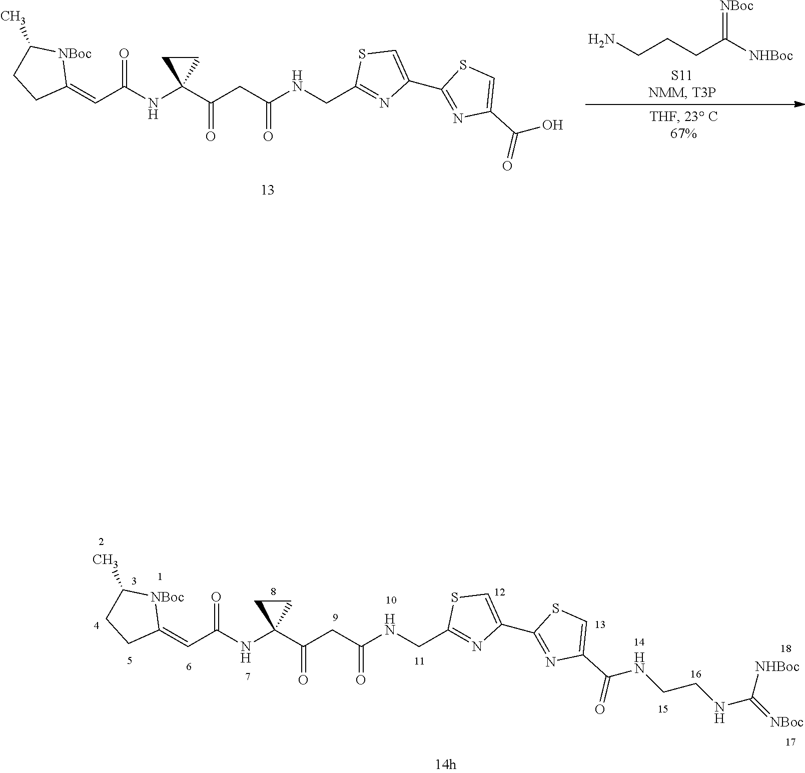

A solution of T3P in ethyl acetate (50 wt %, 22.7 μL, 38.2 μmol, 1.50 equiv) and 4-methylmorpholine (14.0 μL, 127 μmol, 5.00 equiv) were added in sequence to a solution of the acid 13 (15.0 mg, 25.4 μmol, 1 equiv) in tetrahydrofuran (510 μL) at 23° C. The reaction mixture was stirred for 20 min at 23° C. The amine S11 (17.3 mg, 57.2 μmol, 2.25 equiv) was added to the reaction mixture. The resulting mixture was stirred for 7 h at 23° C. The product mixture was concentrated. The residue obtained was purified by flash-column chromatography (eluting with dichloromethane initially, grading to 10% methanol-dichloromethane, linear gradient) to provide the amide 14h as an off-white solid (14.9 mg, 67%).

¹H NMR (500 MHz, CD₂Cl₂) δ 11.50 (bs, 1H), 8.49 (bs, 1H), 8.11-8.03 (m, 2H, H₁₀, H₁₃), 7.96 (s, 1H, H₁₂), 7.70-7.64 (m, 1H, H₁₄), 6.55 (s, 1H, H₆), 6.25 (bs, 1H, H₇), 4.75 (d, J=5.9 Hz, 2H, H₁₁, 4.29-4.14 (m, 1H, H₃), 3.66 (s, 2H, H₉), 3.65-3.58 (m, 4H, H₁₅, H₁₆), 3.47 (dd, J=18.3, 8.7 Hz, 1H, H₅), 2.94-2.82 (m, 1H, H₅), 1.98-1.86 (m, 1, H₄), 1.65-1.59 (m, 2H, H₈), 1.60-1.52 (m, 1H, H₄), 1.49 (s, 9H, H₁), 1.48 (s, 9H, H₁₇), 1.44 (s, 9H, H₁₈), 1.17 (d, J=6.2 Hz, 3H, H₂), 1.18-1.12 (m, 2H, H₈). ¹³C NMR (126 MHz, CD₂Cl₂) δ 206.1 (C), 170.6 (C), 170.3 (C), 167.2 (C), 164.0 (C), 163.0 (C), 161.8 (C), 157.2 (C), 156.9 (C), 153.6 (C), 152.4 (C), 151.3 (C), 148.8 (C), 123.9 (CH), 117.8 (CH), 97.2 (CH), 83.7 (C), 82.2 (C), 79.5 (C), 57.4 (CH), 45.6 (CH₂), 42.0 (C), 41.6 (CH₂), 40.9 (CH₂), 39.2 (CH₂), 30.2 (CH₂), 28.8 (CH₃), 28.8 (CH₂), 28.6 (CH₃), 28.5 (CH₃), 21.6 (CH₂), 21.6 (CH₂), 19.8 (CH₃). IR (ATR-FTIR), cm⁻¹: 3323 (br w), 2976 (w), 2934 (w), 1716 (m), 1639 (s), 1611 (s), 1544 (w), 1316 (m), 1289 (m), 1133 (s), 1024 (m), 771 (w). HRMS-CI (m/z): [M+H]⁺ calcd for C₃₉H₅₆N₉O₁₀S₂, 874.3586; found, 874.3583. [α]_{D}²⁰ +27.0 (c 1.0, CH₂Cl₂).

### Synthesis of the Amide 14i:

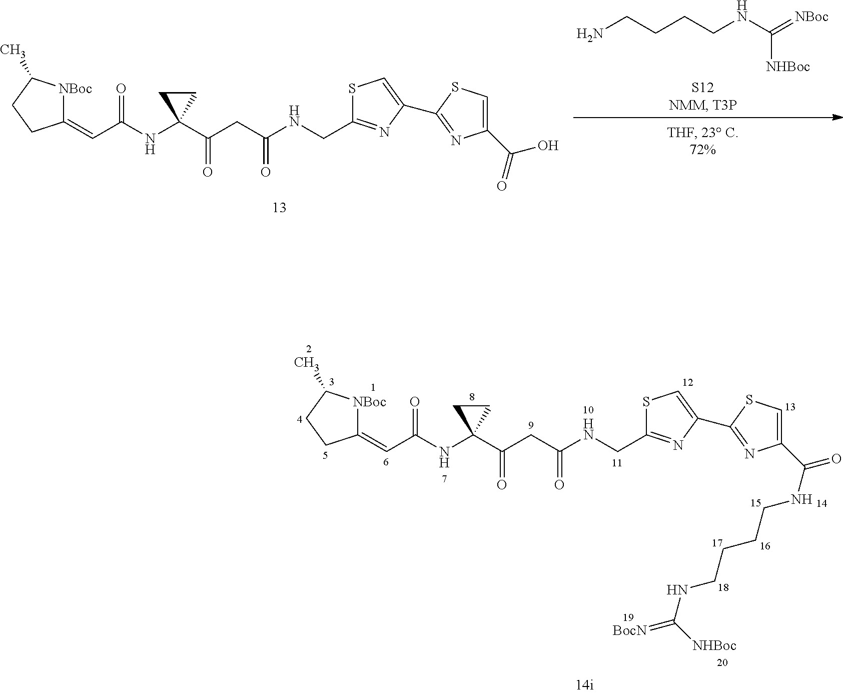

A solution of T3P in ethyl acetate (50 wt %, 30.3 μL, 50.9 μmol, 1.50 equiv) and 4-methylmorpholine (18.6 μL, 170 μmol, 5.00 equiv) were added in sequence to a solution of the acid 13 (20.0 mg, 33.9 μmol, 1 equiv) in tetrahydrofuran (680 μL) at 23° C. The reaction mixture was stirred for 20 min at 23° C. The amine S12 (39.2 mg, 119 μmol, 3.50 equiv) was added to the reaction mixture. The resulting mixture was stirred for 7 h at 23° C. The product mixture was concentrated. The residue obtained was purified by flash-column chromatography (eluting with dichloromethane initially, grading to 10% methanol-dichloromethane, linear gradient) to provide the amide 14i as an off-white solid (22.1 mg, 72%).

¹H NMR (600 MHz, CD₂Cl₂) δ 11.50 (bs, 1H), 8.29 (t, J=5.5 Hz, 1H), 8.11 (t, J=6.0 Hz, 1H, H₁₀), 8.06 (s, 1H, H₁₃), 7.95 (s, 1H, H₁₂), 7.49 (t, J=6.2 Hz, 1H, H₁₄), 6.56 (s, 1H, H₆), 6.39 (bs, 1H, H₇), 4.76 (d, J=6.2 Hz, 2H, H₁₁), 4.34-4.16 (m, 1H, H₃), 3.66 (s, 2H, H₉), 3.56-3.43 (m, 3H, H₅, H₁₅), 3.41 (app q, J=6.1 Hz, 2H, H₁₈), 2.89 (dt, J=18.6, 9.8 Hz, 1H, H₅), 1.92 (ddd, J=20.9, 12.0, 8.4 Hz, 1H, H₄), 1.71-1.63 (m, 4H, H₁₆, H₁₇), 1.64-1.59 (m, 2H, H₈), 1.60-1.52 (m, 1H, H₄), 1.49 (s, 9H, H₁), 1.49 (s, 9H, H₁₉), 1.44 (s, 9H, H₂₀), 1.17 (d, J=6.6 Hz, 3H, H₂), 1.16-1.13 (m, 2H, H₈). ¹³C NMR (126 MHz, CD₂Cl₂) δ 206.1 (C), 170.6 (C), 170.3 (C), 167.2 (C), 164.1 (C), 163.0 (C), 161.4 (C), 156.9 (C), 156.7 (C), 153.8 (C), 152.4 (C), 151.7 (C), 148.8 (C), 123.7 (CH), 117.7 (CH), 97.2 (CH), 83.6 (C), 82.2 (C), 79.3 (C), 57.5 (CH), 45.6 (CH₂), 42.0 (C), 41.7 (CH₂), 41.0 (CH₂), 39.5 (CH₂), 30.2 (CH₂), 28.9 (CH₂), 28.6 (CH₃), 28.5 (CH₃), 28.4 (CH₃), 27.8 (CH₂), 27.2 (CH₂), 21.6 (CH₂), 21.6 (CH₂), 19.9 (CH₃). IR (ATR-FTIR), cm⁻¹: 3324 (br w), 2974 (w), 2935 (w), 1716 (m), 1639 (s), 1612 (s), 1366 (m), 1316 (m), 1290 (m), 1155 (s), 1132 (s), 1051 (m), 1026 (m), 771 (w). HRMS-CI (m/z): [M+H]⁺ calcd for C₄₁H₆₀N₉O₁₀S₂, 902.3899; found, 902.3901. [α]_{D}²⁰ +15.0 (c 1.0, CH₂Cl₂).

### Synthesis of the Lactam 15e:

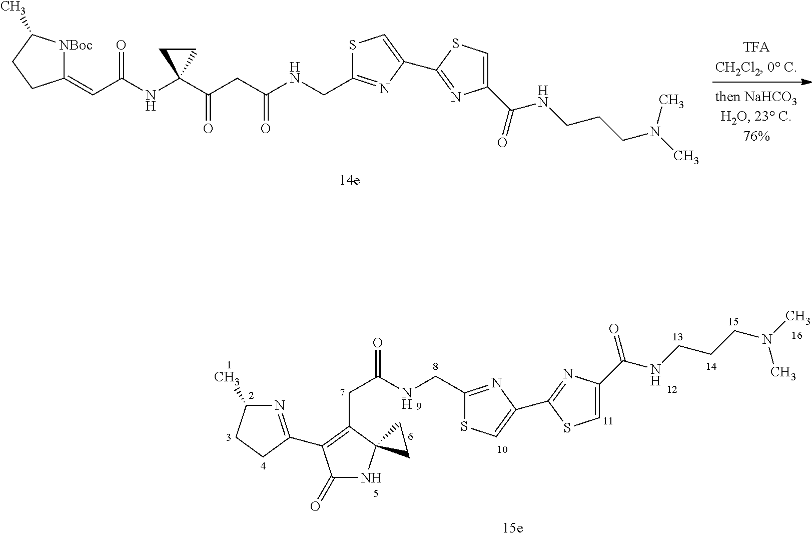

Trifluoroacetic acid (219 μL, 2.87 mmol, 120 equiv) was added dropwise via syringe to a solution of the amide 14e (16.1 mg, 23.9 μmol, 1 equiv) in dichloromethane (600 μL) at 0° C. The reaction mixture was stirred for 14 h at 0° C. The reaction mixture was concentrated. The concentrated reaction mixture was diluted with saturated aqueous sodium bicarbonate solution (1.3 mL). The diluted reaction mixture was stirred for 1 h at 23° C. The product mixture was diluted sequentially with water (1.0 mL) and ethyl acetate (3.0 mL). The diluted product mixture was transferred to a separatory funnel and the layers that formed were separated. The aqueous layer was extracted with ethyl acetate (5×3.0 mL). The organic layers were combined and the combined organic layers were dried over sodium sulfate. The dried solution was filtered and the filtrate was concentrated to provide the lactam 15e as a light yellow solid (10.1 mg, 76%). The product so obtained was used without further purification.

¹H NMR (500 MHz, DMSO-d₆) δ 10.29 (t, J=5.9 Hz, 1H, H₈), 8.70-8.58 (m, 2H, H₅, H₁₂), 8.24 (s, 1H, H₁₁), 8.17 (s, 1H, H₁₀), 4.65-4.57 (m, 2H, H₉), 4.16-4.08 (m, 1H, H₂), 3.35-3.29 (m, 4H, H₇, H₁₃), 3.15-3.02 (m, 1H, H₄), 2.86 (dt, J=17.7, 8.8 Hz, 1H, H₄), 2.28 (t, J=6.9 Hz, 2H, H₁₅), 2.15 (s, 6H, H₁₆), 2.12-2.04 (m, 1H, H₃), 1.71-1.63 (m, 4H, H₆, H₁₄), 1.41-1.31 (m, 3H, H₃, H₆), 1.18 (d, J=6.6 Hz, 3H, H₁). ¹³C NMR (126 MHz, DMSO-d₆) δ 171.2 (C), 169.7 (C), 168.4 (C), 168.3 (C), 161.7 (C), 160.3 (C), 157.7 (C), 151.0 (C), 147.5 (C), 127.4 (C), 123.9 (CH), 117.9 (CH), 66.6 (CH), 57.3 (CH₂), 45.3 (C), 45.2 (CH₃), 40.4 (CH₂), 37.7 (CH₂), 36.6 (CH₂), 33.5 (CH₂), 29.7 (CH₂), 26.9 (CH₂), 21.9 (CH₃), 11.9 (CH₂), 11.8 (CH₂). HRMS-CI (m/z): [M+H]⁺ calcd for C₂₆H₃₄N₇O₃S₂, 556.2159; found, 556.2151. [α]_{D}²⁰ −5.5 (c 3.1, DMSO-d₆).

### Synthesis of the Lactam 15f

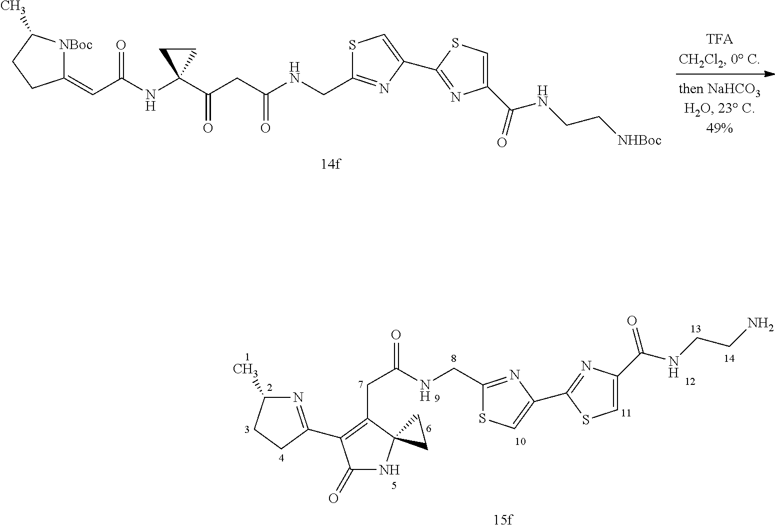

Trifluoroacetic acid (125 μL, 1.64 mmol, 120 equiv) was added dropwise via syringe to a solution of the amide 14f (10.0 mg, 13.7 μmol, 1 equiv) in dichloromethane (340 μL) at 0° C. The reaction mixture was stirred for 14 h at 0° C. The reaction mixture was concentrated. The concentrated reaction mixture was diluted with saturated aqueous sodium bicarbonate solution (700 μL). The diluted reaction mixture was stirred for 1 h at 23° C. The product mixture was diluted sequentially with water (1.0 mL) and ethyl acetate (3.0 mL). The diluted product mixture was transferred to a separatory funnel and the layers that formed were separated. The aqueous layer was extracted with ethyl acetate (5×3.0 mL). The organic layers were combined and the combined organic layers were dried over sodium sulfate. The dried solution was filtered and the filtrate was concentrated to provide the lactam 15f as a light yellow solid (3.4 mg, 49%). The product so obtained was used without further purification.

¹H NMR (500 MHz, DMSO-d₆) δ 10.30 (t, J=6.0 Hz, 1H, H₈), 8.62 (bs, 1H, H₅), 8.44 (t, J=5.9 Hz, 1H, H₁₂), 8.28 (s, 1H, H₁₁), 8.19 (s, 1H, H₁₀), 4.64-4.58 (m, 2H, H₉), 4.17-4.08 (m, 1H, H₂), 3.38-3.29 (m, 4H, H₇, H₁₃), 3.14-3.06 (m, 1H, H₄), 2.87 (dt, H=17.8, 8.7 Hz, 1H, H₄), 2.77 (t, J=6.5 Hz, 2H, H₁₄), 2.10-2.04 (m, 1H, H₃), 1.71-1.66 (m, 2H, H₆), 1.41-1.30 (m, 3H, H₃, H₆), 1.19 (d, J=6.7 Hz, 3H, H₁). ¹³C NMR (126 MHz, DMSO-d₆) δ 171.2 (C), 169.7 (C), 168.4 (C), 168.3 (C), 161.7 (C), 160.6 (C), 157.7 (C), 150.9 (C), 147.4 (C), 127.4 (C), 124.2 (CH), 118.0 (CH), 66.6 (CH), 45.3 (C), 40.9 (CH₂), 40.7 (CH₂), 40.4 (CH₂), 36.6 (CH₂), 33.6 (CH₂), 29.7 (CH₂), 21.9 (CH₃), 11.9 (CH₂), 11.8 (CH₂). HRMS-CI (m/z): [M+H]⁺ calcd for C₂₃H₂₈N₇O₃S₂, 514.1690; found, 514.1690. [α]_{D}²⁰ −1.7 (c 1.2, DMSO-d₆).

### Synthesis of the Lactam 15g:

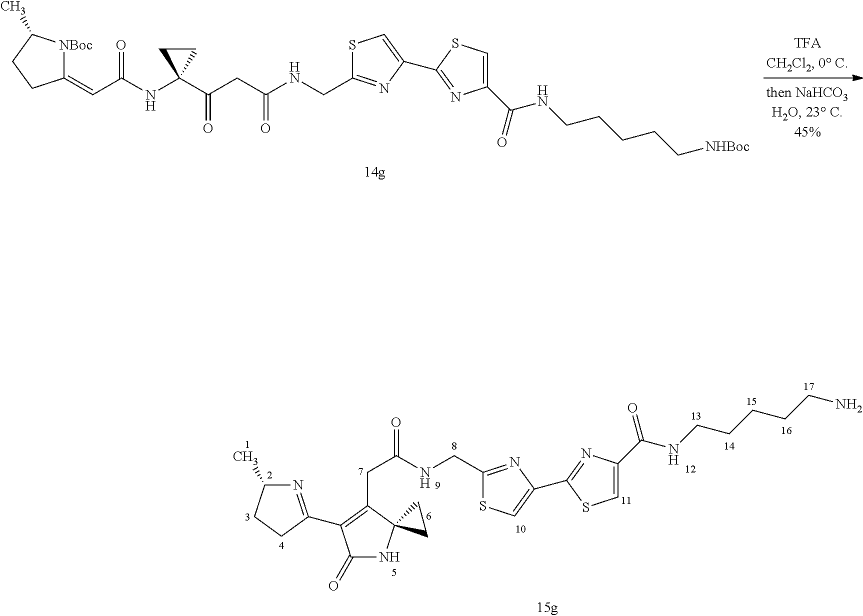

Trifluoroacetic acid (108 μL, 1.41 mmol, 120 equiv) was added dropwise via syringe to a solution of the amide 14g (9.1 mg, 11.8 μmol, 1 equiv) in dichloromethane (290 μL) at 0° C. The reaction mixture was stirred for 14 h at 0° C. The reaction mixture was concentrated. The concentrated reaction mixture was diluted with saturated aqueous sodium bicarbonate solution (650 μL). The diluted reaction mixture was stirred for 1 h at 23° C. The product mixture was diluted sequentially with water (1.0 mL) and ethyl acetate (3.0 mL). The diluted product mixture was transferred to a separatory funnel and the layers that formed were separated. The aqueous layer was extracted with ethyl acetate (5×3.0 mL). The organic layers were combined and the combined organic layers were dried over sodium sulfate. The dried solution was filtered and the filtrate was concentrated to provide the lactam 15g as a light yellow solid (3.4 mg, 45%). The product so obtained was used without further purification.

¹H NMR (500 MHz, DMSO-d₆) δ 10.34-10.26 (m, 1H, H₈), 8.61 (bs, 1H, H₅), 8.44-8.38 (m, 1H, H₁₂), 8.25 (s, 1H, H₁₁), 8.19 (s, 1H, H₁₀), 4.64-4.59 (m, 2H, H₉), 4.17-4.07 (m, 1H, H₂), 3.35-3.26 (m, 4H, H₇, H₁₃), 3.15-3.05 (m, 1H, H₁₄), 2.87 (dt, J=18.0, 8.9 Hz, 1H, H₄), 2.73 (t, J=7.5 Hz, 2H, H₁₇), 2.15-2.05 (m, 1H, H₃), 1.72-1.66 (m, 2H, H₆), 1.58-1.50 (m, 4H, H₁₄, H₁₆), 1.39-1.32 (m, 5H, H₃, H₆, H₁₅), 1.19 (d, J=6.7 Hz, 3H, H₁). ¹³C NMR (126 MHz, DMSO-d₆) δ 171.1 (C), 169.6 (C), 168.4 (C), 168.3 (C), 161.7 (C), 160.3 (C), 157.7 (C), 151.0 (C), 147.5 (C), 127.4 (C), 124.0 (CH), 117.9 (CH), 66.5 (CH), 45.3 (C), 40.4 (CH₂), 39.3 (CH₂), 38.5 (CH₂), 36.5 (CH₂), 33.5 (CH₂), 29.7 (CH₂), 28.8 (CH₂), 27.9 (CH₂), 23.4 (CH₂), 21.9 (CH₃), 11.8 (CH₂) 11.8 (CH₂). HRMS-CI (m/z): [M+H]⁺ calcd for C₂₆H₃₄N₇O₃S₂, 556.2159; found, 556.2167. [α]_{D}²⁰ +2.5 (c 1.2, DMSO-d₆).

### Synthesis of the Lactam 15h:

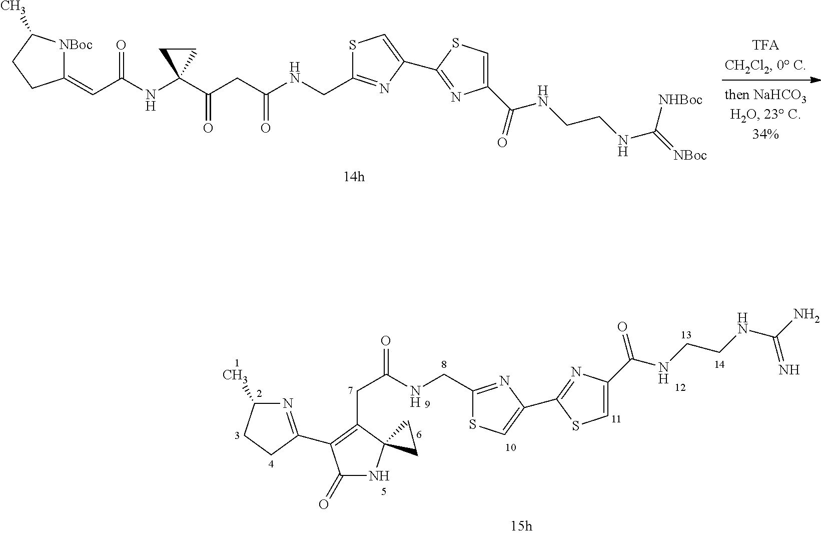

Trifluoroacetic acid (105 μL, 1.37 mmol, 120 equiv) was added dropwise via syringe to a solution of the amide 14h (10.0 mg, 11.4 μmol, 1 equiv) in dichloromethane (290 μL) at 0° C. The reaction mixture was stirred for 14 h at 0° C. The reaction mixture was concentrated. The concentrated reaction mixture was diluted with saturated aqueous sodium bicarbonate solution (650 μL). The diluted reaction mixture was stirred for 1 h at 23° C. The product mixture was diluted sequentially with water (1.0 mL) and ethyl acetate (3.0 mL). The diluted product mixture was transferred to a separatory funnel and the layers that formed were separated. The aqueous layer was extracted with ethyl acetate (5×3.0 mL). The organic layers were combined and the combined organic layers were dried over sodium sulfate. The dried solution was filtered and the filtrate was concentrated to provide the lactam 15h as a light yellow solid (2.2 mg, 34%). The product so obtained was used without further purification.

¹H NMR (600 MHz, DMSO-d₆) δ 10.31 (t, J=6.1 Hz, 1H, H₈), 8.58 (bs, 2H, H₅, H₁₂), 8.31 (s, 1H, H₁₁), 8.16 (s, 1H, H₁₀), 7.42 (bs, 1H), 4.65-4.60 (m, 2H, H₉), 4.17-4.08 (m, 1H, H₂), 3.47-3.41 (m, 2H, H₁₃), 3.36-3.30 (m, 4H, H₇, H₁₄), 3.14-3.07 (m, 1H, H₄), 2.87 (dt, J=18.2, 8.9 Hz, 1H, H₄), 2.12-2.04 (m, 1H, H₃), 1.71-1.65 (m, 2H, H₆), 1.53-1.43 (m, 1H, H₃), 1.38-1.33 (m, 2H, H₆), 1.19 (d, J=6.6 Hz, 3H, H₁). ¹³C NMR (126 MHz, DMSO-d₆) δ 171.2 (C), 169.6 (C), 168.4 (C), 168.3 (C), 161.9 (C), 160.9 (C), 157.6 (C), 157.0 (C), 150.5 (C), 147.4 (C), 127.4 (C), 124.7 (CH), 117.9 (CH), 66.5 (CH), 45.3 (C), 40.4 (CH₂), 40.4 (CH₂), 38.0 (CH₂), 36.5 (CH₂), 33.5 (CH₂), 29.7 (CH₂), 21.9 (CH₃), 11.8 (CH₂), 11.7 (CH₂). HRMS-CI (m/z): [M+H]⁺ calcd for C₂₄H₃₀N₉O₃S₂, 556.1908; found, 556.1913. [α]_{D}²⁰ −5.5 (c 1.3, DMSO-d₆).

### Synthesis of the Lactam 15i:

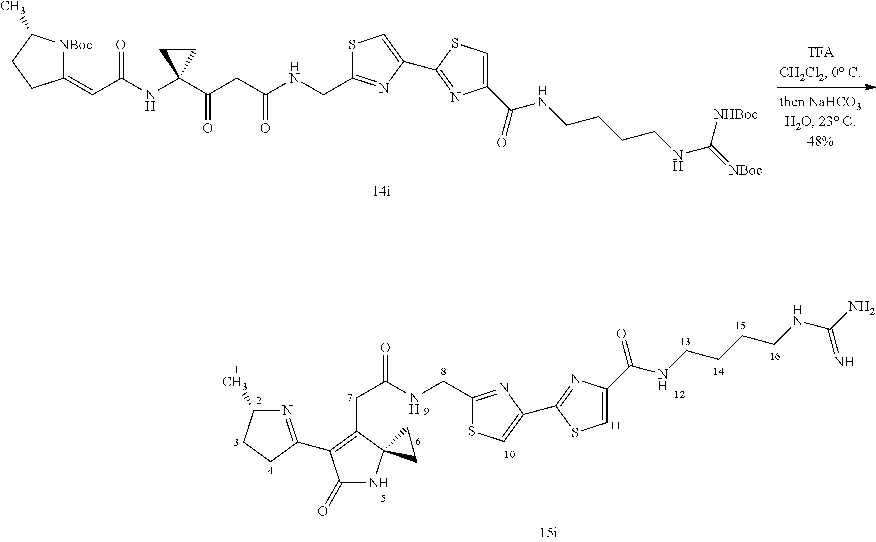

Trifluoroacetic acid (164 μL, 2.14 mmol, 120 equiv) was added dropwise via syringe to a solution of the amide 14i (16.1 mg, 17.8 μmol, 1 equiv) in dichloromethane (450 μL) at 0° C. The reaction mixture was stirred for 14 h at 0° C. The reaction mixture was concentrated. The concentrated reaction mixture was diluted with saturated aqueous sodium bicarbonate solution (1.0 mL). The diluted reaction mixture was stirred for 1 h at 23° C. The product mixture was diluted sequentially with water (1.0 mL) and ethyl acetate (3.0 mL). The diluted product mixture was transferred to a separatory funnel and the layers that formed were separated. The aqueous layer was extracted with ethyl acetate (5×3.0 mL). The organic layers were combined and the combined organic layers were dried over sodium sulfate. The dried solution was filtered and the filtrate was concentrated to provide the lactam 15i as a light yellow solid (5.0 mg, 48%). The product so obtained was used without further purification.

¹H NMR (500 MHz, DMSO-d₆) δ 10.34-10.28 (m, 1H, H₈), 8.63 (bs, 1H, H₅), 8.50 (t, J=6.2 Hz, 1H, H₁₂), 8.26 (s, 1H, H₁₁), 8.19 (s, 1H, H₁₀), 7.88 (bs, 1H), 4.64-4.58 (m, 2H, H₉), 4.16-4.08 (m, 1H, H₂), 3.34-3.27 (m, 4H, H₇, H₁₃), 3.15-3.05 (m, 3H, H₄, H₁₆), 2.87 (dt, J=17.8, 8.8 Hz, 1H, H₄), 2.11-2.04 (m, 1H, H₃), 1.70-1.63 (m, 2H, H₆), 1.60-1.52 (m, 2H, H₁₄), 1.53-1.46 (m, 2H, H₁₅), 1.38-1.33 (m, 3H, H₃, H₆), 1.18 (d, J=6.6 Hz, 3H, H₁). ¹³C NMR (126 MHz, DMSO-d₆) δ 171.2 (C), 169.7 (C), 168.4 (C), 168.3 (C), 161.7 (C), 160.5 (C), 157.7 (C), 156.9 (C), 150.9 (C), 147.5 (C), 127.4 (C), 124.1 (CH), 117.9 (CH), 66.6 (CH), 45.3 (C), 40.4 (CH₂), 40.4 (CH₂), 38.2 (CH₂), 36.6 (CH₂), 33.6 (CH₂), 29.7 (CH₂), 26.5 (CH₂), 26.1 (CH₂), 21.9 (CH₃), 11.9 (CH₂), 11.8 (CH₂). HRMS-CI (m/z): [M+H]⁺ calcd for C₂₆H₃₄N₉O₃S₂, 584.2221; found, 584.2221. [α]_{D}²⁰ +3.1 (c 2.6, DMSO-d₆).

### Synthesis of the Ethyl Ester S14:

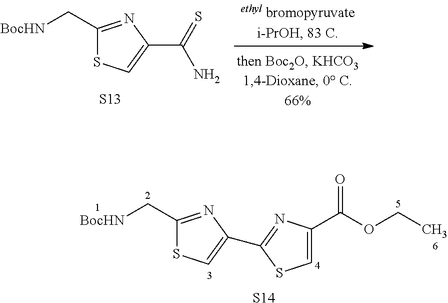 [SMILES: CC(C)(C)OC(=O)NCc1nc(C(N)=S)cs1 ; "S13" ; CCOC(=O)c1csc(-c2csc(CNC(=O)OC(C)(C)C)n2)n1 ; "S14"]

Ethyl bromopyruvate (3.01 g, 15.4 mmol, 1.50 equiv) was added to a solution of the thioamide S13 (2.82 g, 10.3 mmol, 1 equiv) in iso-propanol (100 mL) at 23° C. The reaction mixture was stirred for 16 h at 83° C. The reaction mixture was concentrated. The residue obtained was dissolved in 1,4-dioxane (45 mL) and the resulting solution was cooled to 0° C. Di-tert-butyl dicarbonate (3.60 g, 16.5 mmol, 1.60 equiv) and a solution of aqueous potassium bicarbonate (1N, 15 mL) were then added sequentially to the cooled solution. The reaction mixture was stirred for 16 h at 0° C. The product mixture was concentrated and the residue obtained was applied to a trimethylamine acetate-functionalized silica column (Si-TMA acetate; eluting with 2% acetic acid-methanol) to provide the bithiazole S14 as a white solid (2.51 g, 66%).

¹H NMR (600 MHz, DMSO-d₆) δ 8.55 (s, 1H, H₄), 8.27 (s, 1H, I), 7.87 (t, J 6.2 Hz, 1H), 4.45 (d, J=6.1 Hz, 2H, H₂), 4.34 (q, J=7.1 Hz, 2H, H₅), 1.42 (s, 9H, H₁), 1.33 (t, J=7.1 Hz, 3H, H₆). ¹³C NMR (151 MHz, DMSO-d₆) δ 172.9 (C), 162.5 (C), 160.7 (C), 155.8 (C), 147.1 (C), 147.0 (C), 129.4 (CH), 118.2 (CH), 78.7 (C), 60.9 (CH₂), 41.9 (CH₂), 28.2 (CH₃), 14.2 (CH₃). IR (ATR-FTIR), cm⁻¹: 3343 (m), 3130 (w), 3109 (w), 2983 (w), 1721 (s), 1686 (s), 1526 (s), 1298 (m), 1284 (m), 1204 (s), 1164 (s), 1100 (s), 819 (m), 770 (m), 621 (m). HRMS-CI (m/z): [M+H]⁺ calcd for C₁₅H₂₀N₃O₄S₂, 370.0890; found, 370.0882.

### Synthesis of the Acid S15:

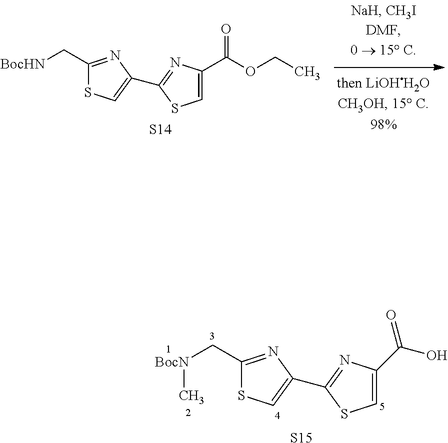 [SMILES: CCOC(=O)c1csc(-c2csc(CNC(=O)OC(C)(C)C)n2)n1 ; "S14" ; CN(Cc1nc(-c2nc(C(=O)O)cs2)cs1)C(=O)OC(C)(C)C ; "S15"]

A dispersion of sodium hydride in mineral oil (60%, 109 mg, 2.83 mmol, 2.00 equiv) was added slowly to a solution of the bithiazole S14 (523 mg, 1.42 mmol, 1 equiv) and iodomethane (793 μL, 12.7 mmol, 9.00 equiv) in N,N-dimethylformamide (6.0 mL) at −5° C. The reaction mixture was stirred for 30 min at −5° C. and then was warmed to 15° C. The warmed mixture was stirred for 14 h at 15° C. Lithium hydroxide (891 mg, 21.2 mmol, 15.0 equiv) and water (6.0 mL) were then added in sequence to the reaction mixture. The reaction mixture was stirred for 3 h at 15° C. The heterogeneous product mixture was filtered through a fritted funnel. The filter cake was washed with methanol (10 mL). The filtrates were combined and the combined filtrates were concentrated. The product mixture was diluted sequentially with saturated aqueous ammonium chloride solution (10 mL) and ethyl acetate (10 mL). The diluted product mixture was transferred to a separatory funnel and the layers that formed were separated. The aqueous layer was extracted with ethyl acetate (3×5.0 mL). The organic layers were combined and the combined organic layers were dried over sodium sulfate. The dried solution was filtered and the filtrate was concentrated. The concentrated product mixture was applied to a column containing trimethylamine acetate-functionalized silica gel (Si-TMA acetate; eluting with 2% acetic acid-methanol). The fractions containing product were collected, combined, and concentrated to provide the acid S15 as a white solid (495 mg, 98%).

In DMSO-d₆ at room temperature the title compound exists as an approximate 1:1 mixture of amide-bond rotamers.

¹H NMR (600 MHz, DMSO-d₆) δ 8.36 (s, 1H, H₅), 8.28 (s, 1H, H₄), 4.71 (s, 2H, H₃), 2.92 (d, 3H, H₂), 1.42 (d, 9H, H₁). ¹³C NMR (151 MHz, DMSO-d₆) δ 169.9 (C), 162.4 (C), 161.6 (C), 154.7 (d, C), 150.1 (C), 147.5 (C), 127.6 (CH), 118.1 (CH), 79.6 (C), 49.8 (d, CH₂), 34.7 (d, CH₃), 27.9 (d, CH₃). IR (ATR-FTIR), cm⁻¹: 3113 (w), 2975 (w), 1681 (s), 1484 (m), 1392 (m), 1295 (m), 1240 (m), 1167 (s), 751 (m), 564 (w). HRMS-CI (m/z): [M+H]⁺ calcd for C₁₄H₁₇N₃NaO₄S2, 378.0553; found, 378.0554.

### Synthesis of the Hydrochloride Salt S16:

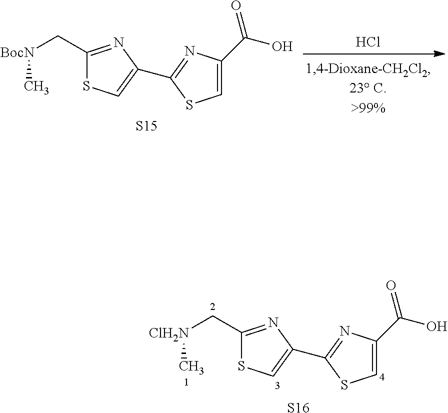

A solution of hydrogen chloride in 1,4-dioxane (4.0 N, 8.0 mL, 32.0 mmol, 24.1 equiv) was added dropwise via syringe to a solution of the bithiazole S15 (472 mg, 1.33 mmol, 1 equiv) in dichloromethane (24 mL) at 23° C. The resulting mixture was stirred for 1 h at 23° C. The product mixture was concentrated to provide the hydrochloride salt S16 as a white solid (387 mg, >99%). The product S16 obtained in this way was used directly in the following step.

¹H NMR (600 MHz, DMSO-d₆) δ 9.72 (bs, 2H), 8.52 (s, 1H, H₄), 8.45 (s, 1H, H₃), 4.61 (t, J=5.8 Hz, 2H, H₂), 2.66 (t, J=5.3 Hz, 3H, H₁). ¹³C NMR (151 MHz, DMSO-d₆) δ 162.0 (C), 161.9 (C), 161.6 (C), 148.2 (C), 147.5 (C), 129.3 (CH), 120.7 (CH), 47.3 (CH₂), 32.5 (CH₃).

### Synthesis of the Acid S18:

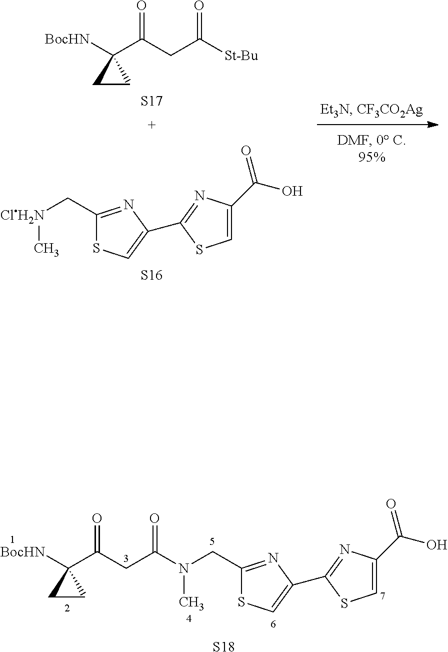

A solution of the β-ketothioester S17 (506 mg, 1.60 mmol, 1.30 equiv) in N,N-dimethylformamide (3.0 mL) was added dropwise via syringe over 20 min to a mixture of silver trifluoroacetate (545 mg, 2.47 mmol, 2.00 equiv), triethylamine (688 μL, 4.94 mmol, 4.00 equiv), and the acid S16 (360 mg, 1.23 mmol, 1 equiv) in N,N-dimethylformamide (12 mL) at 0° C. The reaction mixture was stirred for 1 h at 0° C. The heterogeneous product mixture was filtered through a fritted funnel. The filter cake was washed with methanol (12 mL). The filtrates were combined and the combined filtrates were concentrated. The concentrated product mixture was applied to a column containing trimethylamine acetate-functionalized silica gel (Si-TMA acetate; eluting with 2% acetic acid-methanol). The fractions containing product were collected, combined, and concentrated. The residue obtained was recrystallized from dichloromethane-hexanes (1:4; 50 mL) to provide the acid S18 as a white solid. (565 mg, 95%).

In DMSO-d₆ at room temperature the title compound exists as a mixture (3.5:1) of amide-bond rotamers. The NMR signals are reported for the major isomer.

¹H NMR (600 MHz, DMSO-d₆) δ 13.14 (bs, 1H), 8.48 (s, 1H, H₇), 8.30 (s, 1H, H₆), 7.75 (bs, 1H), 4.83 (s, 2H, H₅), 3.82 (s, 2H, H₃), 2.98 (s, 3H, H₄), 1.41 (s, 9H, H₁), 1.41-1.36 (m, 2H, H₂), 1.09 (q, J=4.4 Hz, 2H, H₂). ¹³C NMR (151 MHz, DMSO-d₆) δ 204.8 (C), 168.9 (C), 167.7 (C), 162.1 (C), 162.0 (C), 156.2 (C), 148.1 (C), 147.1 (C), 129.0 (CH), 118.9 (CH), 78.8 (C), 48.4 (CH₂), 45.3 (CH₂), 41.2 (C), 36.0 (CH₃), 28.1 (CH₃), 19.8 (CH₂). IR (ATR-FTIR), cm⁻¹: 3328 (m), 2935 (w), 1704 (s), 1680 (s), 1643 (s), 1506 (s), 1287 (m), 1236 (m), 1160 (s), 746.2 (m), 457 (m). HRMS-CI (m/z): [M+H]⁺ calcd for C₂₀H₂N₄O₆S₂, 481.1210; found, 481.1215.

### Synthesis of the Hydrochloride Salt S19:

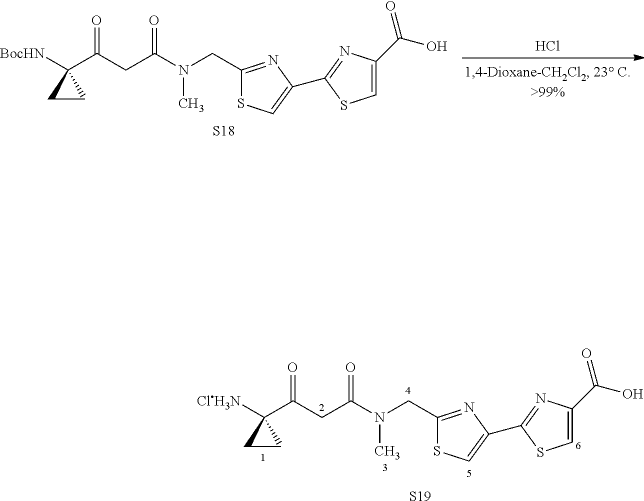

A solution of hydrogen chloride in 1,4-dioxane (4.0 N, 15.0 mL, 60.0 mmol, 54.5 equiv) was added dropwise via syringe to a solution of the bithiazole S18 (527 mg, 1.10 mmol, 1 equiv) in dichloromethane (45.0 mL) at 23° C. The resulting mixture was stirred for 3 h at 23° C. The product mixture was concentrated to provide the hydrochloride salt S19 as a white solid (457 mg, >99%9). The product S19 obtained in this way was used directly in the following step.

In DMSO-d₆ at room temperature the title compound exists as a mixture (3:1) of amide-bond rotamers. The NMR signals are reported for the major isomer.

¹H NMR (600 MHz, DMSO-d₆) δ 8.85 (bs, 3H), 8.49 (s, 1H, H₆), 8.32 (s, 1H, H₅), 4.84 (s, 2H, H₄), 3.78 (s, 2H, H₂), 3.05 (s, 3H, H₃) 1.83-1.74 (m, 2H, H₁), 1.56-1.44 (m, 2H, H₁). ¹³C NMR (151 MHz, DMSO-d₆) δ 199.8 (C), 168.5 (C), 167.2 (C), 162.0 (C), 162.0 (C), 148.1 (C), 147.1 (C), 129.0 (CH), 118.9 (CH), 48.5 (CH₂), 42.0 (C), 41.1 (CH₂), 36.1 (CH₃), 13.6 (CH₂).

### Synthesis of the Lactam S20:

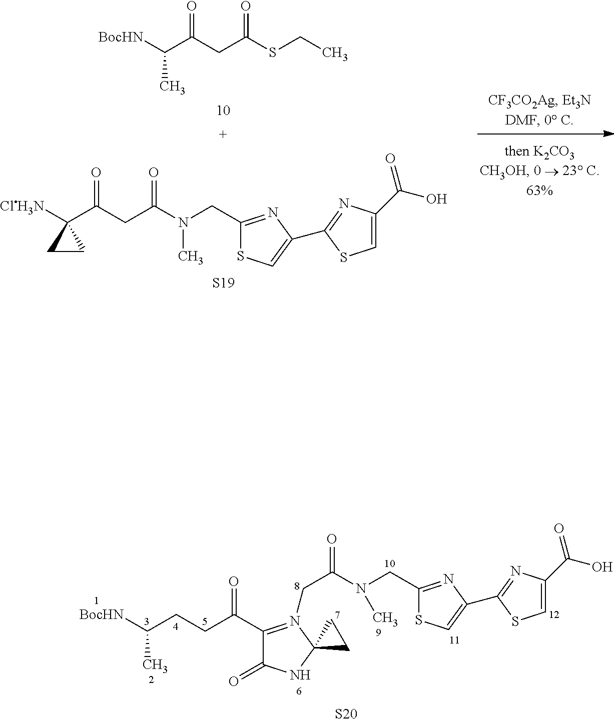

A solution of the thioester 10 (397 mg, 1.31 mmol, 1.30 equiv) in N,N-dimethylformamide (2.0 mL) was added dropwise via syringe over 20 min to a mixture of silver trifluoroacetate (445 mg, 2.01 mmol, 2.00 equiv), triethylamine (562 μL, 4.03 mmol, 4.00 equiv), and the amine S19 (420 mg, 1.01 mmol, 1 equiv) in N,N-dimethylformamide (10 mL) at 0° C. The reaction mixture was stirred for 1 h at 0° C. Potassium carbonate (418 mg, 3.02 mmol, 3.00 equiv) and methanol (10 mL) were then added in sequence to the reaction mixture at 0° C. The reaction mixture was allowed to warm to 23° C. and stirred at this temperature for 6 h. The heterogeneous product mixture was filtered through a fritted funnel. The filter cake was washed with methanol (10 mL). The filtrates were combined and the combined filtrates were concentrated. The residue obtained was applied to a trimethylamine acetate-functionalized silica column (Si-TMA acetate; eluting with 0.5% formic acid-acetonitrile). The fractions containing the product S20 were collected, combined, and concentrated to provide the lactam S20 as a white solid (380 mg, 63%).

In DMSO-d₆ at room temperature the title compound exists as a mixture (3:1) of amide-bond rotamers. The NMR signals are reported for the major isomer.

¹H NMR (600 MHz, DMSO-d₆) δ 8.62 (bs, 1H, H₆), 8.44 (s, 1H, H₁₂), 8.28 (s, 1H, H₁₁), 6.63 (bs, 1H), 4.82 (d, J=15.7 Hz, 1H, H₁₀), 4.78 (d, J=15.8 Hz, 1H, H₁₀), 3.62 (d, J=16.2 Hz, 1H, H₈), 3.55 (d, J=16.2 Hz, 1H, H₈), 3.46-3.38 (m, 1H, H₃), 3.18 (s, 3H, H₉), 2.91-2.83 (m, 2H, H₅), 1.62-1.49 (m, 4H, H₄, H₇), 1.50-1.40 (m, 2H, H₇), 1.36 (s, 9H, H₁), 1.00 (d, J=6.3 Hz, 3H, H₂). ¹³C NMR (151 MHz, DMSO-d₆) δ 197.2 (C), 169.0 (C), 168.9 (C), 168.4 (C), 167.7 (C), 166.8 (C), 162.2 (C), 155.0 (C), 147.2 (C), 130.5 (C), 128.5 (CH), 118.7 (CH), 77.3 (C), 48.8 (CH₂), 45.5 (C), 45.4 (CH), 38.4 (CH₂), 35.9 (CH₃), 29.9 (CH₂), 29.7 (CH₂), 28.3 (CH₃), 20.9 (CH₃), 13.3 (CH₂). IR (ATR-FTIR), cm⁻¹: 3328 (br w), 2975 (w), 1792 (w), 676 (s), 1631 (s), 1501 (m), 1391 (m), 1169 (s), 1152 (s), 674 (w), 556 (m). HRMS-CI (m/z): [M+Na]⁺ calcd for C₂₇H₃₃N₅NaO₇S₂, 626.1714; found, 626.1716. [α]_{D}²⁰ +8.0 (c 1.0, DMSO).

### Synthesis of the Amide 19:

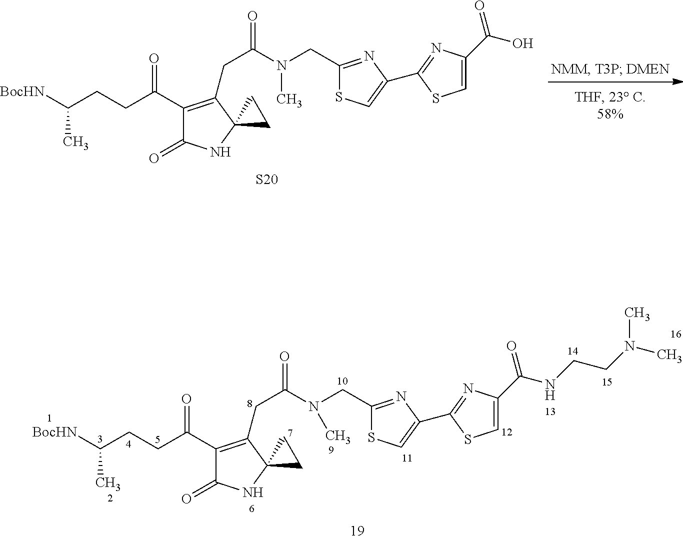

A solution of T3P in ethyl acetate (50 wt %, 118 μL, 199 μmol, 1.50 equiv) and 4-methylmorpholine (72.8 μL, 663 μmol, 5.00 equiv) were added in sequence to a solution of the acid S20 (80.0 mg, 133 μmol, 1 equiv) in tetrahydrofuran (2.7 mL) at 23° C. The reaction mixture was stirred for 20 min at 23° C. N,N-Dimethylethylenediamine (36.2 μL, 331 μmol, 2.50 equiv) was added to the reaction mixture. The resulting mixture was stirred for 14 h at 23° C. The product mixture was concentrated. The concentrated product mixture was diluted with ethyl acetate (10 mL). The diluted product mixture was poured into a separatory funnel that had been charged with saturated aqueous sodium bicarbonate solution (5.0 mL) and the layers that formed were separated. The aqueous layer was extracted with ethyl acetate (2×10 mL). The organic layers were combined and the combined organic layers were dried over sodium sulfate. The dried solution was filtered and the filtrate was concentrated to provide the amide 19 as a white solid (52.0 mg, 58%).

The product 19 obtained in this way was estimated to be of >95% purity by ¹H and ¹³C NMR analysis (see accompanying spectra) and was used without further purification. In DMSO-d₆ at room temperature the title compound exists as a mixture (3:1) of amide-bond rotamers. The NMR signals are reported for the major isomer.

¹H NMR (600 MHz, DMSO-d₆) δ 8.62 (s, 1H, H₆), 8.27 (bs, 1H, H₁₂), 8.24 (s, 1H, H₁₁), 8.23 (bs, 1H, H₁₃), 6.63 (bs, 1H), 4.82 (d, J=15.9 Hz, 1H, H₁₀), 4.79 (d, J=15.9 Hz, 1H, H₁₀), 3.62 (d, J=16.2 Hz, 1H, H₈), 3.55 (d, J=16.1 Hz, 1H, H₈), 3.50-3.40 (m, 1H, H₃), 3.39 (app q, J=6.4 Hz, 2H, H₁₄), 3.18 (s, 3H, H₉), 2.91-2.80 (m, 2H, H₅), 2.42 (td, J=6.6, 1.8 Hz, 2H, H₁₅), 2.18 (s, 6H, H₁₆), 1.64-1.48 (m, 4H, H₄, H₇), 1.47-1.38 (m, 2H, H₇), 1.36 (s, 9H, H₁), 1.00 (d, J=6.5 Hz, 3H, H₂). ¹³C NMR (151 MHz, DMSO-d₆) δ 197.2 (C), 169.1 (C), 169.0 (C), 168.4 (C), 166.8 (C), 161.8 (C), 160.2 (C), 155.0 (C), 150.8 (C), 147.1 (C), 130.5 (C), 124.1 (CH), 118.7 (CH), 77.3 (C), 58.1 (CH₂), 48.8 (CH₂), 45.5 (C), 45.4 (CH), 45.2 (CH₃), 38.4 (CH₂), 36.7 (CH₂), 36.0 (CH₁), 29.9 (CH₂), 29.7 (CH₂), 28.3 (CH₃), 20.9 (CH₃), 13.3 (CH₂). IR (ATR-FTIR), cm⁻¹: 3325 (br w), 2975 (w), 2931 (w), 1681 (s), 1654 (s), 1543 (m), 1453 (w), 1165 (m), 766 (w), 621 (w). HRMS-CI (m/z): [M+H]⁺ calcd for C₃₁H₄₄N₇O₆S₂, 674.2789; found, 674.2795. [α]_{D}²⁰ −33.0 (c 1.0, CH₂Cl₂).

### Synthesis of the Amide 15j:

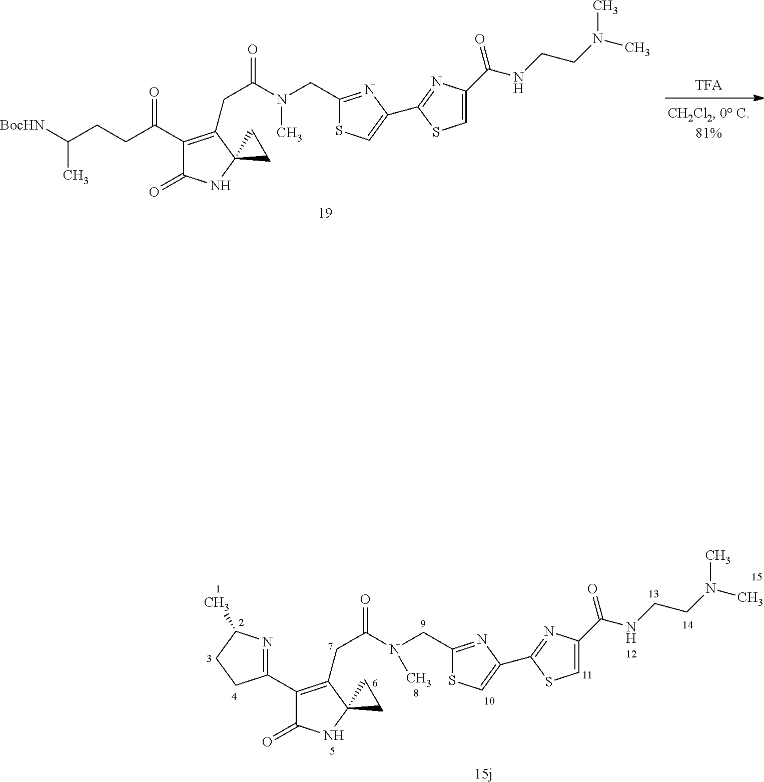

Trifluoroacetic acid (200 μL, 2.61 mmol, 176 equiv) was added dropwise via syringe to a solution of the amide 19 (10.0 mg, 14.8 μmol, 1 equiv) in dichloromethane (400 μL) at 0° C. The reaction mixture was stirred for 1 h at 0° C. The product mixture was concentrated. The concentrated product mixture was diluted with dichloromethane (10 mL). The diluted product mixture was poured into a separatory funnel that had been charged with saturated aqueous sodium bicarbonate solution (5.0 mL) and the layers that formed were separated. The aqueous layer was extracted with dichloromethane (2×10 mL). The organic layers were combined and the combined organic layers were dried over sodium sulfate. The dried solution was filtered and the filtrate was concentrated to provide the amide 15j as a white solid (6.9 mg, 81%).

In DMSO-d₆ at room temperature the title compound exists as a mixture (3:1) of amide-bond rotamers. The NMR signals are reported for the major isomer.

¹H NMR (600 MHz, DMSO-d₆) δ 8.46 (bs, 1H, H₅), 8.27 (s, 1H, H₁₁), 8.25 (bs, 1H, H₁₂), 8.24 (s, 1H, H₁₀), 4.84 (d, J=15.6 Hz, 1H, H₉), 4.70 (d, J=15.6 Hz, 1H, H₉), 3.95-3.88 (m, 1H, H₂), 3.71 (d, J=15.8 Hz, 1H, H₇), 3.60 (d, J=15.8 Hz, 1H, H₇), 3.39 (app q, J=6.4 Hz, 2H, H₁₃), 3.17 (s, 3H, H₈), 3.01-2.92 (m, 1H, H₄), 2.81-2.69 (m, 1H, H₄), 2.41 (t, J=6.6 Hz, 2H, H₁₄), 2.18 (s, 6H, H₁), 2.02-1.92 (m, 1H, H₃), 1.51-1.43 (m, 2H, H₆), 1.39-1.30 (m, 2H, H₆), 1.28-1.21 (m, 1H, H₃), 1.07 (d, J=6.7 Hz, 3H, H₁). ¹³C NMR (151 MHz, DMSO-d₆) δ 170.3 (C), 169.0 (C), 168.9 (C), 166.8 (C), 161.8 (C), 160.2 (C), 159.3 (C), 150.8 (C), 147.1 (C), 127.2 (C), 124.1 (CH), 118.7 (CH), 66.9 (CH), 58.1 (CH₂), 48.8 (CH₂), 45.4 (C), 45.2 (CH₃), 36.7 (CH₂), 36.1 (CH₃), 29.7 (CH₂), 29.6 (CH₂), 22.0 (CH₃), 12.2 (CH₂), 12.0 (CH₂). HRMS-CI (m/z): [M+H]⁺ calcd for C₂₆H₃₄N₇O₃S₂, 556.2159; found, 556.2167. [α]_{D}²⁰ −38.0 (c 1.0, CH₂Cl₂).

### FIRST SET OF REFERENCES (FIRST SET OF EXAMPLES)

(1) Vizcaino, M. I.; Crawford, J. M. Nat. Chem. 2015, 7, 411.
(2) Brotherton, C. A.; Wilson, M.; Byrd, G.; Balskus, E. P. Org. Lett. 201$, 17, 1545.
(3) Vizcaino, M. I.; Engel, P.; Trautman, E.; Crawford, J. M. J. Am. Chem. Soc. 2014, 136, 9244.
(4) Jorgensen, W. L.; Tirado-Rives, J. J. Comput. Chem. 2005, 26, 1689.
(5) Frisch, M. J.; Trucks, G. W.; Schlegel, H. B.; Scuseria, G. E.; Robb, M. A.; Cheeseman, J. R.; Scalmani, G.; Barone, V.; Mennucci, B.; Petersson, G. A.; Nakatsuji, H.; Caricato, M.; Li, X.; Hratchian, H. P.; Izmaylov, A. F.; Bloino, J.; Zheng, G.; Sonnenberg, J. L.; Hada, M.; Ehara, M.; Toyota, K.; Fukuda, R.; Hasegawa, J.; Ishida, M.; Nakajima, T.; Honda, Y.; Kitao, O.; Nakai, H.; Vreven, T.; Montgomery Jr, J. A.; Peralta, J. E.; Ogliaro, F.; Bearpark, M. J.; Heyd, J.; Brothers, E. N.; Kudin, K. N.; Staroverov, V. N.; Kobayashi, R.; Normand, J.; Raghavachari, K.; Rendell, A. P.; Burant, J. C.; Iyengar, S. S.; Tomasi, J.; Cossi, M.; Rega, N.; Millam, N. J.; Klene, M.; Knox, J. E.; Cross, J. B.; Bakken, V.; Adamo, C.; Jaramillo, J.; Gomperts, R.; Stratmann, R. E.; Yazyev, O.; Austin, A. J.; Cammi, R.; Pomelli, C.; Ochterski, J. W.; Martin, R. L; Morokuma, K.; Zakrzewski, V. G.; Voth, G. A.; Salvador, P.; Dannenberg, J. J.; Dapprich, S.; Daniels, A. D.; Farkas, Ö.; Foresman, J. B.; Ortiz, J. V.; Cioslowski, J.; Fox, D. J.; Gaussian, Inc. %! Gaussian 09: Wallingford, Conn., USA, 2009.
(6) Still, W. C.; Kahn, M.; Mitra, A. J. Org. Chem. 1978, 43, 2923.
(7) SiliaBond® TMA Acetate: a NEW strong anion exchange sorbent and its applications in SPE format; François Béland, N. H., Sleeves Potvin and Lynda Tremblay, Ed.; SiliCycle, 2009.
(8) Pangborn, A. B.; Giardello, M. A.; Grubbs, R. H.; Rosen, R. K.; Timmers, F. J. Organometallics 15196, 15, 1518.
(9) Babij, N. R.; Wolfe, J. P. Angew. Chem., Int. Ed. 2012, 51, 4128.
(10) Bae, H. Y.; Sim, J. H.; Lee, J.-W.; List, B.; Song, C. E. Angew. Chem., Int. Ed. 2013, 52, 12143.
(11) Videnov, G.; Kaiser, D.; Kempter, C.; Jung, G. Angew. Chem., Int. Ed. 1996, 35, 1503.
(12) Fustero, S.; Monteagudo, S.; Sanchez-Rosello, M.; Flores, S.; Barrio, P.; del Pozo, C. Chem.—Eur. J. 2010, 16, 9835.
(13) Liu, G.; Cogan, D. A.; Ellman, J. A. J. Am. Chem. Soc. 1997, 119, 9913.
(14) Kaiser, F.; Koerber, K.; Von Deyn, W.; Deshmukh, P.; Narine, A.; Dickhaut, J.; Bandur, N. G.; Langewald, J.; Culbertson, D. L.; Anspaugh, D. D.; BASF SE, Germany. 2012, p 291pp.
(15) Thompson, R. E.; Jolliffe, K. A.; Payne, R. J. Org. Lett. 2011, 13, 680.
(16) Bhansali, P.; Hanigan, C. L.; Casero, R. A.; Tillekeratne, L. M. V. J. Med. Chem. 2011, 54, 7453.
(17) Souto, J. A.; Vaz, E.; Lepore, I.; Pöppler, A.-C.; Franci, G.; Álvarez, R.; Altucci, L.; de Lera, Á. R. J. Med. Chem. 2010, 53, 4654.

### SECOND SET OF REFERENCES (SECOND SET OF EXAMPLES)

(1) Still, W. C.; Kahn, M.; Mitra, A. J. Org. Chem. 1978, 43, 2923.
(2) SiliaBond® TMA Acetate: a NEW strong anion exchange sorbent and its applications in SPE format; François Béland, N. H., Steeves Potvin and Lynda Tremblav, Ed.; SiliCycle, 2009.
(3) Pangborn, A. B.; Giardello, M. A.; Grubbs, R. H.; Rosen, R. K.; Timmers, F. J. Organometallics 1996, 15, 1518.
(4) Tanaka, A.; Usuki, T. Tetrahedron Leu. 2011, 52, 5036.
(5) Healy, A. R.; Vizcaino, M. I.; Crawford, J. M.; Herzon, S. B. J. Am. Chem. Soc. 2016, 138, 5426.
(6) Bae, H. Y.; Sim, J. H.; Lee, J.-W.; List, B.; Song, C. E. Angew. Chem., Int. Ed. 2013, 52, 12143.
(7) Hickey, S. M.; Ashton, T. D.; Khosa, S. K.; Pfeffer, F. M. Synlett 2012, 23, 1779.

### REFERENCES

1. Donia, M. S.; Fischbach, M. A. Science 2015, 349, 395.
2. Xu, J.: Gordon, J. I. Proc Natl Acad Sci USA 2003, 100, 10452.
3. The colibactin gene cluster has also been referred to as “pits” in the literature.
4. Nougayréde, J.-P.; Homburg, S.; Taieb, F.; Boury, M.; Brzuszkiewicz, E.; Gottschalk, G.; Buchrieser, C; Hacker, J.; Dobrindt, U.; Oswald, E. Science 2006, 313, 848.
5. (a) Brotherton, C. A.; Balskus, E. P. J. Am, Chem. Soc. 2013, 135, 3359. (b) Bian, X.; Fu, J.; Plaza, A.; Herrmann, J.; Pistorius, D.; Stewart, A. F.; Zhang, Y.; Muller, R. Chembiochem 2013, 14, 1194. (c) Vizcaino, M. I.; Engel, P.; Trautman, E.; Crawford, J. M. J. Am. Chem. Soc. 2014, 136, 9244.
6. Cuevas-Ramos, G.; Petit, C. R.; Marcq, I.; Boury, M.; Oswald, E.; Nougayréde, J.-P. Proc. Natl. Acad. Sci U.S.A 2010, 107, 11537.
7. Arthur, J. C., et al. Science 2012, 338, 120.
8. Arthur, J. C; Gharaibeh, R. Z.; Mühlbauer, M.; Perez-Chanona, E.; Uronis, J. M.; McCafferty, J.; Fodor, A. A.; Jobin, C. Nat. Common. 2014, J, 4724.
9. (a) Vizcaino, M. I.; Crawford, J. M. Nat. Chem. 2015, 7, 411. (b) Bian, X.; Plaza, A.; Zhang, Y.; Mailer, R. Chem. Sci. 2015, 6, 3154. (c) Brotherton, C. A.; Wilson, M.; Byrd, G.; Balskus, E. P. Org. Lett. 2015, 77, 1545.
10. 0.1 mg of 5 was obtained from a 200 L fermentation of an optimized producing strain, see ref. Li et al.
11. Li, Z. R.; Li, Y.; Lai, J. Y.; Tang, J.; Wang, B.; Lu, L.; Zhu, G.; Wu, X., Xu, Y.; Qian, P. Y. Chembiochem 2015, 16, 1715.
12. Zhang, J.; Walter, J. C. DNA Repair 2014, 19, 135.
13. Tichenor, M. S.; Boger, D. L. Nat. Prod. Rep. 2008, 25, 220.
14. Wu, W.; Vanderwall, D. E.; Turner, C. J., Hoehn, S.; Chen, J.; Kozarich, J. W.; Stubbe, J. Nucleic Acids Ret. 2002, 30, 4881.
15. Supporting Information—please see attached.

1. A compound according to the chemical structure I: 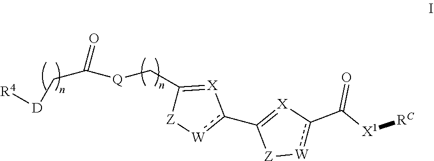
Where X is N or C—R;
W is N, N—R^{N}, C—R or CR(R) (preferably the variable bond between W and the adjacent carbon atom is a double bond and W is N or C—R);
Each Z is independently S, O, N—R^{N} or C—R(R);
Each R is independently H, a C₁-C₆ (preferably C₁-C₃) alkyl group optionally substituted with one or two hydroxyl groups or up to three halogen (F, Cl, Br, I, preferably F or Cl, most often F) groups, or a O—(C₁-C₃) alkoxy group;
Each R^{N} is independently H or a C₁-C₆ (preferably C₁-C₃) alkyl group optionally substituted with one or two hydroxyl groups or up to three halogen groups, preferably H or methyl;
Q is O, S, N(R¹) or C(R^{Q})R^{Q};
X¹ is O, S, N(R³) or C(R^{X})R^{X};
D is 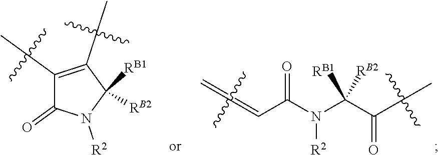
R¹, R² and R³ are each independently H or a C₁-C₆ (preferably C₁-C₃) alkyl group which is optionally substituted with one or two hydroxyl groups;
R⁴ is 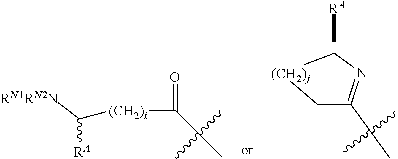
When D is 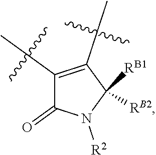
R⁴ is 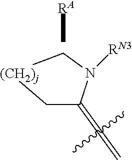
when D is 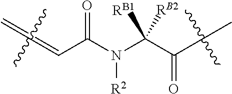
(the double bond is the same in both moieties);
Where R^{A} is H or an optionally substituted C₁-C₈ alkyl or alkene group, preferably H or a C₁-C₃ alkyl, most often methyl;
R^{N1} and R^{N2} are each independently H, a C₁-C₆ (preferably C₁-C₃) alkyl group which is optionally substituted with one or two hydroxyl groups or up to three halogen groups, a protecting group (P_{G}), preferably a BOC group) or a targeting element T_{E} which is linked to the nitrogen by linker group L_{C} which is optionally cleavable;
R^{N3} is absent, H, a C₁-C₆ (preferably C₁-C₃) alkyl group which is optionally substituted with one or two hydroxyl groups, a protecting (P_{G}), preferably a BOC group) or a targeting element T_{E} which is linked to the nitrogen by a linker group L_{C} which is optionally cleavable;
R^{Q} and R^{X} are each independently H or a C₁-C₆ (preferably C₁-C₃) alkyl group which is optionally substituted with one or two hydroxyl groups or up to three halogen groups;
i is 1-4, preferably 2-4;
j is 1-3;
each n is independently 1, 2 or 3 (preferably 1);
R^{B1} and R^{B2} are each independently H, a C₁-C₆ (preferably C₁-C₃) group which is optionally substituted with one or two hydroxyl groups or up to three halogen groups or together R^{B1} and R^{B2} form a cyclopropyl or cyclobutyl group (preferably, R^{B1} and R^{B2} are each independently H, methyl or together form a cyclopropyl group);
R^{C} is H, a C₁-C₁₂ optionally substituted alkyl or alkene group (preferably substituted with one or two hydroxyl groups, up to five halo groups) or a —(CH₂)ₙ₁NR₁R₂ group where R₁ and R₂ are each independently H or a C₁-C₆ optionally substituted alkyl group and n1 is 1-8 (preferably 1, 2, 3, 4 or 5), a protecting group (P_{G}) (preferably a BOC group) or a targeting element T_{E} which is linked to X¹ by a linker group L_{C} which is optionally cleavable, or R^{C} forms a dimer compound through a covalent linker group L which is optionally cleavable, said dimer compound having the general chemical structure: 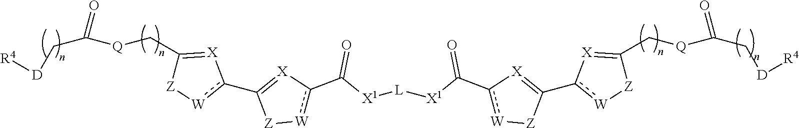
Where X, W, Z, Q, X¹, D, R², R⁴, n, R^{B1} and R^{B2} are the same as above; and
L is a linker group which is optionally cleavable and covalently links the dimeric portions of the molecule to each other, or
a pharmaceutically acceptable salt, stereoisomer, solvate or polymorph thereof.

2. The compound of claim 1 wherein the bond between W and the carbon is a double bond, each n is 1, W is C—R, R is H or methyl, X is N, Z is S or N—R^{N}, R^{N} is H or methyl, Q is C(R^{Q})R^{Q}, each R^{Q} is independently H or methyl, preferably both R^{Q} are H, X¹ is NH or N-methyl, R² is H or methyl (preferably H), R^{A} in R⁴ is methyl, R^{B1} and R^{B2} are each independently H or methyl or together form a cyclopropyl group, LC is a cleavable linker and L is a polyethylene glycol linker having between 2 and 12 ethylene glycol units or a —(CH₂)ₘN(R)(CH₂)ₘ— group where R is H or a C₁-C₃ alkyl group (preferably H or methyl) and each m is independently from 1-12 (preferably, 1-10, more preferably 1, 2, 3, 4, 5, or 6).

3. A compound of claim 1 according to the chemical structure II: 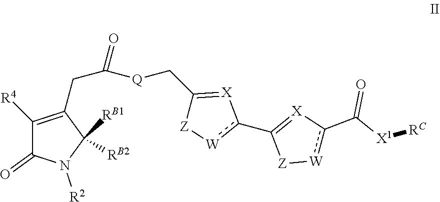
Where X is N or C—R;
W is N, N—R^{N}, C—R or CR(R) (preferably the variable bond between W and the adjacent carbon atom is a double bond and W is N or C—R);
Each Z is independently S, O, N—R^{N} or C—R(R);
Each R is independently H, a C₁-C₆ (preferably C₁-C₃) alkyl group optionally substituted with one or two hydroxyl groups or up to three halogen groups, or a O—(C₁-C₃) alkoxy group;
Each R^{N} is independently H or a C₁-C₆ (preferably C₁-C₃) alkyl group optionally substituted with one or two hydroxyl groups or up to three halogen groups, preferably H or methyl;
Q is O, S, N(R¹) or C(R^{Q})R^{Q};
X¹ is O, S, N(R³) or C(R^{X})R^{X};
R¹, R² and R³ are each independently H or a C₁-C₆ (preferably C₁-C₃) alkyl group which is optionally substituted with one or two hydroxyl groups or up to three halogens groups;
R⁴ is 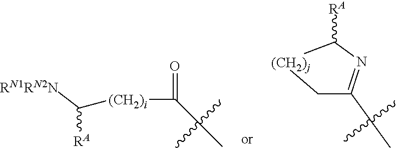
Where R^{A} is H or an optionally substituted C₁-C₈ alkyl or alkene group, preferably H or a C₁-C₃ alkyl, most often methyl;
R^{N1} and R^{N2} are each independently H, a C₁-C₆ (preferably C₁-C₃) alkyl group which is optionally substituted with one or two hydroxyl groups or up to three halogen groups, a protecting (P_{G}) (preferably a BOC group) or a targeting element T_{E} which is linked to the nitrogen by a linker L_{C} which is optionally cleavable;
R^{Q} and R^{X} are each independently H or a C₁-C₆ (preferably C₁-C₃) alkyl group which is optionally substituted with one or two hydroxyl groups or up to three halogen groups;
i is 1-4, preferably 2-4;
j is 1-3;
R^{B1} and R^{B2} are each independently H, a C₁-C₆ (preferably C₁-C₃) group which is optionally substituted with one or two hydroxyl groups or up to three halogen groups, or together R^{B1} and R^{B2} form a cyclopropyl or cyclobutyl group (preferably, R^{B1} and R^{B2} are each independently H, methyl or together form a cyclopropyl group);
R^{C} is H, a C₁-C₁₂ optionally substituted alkyl or alkene group (preferably substituted with one or two hydroxyl groups, up to five halo groups) or a —(CH₂)ₙNR₁R₂ group where R₁ and R₂ are each independently H or a C₁-C₆ optionally substituted alkyl group and n is 1-8 (preferably 1, 2, 3, 4 or 5), a protecting (P_{G}) (preferably a BOC group) or a targeting element T_{E} which is linked to X¹ (preferably through a nitrogen) by a linker L_{C} which is optionally cleavable, or R^{C} forms a dimer compound through a covalent linker group L which is optionally cleavable, said dimer compound having the general chemical structure: 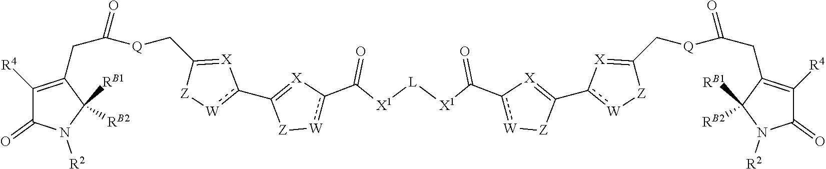
Where X, W, Z, Q, X¹, R², R⁴, R^{B1} and R^{B2} are the same as above; and L is a linker group which is optionally cleavable and which covalently links the dimeric portions of the molecule to each other, or
a pharmaceutically acceptable salt, stereoisomer, solvate or polymorph thereof.

4. The compound of claim 3 wherein W is C—R, R is H or methyl, X is N, Z is S or N—R^{N}, R^{N} is H or methyl, Q is N—H or C(R^{Q})R^{Q} where each R^{Q} is independently H or methyl, preferably both are H, X¹ is NH or N-methyl, R² is H or methyl (preferably H), R^{A} in R⁴ is methyl, R^{B1} and R^{B2} are each independently H or methyl or together form a cyclopropyl group and L is a linker as otherwise described herein, preferably L is a polyethylene glycol linker having between 2 and 12 ethylene glycol units or a —(CH₂)ₘN(R)CH₂)ₘ— group where R is H or a C₁-C₃ alkyl group (preferably H or methyl) and each m is independently from 1-12 (preferably, 1-10, more preferably 1, 2, 3, 4, 5, or 6).

5. A compound of claim 2 according to the chemical structure III: 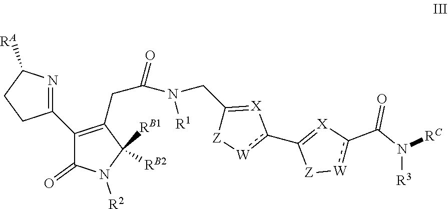
Where X is N or C—R;
W is N, N—R^{N}, C—R or CR(R) (preferably the variable bond between W and the adjacent carbon atom is a double bond and W is N or C—R);
Each Z is independently S, O, N—R^{N} or C—R(R);
Each R is independently H, a C₁-C₃ alkyl group optionally substituted with one or two hydroxyl groups or up to three halogen groups, or a O—(C₁-C₃) alkoxy group;
Each R^{N} is independently H or a C₁-C₃ alkyl group optionally substituted with one or two hydroxyl groups or up to three halogen groups, preferably H or methyl;
R¹, R² and R³ are each independently H or a C₁-C₃ alkyl group which is optionally substituted with one or two hydroxyl groups or up to three halogen groups;
R^{A} is H or an optionally substituted C₁-C₈ alkyl or alkene group, preferably H or a C₁-C₃ alkyl, most often methyl;
R^{B1} and R^{B2} are each independently H, a C₁-C₃ alkyl group which is optionally substituted with one or two hydroxyl groups or up to three halo groups (F, Cl, Br or I, preferably Cl or F, most often F) or together R^{B1} and R^{B2} form a cyclopropyl or cyclobutyl group (preferably, R^{B1} and R^{B2} are each independently H, methyl or together form a cyclopropyl group);
R^{C} is H, a C₁-C₁₂ optionally substituted alkyl or alkene group (preferably substituted with one or two hydroxyl groups, up to five halo groups) or a —(CH₂)ₙNR₁R₂ group where R₁ and R₂ are each independently H or a C₁-C₆ optionally substituted alkyl group and n is 1-8 (preferably 1, 2, 3, 4 or 5), a protecting (P_{G}) (preferably a BOC group) or a targeting element T_{E} which is linked to X¹ (preferably a nitrogen) by a linker L_{C} which is optionally cleavable or R^{C} forms a dimer compound through a covalent linker group L which is optionally cleavable, said dimer compound having the general chemical structure: 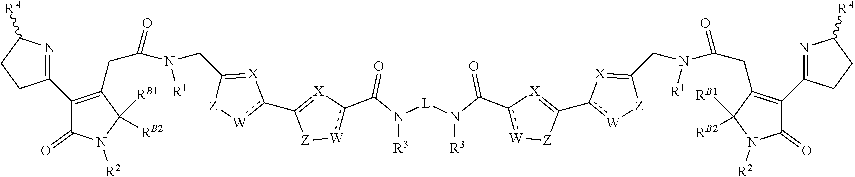
Where X, W, Z, R, R^{N}, R¹, R², R³, R^{A}, R^{B1} and R^{B2} are the same as above; and L is a linker group which covalently links the dimeric portions of the molecule to each other, or
a pharmaceutically acceptable salt, stereoisomer, solvate or polymorph thereof.

6. The compound of claim 5 wherein the variable bond between W and the adjacent carbon atoms in each five-membered ring is a double bond, W is C—R, R is H or methyl, X is N, Z is S or N—R^{N}, R^{N} is H or methyl, Q is C(R^{Q})R^{Q}, each R^{Q} is independently H or methyl, preferably both are H, X¹ is NH or N-methyl, R² is H or methyl (preferably H), R^{A} in R⁴ is methyl, R^{B1} and R^{B2} are each independently H or methyl or together form a cyclopropyl group and L is a linker as otherwise described herein, preferably L is a polyethylene glycol group having between 2 and 12 ethylene glycol units or a —(CH₂)ₘN(R)CH₂)ₘ— group where R is H or a C₁-C₃ alkyl group (preferably H or methyl) and each m is independently from 1-12 (preferably, 1-10, more preferably 1, 2, 3, 4, 5, or 6).

7. A compound of claim 2 according to chemical structure IV: 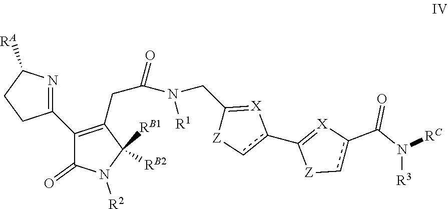
Where X, Z, R¹, R², R³, R^{A}, R^{B1}, R^{B2} and R^{C} are the same as for compound III above,
or a pharmaceutically acceptable salt, stereoisomer thereof.

8. The compound according to claim 7 wherein X is preferably N; Z is preferably S, O, N—H or N—CH₃ (more preferably S); R is preferably H, methyl or OMe; R^{N} is preferably H or methyl; R¹, R² and R³ are each independently preferably H or methyl; R^{A} is preferably H or a C₁-C₃ alkyl, preferably methyl; R^{B1} and R^{B2} are each independently H, methyl or together form a cyclopropyl group and R^{C} is methyl, a —(CH₂)ₙ—N(CH₃)₂ group where n is 1, 2, 3 or 4 (preferably 2), R^{C} forms a guanidine group with the adjacent nitrogen, or R^{C} forms a dimer compound through linker L where L is preferably a
(CH₂)ₘN(R)(CH₂)ₘ— group where R is H or a C₁-C₃ alkyl group (preferably H or methyl) and each m is independently from 1-12 (preferably, 1-10, more preferably 1, 2, 3, 4, 5, or 6).

9. A compound of claim 2 according to chemical structure: 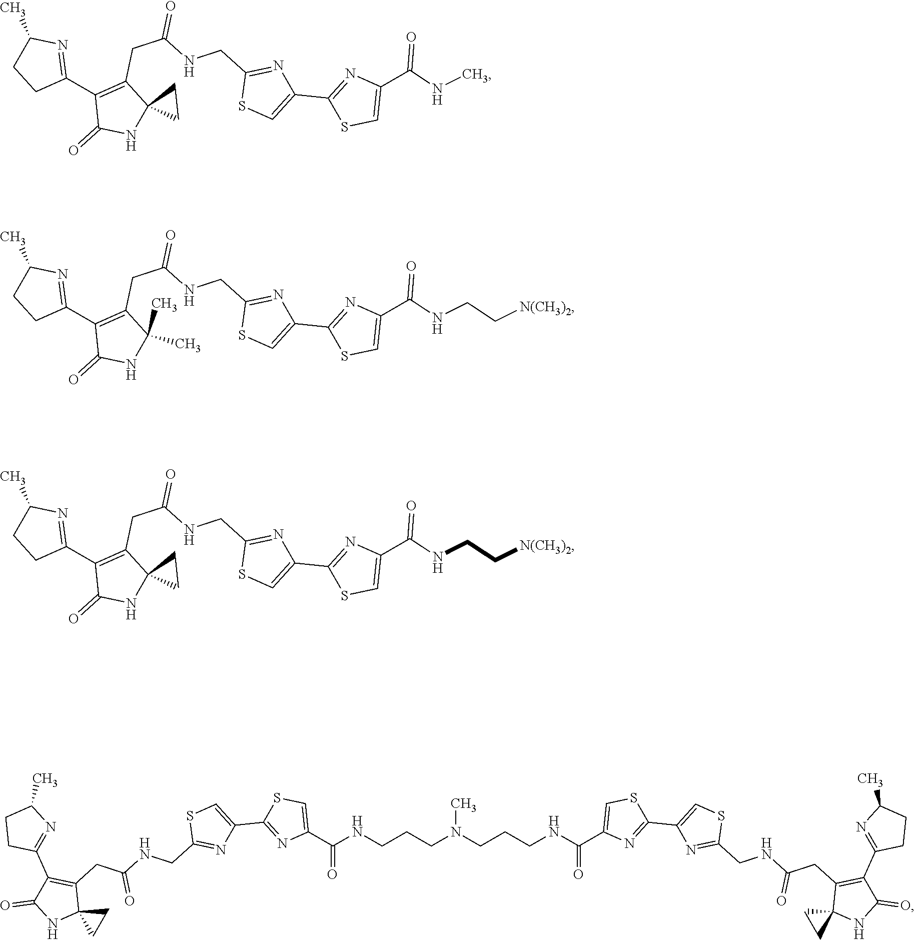
a pharmaceutical salt, stereoisomer, solvate or polymorph thereof.

10. A compound of claim 2 according to chemical structure V: 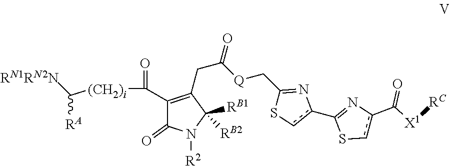
Where Q is CH₂, N—H or N-Me;
X¹ is O, S, N(R³) or C(R^{X})R^{X};
R² and R³ are each independently H or a C₁-C₆ (preferably C₁-C₃) alkyl group which is optionally substituted with one or two hydroxyl groups or up to three halogen groups;
R^{A} is H or an optionally substituted C₁-C₈ alkyl or alkene group, preferably H or a C₁-C₃ alkyl, most often methyl;
R^{N1} and R^{N2} are each independently H, a C₁-C₆ (preferably C₁-C₃) alkyl group which is optionally substituted with one or two hydroxyl groups or up to three halogen groups, a protecting (P_{G}), preferably a BOC group, or a targeting element T_{E} which is linked to the nitrogen by a linker L_{C} which is optionally cleavable;
Each R^{X} is independently H or a C₁-C₆ (preferably C₁-C₃) alkyl group which is optionally substituted with one or two hydroxyl groups or up to three halogen groups (preferably F or Cl, more often F);
i is 1-4, preferably 2-4;
R^{B1} and R^{B2} are each independently H, a C₁-C₆ (preferably C₁-C₃) group which is optionally substituted with one or two hydroxyl groups or up to three halogen groups (preferably F, Cl, Br or I, preferably Cl or F, most often F) or together R^{B1} and R^{B2} form a cyclopropyl or cyclobutyl group (preferably, R^{B1} and R^{B2} are each independently H, methyl or together form a cyclopropyl group);
R^{C} is H, a C₁-C₁₂ optionally substituted alkyl or alkene group (preferably substituted with one or two hydroxyl groups or up to five halo groups), a —(CH₂)ₙ₁NR₁R₂ group where R₁ and R₂ are each independently H or a C₁-C₆ optionally substituted alkyl group and n1 is 1-8 (preferably 1, 2, 3, 4 or 5), a protecting P_{G}, preferably a BOC group, or a targeting element T_{E} which is linked to X¹ (preferably a nitrogen) by linker L_{C} which is optionally cleavable, or R^{C} forms a dimer compound through a covalent linker group L which is optionally cleavable, said dimer compound having the general chemical structure: 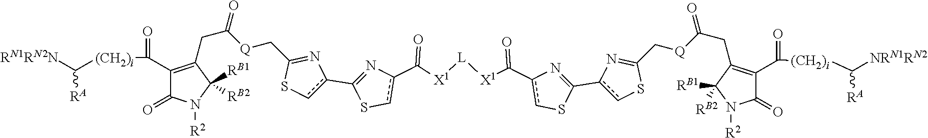
Where Q, R², R^{A}, i, R^{B1}, R^{B2}, R^{N1}, R^{N2}, X¹ and L are the same as for compound V above, and L is a linker group which is optionally cleavable and which covalently links the dimeric portions of the molecule to each other, or
a pharmaceutically acceptable salt, stereoisomer, solvate or polymorph thereof.

11. The compound of claim 10 wherein the variable bond between carbon atoms in each of the five membered rings is a double bond, R² is H or methyl, R^{A} is methyl, i is 1, R^{B1} and R^{B2} are each H, methyl or together form a cyclopropyl group, NR^{N1} and NR^{N2} are each independently H, methyl, a protecting group (preferably a BOC) or a targeting element T_{E} which is linked to the nitrogen by an optionally cleavable linker L_{C}, X¹ is N—H or N-methyl and L is a linker group —(CH₂)ₘN(R)(CH₂)ₘ— where R is H or a C₁-C₃ alkyl group (preferably H or methyl) and each m is independently from 1-12 (preferably, 1-10, more preferably 1, 2, 3, 4, 5, or 6).

12. A compound of claim 2 according to the chemical structure VI: 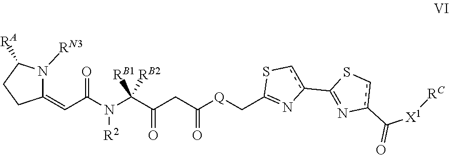
Where Q is CH₂ or N—H;
X¹ is O, S, N(R³) or C(R^{X})R^{X};
R² and R³ are each independently H or a C₁-C₆ (preferably C₁-C₃) alkyl group which is optionally substituted with one or two hydroxyl groups;
Each R^{X} is independently H or a C₁-C₆ (preferably C₁-C₃) alkyl group which is optionally substituted with one or two hydroxyl groups;
R^{A} is H or an optionally substituted C₁-C₈ alkyl or alkene group, preferably H or a C₁-C₃ alkyl, most often methyl;
R^{N3} is H, a C₁-C₆ (preferably C₁-C₃) alkyl group which is optionally substituted with one or two hydroxyl groups, a protecting (P_{G}), preferably a BOC group, or a targeting element T_{E} which is linked to the nitrogen by an optionally cleavable linker L_{C};
R^{B1} and R^{B2} are each independently H, a C₁-C₆ (preferably C₁-C₃) group which is optionally substituted with one or two hydroxyl groups or up to three halo groups (F, Cl, Br or I, preferably Cl or F, most often F) or together R^{B1} and R^{B2} form a cyclopropyl or cyclobutyl group (preferably, R^{B1} and R^{B2} are each independently H, methyl or together form a cyclopropyl group); and
R^{C} is H, a C₁-C₁₂ optionally substituted alkyl or alkene group (preferably substituted with one or two hydroxyl groups, up to five halo groups) or a —(CH₂)ₙ₁NR₁R₂ group where R₁ and R₂ are each independently H or a C₁-C₆ optionally substituted alkyl group and n1 is 1-8 (preferably 1, 2, 3, 4 or 5), a protecting (P_{G}), preferably a BOC group) or a targeting element T_{E} which is linked to X¹ (preferably a nitrogen) by a linker L_{C} which is optionally cleavable, or R^{C} forms a dimer compound through a covalent linker group L which is optionally cleavable, said dimer compound having the general chemical structure: 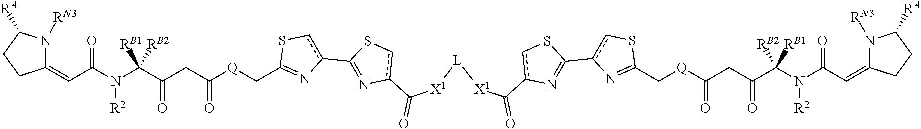
Where Q, R², R^{A}, R^{B1}, R^{B2}, R^{N3} and X¹ are the same as above for compound VI, and L is a linker group which covalently links the dimeric portions of the molecule to each other, or
a pharmaceutically acceptable salt, stereoisomer, solvate or polymorph thereof.

13. The compound according to claim 12 wherein R² is H or methyl, R^{A} is H or methyl, R^{B1} and R^{B2} are each independently H, methyl or together form a cyclopropyl group, R^{N3} is H, methyl, a protecting group (P_{G}), preferably a BOC group, or a targeting element T_{E} which is linked to the nitrogen by an optionally cleavable linker L_{C}, X¹ is N—H or N-methyl and L is a linker group —(CH₂)ₘN(R)(CH₂)ₘ— where R is H or a C₁-C₃ alkyl group (preferably H or methyl) and each m is independently from 1-12 (preferably, 1-10, more preferably 1, 2, 3, 4, 5, or 6).

14. The compound according to claim 1 wherein said targeting element T_{E} is a small molecule which binds to folate receptors (folate receptor binding moiety), a monoclonal antibody, an antibody fragment (FAB) including a single chain variable fragment (scFv) antibody which binds to cancer cells, a PSMA binding moiety, a YSA peptide, a low pH insertion peptide, a group according to the chemical structure 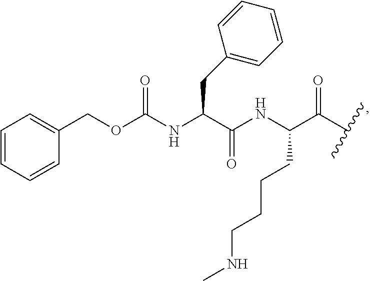 [SMILES: *C(=O)[C@H](CCCCNC)NC(=O)[C@H](Cc1ccccc1)NC(=O)OCc1ccccc1]
or a cysteine-cathepsin moiety.

15. The compound according to claim 1 wherein R^{C} is a protecting group.

16. The compound according to claim 1 wherein L_{C} is a cleavable linker group.

17. The compound according to claim 1 wherein L_{C} is a non-cleavable linker group.

18. The compound according to claim 1 wherein L is a cleavable linker group.

19. The compound according to claim 1 wherein L is a non-cleavable linker group.

20. The compound according to claim 1 wherein L_{C} comprises a group according to the chemical structure: 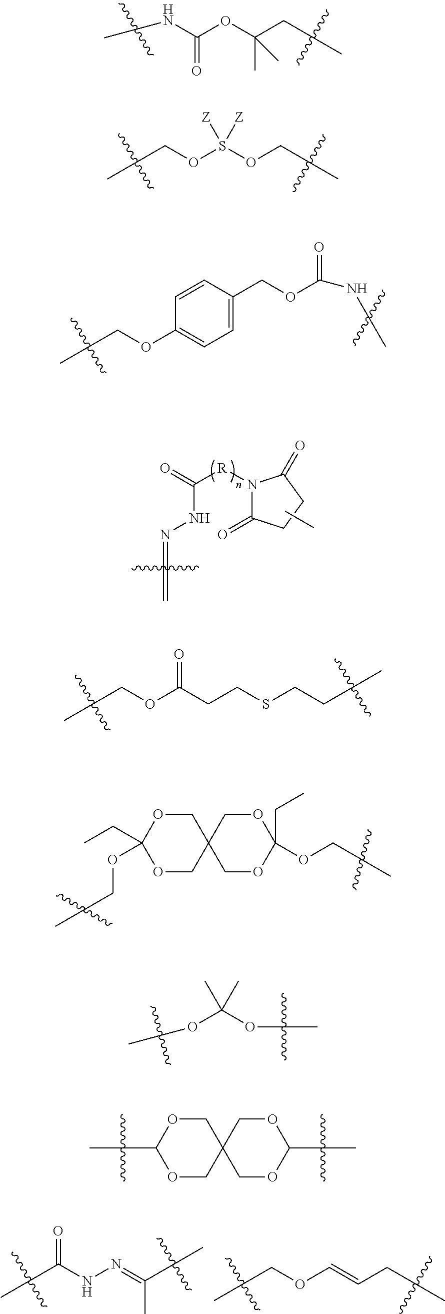 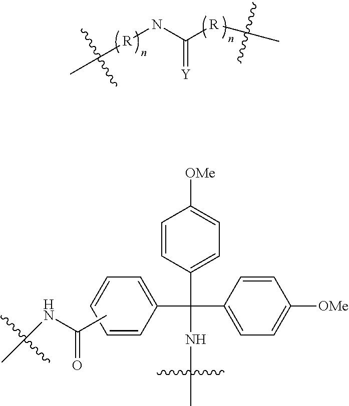
where R is an ethylene glycol group, a methylene group or an amino acid, preferably an ethylene glycol group or an amino acid and n is from 0 to 10, often from 1 to 6, or 1 to 3 and where points of attachment (as indicated) are to other portions of the Linker, a difunctional connector moiety (CON), a non-cleavable (non-labile) linker (L_{N}), or a multifunctional connector molecule [MULTICON], through which an [ACM] functional group and a [CCT_{E}] functional group are linked as otherwise described herein;
X is O, N—R^{AL} or S;
R^{AL} is H or a C₁-C₃ alkyl group (often H or Me, most often H);
Y is O or S and
Z=Me, Et, iPr, tBu, phenyl, each of which may be optionally substituted with one or more halogen groups (especially from three up to five Fs, preferably no more than three Fs) and wherein said phenyl group may be further optionally substituted with a C₁-C₃ alkyl group (which itself may be substituted with up to three halogens, preferably F) or OMe.

21. The compound according to claim 2 wherein L_{C} and/or L comprises a group according to the chemical structure: 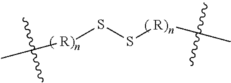
Where R is independently an ethylene glycol group, a methylene group or an amino acid where at least one amino acid (that which provides one of the sulfurs in the disulfide group) is a cysteinyl group and n in this linker is from 0 to 10.

22. The compound according to according to claim 2 and 11-15 wherein L_{C} and/or L comprises a group according to the chemical structure: 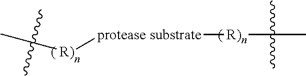
Where the protease substrate is a peptide containing from 2 to 50 amino acid units;
R is an ethylene glycol group or a methylene group and n is from 0 to 10.

23. The compound according to claim 22 wherein said protease substrate consists essentially of the peptide
-Gly-Phe-Leu-Gly-;
-Ala-Leu-Ala-Leu;
-Phe-Arg-;
-Phe-Lys-;
-Val-Cit- (valine-citrillune)
-Val-Lys-; or
-Val-Ala-.

24. The compound according to according to claim 1 wherein L_{C} and/or L comprises a group according to the chemical structure: 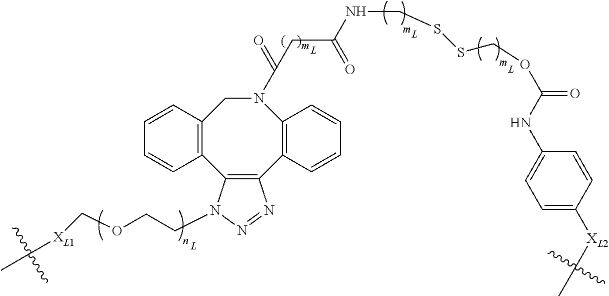
Where X_{L1} is —(CH₂)_{mL′}(C═O)—, —(CH₂)_{mL″}, NR_{1L}, NR_{1L}(C═O), S, S═O or S(O)₂, or a nucleophilic or electrophilic functional group (which can be further reacted to form a covalent link);
X_{L2} is —(CH₂)_{mL′}(C═O)—, —(CH₂)_{mL″}, —(CH₂)_{mL′}NR_{1L}—(CH₂)_{mL″}, NR_{1L}(C═O), S═O or S(O)₂, or a nucleophilic or electrophilic functional group (which can be further reacted to form a covalent link);
R_{1L} is H or a C₁-C₃ alkyl group;
Each m_{L} is independently 1, 2, 3, or 4 (often, each m_{L} is 2);
mL′ is 0, 1, 2, 3, 4, or 5 (preferably 0);
mL″ is 1, 2, 3, 4 or 5; and
n_{L} is 0-20, 1-15, 2-10, 1-6, 1, 2, 3, 4, 5, 6, 7, or 8.

25. The compound according to according to claim 1 wherein L_{C} and/or L comprises a group according to the chemical structure: 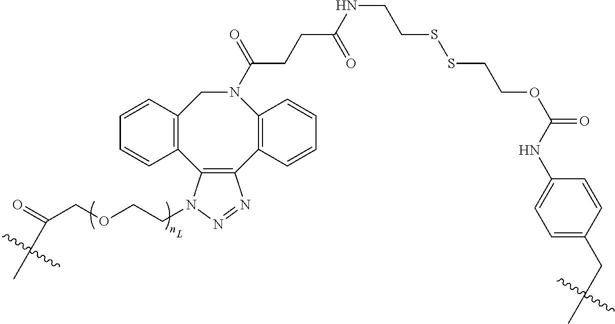
Where n_{L} is 0-20, 1-15, 2-10, 1-6, 1, 2, 3, 4, 5, 6, 7, or 8.

26. The compound according to claim 1 wherein L_{C} and/or L comprises a beta-glucosidase moiety according to the chemical structure:

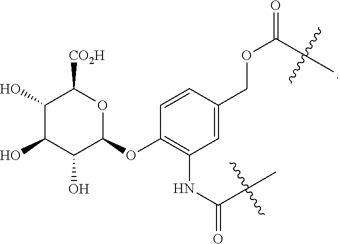 [SMILES: *C(=O)Nc1cc(COC(*)=O)ccc1O[C@@H]1O[C@H](C(=O)O)[C@@H](O)[C@H](O)[C@H]1O]

27. The compound according to claim 1 wherein L_{C} and/or L is a (poly)ethylene glycol linker ranging in length from 2 to about 100 ethylene glycol units or a polyethylene-co-polypropylene (PEG/PPG block copolymer) linker ranging from 2 to about 100 ethylene glycol and propylene glycol units.

28. The compound according to claim 1 wherein L_{C} and/or L comprises a group according to the chemical formula: 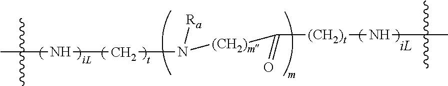
where Rₐ is H or a C₁-C₃ alkyl;
m is an integer from 1 to 12;
m″ is an integer 1, 2, 3, 4, 5, or 6;
t is 0, 1, 2, 3, 4, 5, or 6; and
iL is 0 or 1.

29. The compound according to claim 1 wherein L_{C} and/or L comprises a group according to the chemical structure: 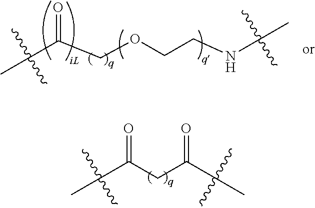
Where q is an integer from 0-12;
q′ is 1 to 12 and
iL is 0 or 1.

30. The compound according to claim 1 wherein L_{C} and/or L comprises a group is according to the chemical structure: 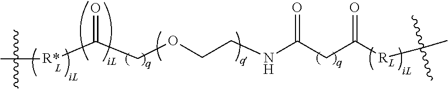
Where q is an integer from 0-12, preferably 0, 1, 2, 3, 4, 5 or 6;
q′ is 1 to 12, often 1, 2, 3, 4, 5 or 6;
iL is 0 or 1; and
R_{L} is an amino acid or an oligopeptide.

31. The compound according to claim 1 wherein L is a group according to the chemical structure: 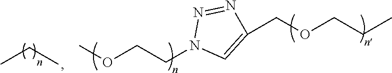
where n and n′ are each independently 0 to 100, preferably 1 to 100, more preferably about 2 to about 20, about 2 to about 10, about 4 to about 10, about 4 to about 8; or
a group according to the chemical structure: 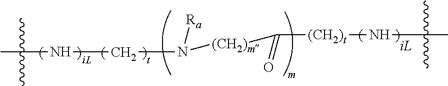
where Rₐ is H or a C₁-C₃ alkyl, preferably CH₃, most often H;
m is an integer from 1 to 12, often 1, 2, 3, 4, 5, or 6;
m″ is an integer 1, 2, 3, 4, 5, or 6, often 6;
t is 0, 1, 2, 3, 4, 5, or 6; and
iL is 0 or 1, often 1; or
a linker according to the structure: 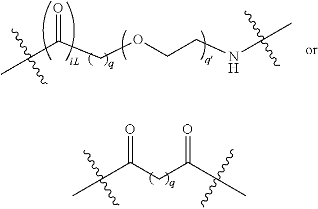
Where q is an integer from 0-12, preferably 1, 2, 3, 4, 5 or 6;
q′ is 1 to 12, often 1, 2, 3, 4, 5 or 6 and
iL is 0 or 1, preferably 1, or
a group according to the chemical structure: 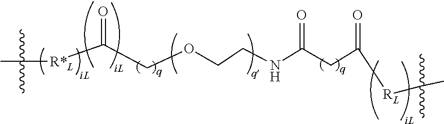
Where q is an integer from 0-12, preferably 0, 1, 2, 3, 4, 5 or 6;
q′ is 1 to 12, often 1, 2, 3, 4, 5 or 6;
iL is 0 or 1; and
R_{L} is an amino acid or an oligopeptide (which term includes a dipeptide) as otherwise described herein, especially including lysine, dilysine, or glycinelysine, or
a group based upon succinimide according to the chemical structure: 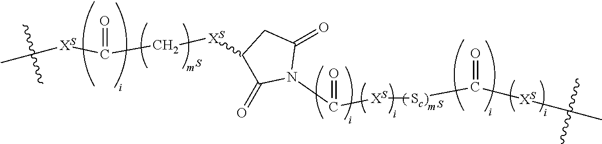
where each X^{S} is independently a bond, S, O or N—R^{S}, preferably S;
R^{S} is H or C₁₋₃ alkyl, preferably H;
S_{c} is CH₂; CH₂O; or CH₂CH₂O;
i is 0 or 1; and
m^{S} is 0, 1, 2, 3, 4, 5, 6, 7, 8, 9 or 10 (preferably 1-5), or
a group which is an amino acid, dipeptide or oligopeptide containing from 1 to 12, preferably 1 to 6 amino acid units, or
a group according to the chemical structure: 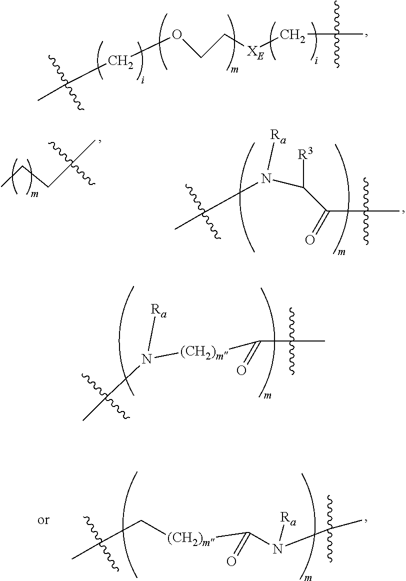
or a polypropylene glycol or polypropylene-co-polyethylene glycol linker having between 1 and 100 glycol units (1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 52 and 50, 3 and 45);
where Rₐ is H, C₁-C₃ alkyl or alkanol or forms a cyclic ring with R³ (to form proline) and R³ is a side chain derived of an amino acid preferably selected from the group consisting of alanine (methyl), arginine (propyleneguanidine), asparagine (methylenecarboxyamide), aspartic acid (ethanoic acid), cysteine (thiol, reduced or oxidized di-thiol), glutamine (ethylcarboxyamide), glutamic acid (propanoic acid), glycine (H), histidine (methyleneimidazole), isoleucine (1-methylpropane), leucine (2-methylpropane), lysine (butyleneamine), methionine (ethylmethylthioether), phenylalanine (benzyl), proline (R³ forms a cyclic ring with Rₐ and the adjacent nitrogen group to form a pyrrolidine group), serine (methanol), threonine (ethanol, 1-hydroxyethane), tryptophan (methyleneindole), tyrosine (methylene phenol) or valine (isopropyl);
X_{E} is a bond, O, N—R_{NA}, or S;
R_{NA} is H or C₁-C₃ alkyl, preferably H;
i is an integer from 0 to 6 (0, 1, 2, 3, 4, 5, or 6);
m″ is an integer from 0 to 25, preferably 1 to 10, 1 to 8, 1, 2, 3, 4, 5, or 6;
m is an integer from 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5; and
n is an integer from 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5; or
a group according to the chemical formula: 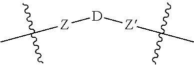
Where Z and Z′ are each independently a bond, —(CH₂)ᵢ—O, —(CH₂)ᵢ—S, —(CH₂)ᵢ—N—R, 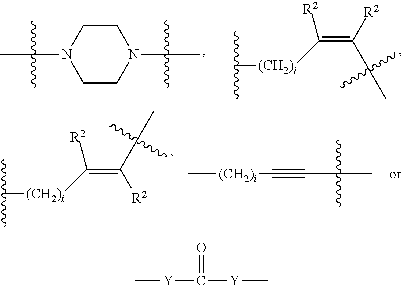
wherein said —(CH₂)ᵢ group, if present in Z or Z′, is bonded to [ACM], [CCT_{E}], or an optional difunctional connector group [CON], if present;
Each R is independently H, or a C₁-C₃ alkyl or alkanol group;
Each R² is independently H or a C₁-C₃ alkyl group;
Each Y is independently a bond, O, S or N—R;
Each i is independently 0 to 100, 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5;
D is 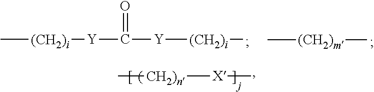
or a bond, or D may be 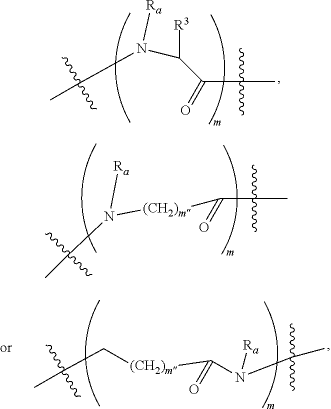
or a polypropylene glycol or polypropylene-co-polyethylene glycol linker having between 1 and 100 glycol units (1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 52 and 50, 3 and 45);
with the proviso that Z, Z′ and D are not each simultaneously bonds;
j is 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5;
m (within this context) is an integer from 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5; and
n (within this context) is an integer from about 1 to 100, about 1 to 75, about 1 to 60, about 1 to 50, about 1 to 45, about 1 to 35, about 1 to 25, about 1 to 20, about 1 to 15, 2 to 10, about 4 to 12, about 5 to 10, about 4 to 6, about 1 to 8, about 1 to 6, about 1 to 5, about 1 to 4, about 1 to 3, etc.).
m′ is 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5;
m″ is an integer between 0 to 25, preferably 1 to 10, 1 to 8, 0, 1, 2, 3, 4, 5, or 6;
n′ is 1 to 100, 1 to 75, 1 to 60, 1 to 55, 1 to 50, 1 to 45, 1 to 40, 2 to 35, 3 to 30, 1 to 15, 1 to 10, 1 to 8, 1 to 6, 1, 2, 3, 4 or 5;
X¹ is O, S or N—R;
R is as described above;
R, is H, C₁-C₃ alkyl or alkanol or forms a cyclic ring with R³ (proline) and R³ is a side chain derived of an amino acid preferably selected from the group consisting of alanine (methyl), arginine (propyleneguanidine), asparagine (methylenecarboxyamide), aspartic acid (ethanoic acid), cysteine (thiol, reduced or oxidized di-thiol), glutamine (ethylcarboxyamide), glutamic acid (propanoic acid), glycine (H), histidine (methyleneimidazole), isoleucine (1-methylpropane), leucine (2-methylpropane), lysine (butyleneamine), methionine (ethylmethylthioether), phenylalanine (benzyl), proline (R³ forms a cyclic ring with Rₐ and the adjacent nitrogen group to form a pyrrolidine group), serine (methanol), threonine (ethanol, 1-hydroxyethane), tryptophan (methyleneindole), tyrosine (methylene phenol) or valine (isopropyl).

32. The compound according to claim 1 wherein L_{C} and/or L contains an optional [CON] group.

33. The compound according to claim 32 wherein said [CON] group is 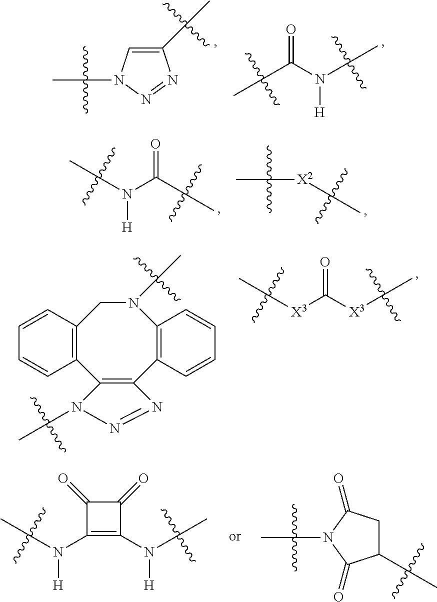
Where X² is CH₂, O, S, NR⁴, S(O), —S(O)₂O, —OS(O)₂, or OS(O)₂O;
X³ is absent, CH₂, O, S, NR⁴; and
R⁴ is H, a C₁-C₃ alkyl or alkanol group, or a —C(O)(C₁-C₃) group.

34. A compound according to the chemical structure: 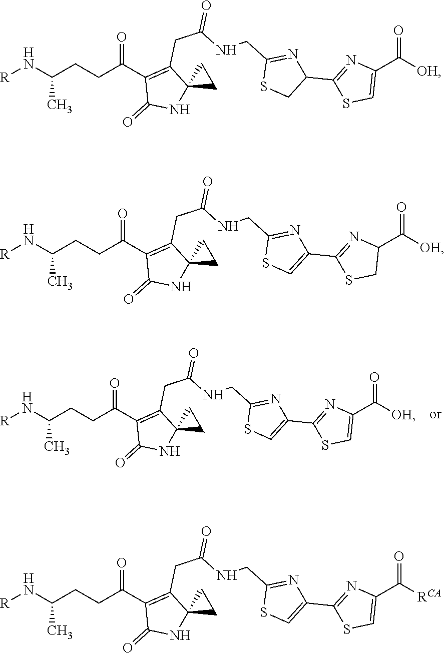
Where R is a targeting element T_{E} such as a non-cleavable or cleavable moiety which optionally has anticancer activity or a moiety such as an acid labile moiety including an acid labile peptide (e.g. a low pH insertion peptide), an antibody or antibody fragment,
a group 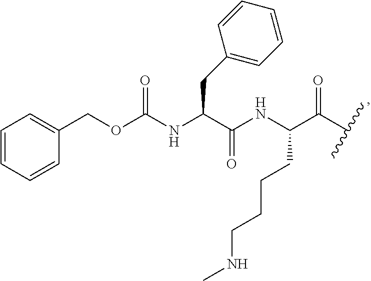 [SMILES: *C(=O)[C@H](CCCCNC)NC(=O)[C@H](Cc1ccccc1)NC(=O)OCc1ccccc1]
or a cysteine-cathepsin labile moiety; and
R^{CA} is an amine group, preferably a diamine, triamine, tetramine, even more preferably a —NH(CH₂)ₘNR^{1D}R^{2D} group where m is an integer from 1-6 (1, 2, 3, 4, 5 or 6) and R^{1D} and R^{2D} are each independently H, C₁-C₆ alkyl which is optionally substituted with one or two hydroxyl groups or is a 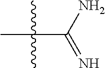 [SMILES: *C(=N)N]
group which forms a guanidine group with the nitrogen to which it is attached,
or a pharmaceutically acceptable salt or stereoisomer thereof.

35. A compound according to the chemical structure: 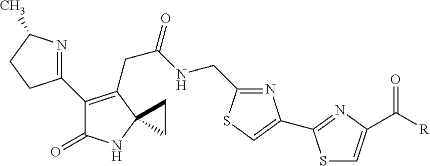
Where R is an amine group, preferably a diamine, triamine, tetramine, even more preferably a s-NH(CH₂)ₘNR^{1D}R^{2D} group where m is an integer from 1-6 (1, 2, 3, 4, 5 or 6) and R^{1D} and R^{2D} are each independently H, C₁-C₆ alkyl which is optionally substituted with one or two hydroxyl groups or is a 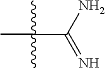 [SMILES: *C(=N)N]
group which forms a guanidine group with the nitrogen to which it is attached,
or a pharmaceutically acceptable salt or stereoisomer thereof

36. A compound according to the chemical structure:

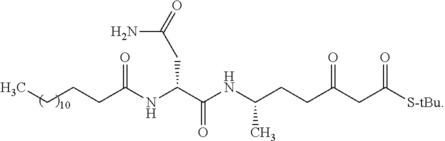

37. A compound according to the chemical structure: 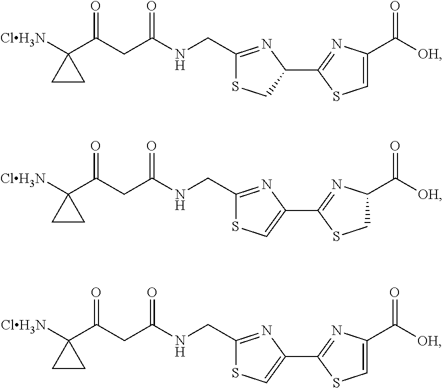
or a non-salt or alternative salt form, including a pharmaceutically acceptable salt form or stereoisomer thereof.

38. A compound according to the chemical structure: 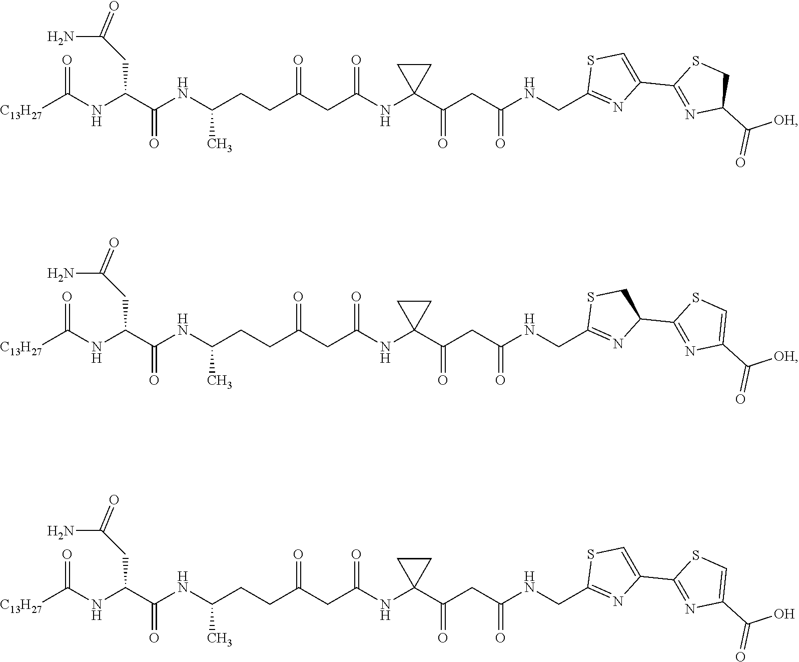
Or a salt form, including a pharmaceutically acceptable salt form thereof.

39. A compound according to the chemical structure: 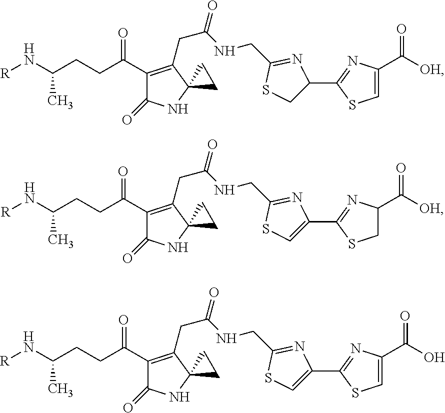
Where R is a non-cleavable or cleavable moiety which optionally exhibits anticancer activity, or an acid labile moiety such as an acid labile peptide (e.g. a low pH insertion peptide), an antibody or antibody fragment,
a group 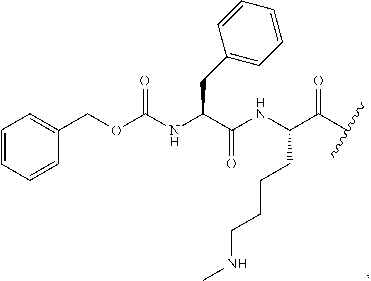 [SMILES: *C(=O)[C@H](CCCCNC)NC(=O)[C@H](Cc1ccccc1)NC(=O)OCc1ccccc1]
or a cysteine-cathepsin labile moiety, or
a pharmaceutically acceptable salt thereof.

40. A pharmaceutical composition comprising an anti-cancer effective amount of a compound according to claim 2 in combination with a pharmaceutically acceptable carrier, additive or excipient.

41. The composition according to claim 40 further comprising an effective amount of at least one additional bioactive agent.

42. The composition according to claim 41 wherein said bioactive is an additional anti-cancer agent.

43. The composition according to claim 42 wherein said additional anticancer agent is an antimetabolite, an inhibitor of topoisomerase I and II, an alkylating agent, a microtubule inhibitor or a mixture thereof.

44. The composition according to claim 42 wherein said additional anticancer agent is everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab (Arzerra), zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, irinotecan, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258,); 3-[5-(methylsulfonylpiperadinemethyl)-indolylj-quinolone, vatalanib, AG-013736, AVE-0005, the acetate salt of [D-Ser(Bu t) 6, Azgly 10] (pyro-Glu-His-Trp-Ser-Tyr-D-Ser(Bu t)-Leu-Arg-Pro-Azgly-NH₂ acetate [C₅₉H₈₄N₁₈Oi₄-(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafamib, BMS-214662, tipifamib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 3-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, irinotecan, topotecan, doxorubicin, docetaxel, vinorelbine, bevacizumab (monoclonal antibody) and erbitux, cremophor-free paclitaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-1, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa or a mixture thereof.

45. A method of treating cancer comprising administering to a patient in need an effective amount of a composition according to claim 40 to said patient.

46. The method according to claim 45 wherein said cancer is a naïve, metastatic, drug resistant, recurrent, DNA repair response deficient (DDR-deficient), hypoxic or multiple drug resistant cancer.

47. The method according to claim 46 wherein said cancer is tumorous.

48. The method according to claim 45 wherein said cancer is selected from the group consisting of carcinomas (e.g., squamous-cell carcinomas, adenocarcinomas, hepatocellular carcinomas, and renal cell carcinomas, carcinomas of the bladder, bowel, breast, cervix, colon, esophagus, head, kidney, liver, lung, neck, ovary, pancreas, prostate, stomach and thyroid; leukemias; benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine/endometrial cancer, ovarian cancer, testicular cancer) lung cancer, thyroid cancer, astrocytoma, esophageal cancer, pancreatic cancer, stomach cancer, liver cancer, colon cancer, melanoma; mixed types of neoplasias, particularly carcinosarcoma and Hodgkin's disease; and tumors of mixed origin, such as Wilms' tumor and teratocarcinomas.

49. The method according to claim 45 wherein said cancer is ovarian, breast, colon, pancreatic prostate, melanoma, head, neck or brain cancer (glioma).

50. The method according to claim 45 wherein said treatment is combined with radiation therapy.

51. A method of inhibiting metastasis of cancer in a patient in need comprising administering to said patient a composition according to claim 40 to said patient.

52. A method of synthesizing compound 23G below or a pharmaceutically acceptable salt or stereoisomer thereof 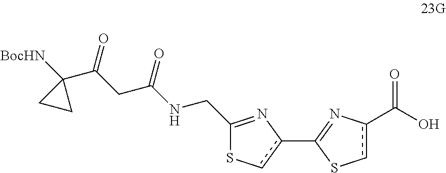
from compound 22G 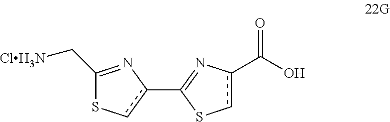
or an alternative pharmaceutical salt, non-salt or stereoisomer thereof, comprising reacting compound 22G and compound 15 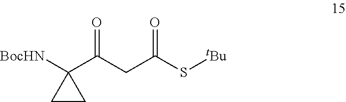 [SMILES: CC(C)(C)OC(=O)NC1(C(=O)CC(=O)SC(C)(C)C)CC1]
in the presence of a silver catalyst and a weak base in solvent at reduced temperature to produce compound 23G in a high yield of at least 50%.

53. The method according to claim 52 wherein said compound which is synthesized is 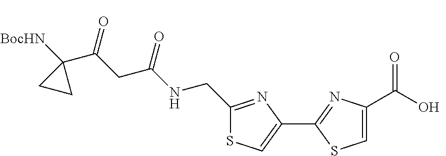 [SMILES: CC(C)(C)OC(=O)NC1(C(=O)CC(=O)NCc2nc(-c3nc(C(=O)O)cs3)cs2)CC1]
comprising reacting a compound according to the chemical structure: 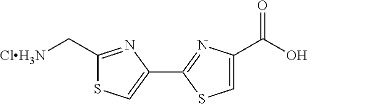
or an alternative pharmaceutical salt or non-salt thereof
and compound 15 in a silver catalyst in weak base in a solvent at reduced temperature.

54. The method according to claim 52 wherein said silver catalyst is AgCF₃CO₂, said weak base is triethylamine and said solvent is DMF.

55. A method of synthesizing compound 24G 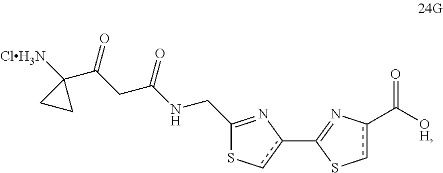
or an alternative pharmaceutical salt from compound 23G 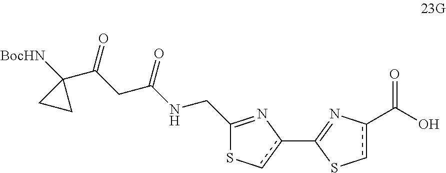
in high yield comprising exposing compound 23G to acid in solvent at about room temperature for a time sufficient to remove said boc group.

56. The method according to claim 55 wherein the compound which is produced is 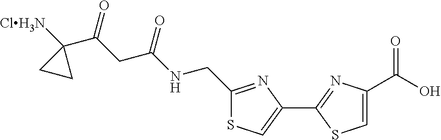
or an alternative pharmaceutical salt thereof by removing the Boc protecting group in acid in solvent at room temperature from the following compound

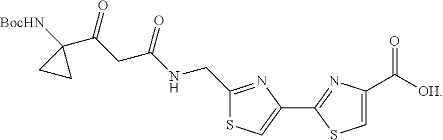 [SMILES: CC(C)(C)OC(=O)NC1(C(=O)CC(=O)NCc2nc(-c3nc(C(=O)O)cs3)cs2)CC1]

57. A method of synthesizing compound 25G 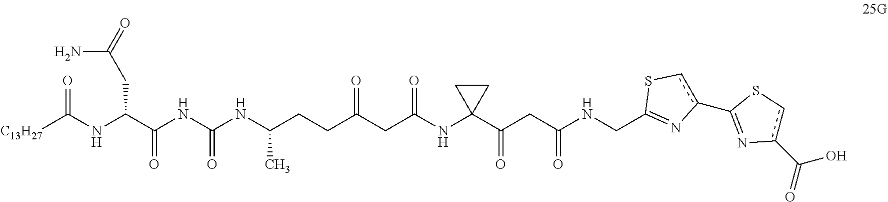
or a pharmaceutical salt thereof,
comprising reacting compound 9 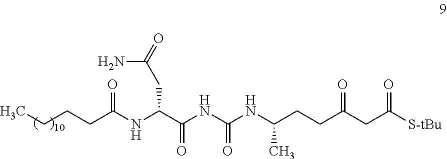
and compound 24G 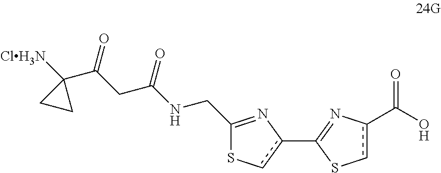
or an alternative salt thereof together in the presence of a silver catalyst in a weak base and solvent at reduced temperature.

58. The method according to claim 57 wherein said compound which is produced is 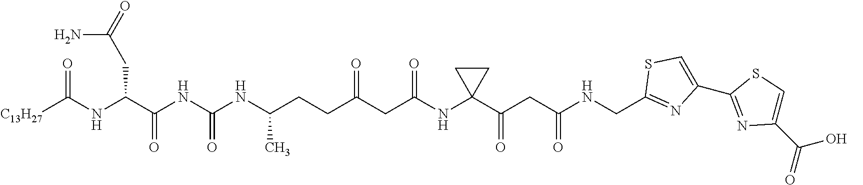
or a pharmaceutical salt thereof
which is prepared by reacting compound 9 from claim 57 above with compound 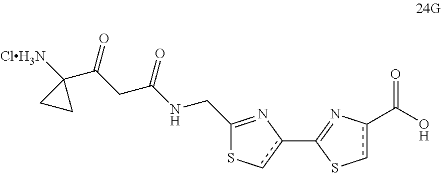
or an alternative pharmaceutical salt thereof together in the presence of a silver catalyst (AgCF₃CO₂), in a weak base (triethylamine) and solvent (DMF) at reduced temperature.

59. (canceled)
